(19) United States
(12) Patent Application Publication
(10) Pub. No.: US 2022/0153869 A1
(43) Pub. Date: May 19, 2022

(54) SHIELDED AND HOMING BISPECIFIC ANTIBODY THAT SIMULTANEOUSLY INHIBITS ANGIOGENIC PATHWAY TARGETS AND HER2 FAMILY PROTEINS AND USES THEREOF

(71) Applicant: Tavotek Biotherapeutics (Hong Kong) Limited, Shung Wan (HK)

(72) Inventors: Di ZHANG, Hillsborough, NJ (US); Lihua SHI, Wynnewood, PA (US); Minseon CHO, Breinigsville, PA (US); Motohiko NISHIDA, Ambler, PA (US); Man-Cheong FUNG, Ringoes, NJ (US); Mark CHIU, Paoli, PA (US); Maria MACWILLIAMS, Paoli, PA (US)

(21) Appl. No.: 17/526,295

(22) Filed: Nov. 15, 2021

Related U.S. Application Data
(60) Provisional application No. 63/114,036, filed on Nov. 16, 2020.

(51) Int. Cl. C07K 16/32; C07K 16/22; C07K 16/46; C12N 15/63

(52) U.S. Cl. CPC C07K 16/32; C07K 16/22; C07K 2317/515; C12N 15/63; C07K 2317/31; C07K 16/468

(57) ABSTRACT

The present disclosure relates to the application of a shielded and homing bispecific antibody as an effective and tissue specific treatment of cancers such as breast, lung, gastric cancers, and other HER2 over-expressed cancers. The homing domain increases the local concentration of the bispecific antibody in the tumor microenvironment, and the shielding employs masking domains that are fused via protease-cleavable linkers to the Fab domains targeting a human epidermal growth factor receptor 2 family protein and Fab domains targeting an angiogenic vascular endothelial growth factor pathway associated target. The unmasking of the shielded bispecific antibody occurs predominantly by proteases and enzymes in the tumor microenvironment. The application of such bispecific antibody minimizes systemic toxicity and expands the therapeutic index.

### CROSS-REFERENCE TO RELATED APPLICATION

This application claims the priority of Provisional Application No. 63/114,036, filed on Nov. 16, 2020, the content of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Nov. 11, 2021, is named 15271_0006-00000_SL.txt and is 535,033 bytes in size.

### FIELD OF DISCLOSURE

The present disclosure relates to biomedicine, particularly, a shielded and homing bispecific antibody that simultaneously targets human epidermal growth factor receptor 2 family proteins and angiogenic vascular endothelial growth factor pathway associated targets and applications of the antibody.

### BACKGROUND OF DISCLOSURE

Malignant tumors are neoplasms induced by tumorigenic factors and characterized by growth of local tissue cells. Tumor molecular biology studies have confirmed that abnormal activation of a variety of genes, including the human epidermal growth factor receptor 2 gene (HER2), MYC gene, and other oncogenes, result in disorders of cell signaling pathway and leads to tumorigenesis (Rubin and Yarden 2001).

HER2, also known as CD340 (cluster of differentiation 340), proto-oncogene Neu, or ERBB2, is a member of the epidermal growth factor receptor (EGER) family. Other family members include EGFR or HER1, HER3 and HER4. HER2 can form heterodimers with any of the other three receptors. Each of these receptors has a similar structure, with an extracellular binding domain, a transmembrane segment, an intracellular tyrosine-kinase domain (except for HER3), and an intracellular C-terminal tail with multiple tyrosine residues. Ligand binding to the extracellular domain (ECD) induces dimerization of two homodimeric or heterodimeric receptors, which activates the tyrosine-kinase domains, phosphorylating the tyrosine residues of its binding partner. Dimerization results in the phosphorylation of tyrosine residues within the cytoplasmic domain and culminates into initiation of a variety of signaling pathways involved in cellular proliferation, transcription, motility, and apoptosis inhibition.

Gene amplification of HER2 can activate other HER family members in the absence of ligands. The overexpression of HER2 leads to cell proliferation and tumorigenesis and occurs in approximately 20% to 30% of breast cancers. The HER2 gene is overexpressed to >10 times more receptor per cell in 15-25% in patients diagnosed as having HER2+ breast cancers (Siegel, DeSantis et al. 2012, Lin, Seah et al. 2013, DeSantis, Ma et al. 2014). Patients with HER2+ breast cancers live one-third shorter, and the HER2 gene amplification is also correlated with the shorter relapsing time of the disease (Nami and Wang 2017). HER2 is also overexpressed in approximately 7% to 34% of patients with gastric cancer (Meza-Junco, Au et al. 2011, Ruschoff, Hanna et al. 2012), and 30% of salivary duct carcinoma and apocrine endometrial carcinoma (Chiosea, Williams et al. 2015). HER2 overexpression is present in lung adenocarcinoma, and aggressive forms of ovarian and uterine cancer (Cretella, Saccani et al. 2014). HER2+ tumors are generally correlated with a faster growth rate, tumorigenesis, and a poorer prognosis.

Therapeutic targeting of HER2 has been exploited to treat breast cancers for several reasons: HER2 expression levels correlate directly with breast cancer invasion and prognosis; HER2 is a receptor tyrosine kinase with potent activation of downstream signaling pathways involving tumor growth; HER2+ tumors exhibit significantly more HER2 receptors on the cell surface, which is a useful biomarker to distinguish cancerous cells from normal cells; (iv) The extracellular domain of HER2 provides very stable epitopes and putative targets to design and test tumor cell-targeting neoadjuvants (Hudis 2007).

Monoclonal antibodies (mAbs) such as trastuzumab and pertuzumab have been developed to block HER2 activity to suppress tumor growth (Abramson and Arteaga 2011). Trastuzumab (Herceptin) is a recombinant humanized monoclonal antibody that binds the juxtamembrane region of HER2 on the C-terminal portion of subdomain IV (Hudis 2007). The interaction formed by Herceptin and HER2 is mediated by three regions on HER2 that form three loops: residues 557-561 (loop 1), 570-573 (loop 2), and 593-603 (loop 3). There are multiple mechanisms that inhibit cell growth: prevention of HER2 dimerization; inhibition of ligand-independent ErbB2 receptor heterodimerization; inhibition of ErbB2 extracellular domain cleavage and the expression of the constitutively active fragment; downregulation of the HER2 receptor by endocytic destruction of the receptor; accumulation of the cyclin-dependent kinase (CDK) inhibitor p27 and cell cycle arrest; induction of antibody-dependent cellular cytotoxicity; interference with DNA repair; and inhibition of constitutive HER2 cleavage/shedding mediated by metalloproteases (Higgins and Baselga 2011). Recognition of tumor cells opsonized ley monoclonal antibodies, such as trastuzumab, is mediated through receptors expressed on effector cells, as well as natural killer cells, monocytes, dendritic cells, and granulocytes. Upon recognition, these effectors induce tumor cell death. Dendritic cells capture the monoclonal antibody conjugated with tumor antigens released by dying cells in the form of immune complexes. Ultimately, these processed cells are presented to cytotoxic and helper T Consequently, both tumor-specific cytotoxic T cells and T-helper cells are activated, leading to tumor B-cell stimulation and expansion. There is a direct correlation between NK cell function and response to trastuzumab in metastatic HER2+ breast cancer patients (Slamon, Clark et al. 1987).

Pertuzumab, (2C4 or Omnitarg®, Genentech), is another recombinant humanized HER2 antibody that inhibits dimerization of HER2 with EGFR and HER-3 by sterically binding to domain II of the HER2 ECD (Cortes, Fumoleau et al. 2012). Pertuzumab binds to HER2 at a different epitope than that of trastuzumab and blocks signaling in cells that express normal levels of HER2 (Cortes, Fumoleau et al. 2012, Pierga, Petit et al. 2012).

When both trastuzumab and pertuzumab are used together, the survival of the BT474 breast cancer cell line is greatly inhibited, demonstrating increased apoptosis. This synergistic inhibition demonstrated that combining HER2-targeting agents may be more effective than using a single HER2 therapeutic strategy (Abramson and Arteaga 2011). Several Phase II trials in breast, non-small-cell lung, ovarian, and prostate cancers show the importance of combination therapy (Cortes, Fumoleau et al. 2012). There are clinical reports that pertuzumab added to the standard docetaxel and trastuzumab combination lead to striking improvements in PFS and OS in a cohort of advanced HER2+ BC patients, reaching the median OS boundary of almost 5 years (Baselga, Cortes et al. 2012).

Alternative constructs of trastuzumab have been explored to expand the therapeutic response. T-DM1, trastuzumab emtansine, is an antibody-drug conjugate composed by the microtubule polymerization inhibitor DM1 (derivative of maytansine) linked with a stable thioether linker to trastuzumab (Verma, Miles et al. 2012). After binding to HER2 receptors, the complex undergoes internalization and lysosomal degradation with the release of DM1 active catabolites that bind to tubulin and suppress microtubule dynamics. The activity of T-DM1 has been extensively studied in several human breast cancer cell lines showing a superior activity compared to trastuzumab in HER2 overexpressing cells. T-DM1 has been recently approved for the treatment of HER2-positive metastatic breast cancer patients previously treated with trastuzumab and taxane (Cretella, Saccani et al. 2014). There is also the Daiichi-Sankyo 8201 (EnHertu) with promising Phase 3 data beating the T-DM1 profile. However this molecule has a higher incidence of interstitial pneumonitis toxicity and has received a Black Box warning in the FDA label.

Alternative HER2 receptor binding proteins have been made to increase the potential efficacy. Trastuzumab has been engineered to have a second binding specificity to HER2 to add a distinct pharmacological activity. However, these dual epitope anti-HER2 molecules have limited clinical success. Another HER2 binding protein, PEPDG278D, was designed to bind to HER2 receptor, disrupt HER2 protection by mucin 4 (MUC4) with other receptor tyrosine kinases, and subsequently direct HER2 for degradation (Yang, Li et al. 2019). This molecule also downregulates epidermal growth factor receptor (EGFR) which contributes to drug resistance in HER2+ breast cancers. Other anti-VEGF bispecific agents inhibit tumor growth and angiogenesis in vivo in xenograft (Yin, Zhu et al. 2018).

In summary, HER2-targeting drugs are available for treating HER2-BC, including monoclonal antibodies trastuzumab and pertuzumab, T-DM1 (trastuzumab coupled to a microtubule inhibitor), and tyrosine kinase inhibitors (TKIs) lapatinib and neratinib. While these agents have greatly improved disease outcomes, primary and acquired drug resistance is common (de Melo Gagliato, Jardim et al. 2016). Most patients with advanced disease show disease progression after some time on treatment. About 60% of patients with HER2+ breast tumor develop de novo resistance to trastuzumab, partially due to the loss of expression of HER2 extracellular domain on their tumor cells (Rexer and Arteaga 2012). This is due to shedding/cleavage of HER2 by metalloproteinases (ADAMs and MMPs). HER2 shedding results in the accumulation of intracellular carboxyl-terminal HER2 (p95HER2), which is a common phenomenon in trastuzumab-resistant tumors and is suggested as a predictive marker for trastuzumab resistance (Pohlmann, Mayer et al. 2009). Compared to trastuzumab, much less is known about development of pertuzumab resistance. In a tamoxifen-resistant breast cancer cell line, pertuzumab promoted rapid formation of HERS/EGFR heterodimers that play a role in the rapid acquisition of resistance. HER2 overexpression also promotes epithelial mesenchymal transition (EMT) that results in the increased expression and activation of metalloproteinases. This stage of EMT leads to proteolytic cleavage and shedding of HER2 receptor, which downregulates HER2 extracellular domain and eventually increases trastuzumab and pertuzumab resistance (Nami and Wang 2017). Even the triple combination of trastuzumab, pertuzumab, and docetaxel produces median progression-free survival of only about 18 months (Kaumaya and Foy 2012). Heterogeneous mechanisms of acquired resistance occur in different mutant HER2+ cancers involving inactive target receptor (like truncated HER2 receptors lacking extracellular trastuzumab-binding domain) or alterations of target downstream components in the PI3K/Akt/m/TOR signaling pathway (Luque-Cabal, Garcia-Teijido et al. 2016).

To obtain a better strategy to contain HER2+ cancers, alternative therapeutics have been developed. Interactions between cancer cells and their microenvironment are critical for the development and progression of solid tumors (Holmgren, O'Reilly et al. 1995). Tumor growth and metastasis are critically dependent on the development and/or remodeling of the microvasculature (Folkman 1995). Tumors cannot grow beyond a certain mass without the formation of new blood vessels (angiogenesis), and a correlation between micro vessel density and tumor invasiveness has been reported for a number of tumors (Folkman 1995). Molecules capable of selectively targeting markers of angiogenesis create clinical opportunities for the diagnosis and therapy of tumors and other diseases characterized by vascular proliferation.

Inflammatory breast cancer is characterized pathologically by high vascularity and increased micro vessel density because of high expression of angiogenic factors such as VEGF, which is a key mediator of angiogenesis and is involved in endothelial and tumor cell growth and motility and blood vessel permeability (Kaumaya and Foy 2012). The transition between dormancy and active growth in tumorigenesis appears to be triggered by an “angiogenic switch” (Holmgren, O'Reilly et al. 1995). This angiogenic switch has recently been documented in several forms of cancer. VEGF constitutes one of the most proangiogenic factors known today (Troiani, Martinelli et al. 2012). In many different types of cancer, the gene expression and levels of secretion of VEGF are elevated, which is a key promoter of metastasis as well as serves as an angiogenesis factor via VEGF receptor (VEGFR)-1 and/or VEGFR-2 signaling (Fukumura, Xavier et al. 1998). Endothelial cells respond via VEGFR-2 activation, while infiltrating cells, such as macrophages, are activated via VEGFR-1 signaling, which is also involved in the recruitment of endothelial progenitor cells in neovascularization (Kaumaya and Foy 2012).

Angiogenesis is implicated in the pathogenesis of a variety of disorders which include solid tumors, intraocular neovascular syndromes such as proliferative retinopathies or age-related macular degeneration (AMD), rheumatoid arthritis, and psoriasis (Klagsbrun and D'Amore 1991, Folkman and Shing 1992). In the case of solid tumors, the neovascularization allows the tumor cells to acquire a growth advantage and proliferative autonomy compared to the normal cells. Accordingly, a correlation has been observed between density of micro vessels in tumor sections and patient survival in breast cancer as well as in several other tumors (Weidner, Semple et al. 1991, Horak, Leek et al. 1992, Macchiarini, Fontanini et al. 1992). Angiogenesis is mediated by the stabilization of the master transcription factor—hypoxia-inducible factor-α (HIFα)—leading to the transcription of several pro-tumorigenic factors, including VEGF and platelet-derived growth factor (PDGF). VEGF is a homodimeric glycoprotein (34-42 kDa) which exhibits several isoforms including VEGF-A, VEGF-B, VEGF-C, VEGF-D, and PlGF. VEGF-A is the matrix-bound isoform that binds to VEGFR-2 and plays a major role in stimulating angiogenesis (Dvorak, Brown et al. 1995, Ferrara and Davis-Smyth 1997). The functions of the other proteins in this family are unrelated to angiogenesis and these proteins can bind only to VEGFR-1, while VEGF-A binds to both VEGFR-1 and VEGFR-2.

The VEGFR signaling mechanism comprises receptor dimerization, activation of the tyrosine kinase and creation of docking sites for signaling VEGFRs. VEGFR-2 is a tyrosine kinase receptor overexpressed in breast, kidney, pancreas, and GI tract cancer cells. Thus, VEGFR is considered an important target for the development of kinase inhibitors aimed at targeting angiogenesis. Many angiogenic inhibitors have been developed, mainly focused on the VEGF pathway, since its inhibition is correlated with suppression of tumor growth and angiogenesis (Kim, Li et al. 1993, Alessi, Ebbinghaus et al. 2004). Thus, an antibody that targets any of the VEGF angiogenic pathway members would block the nutrition supply for cancer and limit the progression and metastasis of cancer. Anti-VEGF neutralizing antibodies suppress the growth of a variety of human tumor cell lines in mice (Kim, Li et al. 1993, Warren, Yuan et al. 1995). WO 94/10202, WO 98/45332, WO 2005/00900, and WO 00/35956 refer to antibodies against VEGF. Humanized monoclonal antibody bevacizumab (sold under the trade name Avastin®) is an anti-VEGF antibody used in tumor therapy and is the only anti-angiogenic agent approved for treatment of cancer (WO 98/45331). FDA approved its use as a first- and second-line therapy in colorectal cancer in 2004 and breast cancer in 2008. While this is one of the more clinically successful antiangiogenic inhibitors, there are concerns related to long-term side effects and development of resistance.

The overexpression of HER2 is associated with increased expression of VEGF at both the RNA and protein level in human breast cancer cells (Olayioye 2001). In addition, exposure of HER2 overexpressing cells to trastuzumab significantly decreases VEGF expression. Taken together, these data suggest that VEGF is a downstream target of the HER2 signaling pathway. A positive association between HER2 and VEGF expression in breast cancer patients has been identified (Le, Mao et al. 2008). A two-pronged approach of targeting cancer cells by co-immunizing with defined tumor-associated antigens and angiogenesis-associated antigens has been shown to have synergistic effects (Martin, Makhson et al. 2012). Combination treatments with defined tumor-associated and angiogenesis-associated antigens are known to produce synergistic effects (Lin, Seah et al. 2013). Combining antiangiogenic therapies with other direct anticancer agents has been shown to be beneficial in various malignancies (Gianni, Romieu et al. 2013).

Thus, bispecific agents were made that could target HER2 and VEGF with superior efficacy. Dual targeting of HER2 with trastuzumab and VEGF with VEGF-Trap produces superior growth inhibition of HER2-positive human breast cancer (Le, Mao et al. 2008). There was greater inhibition of tumor angiogenesis from the additive blockade of VEGF. While trastuzumab decreases tumor VEGF through the PI3K pathway in HER2-overexpressing cancer cells, trastuzumab can also increase anti-angiogenic factor thrombospordin-1 and inhibits additional pro-angiogenic factors such as transforming growth factor-α, angiopoietin-1, plasminogen-activator inhibitor-1 and interleukin. Therefore, trastuzumab could have synergistic interaction with VEGF inhibitors to suppress tumor angiogenesis through modulation of multiple angiogenic factors. In this sense, human cancers that overexpress HER2 may be the ideal targets for dual therapy with agents that inhibit VEGF and HER2 pathway associated proteins.

The trastuzumab effect on cancer cell signal pathway and the anti-angiogenic effect of HER2 blockade are counteracted by the compensatory production of VEGF by tumor stromal cells. In tumor stroma, various cellular components, such as endothelial cells, tumor-associated fibroblasts, and immune cells, can secrete various angiogenic factors that support tumor growth. Therefore, by inhibiting VEGF and other associated angiogenic factors secreted by stromal cells, an antibody that targets these factors can exert additional anti-angiogenic effects. As an example, enhanced inhibition of tumor angiogenesis occurred following combined treatment with trastuzumab and VEGF-Trap compared with treatment using either agent individually. In another example, bispecific antibodies with dual affinity for VEGF and HER2 had a therapeutic benefit by co-targeting the tumor cell proliferation mediated by HER2 and tumor angiogenesis mediated by VEGF (Bostrom, Yu et al. 2009). Alternative architectures with different placement of binding and affinity for the respective receptors have been made (Hu, Fu et al. 2015, Yin, Zhu et al. 2018).

VEGF expression in gastric cancer is correlated with recurrence and poor prognosis. The anti-HER2 antibody trastuzumab has been approved for the treatment of advanced gastric cancer, but with limited success. The anti-VEGF antibody bevacizumab has also shown limited efficacy against gastric cancer in clinical trials. Although trastuzumab and VEGF-Trap each moderately inhibited tumor growth, the combination of these agents exerted greater inhibition compared with either agent alone (Singh, Kim et al. 2013). The combined treatment resulted in fewer proliferating gastric tumor cells, more apoptotic cells and reduced tumor vascular density compared with treatment with trastuzumab or VEGF-Trap alone. Here, the combination of trastuzumab and VEGF-Trap had additive inhibitory effects on the tumor growth and angiogenesis of the gastric cancer xenografts. These examples portend the opportunity to make a superior bispecific antibody that targets the HER2 signaling and VEGFR angiogenesis pathways without having the complications of a combination therapy.

The measure of the therapeutic index compares the efficacy dose response to the safety dose response. Although trastuzumab has demonstrated great efficacy in treating HER2-positive cancers, it has a clinically significant cardiotoxicity concern. A significant incidence of left ventricular (LV) dysfunction, especially when combined with anthracyclines, was widely reported in the literature (Suter, Procter et al. 2007, Bowles, Wellman et al. 2012). Subsequent animal model confirmed cardiotoxicity with LV dysfunction and chamber dilatation was seen in mice with a cardiac-specific deletion of the HER2 gene, confirming a key role of HER2 in maintaining cardiac homeostasis (Crone, Zhao et al. 2002).

Trastuzumab-induced cardiotoxicity generally manifests as a decline in LVEF (Left Ventricular Ejection Fraction) (Ewer and Lippman 2005). Reports from large clinical trials demonstrate an ˜9.8% incidence of LV dysfunction and 2.7% incidence of severe, symptomatic HF (Heart Failure) (Procter, Suter et al. 2010, Bowles, Wellman et al. 2012). However, when used in combination with anthracyclines, the incidence of cardiac dysfunction increases to 16-20%, with a 7-fold increased risk of HF or cardiomyopathy (Bowles, Wellman et al. 2012). Besides prior anthracycline exposure, there are a number of other factors for increased risk including hypertension (Suter, Procter et al. 2007), suggesting that additional cardiac stress may augment cardiotoxicity and trastuzumab likely interferes with the cardiac stress response of the patients (Lemmens, Doggen et al. 2007). Meanwhile, another humanized antibody, pertuzumab, which targets an epitope near the center of the extracellular domain II of ErbB2, also has similar cardiac toxicity concerns (Arteaga, Sliwkowski et al. 2011, Higgins and Baselga 2011). Like trastuzumab, in clinical trials where pertuzumab was used as monotherapy in patients with HER2-negative breast cancer with prior exposure to anthracyclines, 10% of patients experienced a decline in LVEF of 10-50% intensity at a median of 100 days.

One key hypothesis for the pathophysiology of trastuzumab cardiotoxicity is related to alterations in the NRG and ErbB pathway, which is established as a pathway in fetal heart development and the maintenance of adult cardiac function (Zhao, Sawyer et al. 1998, Crone, Zhao et al. 2002, Falls 2003). NRG1 is a signaling protein released from microvascular endothelial cells that acts in a paracrine and juxtacrine fashion to activate the ErbB family of tyrosine kinase receptors expressed in cardiac myocytes (Pentassuglia and Sawyer 2009). In adult cardiomyocytes, NRG1 binds the ErbB4 receptor, resulting in ErbB4/ErbB4 homodimerization or ErbB4/ErbB2 heterodimerization. In response, the PI3-K/Akt, mitogen-activated protein kinase/extracellular signal-regulated kinase pathways, as well as Src/focal adhesion kinase and NO synthase are activated and regulate cardiac stress responses (Baliga, Pimental et al. 1999, Pentassuglia and Sawyer 2009). Recent data demonstrate that stimulation of the NRG1/ErbB4 signaling pathway induces cell cycle reentry of differentiated cardiomyocytes, cardiomyocyte proliferation, and promotion of cardiac repair (Bersell, Arab et al. 2009). Furthermore, in vitro and in vivo models support a cardioprotective role for endogenous, endothelial cell-derived NRG and ErbB4 in response to hypoxic/ischemic injury (Hedhli, Huang et al. 2011). Similarly, the potential relevance of associated ligands such as heparin-binding epidermal growth factor and receptors such as ErbB3, both of which may have a role in the cardiovascular system in trastuzumab cardiotoxicity (Iwamoto, Yamazaki et al. 2003, Camprecios, Lorita et al. 2011).

On the other hand, angiogenesis inhibitors are also known to cause cardiac complications, including hypertension, thrombosis, and heart failure. The most problematic class of these agents are those targeting VEGF and VEGFR (Chu, Rupnick et al. 2007, Schmidinger, Zielinski et al. 2008, Bair, Choueiri et al. 2013). Despite excellent efficacy for many cancers, cardiovascular toxicities are a hallmark of all angiogenesis inhibitors. For example, from a meta-analysis of 5 clinical trials in 3784 patients with breast cancer, it showed an incidence of high-grade CHF (Congestive Heart Failure) of 1.6% in patients treated with bevacizumab compared with only 0.4% in the control or placebo groups (Choueiri, Mayer et al. 2011, Economopoulou, Kotsakis et al. 2015).

Nonetheless, these clinical studies probably underestimated the true incidence of cardiomyopathy for several reasons. First, none of clinical trials prospectively monitored cardiac function, thus relying heavily on investigator judgment of clinical HF (heart failure). Second, diagnosis of HF in cancer patients can be difficult given the nonspecific symptoms that can arise with malignancy (e.g., fatigue or peripheral edema). In fact, cardiomyopathy can also be presented as asymptomatic LV dysfunction, thus underscoring the necessity of cardiac imaging during clinical trials. Third, long-term cardiac consequences of these drugs are not really known. In view of that, the incidences of cardiovascular toxicities could be even higher.

Besides HF, hypertension is actually the most common cardiovascular toxicity with angiogenesis inhibitors with an incidence of hypertension of 19% to 25% (Nazer, Humphreys et al. 2011, Bair, Choueiri et al. 2013). There have been several proposed mechanisms for such induced hypertension. Both functional (inactivation of endothelial NO synthase and production of vasoconstrictors such as endothelin-1) and anatomic changes in the endothelium have been proposed as mechanisms for this inducing hypertension (de Jesus-Gonzalez, Robinson et al. 2012). The resultant hypertension is probably mediated via VEGF signaling and not due to an off-target effect.

There have been several proposed mechanisms for anti-angiogenesis-associated heart failure. The most cited model is in a mouse expressing a tunable transgene encoding a VEGF trap. In that model, the induction of the VEGF trap leads to decreased myocardial capillary density, induction of hypoxia and hypoxia-inducible genes in the myocardium, and cardiac dysfunction (May, Gilon et al. 2008). Such data suggests that induction of hypoxia and hypoxia-inducible genes in the heart may lead to cardiomyopathy. Constitutive VEGF signaling is important in normal adult cardiovascular physiology, and its abrogation by antiangiogenic therapies can result in vasoconstriction and microvascular rarefaction (Vaklavas, Lenihan et al. 2010). Finally, anti-VEGF therapy causes a reduction in nitric oxide and prostacyclin, as well as an increase in blood viscosity via the overproduction of erythropoietin, all of which comprise predisposing factors for increased risk of thromboembolic events (Li and Kroetz 2018).

To minimize potential toxicity, the present disclosure provides a higher local concentration of a bispecific antibody in the disease tissue than in normal tissue. During the normal metabolism, there is a homeostasis of the HER2 and VEGF associated pathways. However, during the development of cancer, there are higher levels of proteins that are linked to the activation of the HER2 and VEGF associated pathways. This disclosure provides a shielded bispecific antibody with a homing domain that can increase its local concentration in the tumor tissue. The homing domain binds to proteins that are present at significantly higher levels in the tumor microenvironment than in normal tissue. By having a higher concentration in the tumor tissue, the bispecific antibody has a stronger efficacy with tumor growth inhibition.

One of the most selective oncofetal markers associated with neo-angiogenesis and tissue remodeling known so far represents the extra domain B (ED-B) of Fibronectin (FN) (Castellani, Viale et al. 1994). FNs are high molecular-weight extracellular matrix (ECM) components abundantly expressed in a range of healthy tissues and body fluids. Various FN isoforms can be generated due to alternative splicing at the level of the primary transcript.

The ED-B, a small domain of 91 amino acids, which is identical in sequence in mouse and man, is usually absent in both plasma and tissue-fibronectin, except for some blood vessels of the regenerating endometrium and the ovaries (Alessi, Ebbinghaus et al. 2004). However, it may become inserted in the fibronectin molecule during active tissue remodeling associated with neo-angiogenesis, thereby accumulating around the neo-vasculature and in the stroma of malignant tumors and in other tissues undergoing remodeling and angiogenesis. Because of specific accumulation around neovascular structures, ED-B represents a target for molecular intervention (Zardi, Carnemolla et al. 1987, Carnemolla, Balza et al. 1989, Castellani, Viale et al. 1994). A human recombinant antibody L19 directed to the ED-B domain has been shown to engage in vivo neo-vasculature targeting and has been demonstrated in different tumor models (Tarli, Balza et al. 1999, Viti, Tarli et al. 1999). Increased binding affinity and valence of recombinant antibody fragments lead to improved targeting of tumoral angiogenesis (Viti, Tarli et al. 1999) and in patients with cancer (Santimaria, Moscatelli et al. 2003).

Another marker found in tumors is the fibroblast-activation protein (FAP) which is a homodimeric single pass type II membrane protein expressed in reactive stromal fibroblasts of more than 90% of epithelial malignancies, including breast, colorectal, and lung cancers and on malignant mesenchymal cells of bone and soft tissue sarcomas (Garin-Chesa, Old et al. 1990, Rettig, Garin-Chesa et al. 1993, Juillerat-Jeanneret, Tafelmeyer et al. 2017). The FAP1 stromal compartment located in close vicinity to the endothelial cells of the tumor capillaries and surrounding the tumor nodules, represents approximately 50% to 90% of the tumor mass. By contrast, FAP shows a limited distribution pattern in normal tissues. In an extensive survey of normal fetal and adult human tissues, FAP was detected only in a subset of endocrine cells (A cells) in the pancreas, and transiently in some fetal mesenchymal cells. In the tumor microenvironment, many of the tumor cells have an overexpression of PD-L1 that allows them to bind to the PD-1 on tumor infiltrating lymphocytes to impair T cell activation. Thus PD-L1 can be used as a homing marker for tumor cells.

Many cancer therapeutic approaches target the specific molecules/signaling pathways that promote the hyperproliferative/antiapoptotic states in tumor cells. The continuing challenge in these methodologies, as opposed to conventional chemotherapeutics that target all rapidly dividing cells, is to specifically discriminate between tumor cells and their healthy counterparts without loss in efficacy. The present disclosure provides a differentiated bispecific antibody, e.g., a shielded bispecific antibody, with effective targeting of both HER2 and VEGF for oncology indications, but with minimal safety concerns. The addition of a homing domain further increases the presence of the shielded bispecific antibody in the tumor stroma. The shielded and homing bispecific antibody targets simultaneously proteins linked to the HER2 and VEGF associated pathways. The bispecific antibody as disclosed herein takes advantage of its added tumor cell growth inhibition efficacy in diseased tissues yet mitigating the increased toxicity concerns by shielding the binding epitopes in normal tissues.

### SUMMARY OF DISCLOSURE

The present disclosure provides a bispecific antibody targeting HER2 and/or proteins associated with the HER2 pathways as well as VEGF and/or proteins associated with the VEGF pathways. This bispecific antibody can be applied for clinical use as an anti-tumor drug. In one embodiment, the bispecific antibody can bind HER2 and VEGF simultaneously and is capable of inhibiting tumor cell proliferation.

The present disclosure provides a bispecific antibody that is capable of binding HER2 and VEGF simultaneously and is capable of inhibiting tumor cell proliferation, wherein the bispecific antibody comprises one or more shielding domains and/or one or more homing domains.

The present disclosure provides a bispecific antibody that is capable of binding an HER2 associated protein and an VEGF associated protein simultaneously and is capable of inhibiting tumor cell proliferation, wherein the bispecific antibody comprises one or more shielding domains and/or one or more homing domains.

The present disclosure provides a method for treating a patient, e.g., suffering from relapsed HER2 positive cancer, comprising the step of administering to the patient a bispecific antibody that targets VEGF and HER2 pathway associated proteins simultaneously.

The present disclosure also provides a method for treating a patient wherein the treatment with the bispecific antibody as disclosed herein prevents or reduces metastasis in the patient.

In some embodiments, the present disclosure provides for a bispecific antibody that comprises an antibody sequence that targets HER2 and antibody sequence that targets VEGF.

In some embodiments, the present disclosure provides a bispecific antibody comprising an antibody sequence targeting HER2 selected from SEQ ID NO: 23-34 and an antibody sequence targeting VEGF selected from SEQ ID No: 35-44.

In some embodiments, a bispecific antibody disclosed herein is synthesized by recombinant molecular biology techniques. The bispecific antibody affinity and blocking efficiency are identified in vitro. The recombinant DNA encoding the parental antibodies is prepared by DNA recombination techniques and then transfected into mammalian cells to express the parental antibodies. After purification, identification, and screening, the bispecific antibody is generated using the controlled Fab arm exchange process to generate a bispecific antibody which shows the biological effects of simultaneous binding to HER2 and VEGF.

In some embodiments, the present disclosure provides various shielding or caps that mask HER2 and VEGF binding as set forth in SEQ ID NO: 1-14. In some embodiments, the present disclosure provides protease substrate linkers that are set forth in SEQ ID NO: 15-22.

In some embodiments, the present disclosure provides a bispecific antibody that comprises one or more, such as two, homing domains. The homing domain can be attached to a heavy chain or light chain.

In some embodiments, the present disclosure provides a bispecific antibody that can be in a human IgG1, IgG2, IgG3, and/or IgG4 framework.

In some embodiments, the present disclosure provides a bispecific antibody that comprises a binding arm that can target HER2, wherein the binding arm comprises a human IgG heavy chain fusion protein comprising amino acid sequences from the N- to the C-terminus, signal sequence A-shield A-linker A-protease sequence A-linker B-IgG heavy chain; and a human IgG light chain fusion protein comprising amino acid sequences from the N- to the C-terminus, the signal sequence B-shield B-linker B-protease sequence B-linker C-IgG light chain.

In some embodiments, the present disclosure provides a bispecific antibody comprising a binding arm that can target HER2, wherein the binding arm comprises a human IgG heavy chain fusion protein comprising amino acid sequences from the N- to the C-terminus, signal sequence A-shield A-linker A-protease sequence A-linker B-IgG heavy chain-linker C-homing domain E; and a human IgG light chain fusion protein comprising amino acid sequences from the N- to the C-terminus, the signal sequence B-shield B-linker B-protease sequence B-linker C-IgG light chain.

In some embodiments, the present disclosure provides a bispecific antibody comprising a binding arm that can target VEGF, wherein the binding arm comprises a human IgG heavy chain fusion protein comprising amino acid sequences from the N- to the C-terminus, signal sequence A-shield A-linker A-protease sequence A-linker B-IgG heavy chain; and a human IgG light chain fusion protein comprising amino acid sequences from the N- to the C-terminus, signal sequence B-shield B-linker B-protease sequence B-linker C-IgG light chain.

In some embodiments, the present disclosure provides a bispecific antibody comprising a binding arm that can target VEGF, wherein the binding arm comprises a human IgG heavy chain fusion protein comprising amino acid sequences from the N- to the C-terminus, the signal sequence A-shield A-linker A-protease sequence A-linker B-IgG heavy chain-linker C-homing domain D; and a human IgG light chain fusion protein comprising amino acid sequences from the N- to the C-terminus, signal sequence B-shield B-linker B-protease sequence B-linker C-IgG light chain.

In some embodiments, the present disclosure provides a bispecific antibody comprising a binding arm that targets HER2 as disclosed herein and a binding arm targets VEGF as disclosed herein. For example, the present disclosure provides a bispecific antibody comprising: a binding arm that can target HER2, wherein the binding arm comprises a human IgG heavy chain fusion protein comprising amino acid sequences from the N- to the C-terminus, signal sequence A-shield A-linker A-protease sequence A-linker B-IgG heavy chain-linker C-homing domain E; and a human IgG light chain fusion protein comprising amino acid sequences from the N- to the C-terminus, the signal sequence B-shield B-linker B-protease sequence B-linker C-IgG light chain; and a binding arm that can target VEGF, wherein the binding arm comprises a human IgG heavy chain fusion protein comprising amino acid sequences from the N- to the C-terminus, the signal sequence A-shield A-linker A-protease sequence A-linker B-IgG heavy chain-linker C-homing domain D; and a human IgG light chain fusion protein comprising amino acid sequences from the N- to the C-terminus, signal sequence B-shield B-linker B-protease sequence B-linker C-IgG light chain.

In various embodiments as disclosed herein, the linker A, linker B, and linker C can be the same or different. The shield A and shield B can be the same or different. The protease sequence A and protease sequence B can be the same or different. The homing domain D and/or homing domain E can be the same or different.

In some embodiments, the present disclosure provides a bispecific antibody comprising a binding arm that can target HER2, which includes heavy chain and light chain sequences chosen from SEQ ID NO: 23-34.

In some embodiments, the present disclosure provides a bispecific antibody comprising a binding arm that can target VEGF, which includes heavy chain and light chain sequences chosen from SEQ ID NO: 35-44.

In some embodiments, the present disclosure provides a bispecific antibody comprising a binding arm that can target HER2, which includes heavy chain and light chain sequences chosen from SEQ ID NO: 23-34, and a binding arm that can target VEGF, which includes heavy chain and light chain sequences chosen from SEQ ID NO: 35-44.

In some embodiments, the present disclosure provides a bispecific antibody that can effectively inhibit HER2 ligand and receptor association and downstream signaling.

In some embodiments, the present disclosure provides a bispecific antibody that can effectively inhibit VEGF ligand and receptor association and downstream signaling.

In some embodiments, the present disclosure provides a bispecific antibody with masking domain that can be removed by one or more proteases and/or other in situ specific enzymes which are found in the tumor microenvironment. The presence of masking domain is to minimize the systemic toxicity of HER2 and VEGF inhibition by a VEGF x HER2 bispecific antibody.

In some embodiments, the present disclosure provides a bispecific antibody that can be used in a combination regimen, e.g., with chemotherapy. For example, a bispecific antibody disclosed herein can be used with higher doses of chemotherapy with HER2 and VEGF inhibitors to determine the best synergistic partners.

In some embodiments, the present disclosure provides a bispecific antibody that can be engineered to have one or more hinge regions with enhanced protease stability. The molecule can also be engineered to have shorter or longer half-life against hinge lability.

In some embodiments, the present disclosure provides a bispecific antibody that targets and binds to human HER2 and VEGF simultaneously, has a high affinity, and is capable of effectively blocking HER2 and VEGF proteins at the protein level. The bispecific antibody binds both HER2 and VEGF proteins or binds to one protein without affecting the binding of another protein, that is, the ability to bind HER2 and VEGF simultaneously. The antibody fills the gap that there is no antibody which simultaneously targets HER2 and VEGF. The bispecific antibody inhibits the proliferation of vascular endothelial cells, human lung cancer cells, human breast cancer cells, and/or human gastric cancer cells.

In some embodiments, the present disclosure provides a bispecific antibody with HER2 binding valency of 1 or 2.

In some embodiments, the present disclosure provides a bispecific antibody with VEGF binding valency of 1 or 2.

In some embodiments, the present disclosure provides a bispecific antibody targeting epitopes that are on domain 2 and domain 4 of HER2.

In some embodiments, the present disclosure provides a bispecific antibody with 1 or more homing domains to allow for higher levels of accumulation in the tumor microenvironment or specified disease tissue. The homing domain comprises an amino acid sequence chosen from SEQ ID NO: 45-52 and 82. There can be more than one type of homing domain attached to the bispecific antibody.

In some embodiments, the present disclosure provides a nucleic acid encoding a bispecific antibody as disclosed herein.

In some embodiments, the present disclosure provides a bispecific antibody that is fused to a domain specifically recognizing an ED-B-fibronectin domain, FAP or single chain Fv form of a PD-L1 antibody (PDL1ScFv), chosen from SEQ ID NO: 45-52 and 82.

In some embodiments, the present disclosure provides a recombinant expression vector comprising the nucleic acid disclosed herein.

In some embodiments, the present disclosure provides a recombinant expression transformant comprising the recombinant expression vector disclosed herein.

In some embodiments, the present disclosure provides a method for producing the bispecific antibody disclosed herein comprising steps of culturing the recombinant expression transformant disclosed herein and obtaining the bispecific antibody from the culture.

In some embodiments, the present disclosure provides an application of a bispecific antibody disclosed herein in the manufacture of a medicament for the treatment or prevention of cancer.

In some embodiments, the present disclosure provides dosage and route of administration of the medicament using a bispecific antibody disclosed herein for treating tumors, preferably, lung cancer, breast cancer, or gastric cancer

In some embodiments, the bispecific antibody disclosed herein can be present in a composition at a concentration of 10 mg/mL to 250 mg/mL.

In some embodiments, the present disclosure provides a composition comprising a bispecific antibody disclosed herein and at least one buffer, at least one stabilizer, and/or at least one surfactant. In some embodiments, the composition is liquid. In some embodiments, the composition is formulated for subcutaneous injection. In some embodiments, the composition is sterile. In some embodiments, the composition further comprises histidine HCl, trehalose dehydrate, methionine and/or polysorbate.

In some embodiments, the present disclosure provides a method of treating a disease or condition, e.g., cancer, in a subject in need thereof, comprising administering to the subject a therapeutically effective amount of a bispecific antibody or a composition comprising a bispecific antibody of the present disclosure. The disclosure also provides a bispecific antibody or a composition comprising a bispecific antibody of the present disclosure for use in a method of treating a disease or condition, e.g., cancer, in a subject in need thereof, comprising administering to the subject a therapeutically effective amount of the bispecific antibody or the composition. The disclosure also provides use of a bispecific antibody or a composition comprising a bispecific antibody of the present disclosure in the manufacture of a medicament for treating a disease or condition, e.g., cancer, comprising administering to the subject a therapeutically effective amount of the bispecific antibody or the composition.

These and other embodiments of the present disclosure will be described in greater detail herein.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1. Synergy of antibody targeting of HER2 and VEGF in HER2+ Cancers. The anti-HER2 antibody inhibits receptor dimerization, tumor cell signaling pathways, and/or generation of angiogenic factors. This inhibition activates apoptotic signals and inhibits cell proliferation and cell motility. The anti-VEGF antibody binds to VEGF thereby preventing tumor angiogenesis and tumor growth.

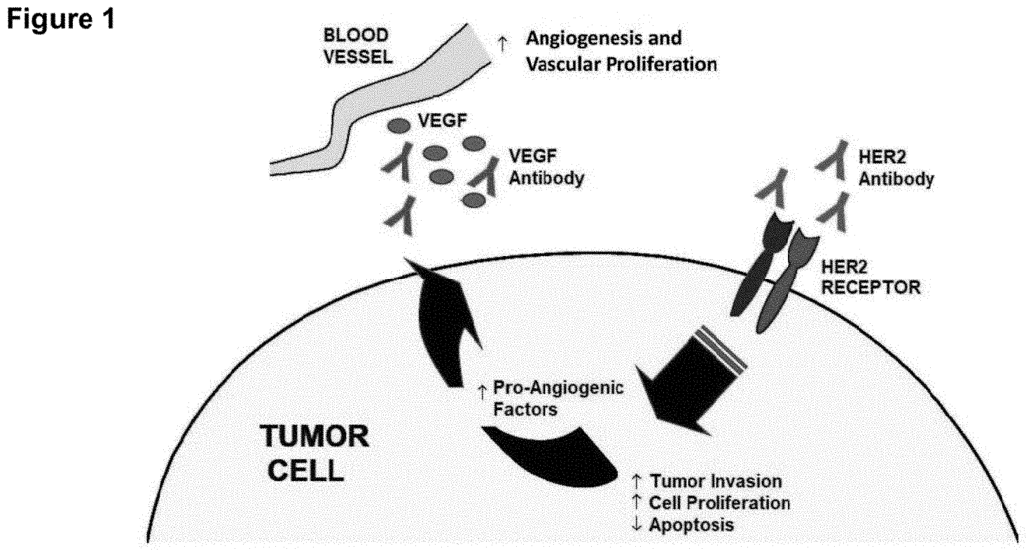

FIG. 2. Overview of toxicities from the treatments of inhibitors of VEGF and HER2. A list of toxicities are linked to inhibitors of VEGF and VEGFR from different clinical observations (Oliveira 2015). HER2 inhibition is associated with diarrhea and rash, which are the most common toxicities associated with tyrosine kinase inhibitors and may also cause left ventricular dysfunction. VEGFR inhibition leads to hypertension, proteinuria, wound healing complications, hand-foot skin reaction (HFSR), and vascular complications such as arterial thromboembolism and left ventricular dysfunction.

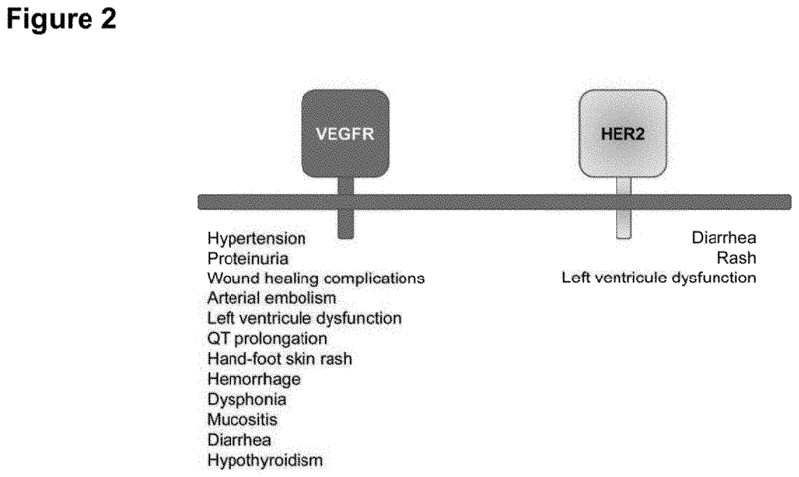

FIG. 3. Schematic drawing of a HER2 x VEGF bispecific antibody having a shielded (masking or capping) domain and a homing domain. The bispecific antibody has two different sets of heavy chain and light chain pairing which are shown with different shading. The dark shading refers to the HER2 binder arm with a light chain and a heavy chain. The light shading refers to the VEGF binder arm with a light chain and a heavy chain. A light chain comprises from the N-terminus to the C-terminus: Mask domain B, protease cleavable linker B, light chain variable region VL, and constant light chain CL. A heavy chain comprises from the N-terminus to the C-terminus: Mask domain A, protease cleavable linker A, heavy chain variable region VH, CH1 domain, Fc region, and homing domain (HD). Different or the same linkers can be put between the Mask A and protease cleavable linker A, protease cleavable linker A and VH, and Fc domain and HD domain. Different or the same linkers can be put between the Mask B and protease cleavable linker B, and protease cleavable linker A and VL. The heavy chain and light chain can have the same or different complementary mask domains, homing domains, and protease cleavable linkers.

FIG. 4. Schematic representation of the removal of the masking domains from a HER2 x VEGF bispecific antibody. Proteases that are in higher concentrations in the tumor microenvironment can cut along the protease cleavable linkers to convert a shielded bispecific antibody to an active bispecific antibody. Such tissue specific activation can minimize systemic distribution of the active bispecific antibody.

FIG. 5. Size exclusion chromatography (SEC) analysis of exemplary shielded and unshielded HER2 x VEGF bispecific antibodies. The Y axes units are in Absorbance values at 273 nm. The x axes units are chromatogram retention times in minutes.

FIG. 6. Cation exchange chromatography (CEX) analysis of exemplary shielded and unshielded HER2 x VEGF bispecific antibodies and their parental antibodies. The Y axes units are in Absorbance values at 273 nm. The x axes units are chromatogram retention times in minutes.

FIG. 7. ELISA assays demonstrating the formation of exemplary HER2 x VEGF bispecific antibodies. Only the HER2 x VEGF bispecific antibody but not the mixture of two parental antibodies bound to HER2 and VEGF simultaneously.

FIG. 8. Reduced SDS-PAGE analysis of the heavy chain and light chain of exemplary shielded HER2 x VEGF bispecific antibodies with IGF2-based masking domain before and after cleavage by the protease MMP2.

FIG. 9. ELISA assay showing binding to human HER2 by exemplary HER2 x VEGF bispecific antibodies and their parental antibodies (FIG. 9A), and shielded HER2 x VEGF bispecific antibodies with IGF2-based masking domain before and after cleavage by protease MMP2 (FIG. 9B and FIG. 9C). The Y axes units are in Absorbance values at 450 nm. The x axes units are the concentration of the respective test articles in ng/mL units.

FIG. 10. Flow cytometry assay showing binding to HER2 expressed on SK-BR-3 cell surface by exemplary HER2 x VEGF bispecific antibodies and their parental antibodies (FIG. 10A), and shielded HER2 x VEGF bispecific antibodies with IGF2-based masking domain before and after cleavage by protease MMP2 (FIG. 10B and FIG. 10C). The Y axes units are Mean Fluorescence Intensity (MFI). The x axes units are the concentration of the respective test articles in ng/mL units.

FIG. 11. Inhibition of the proliferation of breast cancer cell line SK-BR-3 by exemplary HER2 x VEGF bispecific antibodies and their parental antibodies (FIG. 11A), and shielded HER2 x VEGF bispecific antibodies with IGF2-based masking domain and corresponding non-shielded counterparts (FIG. 11B). The Y axes units are in percent cell viability. The x axes units are the concentration of the respective test articles in mg/mL units.

FIG. 12. ADCC activity of exemplary HER2 x VEGF bispecific antibodies and their parental antibodies (FIG. 12A), and shielded HER2 x VEGF bispecific antibodies with IGF2-based masking domain and corresponding non-shielded counterparts (FIG. 12B) towards SK-BR-3 cells. The Y axes units are fold of ADCC reporter gene activation. The x axes units are the concentration of the respective test articles in ng/mL units.

FIG. 13. ADCC activity of exemplary HER2 x VEGF bispecific antibodies and parental anti-HER2 antibodies (FIG. 13A), and another set of exemplary HER2 x VEGF bispecific antibodies (FIG. 13B) towards JIMT-1 cells. The Y axes units are fold of ADCC reporter gene activation. The x axes units are the concentration of the respective test articles in ng/mL units.

FIG. 14. Killing of SK-BR-3 cells (FIG. 14A) and JIMT-1 cells (FIG. 14B) by NK cells mediated by exemplary HER2 x VEGF bispecific antibodies and parental anti-HER2 antibodies. The Y axes units are percentage of cell killing. The x axes units are the concentration of the respective test articles in nM/mL units.

FIG. 15. ADCP activity of exemplary HER2 x VEGF bispecific antibodies and their parental antibodies (FIG. 15A), and shielded HER2 x VEGF bispecific antibodies with IGF2-based masking domain and corresponding non-shielded counterparts (FIG. 15B) towards SK-BR-3 cells. The Y axes units are fold of ADCP reporter gene activation. The x axes units are the concentration of the respective test articles in ng/mL units.

FIG. 16. ADCP activity of exemplary HER2 x VEGF bispecific antibodies and their parental antibodies (FIG. 16A), and shielded HER2 x VEGF bispecific antibodies with IGF2-based masking domain and corresponding non-shielded counterparts (FIG. 16B) towards SK-OV-3 cells. The Y axes units are fold of ADCP reporter gene activation. The x axes units are the concentration of the respective test articles in ng/mL units.

FIG. 17. ADCP activity of exemplary HER2 x VEGF bispecific antibodies and their parental antibodies (FIG. 17A), and shielded HER2 x VEGF bispecific antibodies with IGF2-based masking domain and corresponding non-shielded counterparts (FIG. 17B) towards JIMT-1 cells. The Y axes units are fold of ADCP reporter gene activation. The x axes units are the concentration of the respective test articles in ng/mL units.

FIG. 18. CDC activity of exemplary HER2 x VEGF bispecific antibodies and their parental antibodies (FIG. 18A), and shielded HER2 x VEGF bispecific antibodies with IGF2-based masking domain and corresponding non-shielded counterparts (FIG. 18B) towards BT-474 cells. The Y axes units are percentage of maximum cytotoxicity. The x axes units are the concentration of the respective test articles in ng/mL units.

FIG. 19. CDC activity of exemplary HER2 x VEGF bispecific antibodies and their parental antibodies (FIG. 19A), and shielded HER2 x VEGF bispecific antibodies with IGF2-based masking domain and corresponding non-shielded counterparts (FIG. 19B) towards SK-BR-3 cells. The Y axes units are percentage of maximum cytotoxicity. The x axes units are the concentration of the respective test articles in ng/mL units.

FIG. 20. ELISA assay showing binding to human VEGF by exemplary HER2 x VEGF bispecific antibodies and their parental antibodies (FIG. 20A), and shielded HER2 x VEGF bispecific antibodies with IGF2-based masking domain before and after cleavage by protease MMP2 (FIG. 20B and FIG. 20C). The Y axes units are in Absorbance values at 450 nm. The x axes units are the concentration of the respective test articles in ng/mL units.

FIG. 21. Neutralization of VEGF secreted from SK-OV-3 cells by exemplary HER2 x VEGF bispecific antibodies and their parental antibodies (FIG. 21A), and shielded HER2 x VEGF bispecific antibodies with IGF2-based masking domain before and after cleavage by protease MMP2 (FIG. 21B and FIG. 21C). The Y axes units are percentage of maximum free VEGF. The x axes units are the concentration of the respective test articles in ng/mL units.

FIG. 22. Neutralization of VEGF secreted from LS174T cells (FIG. 22A) and JIMT-1 cells (FIG. 22B) by exemplary HER2 x VEGF bispecific antibodies and their parental antibodies. The Y axes units are percentage of maximum free VEGF. The x axes units are the concentration of the respective test articles in mg/mL units.

FIG. 23. Inhibition of VEGF-mediated reporter gene activation by exemplary HER2 x VEGF bispecific antibodies and their parental antibodies (FIG. 23A), and shielded HER2 x VEGF bispecific antibodies with IGF2-based masking domain before and after cleavage by protease MMP2 (FIG. 23B and FIG. 23C) in VEGF reporter assay. The Y axes units are percentage of maximum reporter gene activation driven by 25 ng/mL VEGF. The x axes units are the concentration of the respective test articles in ng/mL units.

FIG. 24. Inhibition of VEGF-mediated HUVEC cell proliferation by exemplary HER2 x VEGF bispecific antibodies and their parental antibodies (FIG. 24A), and shielded HER2 x VEGF bispecific antibodies with IGF2-based masking domain and their corresponding non-shielded counterparts (FIG. 24B). The Y axes units are percentage of maximum HUVEC cell proliferation driven by 25 ng/mL VEGF. The x axes units are the concentration of the respective test articles in ng/mL units.

FIG. 25. Inhibition of VEGF-mediated HUVEC cell migration by exemplary HER2 x VEGF bispecific antibody and its parental antibodies. Representative images of migrated HUVEC cells and corresponding bar graph with the total cell number of migrated cells quantitated per view were shown.

FIG. 26. Inhibition of HUVEC cell migration driven by VEGF secreted from SK-OV-3 cells by exemplary HER2 x VEGF bispecific antibody and its parental antibodies. Representative images of migrated HUVEC cells and corresponding bar graph with the total cell number of migrated cells per view quantitated were shown.

FIG. 27. Tumor growth inhibition by exemplary HER2 x VEGF bispecific antibody and its parental antibodies evaluated in xenograft models with human breast cancer cell BT474 (FIG. 27A), human ovarian cancer cell SK-OV-3 (FIG. 27B), and human colorectal cancer cell LS-174T (FIG. 27C). The Y axes units are tumor volume in units of mm³. The x axes units are the treatment days.

FIG. 28. Tumor growth inhibition by exemplary shielded HER2 x VEGF bispecific antibodies and corresponding unshielded HER2 x VEGF bispecific antibody and anti-HER2 antibody evaluated in xenograft models with human breast cancer cell BT474 (FIG. 28A). The Y axes units are tumor volume in units of mm³. The x axes units are the treatment days. FIG. 28B shows IHC staining of the expression of MMP2 and uPA proteases in BT474 xenograft tumor.

FIG. 29. Line graphs showing tumor growth inhibition of human breast cancer cell BT474 xenograft in individual mice by anti-HER2 antibody with or without the E345R Fc mutation. The Y axes units are tumor volume in units of mm³. The x axes units are the treatment days.

FIG. 30. Inhibition of the viability of iPSC-derived human ventricular cardiomyocytes by exemplary anti-HER2 antibody with and without IGF2-based masking domain treated at 50 mg/mL for 6 days. The Y axes units are in percent cell viability values.

FIG. 31. Characterization of HER2 x VEGF bispecific antibodies with ScFv of PD-L1 antibody as the homing domain. FIG. 31A: ELISA assay showing binding to human PD-L1 by exemplary HER2 x VEGF bispecific antibodies with ScFv of PD-L1 antibody as the homing domain. The Y axes units are in Absorbance values at 450 nm. The x axes units are the concentration of the respective test articles in ng/mL units. FIG. 31B: PD-L1 blockade reporter assay showing the PD-L1/PD-1 blockade activities of exemplary HER2 x VEGF bispecific antibodies with ScFv of PD-L1 antibody as the homing domain. The Y axes units are fold of reporter gene activation. The x axes units are the concentration of the respective test articles in ng/mL units.

### DETAILED DESCRIPTION

### Definitions

All publications, including but not limited to disclosures and disclosure applications, cited in this specification are herein incorporated by reference as though fully set forth. If certain content of a reference cited herein contradicts or is inconsistent with the present disclosure, the present disclosure controls.

It is to be understood that the terminology used herein is for the purpose of describing particular embodiments only and is not intended to be limiting. Unless defined otherwise, all technical and scientific terms used herein have the same meaning as commonly understood by one of ordinary skill in the art to which the disclosure pertains.

Although any methods and materials similar or equivalent to those described herein may be used in the practice for testing of the present disclosure, exemplary materials and methods are described herein. In describing and claiming the present disclosure, the following terminology will be used.

As used in this specification and the appended claims, the singular forms “a,” “an,” and “the” include plural referents unless the content clearly dictates otherwise. Thus, for example, reference to “a cell” includes a combination of two or more cells, and the like.

“Antibodies” is meant in a broad sense and includes immunoglobulin molecules including monoclonal antibodies including murine, human, humanized and chimeric monoclonal antibodies, antibody fragments, bispecific or multi-specific antibodies, dimeric, tetrameric or multimeric antibodies, single chain antibodies, domain antibodies and any other modified configuration of the immunoglobulin molecule that comprises an antigen binding site of the required specificity.

“Full length antibody molecules” are comprised of two heavy chains (HC) and two light chains (LC) inter-connected by disulfide bonds as well as multimers thereof (e.g. IgM). Each heavy chain is comprised of a heavy chain variable region (V_{H}) and a heavy chain constant region (comprised of domains C_{H1}, hinge, C_{H2} and C_{H3}). Each light chain is comprised of a light chain variable region (V_{L}) and a light chain constant region (C_{L}). The V_{H} and the V_{L} regions may be further subdivided into regions of hyper variability, termed complementarity determining regions (CDR), interspersed with framework regions (FR). Each V_{H} and V_{L} is composed of three CDRs and four FR segments, arranged from amino-to-carboxyl-terminus in the following order: FR1, CDR1, FR2, CDR2, FR3, CDR3 and FR4.

“Complementarity determining regions (CDR)” are “antigen binding sites” in an antibody. CDRs may be defined using various terms: (i) Complementarity Determining Regions (CDRs), three in the V_{H} (HCDR1, HCDR2, HCDR3) and three in the V_{L} (LCDR1, LCDR2, LCDR3) are based on sequence variability (Wu and Kabat 1970) (Kabat et al., Sequences of Proteins of Immunological Interest, 5th Ed. Public Health Service, National Institutes of Health, Bethesda, Md., 1991). (ii) “Hypervariable regions,” “HVR,” or “HV,” three in the V_{H} (H1, H2, H3) and three in the V_{L} (L1, L2, L3) refer to the regions of an antibody variable domains which are hypervariable in structure as defined by Chothia and Lesk (Chothia and Lesk 1987). The International ImMunoGeneTics (IMGT) database (http://www_imgt_org) provides a standardized numbering and definition of antigen-binding sites. The correspondence between CDRs, HVs and IMGT delineations are described (Lefranc, Pommie et al. 2003). The term “CDR,” “HCDR1,” “HCDR2,” “HCDR3,” “LCDR1,” “LCDR2” and “LCDR3” as used herein includes CDRs defined by any of the methods described supra, Kabat, Chothia or IMGT, unless otherwise explicitly stated in the specification.

Immunoglobulins may be assigned to five major classes, IgA, IgD, IgE, IgG and IgM, depending on the heavy chain constant region amino acid sequence. IgA and IgG are further sub-classified as the isotypes IgA₁, IgA₂, IgG₁, IgG₂, IgG₃ and IgG₄. Antibody light chains of any vertebrate species may be assigned to one of two clearly distinct types, namely kappa (κ) and lambda (λ), based on the amino acid sequences of their constant regions.

“Antibody fragments” refers to a portion of an immunoglobulin molecule that retains the heavy chain and/or the light chain antigen binding site, such as heavy chain complementarity determining regions (HCDR) 1, 2 and 3, light chain complementarity determining regions (LCDR) 1, 2 and 3, a heavy chain variable region (V_{H}), or a light chain variable region (V_{L}). Antibody fragments include well known F_{ab}, F(_{ab′})2, F_{d} and Fᵥ fragments as well as domain antibodies (dAb) consisting of one V_{H} domain. V_{H} and V_{L} domains may be linked together via a synthetic linker to form various types of single chain antibody designs where the V_{H}/V_{L} domains may pair intramolecularly, or intermolecularly in those cases when the V_{H} and V_{L} domains are expressed by separate single chain antibody constructs, to form a monovalent antigen binding site, such as single chain Fv (scFv) or diabody; described for example in Int. Disclosure Publ. Nos. WO1998/44001, WO1988/01649, WO1994/13804 and WO1992/01047.

“Monoclonal antibody” refers to an antibody population with single amino acid composition in each heavy and each light chain, except for possible well-known alterations such as removal of C-terminal lysine from the antibody heavy chain. Monoclonal antibodies typically bind one antigenic epitope, except that bispecific monoclonal antibodies bind two distinct antigenic epitopes. Monoclonal antibodies may have heterogeneous glycosylation within the antibody population. Monoclonal antibody may be monospecific or multi-specific, or monovalent, bivalent or multivalent. A bispecific antibody is included in the term monoclonal antibody.

“Isolated antibody” refers to an antibody or antibody fragment that is substantially free of other antibodies having different antigenic specificities. “Isolated antibody” encompasses antibodies that are isolated to a higher purity, such as antibodies that are 80%, 81%, 82%, 83%, 84%, 85%, 86%, 87%, 88%, 89%, 90%, 91%, 92%, 93%, 94%, 95%, 96%, 97%, 98%, 99% or 100% pure.

“Humanized antibody” refers to an antibody in which the antigen binding sites are derived from non-human species and the variable region frameworks are derived from human immunoglobulin sequences. Humanized antibody may include substitutions in the framework so that the framework may not be an exact copy of expressed human immunoglobulin or human immunoglobulin germline gene sequences.

“Human antibody” refers to an antibody having heavy and light chain variable regions in which both the framework and the antigen binding site are derived from sequences of human origin and is optimized to have minimal immune response when administered to a human subject. If the antibody contains a constant region or a portion of the constant region, the constant region also is derived from sequences of human origin.

“Shielded antibody” refers to an antibody with a protein or peptide domain that masks the ability of the antibody in binding to its antigen. Without antigen recognition, the shielded antibody exists in an inactive state without being able to bind and exert its action to its target. The shielded antibody can be converted to an active antibody upon the shielding domain is removed and its antigen-binding capability is restored. The use of “shielded”, “masked”, and “capped” for mAbs or bispecific antibodies are synonymous in this disclosure.

“Homing domain” refers to an antibody or antibody domain or peptide sequence that can target mAb or bispecific antibody proteins to be in a higher local concentration in the disease tissue microenvironment.

“Anti-target” refers to an antibody or antibody domain that can bind to the specified target molecule such as HER2 or VEGF (i.e. anti HER2 is the antibody or antibody domain that can bind to HER2). The style “HER2” refers to the HER2 protein or HER2 gene product. The style “HER2” refers to the HER2 gene.

“HER2 x VEGF” refers to a bispecific antibody or antibody fragments that can bind to HER2 and VEGF. The process of making bispecific antibodies requires the recombinant modifications to parental mAb amino acid sequences. Although the amino acid sequences of the CH1, CL, and Fc domains of each parental mAb will not be the same, there is no significant difference in the binding between the HER2 x VEGF and VEGF x HER2 bispecific antibodies.

The numbering of amino acid residues in the antibody constant region throughout the specification is according to the EU index as described in Kabat et al., Sequences of Proteins of Immunological Interest, 5th Ed. Public Health Service, National Institutes of Health, Bethesda, Md. (1991), unless otherwise explicitly stated.

Conventional one and three-letter amino acid codes are used herein as shown in Table 1.

TABLE 1

| | Three-letter | |
|---|---|---|
| Amino acid | code | One-letter code |
| Alanine | Ala | A |
| Arginine | Arg | R |
| Asparagine | Asn | N |
| Aspartate | Asp | D |
| Cysteine | Cys | C |
| Glutamate | Gln | E |
| Glutamine | Glu | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The polypeptides, nucleic acids, fusion proteins, and other compositions provided herein may encompass polypeptides, nucleic acids, fusion proteins, and the like that have a recited percent identity to an amino acid sequence or DNA sequence provided herein. The term “identity” refers to a relationship between the sequences of two or more polypeptide molecules or two or more nucleic acid molecules, as determined by aligning and comparing the sequences. “Percent identity,” “percent homology,” “sequence identity,” or “sequence homology” and the like mean the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073. In calculating percent identity, the sequences being compared are typically aligned in a way that gives the largest match between the sequences.

The constant region sequences of the mammalian IgG heavy chain are designated in sequence as C_{H1}-hinge-C_{H2}-C_{H3}. The “hinge,” “hinge region” or “hinge domain” of an IgG is generally defined as including Glu216 and terminating at Pro230 of human IgG₁ according to the EU Index but functionally, the flexible portion of the chain may be considered to include additional residues termed the upper and lower hinge regions, such as from Glu216 to Gly237 and the lower hinge has been referred to as residues 233 to 239 of the F_{c} region where F_{c}γR binding was generally attributed. Hinge regions of other IgG isotypes may be aligned with the IgG₁ sequence by placing the first and last cysteine residues forming inter-heavy chain S-S bonds. Although boundaries may vary slightly, as numbered according to the EU Index, the C_{H1} domain is adjacent to the V_{H} domain and amino terminal to the hinge region of an immunoglobulin heavy chain molecule and includes the first (most amino terminal) constant region of an immunoglobulin heavy chain, e.g., from about EU positions 118-215. The F_{c} domain extends from amino acid 231 to amino acid 447; the C_{H2} domain is from about Ala231 to Lys340 or Gly341 and the C_{H3} from about Gly341 or Gln342 to Lys447. The residues of the IgG heavy chain constant region of the C_{H1} region terminate at Lys. The F_{c} domain containing molecule comprises at least the C_{H2} and the C_{H3} domains of an antibody constant region, and therefore comprises at least a region from about Ala231 to Lys447 of IgG heavy chain constant region. The F_{c} domain containing molecule may optionally comprise at least portion of the hinge region.

“Epitope” refers to a portion of an antigen to which an antibody specifically binds. Epitopes typically consist of chemically active (such as polar, non-polar or hydrophobic) surface groupings of moieties such as amino acids or polysaccharide side chains and may have specific three-dimensional structural characteristics, as well as specific charge characteristics. An epitope may be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule. Antibody “epitope” depends on the methodology used to identify the epitope.

A “leader sequence” as used herein includes any signal peptide that can be processed by a mammalian cell, including the human B2M leader. Such sequences are well-known in the art.

A “cleavable linker” (also referred to as “protease cleavable linker” or “protease sequence”) is a peptide substrate cleavable by an enzyme. Operatively, the cleavable linker, upon being cleaved by the enzyme, allows for activation of the present shielded antibody with IGF2-based masking domain. Preferably, the cleavable linker is selected so that activation occurs at the desired site of action, which can be a site in or near the target cells (e.g., carcinoma cells) or tissues. For example, the cleavable linker is a peptide substrate specific for an enzyme that is specifically or highly expressed in the site of action, such that the cleavage rate of the cleavable linker in the target site is greater than that in sites other than the target site.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The terms also include polypeptides that have co-translational (e.g., signal peptide cleavage) and post-translational modifications of the polypeptide, such as, for example, disulfide-bond formation, glycosylation, acetylation, phosphorylation, proteolytic cleavage, and the like.

Furthermore, as used herein, a “polypeptide” refers to a protein that includes modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to PCR amplification or other recombinant DNA methods.

The term “masking domain” (also referred to as “shield” or “cap”) in this disclosure refers to a protein domain that can be fused to an antibody and mask the antibody in binding to its antigen. The shielding domain can mask the antibody from recognizing its target epitope so the antibody is kept as an inactive shielded antibody form. Upon the removal of the shielding domain, the variable domains of the antibody are exposed and can bind and exert actions to its target.

The term “recombinant,” as used herein to describe a nucleic acid molecule, means a polynucleotide of genomic, cDNA, viral, semisynthetic, and/or synthetic origin, which, by virtue of its origin or manipulation, is not associated with all or a portion of the polynucleotide sequences with which it is associated in nature. The term “recombinant,” as used with respect to a protein or polypeptide, refers to a polypeptide produced by expression from a recombinant polynucleotide. The term “recombinant,” as used with respect to a host cell or a virus, refers to a host cell or virus into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

The terms “polynucleotide,” “oligonucleotide,” “nucleic acid” and “nucleic acid molecule” are used interchangeably herein to include a polymeric form of nucleotides, either ribonucleotides or deoxyribonucleotides. This term refers only to the primary structure of the molecule.

“Vector” refers to a polynucleotide capable of being duplicated within a biological system or that can be moved between such systems. Vector polynucleotides typically contain elements, such as origins of replication, polyadenylation signal or selection markers, that function to facilitate the duplication or maintenance of these polynucleotides in a biological system, such as a cell, virus, animal, plant, and reconstituted biological systems utilizing biological components capable of duplicating a vector. The vector polynucleotide may be DNA or RNA molecules, cDNA, or a hybrid of these, single stranded or double stranded.

“Expression vector” refers to a vector that can be utilized in a biological system or in a reconstituted biological system to direct the translation of a polypeptide encoded by a polynucleotide sequence present in the expression vector.

As used herein, the term “heterologous” used in reference to nucleic acid sequences, proteins or polypeptides, means that these molecules are not naturally occurring in the cell from which the heterologous nucleic acid sequence, protein or polypeptide was derived. For example, the nucleic acid sequence coding for a human polypeptide that is inserted into a cell that is not a human cell is a heterologous nucleic acid sequence in that particular context. Whereas heterologous nucleic acids may be derived from different organism or animal species, such nucleic acid need not be derived from separate organism species to be heterologous. For example, in some instances, a synthetic nucleic acid sequence or a polypeptide encoded therefrom may be heterologous to a cell into which it is introduced in that the cell did not previously contain the synthetic nucleic acid. As such, a synthetic nucleic acid sequence or a polypeptide encoded therefrom may be considered heterologous to a human cell, e.g., even if one or more components of the synthetic nucleic acid sequence or a polypeptide encoded therefrom was originally derived from a human cell.

A “host cell,” as used herein, denotes an in vivo or in vitro eukaryotic cell or a cell from a multicellular organism (e.g., a cell line) cultured as a unicellular entity, which eukaryotic cells can be, or have been, used as recipients for a nucleic acid (e.g., an expression vector that comprises a nucleotide sequence encoding a multimeric polypeptide of the present disclosure), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a genetically modified eukaryotic host cell is genetically modified by virtue of introduction into a suitable eukaryotic host cell a heterologous nucleic acid, e.g., an exogenous nucleic acid that is foreign to the eukaryotic host cell, or a recombinant nucleic acid that is not normally found in the eukaryotic host cell.

“Specific binding” or “specifically binds” or “binds” refer to an antibody binding to a specific antigen with greater affinity than for other antigens. Typically, the antibody “specifically binds” when the equilibrium dissociation constant (K_{D}) for binding is about 1×10⁻⁸ M or less, for example about 1×10⁻⁹ M or less, about 1×10⁻¹⁰ M or less, about 1×10⁻¹¹ M or less, or about 1×10⁻¹² M or less, typically with the K_{D} that is at least one hundred-fold less than its K_{D} for binding to a non-specific antigen (e.g., BSA, casein). The K_{D} may be measured using standard procedures.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (e.g., rats, mice), lagomorphs (e.g., rabbits), non-human primates, humans, canines, felines, ungulates (e.g., equines, bovines, ovines, porcines, caprines), etc.

A “therapeutically effective amount” or “efficacious amount” refers to the amount of an agent, or combined amounts of two agents, that, when administered to a mammal or other subject for treating a disease, is sufficient to affect such treatment for the disease. The “therapeutically effective amount” will vary depending on the agent(s), the disease and its severity and the age, weight, etc., of the subject to be treated.

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

The following examples described the invention in further details which are not intended to limit the scope of protection for the invention.

### HER2 and VEGF Bispecific Antibody

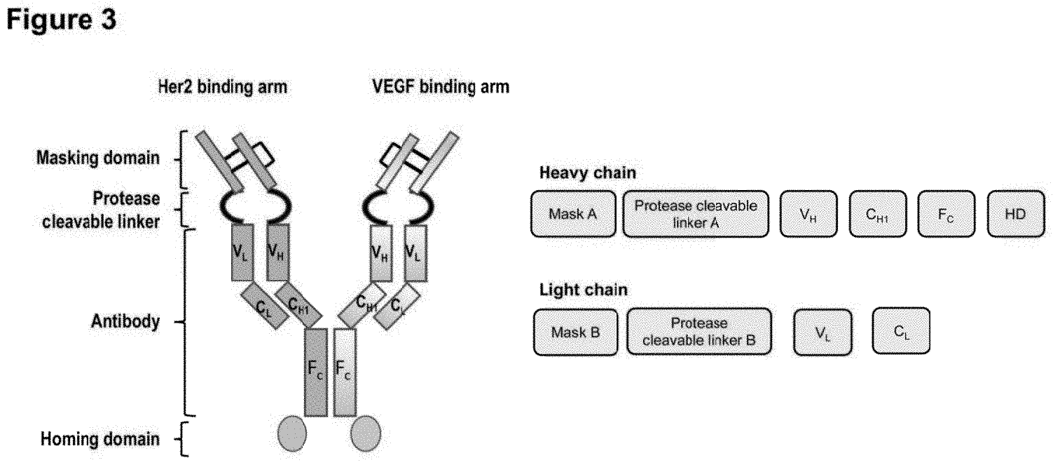

In some embodiments, the present disclosure provides a bispecific antibody that simultaneously targets human HER2 and VEGF. The bispecific antibody consists of two sets of light chains and two sets of heavy chains. The structure of the light chains and the heavy chains for the respective parental antibodies are shown in FIG. 3. The human IgG heavy chain fusion comprises amino acid sequences from N- to the C-terminus: signal sequence A-shield A-linker A-protease sequence A-linker B-IgG heavy chain; and the human IgG light chain fusion comprises amino acid sequences from the N- to the C-terminus, the signal sequence B-shield B-linker B-protease sequence B-linker C-IgG light chain. The shield A can be the same or different from shield B. Linker A can be the same or different from linker B. Protease sequence B can be same or different from protease sequence A.

In some embodiments, the present disclosure provides a bispecific antibody comprising a homing domain that simultaneously targets human HER2 and VEGF. The bispecific antibody can include a human IgG heavy chain fusion comprising amino acid sequences from the N- to the C-terminus, signal sequence A-shield A-linker A-protease sequence A-linker B-IgG heavy chain-linker C-homing domain D; and a human IgG light chain fusion comprising amino acid sequences from the N- to the C-terminus, signal sequence B-shield B-linker B-protease sequence B-linker C-IgG light chain. The shield A can be the same or different from shield B. Linker A can be the same or different from linker B. Protease sequence B can be same or different from protease sequence A. The homing domain D can be a fusion on either of or both the heavy chain of the HER2 binding arm and the heavy chain of the VEGF binding arm. The homing domain of the HER2 binding arm can be the same or different from the homing domain on the heavy chain of the VEGF binding arm. The homing domain D can be a fusion on either of or both the light chain of the HER2 binding arm and the light chain of the VEGF binding arm. The homing domain of the HER2 binding arm can be the same or different from the homing domain on the light chain of the VEGF binding arm.

The present disclosures provide for a combination of shields that can form intermolecular interactions to block Fab arm engagement to their respective epitopes. These intermolecular interactions can involve association of the regions of the heavy chain shield fusion with the regions of the light chain shield fusion.

The present disclosure provides a bispecific antibody that can be generated using well established point mutations in the CH1, CH2, and CH3 domains via controlled Fab arm exchange or via co-expression. In some embodiments, all constructs are symmetric so that there is no preference for the selection of point mutations of the respective parental antibodies.

### Leader Sequences

In certain embodiments, a leader peptide is chosen to drive the secretion of the shielded HER2 x VEGF bispecific antibody described in this disclosure into the cell culture supernatant as a secreted respective parental antibody protein. Any leader peptide for any known secreted proteins/peptides can be used.

As used herein, a “leader peptide” or “signal peptide” or “leader sequence” includes a short peptide, usually 16-30 amino acids in length, that is present at the N-terminus of most of newly synthesized proteins that are destined towards the secretory pathway. Although lead peptides are extremely heterogeneous in sequence, and many prokaryotic and eukaryotic lead peptides are functionally interchangeable even between different species, the efficiency of protein secretion may be strongly determined by the sequence of the lead/signal peptide.

In certain embodiments, the leader peptide is from a protein residing either inside certain organelles (such as the endoplasmic reticulum, Golgi, or endosomes), secreted from the cell, or inserted into most cellular membranes may be used.

In certain embodiments, the leader peptide is from a eukaryotic protein.

In certain embodiments, the leader peptide is from a secreted protein, e.g., a protein secreted outside a cell.

In certain embodiments, the leader peptide is from a transmembrane protein.

In certain embodiments, the leader peptide contains a stretch of amino acids that is recognized and cleaved by a signal peptidase.

In certain embodiments, the leader peptide does not contain a cleavage recognition sequence of a signal peptidase.

In certain embodiments, the leader peptide is a signal peptide for tissue plasminogen activator (tPA), herpes simplex virus glycoprotein D (HSV gD), a growth hormone, a cytokine, a lipoprotein export signal, CD2, CD3δ, CD3ε, CD3γ, CD3′, CD4, CD8α, CD19, CD28, 4-1BB or GM-CSFR, or S. cerevisiae mating factor α-1 signal peptide.

In some embodiments, a leader sequence as described herein may be a mammalian CD4 or CD8 leader sequence, including but not limited to, e.g., a human CD4 or CD8 leader sequence, a non-human primate CD4 or CD8 leader sequence, a rodent CD4 or CD8 leader sequence, and the like. In some embodiments, a CD4 or CD8 leader comprises an amino acid sequence having at least 75%, at least 80%, at least 85%, at least 90%, at least 95%, at least 98%, at least 99%, or 100%, amino acid sequence identity with the human CD4 or CD8 leader sequences.

### Shields

The shield (masking domain or masking peptide) is a sequence that can block bispecific antibody CDRs from binding to HER2 and VEGF. Several different masking strategies have been developed in recent years for antibody prodrug development (Kavanaugh 2020, Lin, Lu et al. 2020). These masking strategies can be broadly categorized into two types. The first type is a spatial hindrance-based approach which relies on the fusion of a masking domain to sterically interfere with the antigen-binding capability of the antibody. Certain antibody domains have been developed by AbbVie and Roche to sterically hinder the antibody binding to its target. Besides, autologous IgG hinge domain, coiled-coil domain, non-antibody protein fragment, such as latency-associated peptide, and amino acid polymer have also been developed as such masking domains (Metz, Panke et al. 2012, Chen, Chuang et al. 2017, Lu, Chuang et al. 2019, Trang, Zhang et al. 2019). Another type of strategies is affinity peptide-based which relies on affinity peptide or mutated antigen to occupy the antigen-binding, and thus exclude the antibody binding to its target antigen (Donaldson, Kari et al. 2009, Autio, Boni et al. 2020). For example, affinity peptides are used to mask the antibody as Probody developed by Cytomx, and Akrevia and Harpoon have similar strategies to mask the antibody known as Xilio and ProTriTac respectively. As non-limiting examples, the disclosure provides for the shield or masking domain or peptide sequence set forth as SEQ ID NO: 1-14.

In some embodiments, the disclosure provides a masking domain comprising two peptide chains, referred to as the A chain and B chain, of mature Insulin Growth Factor 2 (IGF2). The IGF2 A chain and B chain are linked together by one or more, e.g., two, inter-molecular disulfide bonds, and an additional intra-molecular disulfide bond is formed within the A chain. A shielded antibody is formed with the A chain and B chain of IGF2 fused to the N-terminus of the antibody heavy chain and light chain respectively via protease-cleavable linker sequences. Due to the size of heterodimerized IGF2 peptides and close proximity to the Fab domains of the antibody, the IGF2-based masking domain interferes or blocks the binding of the antibody to its antigen.

In certain embodiments, the IGF2-based masking domain comprises the human IGF2 A chain with V43L mutation and human IGF2 B chain with Y27A mutation that disrupt IGF2 binding to its receptors, and thus reduce any toxic effects, including aberrant modulation of glucose metabolism and cell proliferation, due to IGF2 signaling.

### Protease-Cleavable Linker

The protease-cleavable linker is a peptide substrate cleavable by a protease linking the shielding domain to the antibody heavy and light chains. The sequence comprises one or more protease substrate sequence and optional linker spacer sequences (FIG. 3). The shielding sequences can exist as pairs of sequences that can be fused to either the heavy chain or light chain. For each of the two Fab arm domains of the antibody, a shielding sequence is fused to the N-terminus of the antibody heavy chain via one protease-cleavable linker and the complement sequence is fused to the N-terminus of the antibody light chain via another protease-cleavable linker.

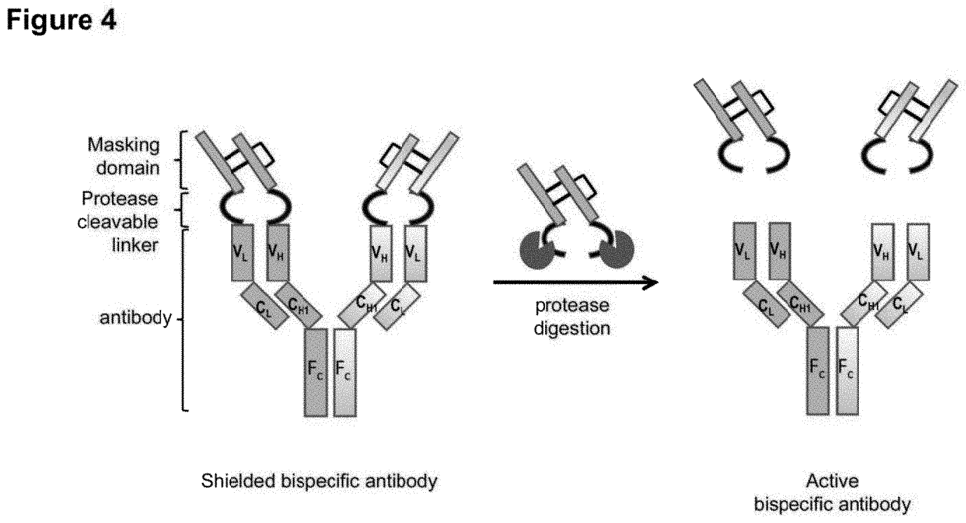

Many disease tissues, including tumor microenvironment and inflammation site, are abundant with various types of proteases whose overexpression correlate with the disease progression. In disease tissues, the protease-cleavable linker sequences of the shielded antibody are recognized by appropriate type of protease and the release the shield from the antibody chains. The protease may cleave both of the two protease-cleavable linkers or one of the two protease-cleavable linker sequences so the shielding domain is inactive. In either case, the shielding domain would not be able to interfere or block the binding of the Fab arm to its target antigen. As a result, the shielded antibody is converted into active antibody to bind and exert its functional activity to its target (FIG. 4).

In some embodiments, the protease-cleavable linker sequences linking the two shielding domains to the two Fab domains in the shielded antibody comprise the same sequences so as to be cleaved by the same type of protease.

In some embodiments, the protease-cleavable linker sequences linking the two masking domains and the two Fab domains in shielded antibody comprise different sequences with substrate sequences cleaved by different types of protease.

In some instances, the protease-cleavable linker sequence between the shield and antibody heavy chain comprises substrate sequences that can cleaved by one type of protease while the protease-cleavable linker sequence between the shield and antibody light chain comprises substrate sequence cleaved by a different type of protease. This design of shielded antibody allows the shielded antibody to be activated in a disease tissue in which at least one of the two types of proteases is present.

In some instances, the protease-cleavable linker sequence between the shield and one Fab arm comprises substrate sequence cleaved by one type of protease while the protease-cleavable linker sequence between the shield and the other Fab arm of the same antibody comprises substrate sequence cleaved by a different type of protease. This type of molecule can be generated by bispecific antibody generation technology. In this configuration in which the two Fab arms of the same antibody are linked to the shields via two different types of protease-cleavable linkers, either type of the proteases may only cleave off just one of the two shields while the presence of both types of proteases is needed to cleave off both shielding domains. This particular design of shielded antibody allows the shielded antibody to be fully activated in a disease tissue in which both types of proteases need to be present while the presence of just one of them may only partially activate the shielded antibody.

Proteases are enzymes that catalyze the hydrolytic cleavage of peptide bonds of target proteins. Depending on the catalytic mechanisms, they can be divided into five distinct classes: serine, cysteine, aspartic, metallo- and threonine proteases. Physiologically, proteases are involved in numerous important processes including protein turnover, nutrient digestion, fertilization, cell differentiation and growth, the immune response, and apoptosis. The activity of proteases is normally tightly controlled through multiple redundant mechanisms, including regulation of biosynthesis, activation of inactive precursors known as pro-enzymes or zymogens, and by the binding of endogenous inhibitors and cofactors. However, during pathological conditions including cancer, autoimmune diseases and chronic inflammation, the expression and activity of proteases can be significantly up-regulated and inappropriate proteolysis can have a major role in the development of these diseases. For example, multiple proteases, including metalloproteinases, serine and cysteine proteases have up-regulation in the cancer microenvironment and execution of diverse functions at different stages of malignant progression, including tumor angiogenesis, invasion, and metastasis.

Due to the significant difference in the expression levels of certain types of protease in the disease lesion sites and normal tissues, engineering protease substrate sites in the shielded antibody in this disclosure allows the conversion of shielded antibody into active antibody selectively in the disease lesion site by protease-mediated cleave off of the shielding domains. The disclosure provides for the protease-cleavable linker sequence comprising substrate peptide sequence cleaved by any types of protease whose expression level or activity is significantly higher in disease lesion site, including cancer, inflammation, autoimmune, cardiovascular, neurodegenerative, and bacterial and viral infection diseases.

Matrix metallopeptidases (MMPs), also known as matrix metalloproteinases or matrixins, are metalloproteinases that are calcium-dependent zinc-containing endopeptidases. Collectively, these enzymes are capable of degrading all kinds of extracellular matrix proteins. MMPs play critical roles during tissue remodeling in normal physiological processes, such as embryonic development and reproduction, as well as in disease processes, such as arthritis, and tumour metastasis. MMPs have been reported as one of the main factors of cancer progression and metastasis formation. MMPs have also been reported as major proteases in the inflammation sites during autoimmune and chronical inflammation diseases. The disclosure provides for the protease-cleavable linker sequence comprising substrate peptide sequence cleaved by different types of matrix metallopeptidases whose expression level or activity is significantly higher in disease lesion site relative to normal tissues.

Among the family of matrix metalloproteinases (MMPs), MMP2, MMP3 and MMP9 are up-regulated in many types of cancers, including breast, colorectal and lung cancers. Besides, the expression and activity of MMP2, MMP3 and MMP9 also correlates to the progression of many autoimmune disorders and inflammatory diseases, including rheumatoid arthritis, psoriasis, multiple sclerosis, chronic obstructed pulmonary disease, inflammatory bowel disease and osteoporosis (Lin, Lu et al. 2020). The disclosure provides for the protease-cleavable linker sequence comprising substrate peptide sequence cleaved by MMP2, MMP3 and MMP9. As non-limiting examples, the disclosure provides for the MMP2, MMP3 and MMP9 cleavable substrate peptide sequences set forth as SEQ ID NO: 15-20.

The urokinase plasminogen activator (uPA) has been reported to be overexpressed in many types of cancer, especially the breast cancer (Banys-Paluchowski, Witzel et al. 2019). uPA is a serine protease that can catalyze the conversion of plasminogen to plasmin which can degrade the basement membrane or extracellular matrix. The matrix degradation can facilitate tumor cells migration and invasion into the surrounding tissue. The disclosure provides for the protease-cleavable linker sequence comprising substrate peptide sequence cleaved by uPA. As non-limiting examples, the disclosure provides for the uPA-cleavable substrate peptide sequence set forth as SEQ ID NO: 21 and 22.

The protease-cleavable linker of the present disclosure can include one or more linker peptides interposed between, e.g., shielding sequence and protease substrate peptide sequence, and/or between protease substrate peptide sequence and antibody chains.

### Linker

Suitable linkers (also referred to as “spacers”) can be readily selected, and can be of any of a number of suitable lengths, such as from 1 amino acid to 30 amino acids (e.g., any specific integer between 1 and 30, or from 1 amino acid (e.g., Gly) to about 20 amino acids, from 2-15, 3-12, 4-10, 5-9, 6-8, or 7-8 amino acids).

Exemplary linkers include glycine polymers (G)ₙ (SEQ ID NO: 249), glycine-serine polymers (including, for example, (GS)ₙ (SEQ ID NO: 250), (GSGGS)ₙ (SEQ ID NO: 251) and (GGGS)ₙ (SEQ ID NO: 252), where n is an integer of at least one, e.g., 1, 2, 3, 4, 5, 6, 7, 8, 9, 10, 11, 12, 13, 14, 15, 16, 17, 18, 19, 20), glycine-alanine polymers, alanine-serine polymers, alanine-proline, immunoglobulin isotype and subtype hinge that can comprise IgG₁, IgG₂, IgG₃, IgG₄, IgA, IgE, IgM, and other flexible linkers known in the art. Both Gly and Ser are relatively unstructured, and therefore can serve as a neutral tether between components.

In certain embodiments, the linker is a Glycine polymer. Glycine accesses significantly more phi-psi space than even alanine and is much less restricted than residues with longer side chains (Scheraga 2008). Exemplary linkers can comprise amino acid sequences including, but not limited to: GGS; GGSG (SEQ ID NO: 253); GGSGG (SEQ ID NO: 254); GGGGS (SEQ ID NO: 255); GSGSG (SEQ ID NO: 256); GSGGG (SEQ ID NO: 257); GGGSG (SEQ ID NO: 258); GSSSG (SEQ ID NO: 259), and the like.

In certain embodiments, the linker is an Alanine-Proline polymer. Exemplary linkers can comprise amino acid sequences including, but not limited to (AP)ₙ (SEQ ID NO: 260), where n is an integer of at least one, e.g., 1, 2, 3, 4, 5, 6, 7, 8, 9, 10, 11, 12, 13, 14, 15, 16, 17, 18, 19, 20).

In certain embodiments, the linker is a rigid linker (Chen, Zaro et al. 2013). Exemplary rigid linkers can comprise amino acid sequences including, but not limited to, proline-rich sequence, (XP)ₙ, with X designating any amino acid, preferably Ala, Lys, or Glu, where n is an integer of at least one, e.g., 1, 2, 3, 4, 5, 6, 7, 8, 9, 10, 11, 12, 13, 14, 15, 16, 17, 18, 19, 20). Exemplary rigid linkers can also comprise amino acid sequences including, but not limited to, alpha helix-forming linkers with the sequence of (EAAAK)ₙ (SEQ ID NO: 261), where n is an integer of at least one, e.g., 1, 2, 3, 4, 5, 6, 7, 8, 9, 10, 11, 12, 13, 14, 15, 16, 17, 18, 19, 20).

### HER2 and VEGF Antibodies and Fragments Applicable for Shielded HER2 x VEGF Bispecific Antibody Design

The disclosure provides for therapeutic HER2 and VEGF antibodies and fragments to which shields are attached to their respective Fab domains via protease-cleavage linker sequences to make shielded HER2 x VEGF bispecific antibodies. The HER2 and VEGF targets of such therapeutic antibodies have differential expression levels in pathological sites and normal tissues. The shielded HER2 x VEGF bispecific antibody remains inactive in normal tissue due to the inhibitory effects of the masking domain on the CDR binding domains. The masking domains are cleaved off by proteases in disease sites and the shielded HER2 x VEGF bispecific antibody is converted to an active HER2 x VEGF bispecific antibody.

The therapeutic antibodies and fragments applicable for a shielded HER2 x VEGF bispecific antibody design of the present disclosure encompass full length antibody comprising two heavy chains and two light chains. The antibodies can be human or humanized antibodies. Humanized antibodies include chimeric antibodies and CDR-grafted antibodies. Chimeric antibodies are antibodies that include a non-human antibody variable region linked to a human constant region. CDR-grafted antibodies are antibodies that include the CDRs from a non-human “donor” antibody linked to the framework region from a human “recipient” antibody. Exemplary human or humanized antibodies include IgG, IgM, IgE, IgA, and IgD antibodies. The present antibodies can be of any class (IgG, IgM, IgE, IgGA, IgD, etc.) or isotype. For example, a human antibody can comprise an IgG Fc domain, such as at least one of isotypes, IgG1, IgG2, IgG3 or IgG4.

Human antibody heavy and light chain sequences are provided that form the CDR binding regions that bind to HER2. As non-limiting examples, the disclosure provides for the anti-HER2 heavy and light chain variable region amino acid sequences set forth as SEQ ID NO: 23-34, including several variants of heavy and light chain variable region amino acid sequences of trastuzumab. As non-limiting examples, the disclosure provides for the anti-HER2 antibodies (e.g., as listed in Table 2) with combinations of different heavy chain and different light chain sequences set forth as SEQ ID NO: 53-65.

TABLE 2
Anti-HER2 antibody

| | Heavy | Light | |
|---|---|---|---|
| Anti-HER2 Antibody | chain | chain | Fc |
| Tras_IgG1_LS | EAC289 | EAC203 | IgG1 with F405L, M428L, |
| | (SEQ: 55) | (SEQ: 54) | N434S mutations |
| Pertuzu_IgG1_LS | EAC505 | EAC506 | IgG1 with K409R, M428L, |
| | (SEQ: 56) | (SEQ: 57) | N434S mutations |
| Tras_IgG1_LS_E345R | EAC509 | EAC203 | IgG1 with E345R, F405L, |
| | (SEQ: 58) | (SEQ: 54) | M428L, N434S mutations |
| TrasVH1VL1_IgG1_LS | EAC562 | EAC564 | IgG1 with F405L, M428L, |
| | (SEQ: 59) | (SEQ: 61) | N434S mutations |
| TrasVH1VL1_IgG1_LS_E345R | EAC563 | EAC564 | IgG1 with E345R, F405L, |
| | (SEQ: 60) | (SEQ: 61) | M428L, N434S mutations |
| TrasVH1VL1_IgG1_LS_T366W | EAC1040 | EAC564 | IgG1 with T366W, M428L, |
| | (SEQ: 63) | (SEQ: 61) | N434S mutations |
| TrasVH1VL1_IgG1_F243L_R292P_{—} | EAC1047 | EAC564 | IgG1 with F243L, R292P, |
| Y300L_V305I_P396L | (SEQ: 64) | (SEQ: 61) | Y300L, V305I, P396L |
| | | | mutations |
| TrasVH1VL1_IgG1_F243L_R292P_{—} | EAC1048 | EAC564 | IgG1 with F243L, R292P, |
| Y300L_V305I_T366W_P396L | (SEQ: 65) | (SEQ: 61) | Y300L, V305I, T366W, P396L |
| | | | mutations |

Human antibody heavy and light chain sequences are provided that form the CDR binding regions that bind to VEGF. As non-limiting examples, the disclosure provides for the anti-VEGF heavy and light chain variable region amino acid sequences set forth as SEQ ID NO: 35-44, including several variants of heavy and light chain variable region amino acid sequences of bevacizumab. As non-limiting examples, the disclosure provides for the anti-VEGF antibodies (e.g., as listed in Table 3) with combinations of different heavy chain and different light chain sequences set forth as SEQ ID NO: 66-81.

TABLE 3
Anti-VEGF antibody

| | Heavy | Light | |
|---|---|---|---|
| Anti-VEGF Antibody | chain | chain | Fc |
| Beva_IgG1_LS | EAC291 | EAC207 | IgG1 with K409R, M428L, |
| | (SEQ: 68) | (SEQ: 67) | N434S mutations |
| Beva_IgG1_LS_E345R | EAC513 | EAC207 | IgG1 with E345R, K409R, |
| | (SEQ: 69) | (SEQ: 67) | M428L, N434S mutations |
| BevaVH2VL1_IgG1_LS | EAC828 | EAC833 | IgG1 with K409R, M428L, |
| | (SEQ: 71) | (SEQ: 75) | N434S mutations |
| BevaVH3VL1_IgG1_LS | EAC829 | EAC833 | IgG1 with K409R, M428L, |
| | (SEQ: 72) | (SEQ: 75) | N434S mutations |
| BevaVH2VL1_IgG1_LS_E345R | EAC855 | EAC833 | IgG1 with E345R, K409R, |
| | (SEQ: 78) | (SEQ: 75) | M428L, N434S mutations |
| BevaVH3VL1_IgG1_LS_E345R | EAC846 | EAC833 | IgG1 with E345R, K409R, |
| | (SEQ: 77) | (SEQ: 75) | M428L, N434S mutations |
| BevaVH3VL1_IgG1_LS_T366S_{—} | EAC1042 | EAC833 | IgG1 with T366S, L368A, |
| L368A_Y407V | (SEQ: 80) | (SEQ: 75) | Y407V, M428L, N434S |
| | | | mutations |
| BevaVH3VL1_IgG1_F243L_{—} | EAC1049 | EAC833 | IgG1 with F243L, R292P, |
| R292P_Y300L_V305I_P396L_{—} | (SEQ: 81) | (SEQ: 75) | Y300L, V305I, P396L, T366S, |
| T366S_L368A_Y407V | | | L368A, Y407V mutations |

In some embodiments of the disclosure, a therapeutic HER2xVEGF bispecific antibody comprises an anti-HER2 antibody arm and an anti-VEGF antibody arm. As non-limiting examples, the disclosure provides for the HER2 x VEGF bispecific antibodies (e.g., as listed in Table 4) with combinations of different heavy chain and different light chain sequences of anti-HER2 and anti-VEGF antibodies set forth as SEQ ID NO: 53-81.

TABLE 4
HER2 × VEGF bispecific antibody

| | HER2 | HER2 | VEGF | VEGF |
|---|---|---|---|---|
| | Heavy | Light | Heavy | Light |
| HER2 × VEGF Antibody | chain | chain | chain | chain |
| Tras × Beva_IgG1_LS | EAC289 | EAC203 | EAC291 | EAC207 |
| | (SEQ: 55) | (SEQ: 54) | (SEQ: 68) | (SEQ: 67) |
| Tras × Beva_IgG1_LS_E345R | EAC509 | EAC203 | EAC513 | EAC207 |
| | (SEQ: 58) | (SEQ: 54) | (SEQ: 69) | (SEQ: 67) |
| TrasVH1VL1 × BevaVH2VL1_IgG1_LS | EAC562 | EAC564 | EAC828 | EAC833 |
| | (SEQ: 59) | (SEQ: 61) | (SEQ: 71) | (SEQ: 75) |
| TrasVH1VL1 × BevaVH3VL1_IgG1_LS | EAC562 | EAC564 | EAC829 | EAC833 |
| | (SEQ: 59) | (SEQ: 61) | (SEQ: 72) | (SEQ: 75) |
| TrasVH1VL1 × | EAC563 | EAC564 | EAC855 | EAC833 |
| BevaVH2VL1_IgG1_LS_E345R | (SEQ: 60) | (SEQ: 61) | (SEQ: 78) | (SEQ: 75) |
| TrasVH1VL1 × | EAC563 | EAC564 | EAC846 | EAC833 |
| BevaVH3VL1_IgG1_LS_E345R | (SEQ: 60) | (SEQ: 61) | (SEQ: 77) | (SEQ: 75) |
| TrasVH1VL1 × | EAC1040 | EAC564 | EAC1042 | EAC833 |
| BevaVH3VL1_IgG1_kih_LS | (SEQ: 63) | (SEQ: 61) | (SEQ: 80) | (SEQ: 75) |
| TrasVH1VL1 × | EAC1048 | EAC564 | EAC1049 | EAC833 |
| BevaVH3VL1_IgG1_kih_F243L_{—} | (SEQ: 65) | (SEQ: 61) | (SEQ: 81) | (SEQ: 75) |
| R292P_Y300L_V305I_P396L | | | | |

Long term administration of anti-HER2 or anti-VEGF biologics drugs pose a great risk factor for patients. The conversion of anti-HER2 and/or anti-VEGF antibody arms into shielded arms with masking domains may increase the safety profile and therapeutic window of the respective antibody arms.

As non-limiting examples, the disclosure provides for the shielded anti-HER2 antibodies (e.g., as listed in Table 5) with combinations of HER2 heavy chain and light chain with masking domains and protease substrate sequences set forth as SEO ID NO: 101-145.

TABLE 5
Shielded anti-HER2 antibody

| | Masking | Protease | Heavy | Light |
|---|---|---|---|---|
| Shielded anti-HER2 Antibody | domain | substrate | chain | chain |
| Hinge_MMP2_Tras_IgG1_LS_{—} | hinge | MMP2 | EAC204 | EAC205 |
| E233P_G236del | | | (SEQ: 101) | (SEQ: 102) |
| IGF2_MMP2_Tras_IgG1_LS | IGF2 | MMP2 | EAC293 | EAC294 |
| | | | (SEQ: 110) | (SEQ: 111) |
| Collagen1-fibronectin_MMP2_{—} | Collagen1- | MMP2 | EAC290 | EAC261 |
| Tras_IgG1_LS | fibronectin | | (SEQ: 109) | (SEQ: 108) |
| IGF2_MMP2_Pertuzu_IgG1_LS | IGF2 | MMP2 | EAC507 | EAC508 |
| | | | (SEQ: 112) | (SEQ: 113) |
| IGF2_MMP2_Tras_IgG1_LS_{—} | IGF2 | MMP2 | EAC510 | EAC294 |
| E345R | | | (SEQ: 114) | (SEQ: 111) |
| Collagen2-fibronectin_MMP2_{—} | Collagen2- | MMP2 | EAC515 | EAC261 |
| Tras_IgG1_LS | fibronectin | | (SEQ: 115) | (SEQ: 108) |
| IGF2_MMP2_TrasVH1VL1_{—} | IGF2 | MMP2 | EAC527 | EAC532 |
| IgG1_LS | | | (SEQ: 116) | (SEQ: 119) |
| IGF2_MMP2_TrasVH1VL1_{—} | IGF2 | MMP2 | EAC561 | EAC532 |
| IgG1_LS_E345R | | | (SEQ: 126) | (SEQ: 119) |
| IGF2_MMP3_TrasVH1VL1_{—} | IGF2 | MMP3 | EAC809 | EAC810 |
| IgG1_LS | | | (SEQ: 127) | (SEQ: 128) |
| IGF2_uPA-G_TrasVH1VL1_{—} | IGF2 | uPA-G | EAC811 | EAC813 |
| IgG1_LS | | | (SEQ: 129) | (SEQ: 131) |
| IGF2_uPA-G_TrasVH1VL1_{—} | IGF2 | uPA-G | EAC812 | EAC813 |
| IgG1_LS_E345R | | | (SEQ: 130) | (SEQ: 131) |
| Collagen2-fibronectin_MMP2_{—} | Collagen2- | MMP2 | EAC535 | EAC540 |
| TrasVH1VL1_IgG1_LS | fibronectin | | (SEQ: 121) | (SEQ: 124) |
| IGF2_MMP2_TrasVH1VL1_{—} | IGF2 | MMP2 | EAC1041 | EAC532 |
| IgG1_kih_LS | | | (SEQ: 133) | (SEQ: 119) |
| IGF2_MMP2_TrasVH1VL1_{—} | IGF2 | MMP2 | EAC1050 | EAC532 |
| IgG1_kih_F243L_{—} | | | (SEQ: 134) | (SEQ: 119) |
| R292P_Y300L_V305I_P396L | | | | |
| IGF2_MMP2_TrasVH1VL1_{—} | IGF2 | MMP2 | EAC527 | EAC560 |
| IgG1_LS_PDL1ScFv | | | (SEQ: 116) | (SEQ: 145) |
| IGF2_MMP2_TrasVH1VL1_{—} | IGF2 | MMP2 | EAC561 | EAC560 |
| IgG1_LS_E345R_PDL1ScFv | | | (SEQ: 126) | (SEQ: 145) |

As non-limiting examples, the disclosure provides for the shielded anti-VEGF antibodies as listed in Table 6 with combinations of VEGF heavy chain and light chain with masking domains and protease substrate sequences set forth as SEQ ID NO: 201-248.

TABLE 6
Shielded anti-VEGF antibody

| | Masking | Protease | Heavy | Light |
|---|---|---|---|---|
| Shielded anti-VEGF Antibody | domain | substrate | chain | chain |
| Hinge_MMP2_Beva_IgG1_LS_{—} | hinge | MMP2 | EAC208 | EAC209 |
| E233P_G236del | | | (SEQ: 201) | (SEQ: 202) |
| IGF2_MMP2_Beva_IgG1_LS | IGF2 | MMP2 | EAC847 | EAC848 |
| | | | (SEQ: 234) | (SEQ: 235) |
| Collagen1-fibronectin_MMP2_{—} | Collagen1- | MMP2 | EAC292 | EAC263 |
| Beva_IgG1_LS | fibronectin | | (SEQ: 209) | (SEQ: 208) |
| IGF2_MMP2_Broluci_IgG1_LS | IGF2 | MMP2 | EAC297 | EAC298 |
| | | | (SEQ: 210) | (SEQ: 211) |
| Collagen2-fibronectin_MMP2_{—} | Collagen2- | MMP2 | EAC516 | EAC263 |
| Beva_IgG1_LS | fibronectin | | (SEQ: 212) | (SEQ: 208) |
| IGF2_MMP2_BevaVH3VL1_{—} | IGF2 | MMP2 | EAC596 | EAC534 |
| IgG1_LS | | | (SEQ: 219) | (SEQ: 215) |
| IGF2_MMP2_BevaVH3VL1_{—} | IGF2 | MMP2 | EAC845 | EAC534 |
| IgG1_LS_E345R | | | (SEQ: 233) | (SEQ: 215) |
| Collagen2-fibronectin_MMP2_{—} | Collagen2- | MMP2 | EAC539 | EAC542 |
| BevaVH2VL1_IgG1_LS | fibronectin | | (SEQ: 217) | (SEQ: 218) |
| Collagen2-fibronectin_MMP2_{—} | Collagen2- | MMP2 | EAC835 | EAC542 |
| BevaVH2VL1_IgG1_LS_E345R | fibronectin | | (SEQ: 232) | (SEQ: 218) |
| IGF2_MMP2_BevaVH3VL1_{—} | IGF2 | MMP2 | EAC1043 | EAC534 |
| IgG1_kih_LS | | | (SEQ: 238) | (SEQ: 215) |
| IGF2_MMP2_BevaVH3VL1_{—} | IGF2 | MMP2 | EAC1051 | EAC534 |
| IgG1_kih_F243L_{—} | | | (SEQ: 239) | (SEQ: 215) |
| R292P_Y300L_V305I_P396L | | | | |
| Collagen2- | Collagen2- | MMP2 | EAC1052 | EAC542 |
| fibronectin_MMP2_BevaVH3VL1_{—} | fibronectin | | (SEQ: 240) | (SEQ: 218) |
| IgG1_kih_F243L_{—} | | | | |
| R292P_Y300L_V305I_P396L | | | | |
| IGF2_MMP2_BevaVH3VL1_{—} | IGF2 | MMP2 | EAC596 | EAC844 |
| IgG1_LS_PDL1ScFv | | | (SEQ: 219) | (SEQ: 247) |
| IGF2_MMP2_BevaVH3VL1_{—} | IGF2 | MMP2 | EAC845 | EAC844 |
| IgG1_LS_E345R_PDL1ScFv | | | (SEQ: 233) | (SEQ: 247) |
| Collagen2-fibronectin_MMP2_{—} | Collagen2- | MMP2 | EAC539 | EAC836 |
| BevaVH2VL1_IgG1_LS_PDL1ScFv | fibronectin | | (SEQ: 217) | (SEQ: 248) |
| Collagen2-fibronectin_MMP2_{—} | Collagen2- | MMP2 | EAC835 | EAC836 |
| BevaVH2VL1_IgG1_LS_E345R_PDL1ScFv | fibronectin | | (SEQ: 232) | (SEQ: 248) |

As non-limiting examples, the disclosure provides for the shielded HER2xVEGF bispecific antibodies (e.g., as listed in Table 7) with combinations of different heavy chain and different light chain sequences of shielded anti-HER2 and shielded anti-VEGF antibodies set forth as SEQ ID NO: 101-144 and 201-246 respectively.

TABLE 7
Shielded HER2 × VEGF bispecific antibody

| | HER2 | HER2 | VEGF | VEGF |
|---|---|---|---|---|
| | Heavy | Light | Heavy | Light |
| Shielded HER2 × VEGF Antibody | chain | chain | chain | chain |
| Hinge_MMP2_Tras × Beva_IgG1_LS_{—} | EAC204 | EAC205 | EAC208 | EAC209 |
| E233P_G236del | (SEQ: 101) | (SEQ: 102) | (SEQ: 201) | (SEQ: 202) |
| IGF2_MMP2_Tras × Beva_IgG1_LS | EAC293 | EAC294 | EAC847 | EAC848 |
| | (SEQ: 110) | (SEQ: 111) | (SEQ: 234) | (SEQ: 235) |
| Collagen1-fibronectin_MMP2_{—} | EAC290 | EAC261 | EAC292 | EAC263 |
| Tras × Beva_IgG1_LS | (SEQ: 109) | (SEQ: 108) | (SEQ: 209) | (SEQ: 208) |
| IGF2_MMP2_Pertuzu × | EAC507 | EAC508 | EAC297 | EAC298 |
| Broluci_IgG1_LS | (SEQ: 112) | (SEQ: 113) | (SEQ: 210) | (SEQ: 211) |
| Collagen2-fibronectin_MMP2_{—} | EAC515 | EAC261 | EAC516 | EAC263 |
| Tras × Beva_IgG1_LS | (SEQ: 115) | (SEQ: 108) | (SEQ: 212) | (SEQ: 208) |
| IGF2_MMP2_TrasVH1VL1 × | EAC527 | EAC532 | EAC596 | EAC534 |
| BevaVH3VL1_IgG1_LS | (SEQ: 116) | (SEQ: 119) | (SEQ: 219) | (SEQ: 215) |
| IGF2_MMP2_TrasVH1VL1 × | EAC561 | EAC532 | EAC845 | EAC534 |
| BevaVH3VL1_IgG1_LS_E345R | (SEQ: 126) | (SEQ: 119) | (SEQ: 233) | (SEQ: 215) |
| IGF2_MMP2_TrasVH1VL1 × | EAC527 | EAC532 | EAC539 | EAC542 |
| Collagen2-fibronectin_MMP2_{—} | (SEQ: 116) | (SEQ: 119) | (SEQ: 217) | (SEQ: 218) |
| BevaVH2VL1_IgG1_LS | | | | |
| IGF2_MMP2_TrasVH1VL1 × | EAC561 | EAC532 | EAC835 | EAC542 |
| Collagen2-fibronectin_MMP2_{—} | (SEQ: 126) | (SEQ: 119) | (SEQ: 232) | (SEQ: 218) |
| BevaVH2VL1_IgG1_LS_E345R | | | | |
| IGF2_MMP2_TrasVH1VL1 × | EAC1041 | EAC532 | EAC1043 | EAC534 |
| BevaVH3VL1_IgG1_kih_LS | (SEQ: 133) | (SEQ: 119) | (SEQ: 238) | (SEQ: 215) |
| IGF2_MMP2_TrasVH1VL1 × | EAC1050 | EAC532 | EAC1051 | EAC534 |
| BevaVH3VL1_IgG1_kih_F243L_{—} | (SEQ: 134) | (SEQ: 119) | (SEQ: 239) | (SEQ: 215) |
| R292P_Y300L_V305I_P396L | | | | |
| IGF2_MMP2_TrasVH1VL1 × | EAC1050 | EAC532 | EAC1052 | EAC542 |
| Collagen2- | (SEQ: 134) | (SEQ: 119) | (SEQ: 240) | (SEQ: 218) |
| fibronectin_MMP2_BevaVH3VL1_{—} | | | | |
| IgG1_kih_F243L_{—} | | | | |
| R292P_Y300L_V305I_ P396L | | | | |

### IgG Fc of Shielded HER2 x VEGF Bispecific Antibody

The present shielded HER2 x VEGF bispecific antibody may comprise with a modified F_{c} region, wherein the modified F_{c} region comprises at least one amino acid modification relative to a native F_{c} region. In some embodiments, the present shielded HER2 x VEGF bispecific antibody is provided with a modified F_{c} region where a naturally-occurring F_{c} region is modified to extend the half-life of the antibody when compared to the parental native antibody in a biological environment, for example, the serum half-life or a half-life measured by an in vitro assay. Exemplary mutations that may be made singularly or in combination are T250Q, M252Y, I253A, S254T, T256E, P257I, T307A, D376V, E380A, M428L, H433K, N434S, N434A, N434H, N434F, H435A and H435R mutations.

In certain embodiments, the extension of half-life can be realized by engineering the M252Y/S254T/T256E mutations in IgG1 F_{c} residue numbering according to the EU Index (Dall'Acqua, Kiener et al. 2006).

In certain embodiments, the extension of half-life can also be realized by engineering the M428L/N434S mutations in IgG₁ F_{c} (Zalevsky, Chamberlain et al. 2010).

In certain embodiments, the extension of half-life can also be realized by engineering the T250Q/M428L mutations in IgG₁ F_{c} (Hinton, Xiong et al. 2006).

In certain embodiments, the extension of half-life can also be realized by engineering the N434A mutations in IgG₁ F_{c} (Shields, Namenuk et al. 2001).

In certain embodiments, the extension of half-life can also be realized by engineering the T307A/E380A/N434A mutations in IgG₁ F_{c} (Petkova, Akilesh et al. 2006).

The effect of F_{c} engineering on the extension of antibody half-life can be evaluated in PK studies in mice relative to antibodies with native IgG F_{c}.

In some embodiments, the present shielded HER2 x VEGF bispecific antibody is provided with a modified F_{c} region where a naturally-occurring F_{c} region is modified to enhance the antibody resistance to proteolytic degradation by a protease that cleaves the wild-type antibody between or at residues 222-237 (EU numbering).

In certain embodiments, the resistance to proteolytic degradation can be realized by engineering E233P/L234A/L235A mutations in the hinge region with G236 deleted when compared to a parental native antibody, residue numbering according to the EU Index (Kinder, Greenplate et al. 2013).

In instances where effector functionality is to be enhanced, the antibodies of the disclosure may further be engineered to introduce at least one mutation in the antibody F_{c} that increases the binding of the antibody to an activating F_{c}γ receptor (F_{c}γR) and/or increases F_{c} effector functions such as C1q binding, complement dependent cytotoxicity (CDC), antibody-dependent cell-mediated cytotoxicity (ADCC) or phagocytosis (ADCP). Such modifications can comprise low or null Fc fucosylation, and/or engineering of Fc mutations such as S239E/I332E/A330L, S239D/I332E/A330L, S239D/I332E, S239D, I332E, S298A/E333A/K334A.

In certain embodiments, the enhancement of effector functions can be realized by engineering F243L/R292P/Y300L/V305I/P396L mutations when compared to a parental native antibody, residue numbering according to the EU Index (Stavenhagen, Gorlatov et al. 2008). In certain embodiments, the enhancement of the effector functions can be realized by engineering E345R or other Fc clustering mutations on the bispecific antibody using residue numbering according to the EU Index (Stavenhagen, Gorlatov et al. 2008).

In some embodiments, the shielded HER2 x VEGF bispecific antibody is provided with a modified F_{c} region where a naturally-occurring Fc region is modified to facilitate the generation of bispecific antibody by Fc heterodimerization.

In certain embodiments, the Fc heterodimerization can be realized by engineering F405L and K409R mutations on two parental antibodies and the generation of bispecific antibody in a process known as Fab arm exchange (Labrijn, Meesters et al. 2014).

In certain embodiments, the Fc heterodimerization can also be realized by Fc mutations to facilitate Knob-in-Hole strategy (see, e.g., Intl. Publ. No. WO 2006/028936). An amino acid with a small side chain (hole) is introduced into one Fc domain and an amino acid with a large side chain (knob) is introduced into the other Fc domain. After co-expression of the two heavy chains, a heterodimer is formed as a result of the preferential interaction of the heavy chain with a “hole” with the heavy chain with a “knob” (Ridgway, Presta et al. 1996). Exemplary Fc mutation pairs forming a knob and a hole are: T366Y/F405A, T366W/F405W, F405W/Y407A, T394W/Y407T, T394S/Y407A, T366W/T394S, F405W/T394S and T366W/T366S/L368A/Y407V.

In certain embodiments, the Fc heterodimerization can be realized by engineering T366W and T366S/L368A/Y407V mutations on two parental antibodies and the generation of HER2 x VEGF bispecific antibody by co-expression or by a process known as Fab arm exchange.

In certain embodiments, the Fc heterodimerization can also be realized by Fc mutations to facilitate the electrostatically-matched interactions strategy (Gunasekaran, Pentony et al. 2010). Mutations can be engineered to generate positively charged residues at one Fc domain and negatively charged residues at the other Fc domain as described in US Patent Publ. No. US2010/0015133; US Patent Publ. No. US2009/0182127; US Patent Publ. No. US2010/028637 or US Patent Publ. No. US2011/0123532. Heavy chain heterodimerization can be formed by electrostatically-matched interactions between two mutated Fc.

In some embodiments, the present shielded HER2 x VEGF bispecific antibody is provided with a modified F_{c} region where a naturally-occurring Fc region is modified to facilitate the multimerization of the antibody upon interaction with cell surface receptors, although such engineered antibody exist as monomer in solution. The F_{c} mutations that facilitate antibody multimerization include, but not limited to, E345R mutation, E430G mutation, E345R/E430G mutations, E345R/E430G/Y440R mutations as described in (Diebolder, Beurskens et al. 2014). Such mutations may also include, but not limited to, T437R mutation, T437R/K248E mutations, T437R/K338A mutations as described in (Zhang, Armstrong et al. 2017).

Antibodies of the disclosure further comprising conservative modifications are within the scope of the disclosure. “Conservative modifications” refer to amino acid modifications that do not significantly affect or alter the binding characteristics of the antibody containing the amino acid sequences. Conservative modifications include amino acid substitutions, additions and deletions. Conservative substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis. Amino acid substitutions to the antibodies of the disclosure may be made by known methods for example by PCR mutagenesis (U.S. Pat. No. 4,683,195). Alternatively, libraries of variants may be generated for example using random (NNK) or non-random codons, for example DVK codons, which encode 11 amino acids (Ala, Cys, Asp, Glu, Gly, Lys, Asn, Arg, Ser, Tyr, Trp). The resulting antibody variants may be tested for their characteristics using assays described herein.

The antibodies of the disclosure may be post-translationally modified by processes such as glycosylation, isomerization, deglycosylation or non-naturally occurring covalent modification such as the addition of polyethylene glycol moieties (pegylation) and lipidation. Such modifications may occur in vivo or in vitro. For example, the antibodies of the disclosure may be conjugated to polyethylene glycol (PEGylated) to improve their pharmacokinetic profiles. Conjugation may be carried out by techniques known to those skilled in the art. Conjugation of therapeutic antibodies with PEG has been shown to enhance pharmacodynamics while not interfering with function.

Antibodies of the disclosure may be modified to improve stability, selectivity, cross-reactivity, affinity, immunogenicity or other desirable biological or biophysical property are within the scope of the disclosure. Stability of an antibody is influenced by a number of factors, including (1) core packing of individual domains that affects their intrinsic stability, (2) protein/protein interface interactions that have impact upon the HC and LC pairing, (3) burial of polar and charged residues, (4) H-bonding network for polar and charged residues; and/or (5) surface charge and polar residue distribution among other intra- and inter-molecular forces (Worn and Pluckthun 2001). Potential structure destabilizing residues may be identified based upon the crystal structure of the antibody or by molecular modelling in certain cases, and the effect of the residues on antibody stability may be tested by generating and evaluating variants harboring mutations in the identified residues. One of the ways to increase antibody stability is to raise the thermal transition midpoint (Tₘ) as measured by differential scanning calorimetry (DSC). In general, the protein Tₘ is correlated with its stability and inversely correlated with its susceptibility to unfolding and denaturation in solution and the degradation processes that depend on the tendency of the protein to unfold. A number of studies have found correlation between the ranking of the physical stability of formulations measured as thermal stability by DSC and physical stability measured by other methods. Formulation studies suggest that a Fab Tₘ has implication for long-term physical stability of a corresponding mAb.

Antibodies of the disclosure may have amino acid substitutions in the F_{c} region that improve manufacturing and drug stability. An example for IgG₁ is H224S (or H224Q) in the hinge 221-DKTHTC-226 (EU numbering) which blocks radically induced cleavage; and for IgG₄, the S228P mutation blocks half-antibody exchange.

### Homing Domain

The homing domain amino acid sequence can bind to matrix proteins found more prevalently in the tumor microenvironment. The homing domains are based on antibody single chain Fvs which can bind either ED-B, F19 or PD-L1. As non-limiting examples, the disclosure provides for the homing domain amino acid sequences set forth as SEQ ID NO: 45-52 and 82. These sequences can be fused to the C-termini of either the heavy chains or light chains of the anti-HER2 and anti-VEGF constructs.

As non-limiting examples, the disclosure provides for examples of shielded or unshielded HER2xVEGF bispecific antibodies with the homing domain as listed in Table 8 with combinations of different heavy chain and different light chain sequences of anti-HER2 and anti-VEGF antibodies with the homing domain set forth as SEQ ID NO: 83-84, 141-145 and 243-248.

TABLE 8
HER2 × VEGF bispecific antibody with homing domain

| | HER2 | HER2 | VEGF | VEGF |
|---|---|---|---|---|
| HER2 × VEGF Antibody with homing | Heavy | Light | Heavy | Light |
| domain | chain | chain | chain | chain |
| epitope_MMP2_Tras × | SEQ: 142 | SEQ: 141 | SEQ: 244 | SEQ: 243 |
| Beva_IgG1_LS_F19-2 | | | | |
| epitope_MMP2_Tras × | SEQ: 144 | SEQ: 143 | SEQ: 246 | SEQ: 245 |
| Beva_IgG1_LS_FAP-2 | | | | |
| Tras × Beva_IgG1_LS_PDL1ScFv | EAC289 | EAC511 | EAC291 | EAC514 |
| | (SEQ: 55) | (SEQ: 82) | (SEQ: 68) | (SEQ: 83) |
| Tras × Beva_{—} | EAC509 | EAC511 | EAC513 | EAC514 |
| IgG1_LS_E345R_PDL1ScFv | (SEQ: 58) | (SEQ: 82) | (SEQ: 69) | (SEQ: 83) |
| IGF2_MMP2_TrasVH1VL1 × | EAC527 | EAC560 | EAC596 | EAC844 |
| BevaVH3VL1_IgG1_LS_PDL1ScFv | (SEQ: 116) | (SEQ: 145) | (SEQ: 219) | (SEQ: 247) |
| IGF2_MMP2_TrasVH1VL1 × | EAC561 | EAC560 | EAC845 | EAC844 |
| BevaVH3VL1_IgG1_LS_E345R_{—} | (SEQ: 126) | (SEQ: 145) | (SEQ: 233) | (SEQ: 247) |
| PDL1ScFv | | | | |
| IGF2_MMP2_TrasVH1VL1 × | EAC527 | EAC560 | EAC539 | EAC836 |
| Collagen2-fibronectin_MMP2_{—} | (SEQ: 116) | (SEQ: 145) | (SEQ: 217) | (SEQ: 248) |
| BevaVH2VL1_IgG1_LS_PDL1ScFv | | | | |
| IGF2_MMP2_TrasVH1VL1 × | EAC561 | EAC560 | EAC835 | EAC836 |
| Collagen2-fibronectin_MMP2_{—} | (SEQ: 126) | (SEQ: 145) | (SEQ: 232) | (SEQ: 248) |
| BevaVH2VL1_IgG1_LS_E345R_{—} | | | | |
| PDL1ScFv | | | | |

### Expression and Purification of Shielded HER2 x VEGF Bispecific Antibody

A shielded HER2 x VEGF bispecific antibody of the present disclosure can be encoded by a single nucleic acid (e.g., a single nucleic acid comprising nucleotide sequences that encode the light and heavy chain polypeptides of the shielded antibody), or by two or more separate nucleic acids, each of which encode a different part of the shielded parental antibody.

The nucleic acids described herein can be inserted into vectors, e.g., nucleic acid expression vectors and/or targeting vectors. Such vectors can be used in various ways, e.g., for the expression of an antibody with IGF2-based masking domain described herein in a cell or transgenic animal. Vectors are typically selected to be functional in the host cell in which the vector will be used. A nucleic acid molecule encoding an antibody with IGF2-based masking domain described herein may be amplified/expressed in prokaryotic, yeast, insect (baculovirus systems) and/or eukaryotic host cells. Selection of the host cell will depend in part on whether a shielded HER2 x VEGF bispecific antibody described herein is to be post-translationally modified (e.g., glycosylated and/or phosphorylated). If so, yeast, insect, or mammalian host cells are preferable. Expression vectors typically contain one or more of the following components: a promoter, one or more enhancer sequences, an origin of replication, a transcriptional termination sequence, a complete intron sequence containing a donor and acceptor splice site, a leader sequence for secretion, a ribosome binding site, a polyadenylation sequence, a polylinker region for inserting the nucleic acid encoding the polypeptide to be expressed, and a selectable marker element.

In most cases, a leader or signal sequence is engineered at the N-terminus of a shielded HER2 x VEGF bispecific antibody described herein to guide its secretion. The secretion of the shielded HER2 x VEGF bispecific antibody from a host cell will result in the removal of the signal peptide from the antibody. Thus, the mature shielded HER2 x VEGF bispecific antibody will lack any leader or signal sequence. In some cases, such as where glycosylation is desired in a eukaryotic host cell expression system, one may manipulate the various pre-sequences to improve glycosylation or yield. For example, one may alter the peptidase cleavage site of a signal peptide, or add pre-sequences, which also may affect glycosylation.

The disclosure further provides a cell (e.g., an isolated or purified cell) comprising a nucleic acid or vector of the disclosure. The cell can be any type of cell capable of being transformed with the nucleic acid or vector of the disclosure so as to produce a polypeptide encoded thereby. To express a shielded HER2 x VEGF bispecific antibody described herein, DNAs encoding partial or full-length light and heavy chains, obtained as described above, are inserted into expression vectors such that the genes are operatively linked to transcriptional and translational control sequences.

Methods of introducing nucleic acids and vectors into isolated cells and the culture and selection of transformed host cells in vitro are known in the art and include the use of calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, and electroporation.

After introducing the nucleic acid or vector of the disclosure into the cell, the cell is cultured under conditions suitable for expression of the encoded sequence. The antibody, antigen binding fragment, or portion of the antibody then can be isolated from the cell.

In certain embodiments, two or more vectors that together encode a shielded HER2 x VEGF bispecific antibody described herein, can be introduced into the cell.

Purification of the shielded HER2 x VEGF bispecific antibody described herein which has been secreted into the cell media can be accomplished using a variety of techniques including affinity, immunoaffinity or ion exchange chromatography, molecular sieve chromatography, preparative gel electrophoresis or isoelectric focusing, chromatofocusing, and high-pressure liquid chromatography. For example, antibodies comprising an F_{c} region may be purified by affinity chromatography with Protein A, which selectively binds the F_{c} region.

Modified forms of a shielded HER2 x VEGF bispecific antibody may be prepared with affinity tags, such as hexahistidine (SEQ ID NO: 262) or other small peptide such as FLAG (Eastman Kodak Co., New Haven, Conn.) or myc (Invitrogen) at either its carboxyl or amino terminus and purified by a one-step affinity column. For example, polyhistidine binds with great affinity and specificity to nickel, thus an affinity column of nickel (such as the Qiagen® nickel columns) can be used for purification of polyhistidine-tagged selective binding agents. In some instances, more than one purification step may be employed.

### Generation of HER2 x VEGF Bispecific Antibody

The bispecific antibody can be generated by a process known as controlled F_{ab} arm exchange from two parental antibodies with F405L and K409R (EU numbering) mutation in IgG Fc respectively (Labrijn, Meesters et al. 2014). The controlled Fab arm exchange reaction is the result of a disulfide-bond isomerization reaction and dissociation-association of C_{H3} domains. First, two parental antibodies are generated, one bearing the F405L F_{c} mutation, and one bearing the K409R F_{c} mutation. The heavy chain disulfide bonds in the hinge regions of the parental antibodies are reduced and the heavy chains of the parental antibodies are separated. The F405L and K409R mutations favor heterodimerization over homodimerization of the heavy chains. Therefore, the resulting free cysteines of one of the parental antibodies form an inter heavy-chain disulfide bond with cysteine residues of a second parental antibody. The resulting product is a heterodimerized antibody with one half coming from one parental antibody and the other half coming from another parental antibody.

In the present disclosure, a HER2 x VEGF bispecific antibody with dual specificity to both HER2 and VEGF is generated from one parental antibody to HER2 with F405L Fc mutation and another parental antibody to VEGF with K409R Fc mutation by controlled Fab arm exchange.

A HER2 x VEGF bispecific antibody of the present disclosure may be generated by other F_{c} mutations and engineering processes that facilitate F_{c} heterodimerization, including, but not limited to, Knob-in-Hole and the electrostatically-matched interactions.

In the Knob-in-Hole strategy (see, e.g., Intl. Publ. No. WO 2006/028936, incorporated by reference), selected amino acids forming the interface of the C_{H3} domains in human IgG can be mutated at positions affecting C_{H3} domain interactions to promote heterodimer formation. An amino acid with a small side chain (hole) is introduced into one F_{c} domain and an amino acid with a large side chain (knob) is introduced into the other F_{c} domain of the parental antibodies. After co-expression of the two heavy chains, a heterodimer is formed because of the preferential interaction of the heavy chain with a “hole” with the heavy chain with a “knob.” Exemplary C_{H3} substitution pairs forming a knob and a hole include: T366Y/F405A, T366W/F405W, F405W/Y407A, T394W/Y407T, T394S/Y407A, T366W/T394S, F405W/T394S and T366W/T366S_L368A/Y407V.

In the electrostatically-matched interactions strategy, mutations can be engineered to generate positively charged residues at one C_{H3} surface and negatively charged residues at a second C_{H3} surface as described in US 2010/0015133 A1; US 2009/0182127 A1; US 2010/028637 A1, or US 2011/0123532 A1. Heterodimerization of heavy chain can be formed by electrostatically-matched interactions between two mutated F_{c}.

The formation of bispecific antibody can be assessed by an ELISA assay. In one embodiment, VEGF is coated on the ELISA plate and then the bispecific antibody and HER2 with His tag are added. After washing the non-specific binding, the presence of HER2 is detected by an anti-His antibody followed by an HRP-conjugated secondary antibody. The formation of bispecific antibody is reflected by the ELISA signal since only the bispecific antibody is capable of binding HER2 and VEGF simultaneously with both arms.

The formation of bispecific antibody can also be assessed by analytical HPLC if there is a detectable difference in the biophysical properties of the two parental antibodies. A difference in pI may leads to two separate peaks for the two parental antibodies on Cation Exchange chromatography and the bispecific antibody may migrate as a peak in between. A difference in hydrophobicity may leads to two separate peaks for the two parental antibodies on hydrophobic interaction chromatography and the bispecific antibody may migrate as a peak in between. The analytical HPLC not only demonstrates the formation of bispecific antibody, but also allows the quantitation of percentage of bispecific antibody formed.

### Effects of Shielded HER2 x VEGF Bispecific Antibody on Binding and Functional Activity

A shielded HER2 x VEGF bispecific antibody can inhibit or block the capability of the Fab arm to bind to the respective antigens, HER2 and VEGF. The masking domains may reduce the maximum binding capacity of the shielded bispecific antibody in binding to the respective antigens. The masking domains may also reduce the binding affinity of the shielded bispecific antibody in binding to the respective antigens.

When the masking domains are cleaved off by protease, the shielded antibody is converted to an active bispecific antibody with the restoration of the capability of the antibody in binding to its antigen. The removal of masking domains from the shielded bispecific antibody can be realized by in vitro protease cutting assay using recombinant or purified protease. The removal of the masking domains from the shielded bispecific antibody can also be realized in vivo by proteases overexpressed in disease site. The removal of the masking domains can be assessed by comparing the molecular weight of heavy chain and light chain of shielded antibodies with the masking domain to the active antibody without the masking domain by SDS-PAGE, IEX, or HIC analyses.

In vitro binding and cell-based assays are well described in the art for use in determining a shielded bispecific antibody, active antibody and converted antibody after protease cleavage in binding to its antigen. For example, the binding of antibody may be determined by ELISA by immobilizing recombinant or purified antigen, sequestering the antibody with the immobilized antigen and determining the amount of bound antibody. This can also be performed using a Biacore® instrument for kinetic analysis of binding interactions. For cell-based binding assay, the binding of antibody may be determined by flow cytometry by incubating the antibody with cells expressing antigens on cell surface and determining the amount of antibody bound to cell surface antigen.

### Pharmaceutical Compositions

A shielded HER2 x VEGF bispecific antibody for use according to the present disclosure can be formulated in compositions, especially pharmaceutical compositions, for use in the methods herein. Such compositions comprise a therapeutically or prophylactically effective amount of the bispecific antibody described in this disclosure in mixture with a suitable carrier, e.g., a pharmaceutically acceptable agent. Typically, the bispecific antibody described in this disclosure is sufficiently purified for administration to an animal before formulation in a pharmaceutical composition.

Pharmaceutically acceptable agents include carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and surfactants.

The composition can be in liquid form or in a lyophilized or freeze-dried form and may include one or more lyoprotectants, excipients, surfactants, high molecular weight structural additives and/or bulking agents.

Compositions can be suitable for parenteral administration. Exemplary compositions are suitable for injection or infusion into an animal by any route available to the skilled worker, such as intraarticular, subcutaneous, intravenous, intramuscular, intraperitoneal, intracerebral (intraparenchymal), intracerebroventricular, intramuscular, intraocular, intraarterial, intralesional, intrarectal, transdermal, oral, and inhaled routes.

Pharmaceutical compositions described herein can be formulated for controlled or sustained delivery in a manner that provides local concentration of the product (e.g., bolus, depot effect) sustained release and/or increased stability or half-life in a particular local environment.

### Methods of Use

A shielded HER2 x VEGF bispecific antibody described herein is useful for the treatment or prevention of cancers, such as gastric, lung, and other cancers. In contrast to corresponding therapeutic antibodies, the shielded HER2 x VEGF bispecific antibody may have comparable efficacy in treating these diseases due to the conversion of the shielded antibody to active antibody specifically in disease sites by the removal of the shielding domain by proteases overexpressed in disease sites. However, the shielded antibody may have reduced systematic toxicity due to the masking of the antibody activity by the shielding domain in normal tissues that lack sufficient amounts of proteases needed to cleave off the masking domain. In short, the shielded bispecific antibody described herein may be as efficacious as the corresponding therapeutic antibody in treating diseases but with much improved safety profile. Due to the improved safety profile, increased levels of dosing comprising a shielded bispecific antibody may be administered to the patient with improved treatment efficacy.

In some embodiments, the present disclosure provides a method of treating or preventing a disease or condition, e.g., cancer, in a subject in need thereof, comprising administering to the subject a therapeutically effective amount of a shielded HER2 x VEGF bispecific antibody or a composition comprising a shielded HER2 x VEGF bispecific antibody of the present disclosure. The disclosure also provides a shielded HER2 x VEGF bispecific antibody or a composition comprising a shielded HER2 x VEGF bispecific antibody of the present disclosure for use in a method of treating or preventing a disease or condition, e.g., cancer, in a subject in need thereof, comprising administering to the subject a therapeutically effective amount of the bispecific antibody or the composition. The disclosure also provides use of a shielded HER2 x VEGF bispecific antibody or a composition comprising a shielded HER2 x VEGF bispecific antibody of the present disclosure in the manufacture of a medicament for treating or preventing a disease or condition, e.g., cancer, comprising administering to the subject a therapeutically effective amount of the bispecific antibody or the composition. Exemplary cancers include, but are not limited to: multiple myeloma, non-small cell lung cancer, acute myeloid leukemia, female breast cancer, pancreatic cancer, colorectal cancer and peritoneum cancer.

All combinations of the various elements described herein are within the scope of the disclosure unless otherwise indicated herein or otherwise clearly contradicted by context.

This disclosure will be better understood from the following Experimental Details. However, one skilled in the art will readily appreciate that the specific methods and results discussed are merely illustrative of the disclosure.

### Example 1: Generation of HER2 x VEGF Bispecific Antibodies

A capped anti-HER2 and capped anti-VEGF antibody were employed to evaluate the feasibility of preparing a bispecific antibody. Heavy chain and light chain constructs expressing anti-HER2 and anti-VEGF shielded parental mAbs were prepared. Plasmids encoding heavy chains and light chains of these anti-HER2 and anti-VEGF masked antibodies were co-transfected into Expi293F cells following the transfection kit instructions (Thermo Scientific). Cells were spun down five days post transfection, and the supernatant were passed through a 0.2 μm filter. The purifications of expressed masked antibody supernatants were carried out by affinity chromatography over protein A agarose columns (GE Healthcare Life Sciences). The purified masked antibodies were buffer-exchanged into DPBS, pH7.2 by dialysis, and protein concentrations were determined by UV absorbance at 280 nm.

For controlled Fab-arm exchange, equal molar amounts of both parental antibodies were mixed together and reduced for 5 hours in the presence of 75 mM 2-mercaptoethylamine (2-MEA). The reaction mixture was dialyzed against DPBS to allow the bispecific antibody formation.

As controls, the corresponding anti-HER2 parental antibodies without the masking domain listed in Table 2 and anti-VEGF parental antibodies without the masking domain listed in Table 3 were also prepared. These parental antibodies were employed to prepare anti-HER2 and anti-VEGF bispecific antibodies listed in Table 4.

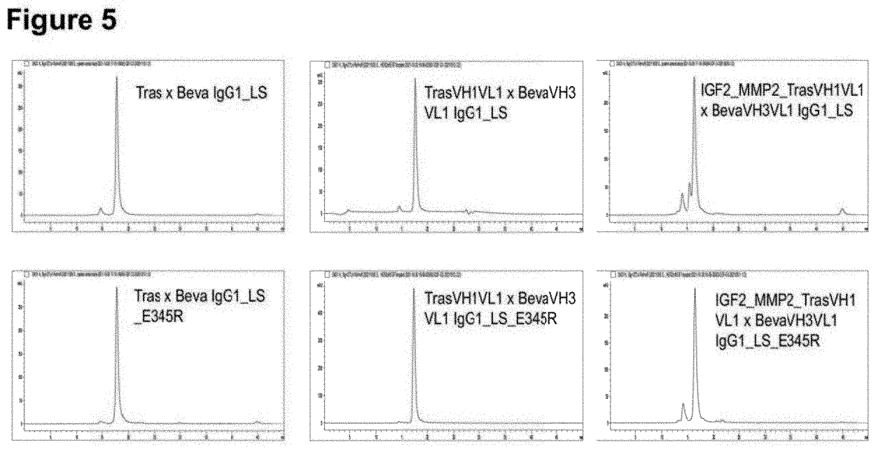

Exemplary shielded anti-HER2 and anti-VEGF bispecific antibodies and their corresponding anti-HER2 and anti-VEGF bispecific antibodies without the masking domain were subjected to Size Exclusion Chromatography (SEC) analysis by loading 20 μg of antibodies onto AdvanceBio SEC300 column (Agilent). The profiles of peak migration for these antibodies were shown in FIG. 5. All shielded and unshielded HER2 x VEGF bispecific antibodies showed a dominant major monomer peak with minor aggregation fraction especially for the shielded antibodies. Both IGF2_MMP2_TrasVH1VL1x BevaVH3VL1_IgG1_LS and IGF2_MMP2_TrasVH1VL1x BevaVH3VL1_IgG1_LS_E345R antibodies have the IGF2 masking domains fused to the N-terminus of antibody heavy chains and light chains with the substrate sequences for MMP2. Judging from the retention time of the major protein peaks, both shielded bispecific antibodies have a slightly larger molecular weight than the corresponding bispecific antibodies without the masking domain due to the presence of the IGF2 masking domain with a molecular weight of about 10 kDa.

### Example 2: Confirmation of Formation of HER2 x VEGF Bispecific Antibody

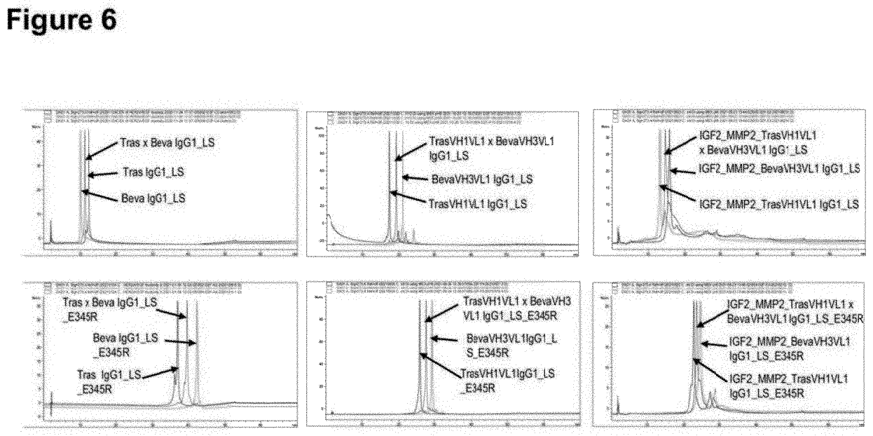

The formation of shielded HER2 x VEGF bispecific antibodies and their corresponding HER2 x VEGF antibodies without the masking domain were assessed by Cation Exchange (CEX) chromatography. 20 μg of bispecific antibodies and their corresponding parental antibodies were loaded onto Bio SCX NP5 ion exchange column (Agilent). The profiles of peak migration for these antibodies were shown in FIG. 6. In all the cases, the bispecific antibody migrated as a major protein peak with the retention time in between the migrated major peaks of the two parental antibodies, indicating the formation of bispecific antibodies. Further calculation of the area under the curve (AUC) indicated that over 90% of the parental antibodies formed the bispecific antibodies by the Fab-arm exchange (FIG. 6).

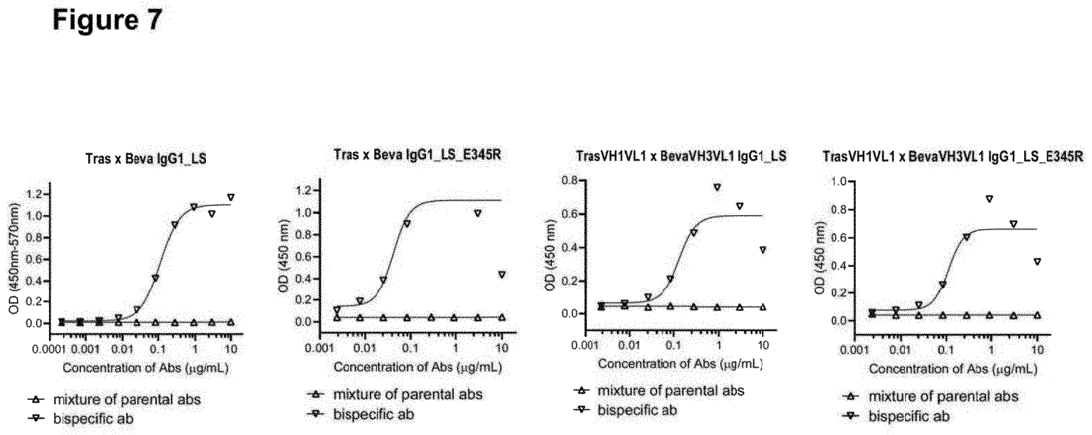

ELISA-based binding assays were also employed to evaluate the formation of HER2 x VEGF bispecific antibodies without the masking domain. In this assay, human VEGF was coated on the plate and then the HER2 x VEGF bispecific antibody or a mixture of HER2 antibody and VEGF antibody, along with recombinant HER2 with a His tag were added. After washing off the non-specific binding, the presence of HER2 was detected by an HRP-conjugated anti-his secondary antibody. It was observed that the HER2 x VEGF bispecific antibody, but not by a mixture of the two parental antibodies, dose-dependently recruited HER2 (FIG. 7). This data suggested the formation of bispecific antibody which is capable of binding HER2 and VEGF simultaneously with both arms.

### Example 3: Digestion of Shielded HER2 x VEGF Bispecific Antibodies with IGF2-Based Masking Domain with Protease

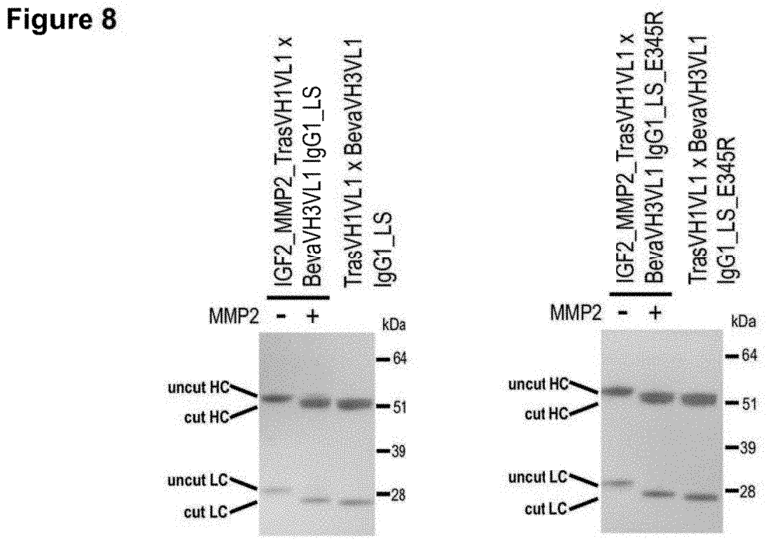

In vitro protease cutting assays were set up to evaluate whether the IGF2-based masking domain can be removed from the shielded HER2 x VEGF bispecific antibodies by proteases. For protease MMP2, recombinant human MMP2 was activated by incubating with p-aminophenylmercuric acetate (APMA) according to manufacturer's instruction (R&D Systems). 10 μg of shielded HER2 x VEGF bispecific antibodies were incubated with 50 ng of activated MMP2 overnight at 37° C. The digestions of the shielded antibodies were evaluated by SDS-PAGE under the reduced condition as shown in FIG. 8.

It was observed that all the shielded antibodies had heavy chains and light chains with the molecular weights slightly bigger than the respective unshielded antibodies because of the fusion of the IGF2-based masking domain with 10 kDa molecular weight to the native antibody heavy chain and light chain, respectively. By MMP2 digestion, the molecular weights of the heavy chain and light chain for the digested shielded HER2 x VEGF bispecific antibodies became smaller relative to the corresponding undigested shielded antibodies, with size comparable to corresponding HER2 x VEGF bispecific antibodies without the IGF2-based masking domain (FIG. 8). This indicated the efficient cleavage of the MMP2-cleavable linker sequences of the shielded HER2 x VEGF bispecific antibodies by MMP2 and the release of the IGF2 masking domain.

### Example 4: Binding to HER2 by HER2 x VEGF Bispecific Antibodies

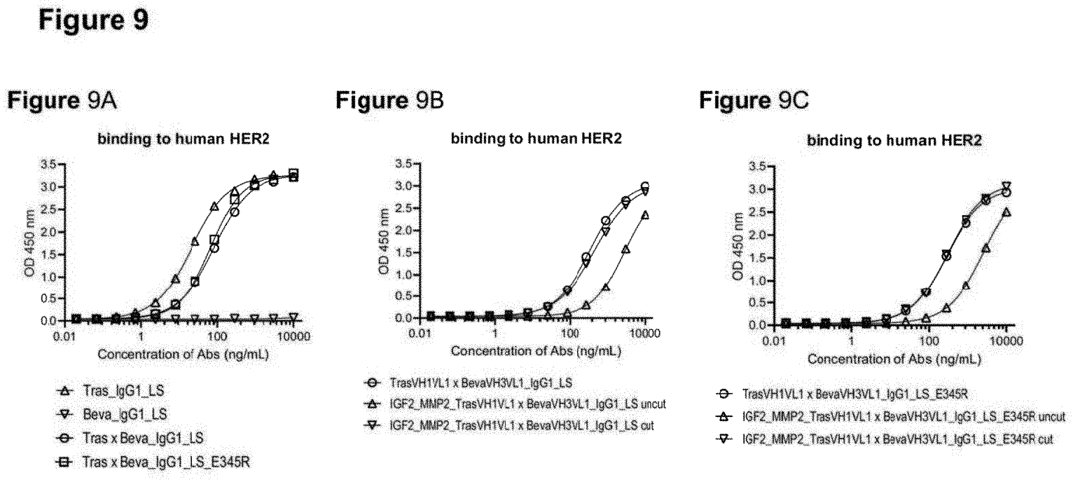

ELISA-based binding assays were employed to evaluate the binding to HER2 by exemplary HER2 x VEGF bispecific antibodies with or without IGF2 masking domain (FIG. 9). In these assays, 1 μg/mL recombinant human HER2 (R&D systems) were coated on ELISA plate. Increasing concentrations of exemplary HER2 x VEGF bispecific antibodies were applied on the plate and their bindings to the recombinant human HER2 were detected by HRP-conjugated anti-human IgG secondary antibody.

It was observed that anti-HER2 antibody Tras_IgG1_LS and HER2 x VEGF bispecific antibodies Tras x Beva IgG1_LS and Tras x Beva IgG1_LS_E345R dose-dependently bound to HER2 while anti-VEGF antibody Beva_IgG1_LS did not (FIG. 9A). In FIGS. 9B and 9C, the shielded HER2 x VEGF bispecific antibodies, IGF2_MMP2_TrasVH1VL1 x BevaVH3VL1_IgG1_LS uncut and IGF2_MMP2_TrasVH1VL1 x BevaVH3VL1 IgG1_LS_E345R uncut, showed much reduced potency in binding to HER2 relative to the corresponding HER2 x VEGF bispecific antibodies without the IGF2 masking domain TrasVH1VL1 x BevaVH3VL1_IgG1_LS and TrasVH1VL1 x BevaVH3VL1_IgG1_LS_E345R. Upon cut by MMP2 protease, the shielded antibodies with the IGF2 masking domain removed, IGF2_MMP2_TrasVH1VL1 x BevaVH3VL1_IgG1_LS cut and IGF2_MMP2_TrasVH1VL1 x BevaVH3VL1_IgG1_LS_E345R cut, showed restored HER2 binding potency comparable to HER2 x VEGF bispecific antibodies without the IGF2 masking domain.

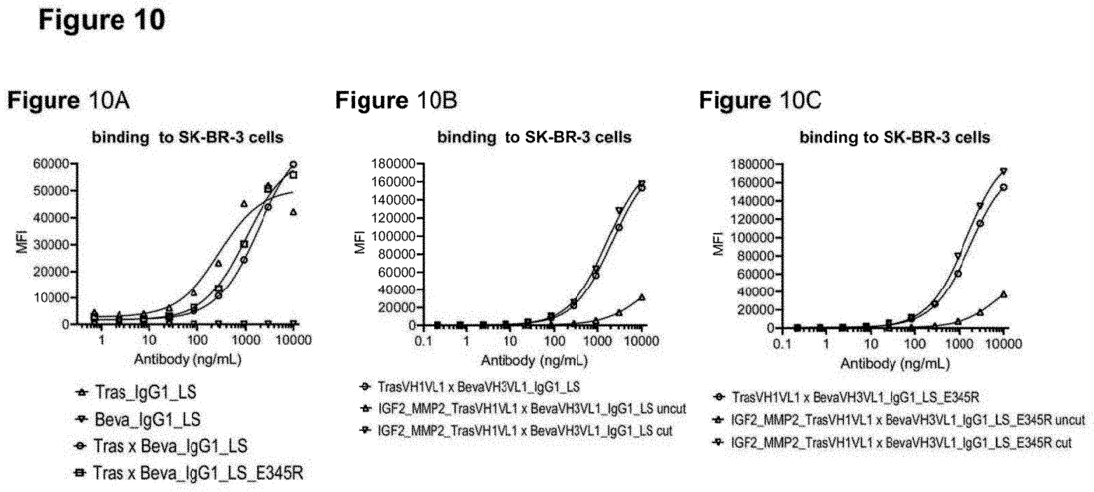

Flow cytometry-based binding assays were also employed to evaluate the binding to HER2 expressed on cell surface by exemplary HER2 x VEGF bispecific antibodies with or without IGF2 masking domain (FIG. 10). In these assays, increasing concentrations of exemplary HER2 x VEGF bispecific antibodies were applied to HER2-expressing SK-BR-3 cells and their bindings to cell surface HER2 were detected by PE-conjugated anti-human secondary antibody by MacsQuant flow cytometer.

It was observed that anti-HER2 antibody Tras_IgG1_LS and HER2 x VEGF bispecific antibodies Tras x Beva IgG1_LS and Tras x Beva IgG1_LS_E345R dose-dependently bound to cell surface HER2 while anti-VEGF antibody Beva_IgG1_LS did not (FIG. 10A). The shielded HER2 x VEGF bispecific antibodies, IGF2_MMP2_TrasVH1VL1 x BevaVH3VL1 IgG1_LS uncut and IGF2_MMP2_TrasVH1VL1 x BevaVH3VL1_IgG1_LS_E345R uncut, showed much reduced potency in binding to HER2 relative to the corresponding HER2 x VEGF bispecific antibodies without the IGF2 masking domain TrasVH1VL1 x BevaVH3VL1_IgG1_LS and TrasVH1VL1 x BevaVH3VL1_IgG1_LS_E345R (FIGS. 10B and 10C). Upon cut by MMP2 protease, the shielded antibodies with the IGF2 masking domain removed, IGF2_MMP2_TrasVH1VL1 x BevaVH3VL1_IgG1_LS cut and IGF2_MMP2_TrasVH1VL1 x BevaVH3VL1_IgG1_LS_E345R cut, showed restored comparable HER2 binding potency to HER2 x VEGF bispecific antibodies without the IGF2 masking domain.

### Example 5: Inhibition of HER2+ Cancer Cell Proliferation by HER2 x VEGF Bispecific Antibody

In vitro cell proliferation assays were set up to evaluate HER2 x VEGF bispecific antibodies in inhibition of the proliferation of HER2-expressing cells, such as breast cancer cell line SK-BR-3. Increasing amounts of test molecules were applied to the SK-BR-3 cancer cells for six days and their effects on cell viability were assessed by Celltiter-Glo luminescent cell viability assay kit (Promega).

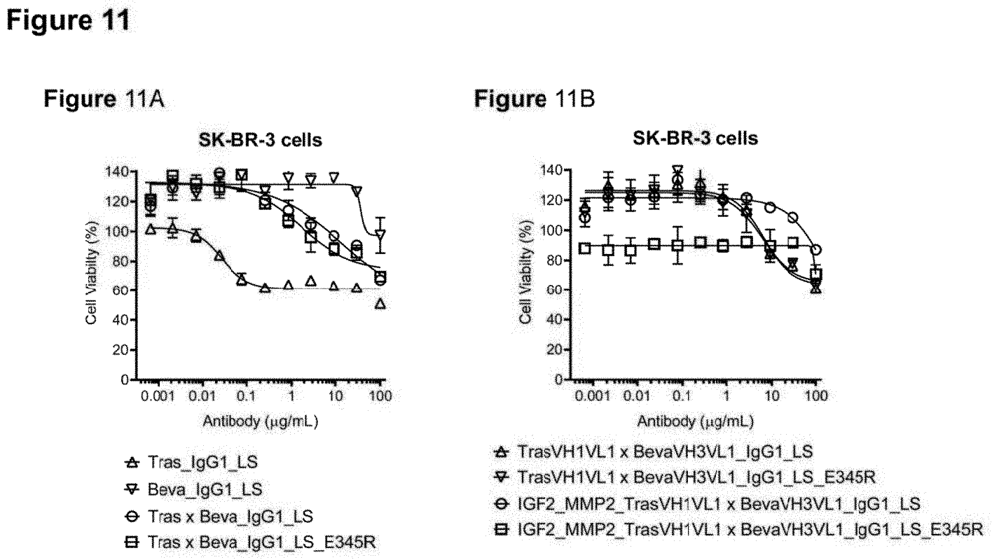

It was observed that anti-HER2 antibody Tras_IgG1_LS dose-dependently inhibited SK-BR-3 cell viability while the anti-VEGF antibody Beva_IgG1_LS did not. The HER2 x VEGF bispecific antibodies, Tras x Beva IgG1_LS and Tras x Beva IgG1_LS_E345R, also dose-dependently inhibited SK-BR-3 cell proliferation but with much less potency than anti-HER2 antibody Tras_IgG1_LS (FIG. 11A). The shielded HER2 x VEGF bispecific antibodies, IGF2_MMP2_TrasVH1VL1 x BevaVH3VL1_IgG1_LS and IGF2_MMP2_TrasVH1VL1 x BevaVH3VL1_IgG1_LS_E345R, showed insignificant inhibition of SK-BR-3 cell proliferation while their corresponding HER2 x VEGF bispecific antibodies without the IGF2 masking domain, TrasVH1VL1 x BevaVH3VL1_IgG1_LS and TrasVH1VL1 x BevaVH3VL1 IgG1_LS_E345R, showed dose-dependent inhibition (FIG. 11B).

### Example 6: ADCC Activities of HER2 x VEGF Bispecific Antibodies

An ADCC reporter assay (Invivogen) was set up to assess HER2 x VEGF bispecific antibodies in the activation of ADCC. Increasing amounts of HER2 x VEGF bispecific antibodies were incubated with ADCC reporter cells along with HER2-expressing SK-BR-3 cells. The antibody-mediated ADCC activity was measured by luciferase gene expression in ADCC reporter cells (Invivogen).

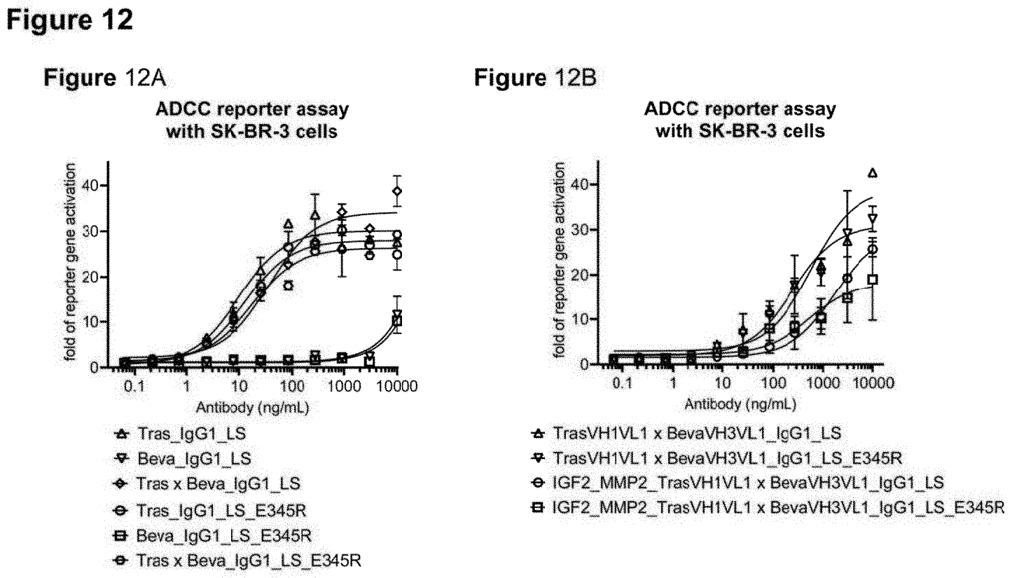

It was observed that anti-HER2 antibodies, Tras_IgG1_LS and Tras_IgG1_LS_E345R, dose-dependently activated reporter gene expression in ADCC reporter cells while the anti-VEGF antibodies, Beva_IgG1_LS and Beva_IgG1_LS_E345R, did not (FIG. 12A). The HER2 x VEGF bispecific antibodies, Tras x Beva IgG1_LS and Tras x Beva IgG1_LS_E345R, also dose-dependently activated reporter gene expression with potency similar to those anti-HER2 antibodies. The antibodies with E345R Fc mutation did not show significantly improved ADCC activities than the antibodies without such clustering mutation (FIG. 12A). The shielded HER2 x VEGF bispecific antibodies, IGF2_MMP2_TrasVH1VL1 x BevaVH3VL1_IgG1_LS and IGF2_MMP2_TrasVH1VL1 x BevaVH3VL1_IgG1_LS_E345R, showed much less potency in the activation of ADCC than their corresponding HER2 x VEGF bispecific antibodies without the IGF2 masking domain, TrasVH1VL1 x BevaVH3VL1 IgG1_LS and TrasVH1VL1 x BevaVH3VL1_IgG1_LS_E345R. (FIG. 12B).

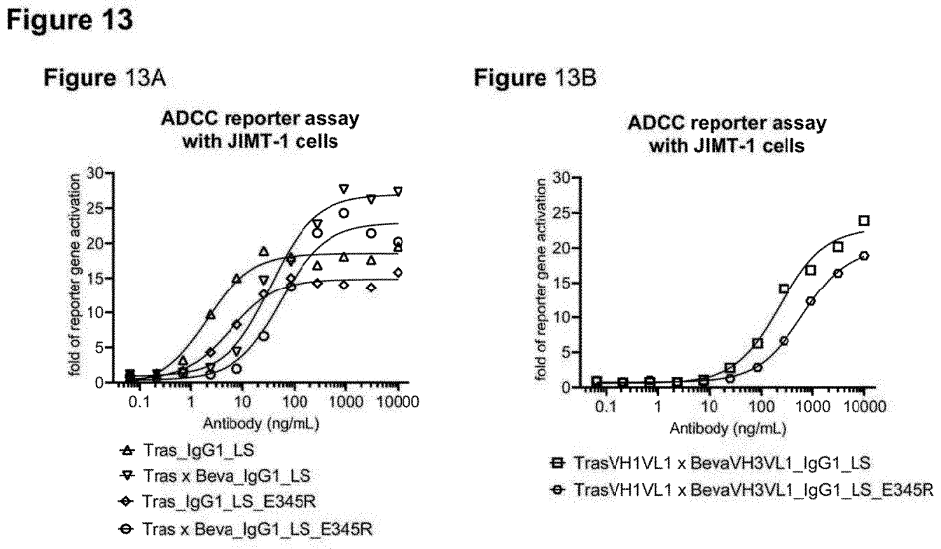

Besides SK-BR-3 cells with high HER2 expression, the effects of HER2 x VEGF bispecific antibodies in the activation of ADCC were also assessed with JIMT-1 cells with low HER2 expression. The anti-HER2 antibodies, Tras_IgG1_LS and Tras_IgG1_LS_E345R, showed much potent but less efficacious ADCC activation activities than the HER2 x VEGF bispecific antibodies, Tras x Beva IgG1_LS and Tras x Beva IgG1_LS_E345R (FIG. 13A). Another set of exemplary HER2 x VEGF bispecific antibodies, TrasVH1VL1 x BevaVH3VL1_IgG1_LS and TrasVH1VL1 x BevaVH3VL1_IgG1_LS_E345R, also showed dose-dependent ADCC activities on JIMT-1 cells (FIG. 13B).

Besides ADCC reporter assay, a NK-cell mediated cell killing assay was also set up to assess the ADCC activities of HER2 x VEGF bispecific antibodies. Increasing amounts of HER2 x VEGF bispecific antibodies were incubated with NK cells along with HER2-expressing cells such as SK-BR-3 cells and JIMT-1 cells labelled by CFSE fluorescent dye (Abcam). The antibody-mediated ADCC activity was measured by the killing of labelled target cells by flow cytometry.

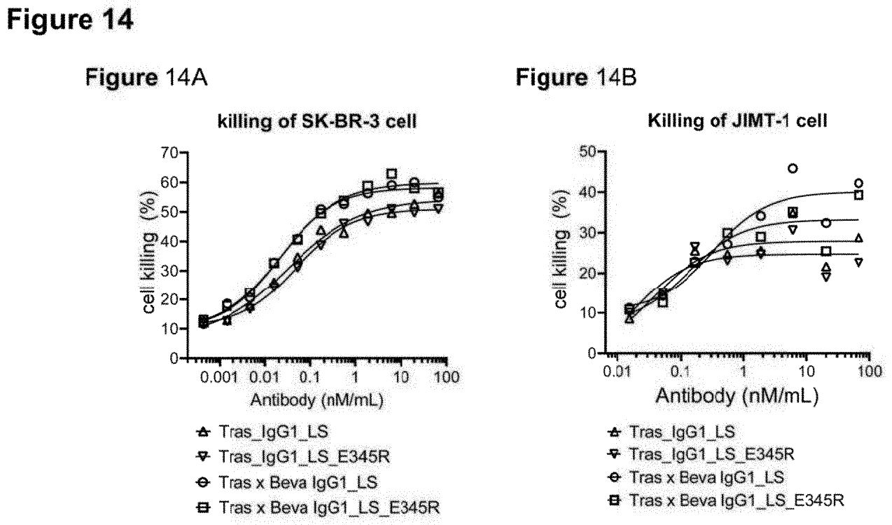

Both anti-HER2 antibodies and HER2 x VEGF bispecific antibodies mediated dose-dependent killing of SK-BR-3 cells or JIMT-1 cells by NK cells (FIGS. 14A and 14B), although the killing of SK-BR-3 cells with high HER2 expression is much more efficient than the killing of JIMT-1 cells with low HER2 expression. For SK-BR-3 cells, the HER2 x VEGF bispecific antibodies, Tras x Beva IgG1_LS and Tras x Beva_IgG1_LS_E345R, showed slightly higher potency and efficacy in mediating cell killing than the anti-HER2 antibodies Tras_IgG1_LS and Tras_IgG1_LS_E345R (FIG. 14A). In contrast, the anti-HER2 antibodies showed slightly better potency but less efficacious in mediating the killing of JIMT-1 cells than the HER2 x VEGF bispecific antibodies (FIG. 14B).

### Example 7: ADCP Activities of HER2 x VEGF Bispecific Antibodies

An ADCP reporter assay (Invivogen) was set up to assess HER2 x VEGF bispecific antibodies in the activation of ADCP. Increasing amounts of HER2 x VEGF bispecific antibodies were incubated with ADCP reporter cells along with HER2-expressing SK-BR-3 cells. The antibody-mediated ADCP activity was measured by luciferase gene expression in ADCP reporter cells (Invivogen).

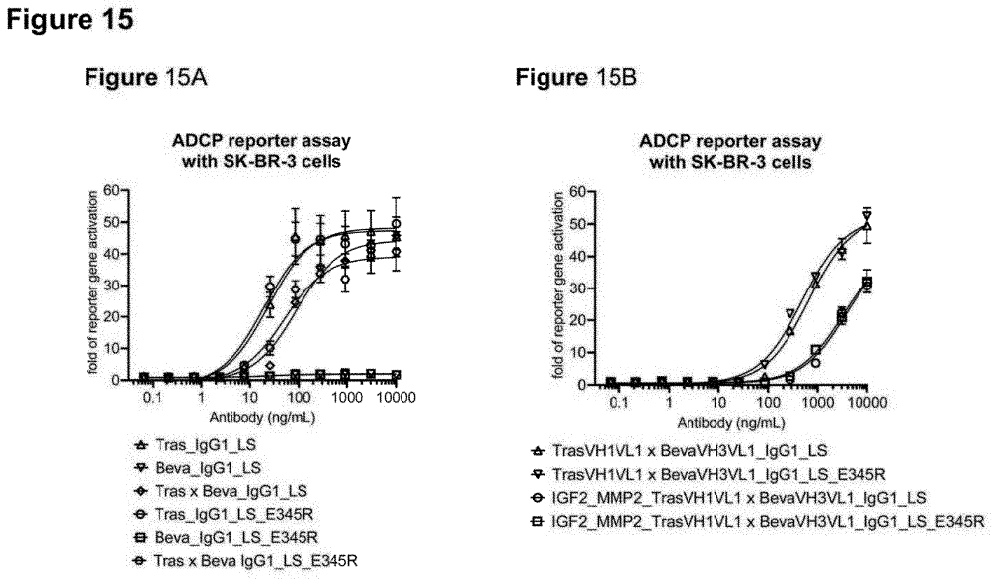

It was observed that anti-HER2 antibodies, Tras_IgG1_LS and Tras_IgG1_LS_E345R, dose-dependently activated reporter gene expression in ADCP reporter cells while the anti-VEGF antibodies, Beva_IgG1_LS and Beva_IgG1_LS_E345R, did not (FIG. 15A). The HER2 x VEGF bispecific antibodies, Tras x Beva IgG1_LS and Tras x Beva IgG1_LS_E345R, also dose-dependently activated reporter gene expression, although with less potency than those anti-HER2 antibodies. The antibodies with E345R Fc mutation did not show significantly improved ADCP activities than the antibodies without such clustering mutation (FIG. 15A). The shielded HER2 x VEGF bispecific antibodies, IGF2_MMP2_TrasVH1VL1 x BevaVH3VL1_IgG1_LS and IGF2_MMP2_TrasVH1VL1 x BevaVH3VL1_IgG1_LS_E345R, showed much less potency in the activation of ADCP than their corresponding HER2 x VEGF bispecific antibodies without the IGF2 masking domain, TrasVH1VL1 x BevaVH3VL1 IgG1_LS and TrasVH1VL1 x BevaVH3VL1_IgG1_LS_E345R. (FIG. 15B).

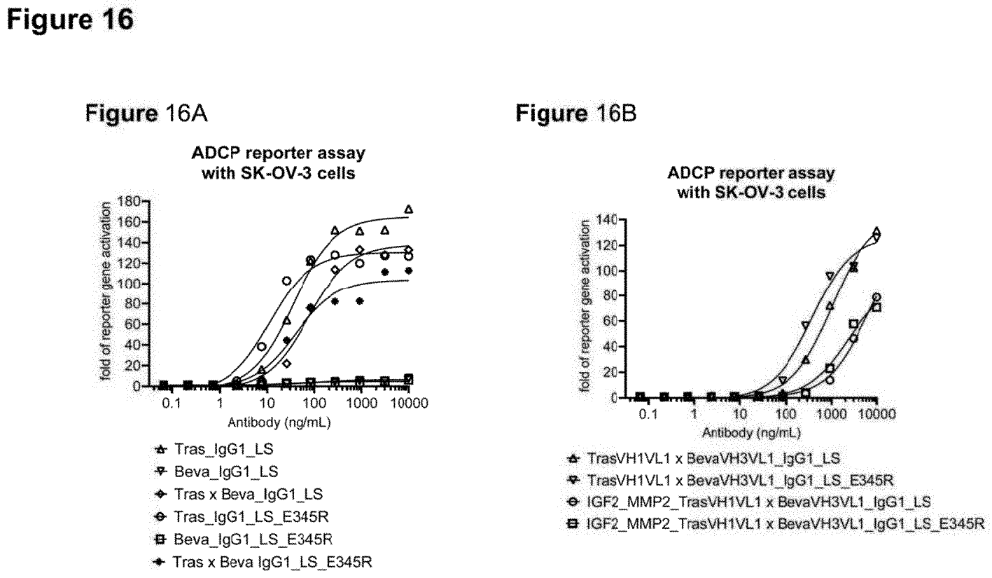

Besides SK-BR-3 cells with high HER2 expression, the effects of HER2 x VEGF bispecific antibodies in the activation of ADCP were also assessed with SK-OV-3 cells also with relatively high HER2 expression. The effects of HER2 x VEGF bispecific antibodies in mediating ADCP activation were similar between SK-OV-3 cells and SK-BR-3 cells, except that the antibodies with E345R mutations showed slightly better potencies in activating ADCP with SK-OV-3 cells than the antibodies without such clustering mutations (FIGS. 16A and 16B).

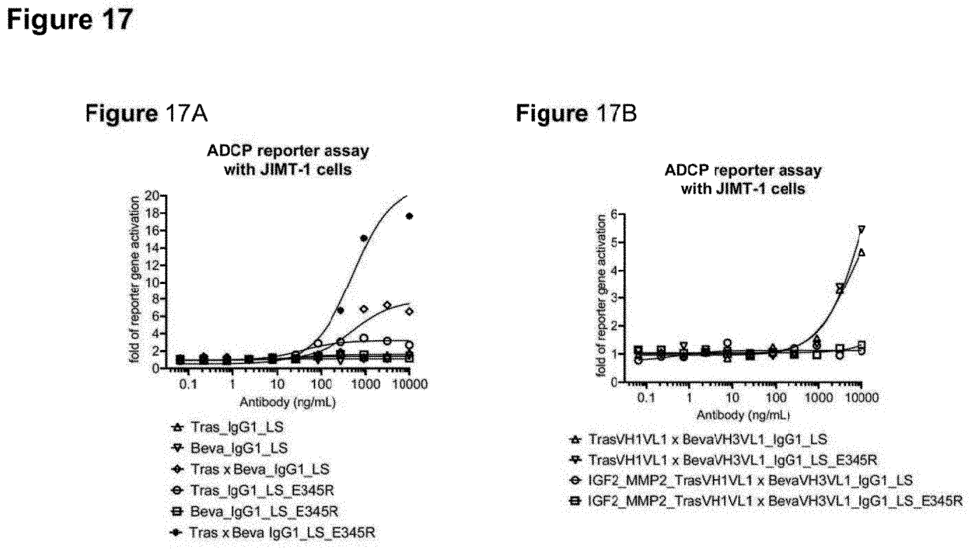

ADCP reporter assays were also assessed with JIMT-1 cells with low HER2 expression. While the anti-HER2 antibody Tras_IgG1_LS showed no ADCP activity, the anti-HER2 antibody with E345R Fc mutation, Tras_IgG1_LS_E345R, could mediate low ADCP activity to JIMT-1 cells (FIG. 17A). In contrast, the HER2 x VEGF bispecific antibody Tras x Beva IgG1_LS showed certain ADCP activity and its counterpart with the E345R mutation, Tras x Beva_IgG1_LS_E345R, showed significantly higher ADCP activity to JIMT-1 cells (FIG. 17A). This data revealed more significant ADCP enhancing effect for E345R clustering mutation on cells with low HER2 expression than those with high HER2 expression. Another set of exemplary HER2 x VEGF bispecific antibodies, TrasVH1VL1 x BevaVH3VL1_IgG1_LS and TrasVH1VL1 x BevaVH3VL1_IgG1_LS_E345R, also showed dose-dependent ADCP activities on JIMT-1 cells while their corresponding shielded HER2 x VEGF bispecific antibodies, IGF2_MMP2_TrasVH1VL1 x BevaVH3VL1_IgG1_LS and IGF2_MMP2_TrasVH1VL1 x BevaVH3VL1_IgG1_LS_E345R, showed no ADCP activities on JIMT-1 cells (FIG. 17B).

### Example 8: CDC Activities of HER2 x VEGF Bispecific Antibodies

A complement dependent cytotoxicity (CDC) assay was set up to assess HER2 x VEGF bispecific antibodies in the activation of CDC on target cells. Increasing amounts of HER2 x VEGF bispecific antibodies were incubated with baby rabbit complement (Cedarlane Lab) along with HER2-expressing BT-474 cells. The antibody-mediated killing of BT-474 cells by the activated complement was measured by CyQUANT LDH cytotoxicity assay kit (Invitrogen).

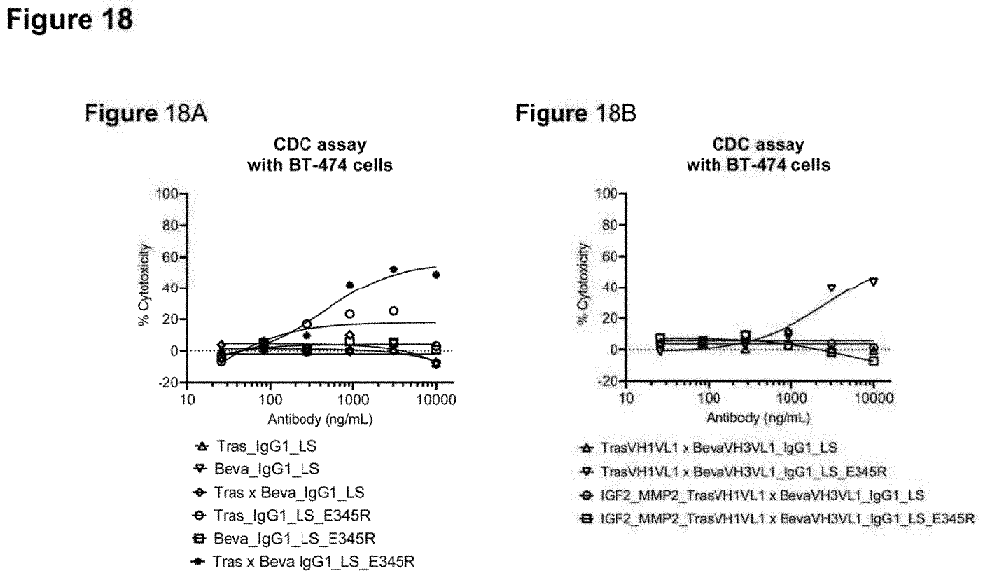

While the anti-HER2 antibody Tras_IgG1_LS and anti-VEGF antibodies showed no CDC activity to BT-474 cells, the anti-HER2 antibody with E345R Fc mutation, Tras_IgG1_LS_E345R, mediated certain CDC activity to BT-474 cells (FIG. 18A). In contrast, the HER2 x VEGF bispecific antibody with the E345R mutation, Tras x Beva_IgG1_LS_E345R, showed even higher CDC activity while its counterpart without the E345R mutation, Tras x Beva IgG1_LS, showed no CDC activity to BT-474 cells (FIG. 18A). Another set of exemplary HER2 x VEGF bispecific antibody with E345R mutation, TrasVH1VL1 x BevaVH3VL1 IgG1_LS_E345R also showed CDC activity to BT-474 cells while its counterpart without the E345R mutation showed no CDC activity (FIG. 18B). This data revealed a significant CDC initiation effect for the E345R clustering mutation on HER2 x VEGF bispecific antibodies. On the other hand, the shielded HER2 x VEGF bispecific antibodies, IGF2_MMP2_TrasVH1VL1 x BevaVH3VL1_IgG1_LS_E345R, showed no CDC activity compared with its corresponding HER2 x VEGF bispecific antibody without the IGF2 masking domain TrasVH1VL1 x BevaVH3VL1_IgG1_LS_E345R (FIG. 18B).

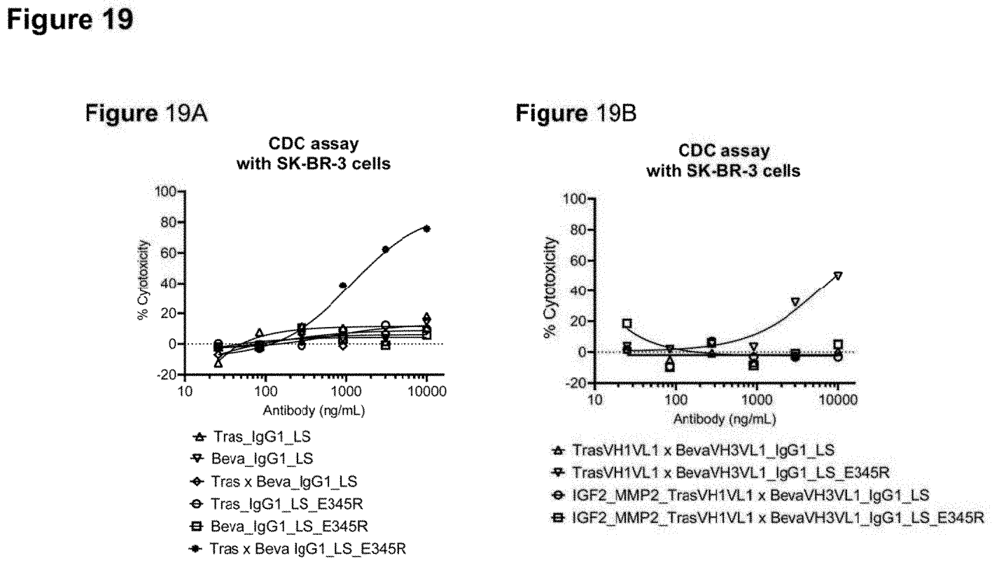

Besides BT-474 cells, the CDC activities of HER2 x VEGF bispecific antibodies were also assessed on SK-BR-3 cells with high HER2 expression. Similarly, the E345R Fc mutation enabled HER2 x VEGF bispecific antibodies with CDC activities to SK-BR-3 cells (FIGS. 19A and 19B).

### Example 9: Binding to VEGF by HER2 x VEGF Bispecific Antibodies

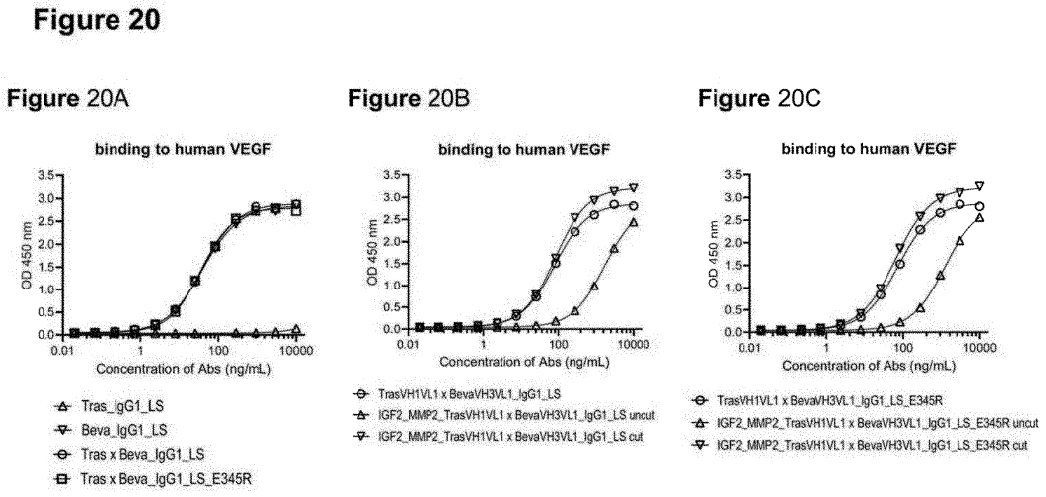

ELISA-based binding assays were employed to evaluate the binding to VEGF by exemplary HER2 x VEGF bispecific antibodies with or without IGF2 masking domain (FIG. 20). In these assays, 1 μg/mL recombinant human VEGF (R&D systems) were coated on the ELISA plate. Increasing concentrations of exemplary HER2 x VEGF bispecific antibodies were applied on the plate and their bindings to the recombinant human VEGF were detected by HRP-conjugated anti-human IgG secondary antibody.

It was observed that anti-VEGF antibody Beva_IgG1_LS and HER2 x VEGF bispecific antibodies Tras x Beva IgG1_LS and Tras x Beva_IgG1_LS_E345R dose-dependently bound to VEGF while anti-HER2 antibody Tras_IgG1_LS did not (FIG. 20A). In FIGS. 20B and 20C, the shielded HER2 x VEGF bispecific antibodies, IGF2_MMP2_TrasVH1VL1 x BevaVH3VL1_IgG1_LS uncut and IGF2_MMP2_TrasVH1VL1 x BevaVH3VL1 IgG1_LS_E345R uncut, showed much reduced potency in binding to VEGF relative to the corresponding HER2 x VEGF bispecific antibodies without the IGF2 masking domain TrasVH1VL1 x BevaVH3VL1_IgG1_LS and TrasVH1VL1 x BevaVH3VL1_IgG1_LS_E345R. Upon cut by MMP2 protease, the shielded antibodies with the IGF2 masking domain removed, IGF2_MMP2_TrasVH1VL1 x BevaVH3VL1_IgG1_LS cut and IGF2_MMP2_TrasVH1VL1 x BevaVH3VL1_IgG1_LS_E345R cut, showed restored VEGF binding potency comparable to HER2 x VEGF bispecific antibodies without the IGF2 masking domain.

### Example 10: Neutralization of VEGF Secreted from HER2-Expressing Cancer Cells by HER2 x VEGF Bispecific Antibodies

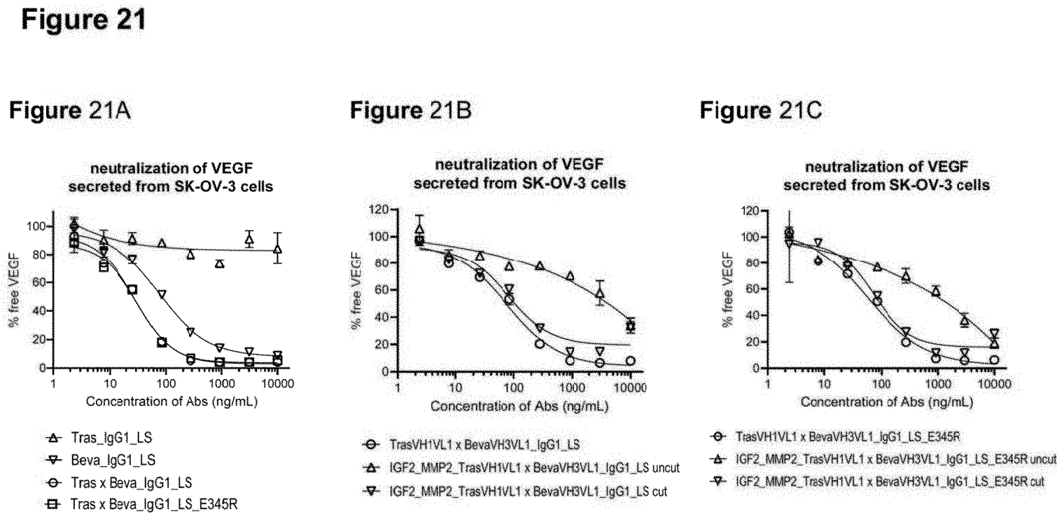

HER2 positive cancer cells secrete VEGF to promote angiogenesis for the survival of cancer cells. ELISA-based binding assays were employed to evaluate the neutralization of secreted VEGF from cancer cells by exemplary HER2 x VEGF bispecific antibodies with or without IGF2 masking domain (FIG. 21). In these assays, increasing concentrations of exemplary HER2 x VEGF bispecific antibodies were applied to SK-BR-3 cells for three days and the remaining free VEGF not neutralized by antibodies were quantitated by VEGF quantitation kit (R&D System).

It was observed that anti-VEGF antibody Beva_IgG1_LS and HER2 x VEGF bispecific antibodies Tras x Beva IgG1_LS and Tras x Beva_IgG1_LS_E345R dose-dependently neutralized secreted VEGF while anti-HER2 antibody Tras_IgG1_LS did not (FIG. 21A). The potencies of neutralization by the bispecific antibodies were better than the anti-VEGF antibody.

In FIGS. 21B and 21C, the shielded HER2 x VEGF bispecific antibodies, IGF2_MMP2_TrasVH1VL1 x BevaVH3VL1_IgG1_LS uncut and IGF2_MMP2_TrasVH1VL1 x BevaVH3VL1_IgG1_LS_E345R uncut, showed much reduced potency in the neutralization of secreted VEGF relative to the corresponding HER2 x VEGF bispecific antibodies without the IGF2 masking domain TrasVH1VL1 x BevaVH3VL1_IgG1_LS and TrasVH1VL1 x BevaVH3VL1_IgG1_LS_E345R. Upon cut by MMP2 protease, the shielded antibodies with the IGF2 masking domain removed, IGF2_MMP2_TrasVH1VL1 x BevaVH3VL1_IgG1_LS cut and IGF2_MMP2_TrasVH1VL1 x BevaVH3VL1_IgG1_LS_E345R cut, showed restored potency in VEGF neutralization comparable to HER2 x VEGF bispecific antibodies without the IGF2 masking domain.

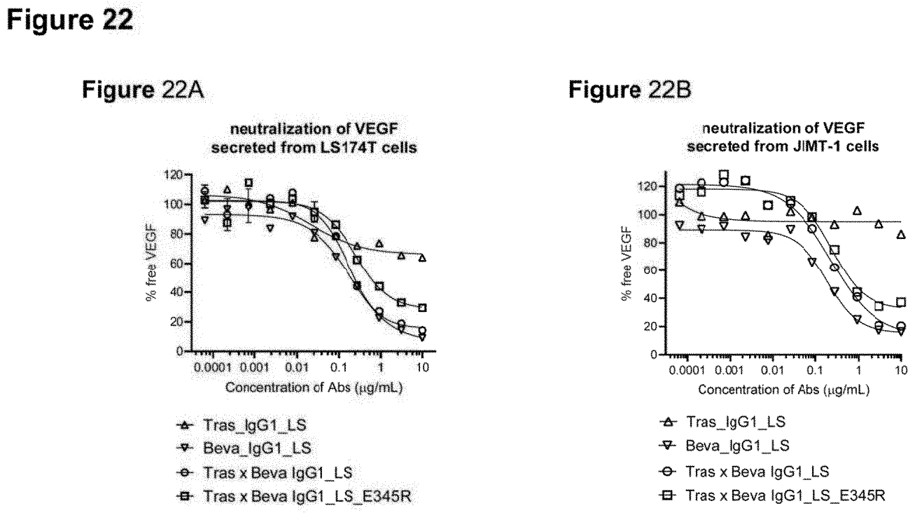

Besides SK-BR-3 cells, neutralization of VEGF secreted from two other HER2 positive cells, LS174T and JIMT-1 cells, were also assessed in similar assays. It was observed that anti-VEGF antibody Beva_IgG1_LS and HER2 x VEGF bispecific antibodies Tras x Beva IgG1_LS and Tras x Beva_IgG1_LS_E345R dose-dependently neutralized VEGF secreted from LS174T cells (FIG. 22A) and JIMT-1 cells (FIG. 22B), while anti-HER2 antibody Tras_IgG1_LS did not.

### Example 11: Blockade of VEGF Signalling by HER2 x VEGF Bispecific Antibodies

VEGF binds to its receptors and activates the signal transduction pathways leading to many cellular responses including angiogenesis. A VEGF reporter assay (Promega) was adopted to assess the blockade of VEGF signalling by exemplary HER2 x VEGF bispecific antibodies with or without IGF2 masking domain. In these assays, increasing concentrations of exemplary HER2 x VEGF bispecific antibodies were incubated with 25 ng/mL VEGF along with the VEGF reporter cell line. The blockade of luciferase reporter gene was quantitated by Bio-Glo luminescence kit (Promega).

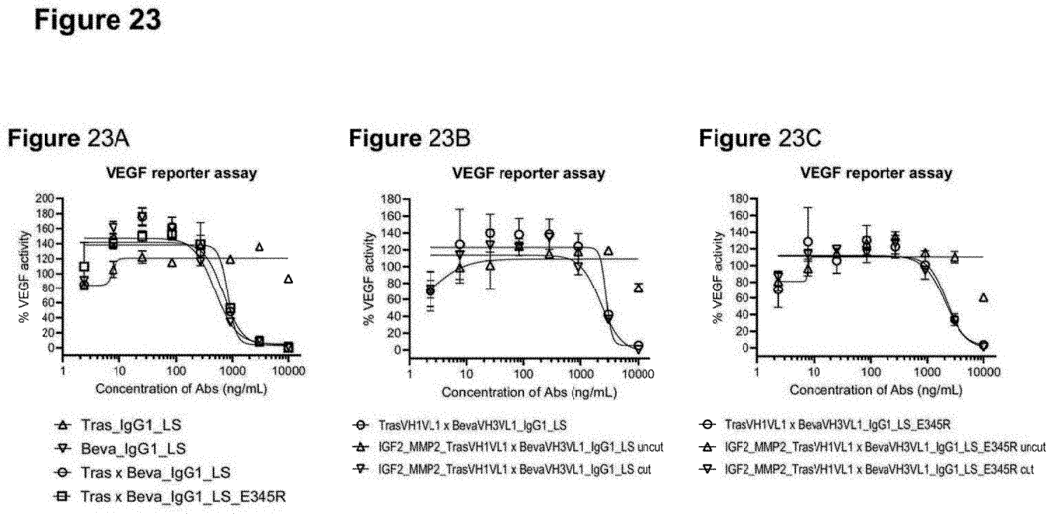

It was observed that anti-VEGF antibody Beva_IgG1_LS and HER2 x VEGF bispecific antibodies Tras x Beva IgG1_LS and Tras x Beva_IgG1_LS_E345R dose-dependently blocked VEGF signaling while anti-HER2 antibody Tras_IgG1_LS did not (FIG. 23A). The potencies of blockade by the bispecific antibodies were similar to the anti-VEGF antibody. In FIGS. 23B and 23C, the shielded HER2 x VEGF bispecific antibodies, IGF2_MMP2_TrasVH1VL1 x BevaVH3VL1_IgG1_LS uncut and IGF2_MMP2_TrasVH1VL1 x BevaVH3VL1_IgG1_LS_E345R uncut, showed insignificant potency in the blockade of VEGF signaling relative to the corresponding HER2 x VEGF bispecific antibodies without the IGF2 masking domain TrasVH1VL1 x BevaVH3VL1_IgG1_LS and TrasVH1VL1 x BevaVH3VL1 IgG1_LS_E345R. Upon cut by MMP2 protease, the shielded antibodies with the IGF2 masking domain removed, IGF2_MMP2_TrasVH1VL1 x BevaVH3VL1_IgG1_LS cut and IGF2_MMP2_TrasVH1VL1 x BevaVH3VL1_IgG1_LS_E345R cut, showed restored potency in the blockade of VEGF signaling comparable to HER2 x VEGF bispecific antibodies without the IGF2 masking domain.

### Example 12: Inhibition of HUVEC Cell Proliferation by HER2 x VEGF Bispecific Antibodies

VEGF can stimulate the proliferation of endothelial cells such as human umbilical vein endothelial cells (HUVECs). In vitro cell proliferation assays were set up to evaluate HER2 x VEGF bispecific antibodies in the inhibition of the proliferation of HUVEC cells driven by VEGF. Increasing amounts of test molecules along with 25 ng/mL VEGF were applied to the HUVEC cells for three days and their effects on cell proliferation were assessed by Celltiter-Glo luminescent cell viability assay kit (Promega).

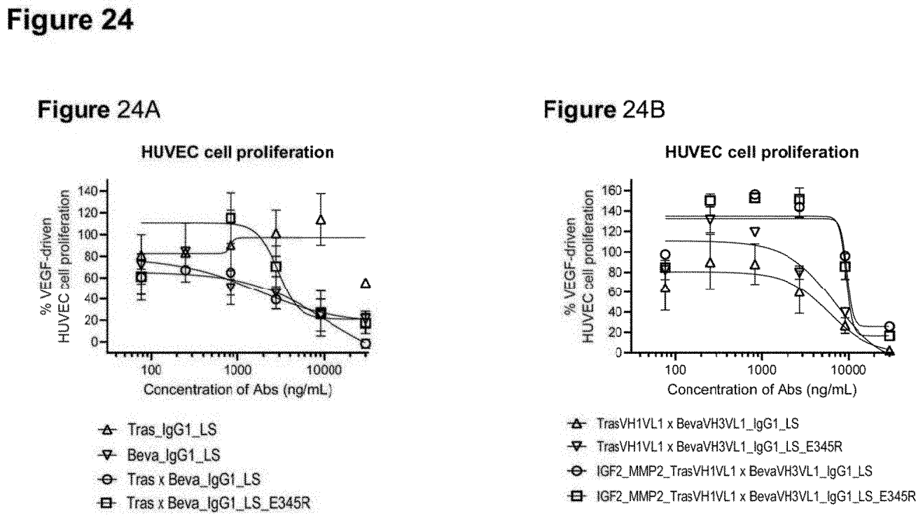

It was observed that anti-VEGF antibody Beva_IgG1_LS and HER2 x VEGF bispecific antibodies Tras x Beva IgG1_LS and Tras x Beva_IgG1_LS_E345R dose-dependently inhibited HUVEC cell proliferation while anti-HER2 antibody Tras_IgG1_LS did not (FIG. 24A). In FIG. 24B, the shielded HER2 x VEGF bispecific antibodies, IGF2_MMP2_TrasVH1VL1 x BevaVH3VL1_IgG1_LS and IGF2_MMP2_TrasVH1VL1 x BevaVH3VL1_IgG1_LS_E345R, showed reduced potency in the inhibition of HUVEC cell proliferation relative to the corresponding HER2 x VEGF bispecific antibodies without the IGF2 masking domain TrasVH1VL1 x BevaVH3VL1_IgG1_LS and TrasVH1VL1 x BevaVH3VL1 IgG1_LS_E345R (FIG. 24B).

### Example 13: Inhibition of HUVEC Cell Migration by HER2 x VEGF Bispecific Antibodies

VEGF can stimulate the migration of endothelial cells such as human umbilical vein endothelial cells (HUVECs). Transwell cell migration assays were set up to evaluate HER2 x VEGF bispecific antibodies in the inhibition of the migration of HUVEC cells driven by VEGF. Test antibodies at 10 μg/mL along with 10 ng/mL VEGF were applied to the lower transwell chamber while 50,000 HUVEC cells were seeded in the upper transwell chamber. The migrated HUVEC cells along with the VEGF gradient on the bottom side of the upper chamber were fixed, stained with DAPI, and quantitated.

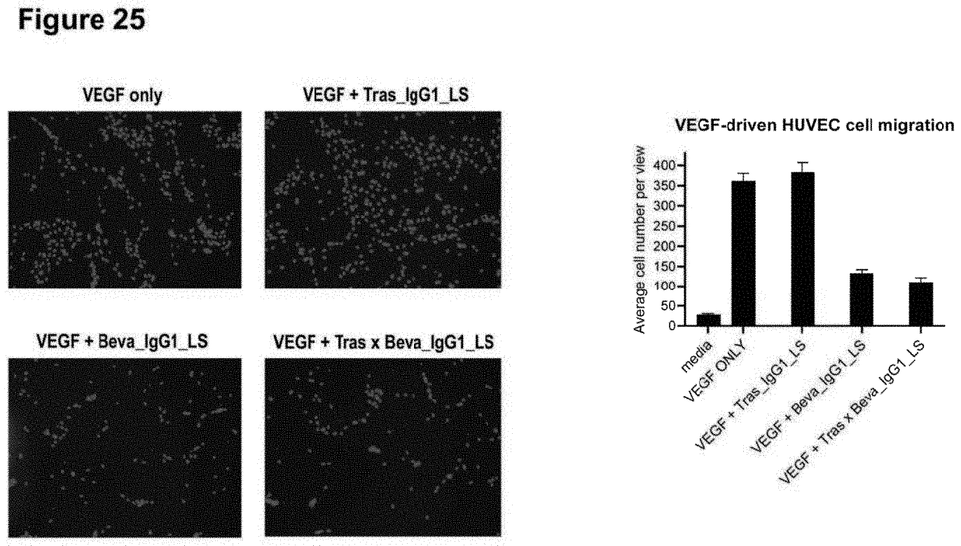

It was observed that 10 ng/mL VEGF stimulated the migration of HUVEC cells. Both anti-VEGF antibody Beva_IgG1_LS and HER2 x VEGF bispecific antibody Tras x Beva IgG1_LS at 10 μg/mL inhibited HUVEC cell migration while anti-HER2 antibody Tras_IgG1_LS did not (FIG. 25).

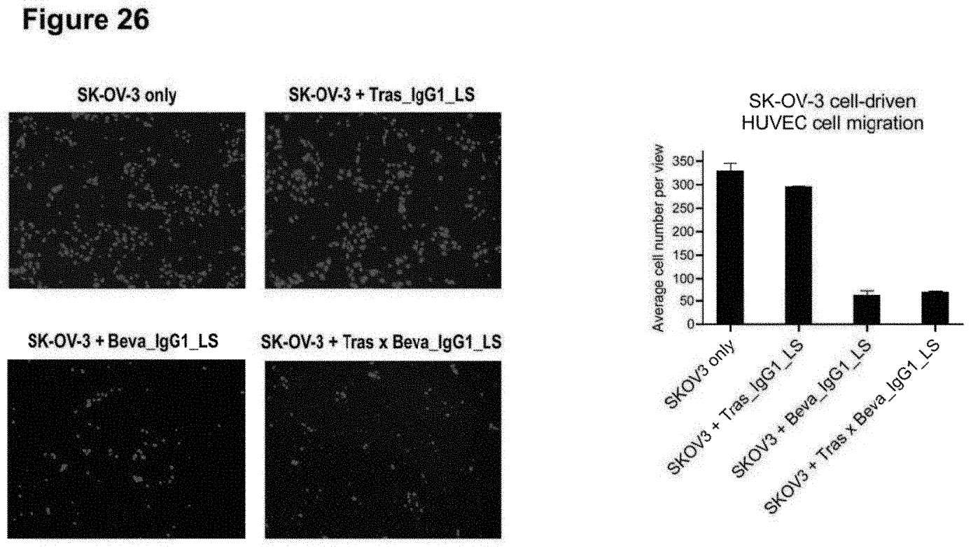

Likewise, the VEGF secreted from the 200,000 SK-OV-3 cells seeded in the lower transwell chamber were also used to stimulate the migration of HUVEC cells. Similarly, both anti-VEGF antibody Beva_IgG1_LS and HER2 x VEGF bispecific antibody Tras x Beva IgG1_LS at 10 μg/mL inhibited HUVEC cell migration while anti-HER2 antibody Tras_IgG1_LS did not inhibit HUVEC cell migration (FIG. 26).

### Example 14: Anti-Tumor Efficacy of HER2 x VEGF Bispecific Antibodies in Human HER2 Positive Cell Xenograft Models

The efficacy of the HER2 x VEGF bispecific antibodies in tumor cell killing was evaluated in mouse tumor xenograft models with human HER2 positive cells. HER2-expressing BT-474 cells were inoculated in NOD/SCID mice for the establishment of orthotopic BT-474 breast cancer xenograft model. Tras_IgG1_LS and Beva_IgG1_LS at 1 mg/kg and Tras x Beva_IgG1_LS at 2 mg/kg were intraperitoneally administered to the mice twice per week and the antibody-mediated tumor shrinkages were assessed.

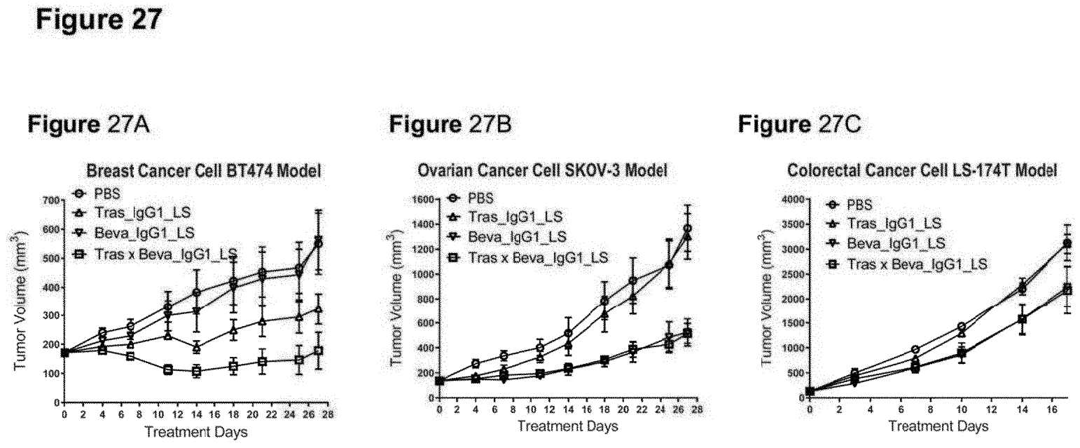

After 27-day treatments, anti-HER2 antibody Tras_IgG1_LS dosed at 1 mg/kg showed 41% tumor growth inhibition while insignificant tumor growth inhibition was observed for anti-VEGF antibody Beva_IgG1_LS. However, HER2 x VEGF bispecific antibody Tras x Beva_IgG1_LS dosed at 2 mg/kg showed 67.66% tumor growth inhibition, indicating synergistic effect of blocking both HER2 and VEGF in tumor inhibition (FIG. 27A).

Besides BT-474 xenograft model, the efficacy of the HER2 x VEGF bispecific antibodies in tumor cell killing were evaluated in subcutaneous SK-OV-3 human ovarian cancer xenograft model. SK-OV-3 cells were inoculated in Balb/c nude mice for the establishment of SK-OV-3 human ovarian cancer xenograft model and test antibodies were intraperitoneally administered to the mice twice per week and the antibody-mediated tumor shrinkage was assessed. After 27-day treatments, anti-HER2 antibody Tras_IgG1_LS dosed at 1 mg/kg showed 5% tumor growth inhibition while 68% tumor growth inhibition was observed for anti-VEGF antibody Beva_IgG1_LS. HER2 x VEGF bispecific antibody Tras x Beva_IgG1_LS dosed at 2 mg/kg showed 69% tumor growth inhibition (FIG. 27B).

The efficacy of the HER2 x VEGF bispecific antibodies in tumor cell killing were also evaluated in subcutaneous LS174T human colon cancer xenograft model. LS174T cells were inoculated in Balb/c nude mice for the establishment of LS174T human colon cancer xenograft model and test antibodies were intraperitoneally administered to the mice twice per week and the antibody-mediated tumor shrinkage were assessed. After 17-day treatments, anti-HER2 antibody Tras_IgG1_LS dosed at 1 mg/kg showed insignificant tumor growth inhibition while 28% tumor growth inhibition was observed for anti-VEGF antibody Beva_IgG1_LS. HER2 x VEGF bispecific antibody Tras x Beva_IgG1_LS dosed at 2 mg/kg showed 31% tumor growth inhibition (FIG. 27C).

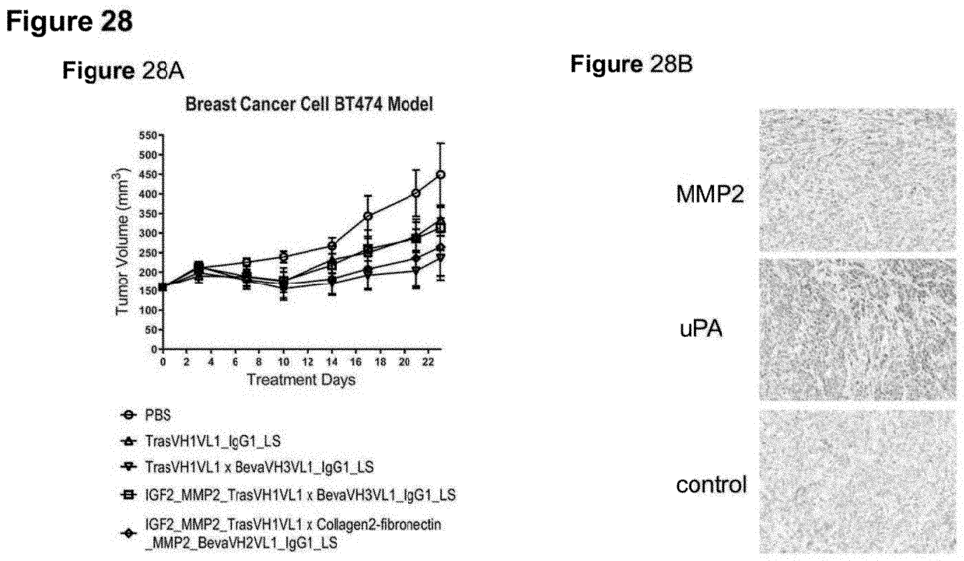

The tumor killing efficacy of another set of HER2 x VEGF bispecific antibodies with and without masking domains was also assessed in orthotopic BT-474 breast cancer xenograft model. After 24-day treatments, anti-HER2 antibody TrasVH1VL1_IgG1_LS dosed at 1 mg/kg showed 25.61% tumor growth inhibition while HER2 x VEGF bispecific antibody TrasVH1VL1 x BevaVH3VL1_IgG1_LS dosed at 1 mg/kg showed 47.41% tumor growth inhibition (FIG. 28A). The shielded HER2 x VEGF bispecific antibodies, IGF2_MMP2_TrasVH1VL1 x BevaVH3VL1_IgG1_LS and IGF2_MMP2_TrasVH1VL1 x Collagen2-fibronectin_MMP2_BevaVH2VL1_IgG1_LS, dosed at 1 mg/kg showed 30.18% and 41.12% tumor growth inhibition respectively (FIG. 28A), indicating robust tumor killing efficacy for the shielded antibodies. In consistent, further IHC studies revealed abundant MMP2 and uPA proteases expression in the BT-474 xenograft tumor (FIG. 28B), supporting the removal of the masking domains from the shielded HER2 x VEGF bispecific antibodies by protease cutting.

### Example 15: Anti-Tumor Efficacy of HER2 x VEGF Bispecific Antibodies in EMT-6-hHER2 Syngeneic Breast Cancer Model

In vitro assays revealed that antibody clustering facilitated by the E345R Fc mutation may help to promote higher antibody effector functions especially ADCP and CDC. To evaluate whether this may translate into better tumor killing efficacy in vivo, the efficacies of anti-HER2 antibody with E345R mutation (Tras_IgG1_LS_E345R) and without E345R mutation (Tras_IgG1_LS) in tumor cell killing were evaluated in EMT-6-hHER2 syngeneic breast cancer model. In this model, EMT-6 cells expressing human HER2 were inoculated in female Balb/c mice for the establishment of subcutaneous EMT-6-hHER2 syngeneic breast cancer model. Tras_IgG1_LS and Tras_IgG1_LS_E345R at 10 mg/kg were intraperitoneally administered to the mice twice per week and the antibody-mediated tumor shrinkage were assessed.

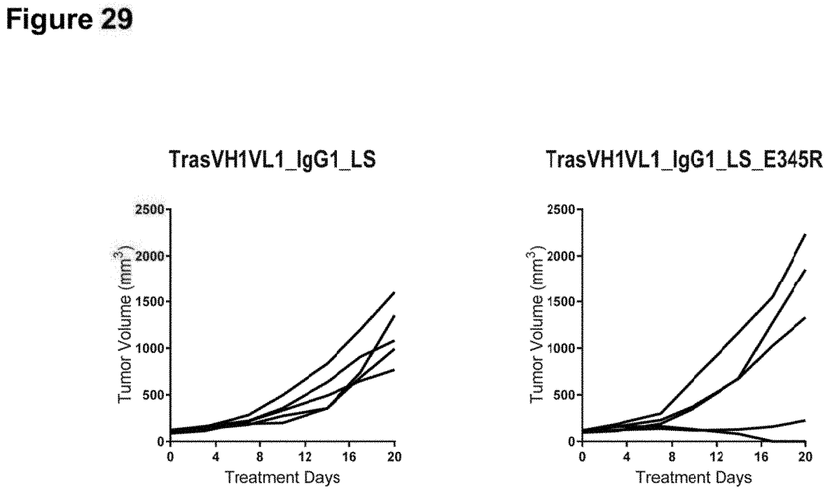

FIG. 29 showed the line graphs of tumor size over a 20-day treatment for each of the five mice in the two test groups. While the tumors for each of the five mice in the Tras_IgG1_LS antibody treatment group continued to grow with tumor sizes more than 5-fold larger than those at the start of treatment, the Tras_IgG1_LS_E345R antibody treatment group had a complete responder with complete tumor shrinkage and a partial responder with tumor size at day 20 about two fold relative to that at the start of the treatment (FIG. 29). This data demonstrated improved efficacy of an anti-HER2 antibody with E345R Fc mutation that can facilitate tumor killing efficacy in an EMT-6-hHER2 syngeneic breast cancer model.

### Example 16: Cardiotoxicity of Shielded HER2 x VEGF Bispecific Antibody

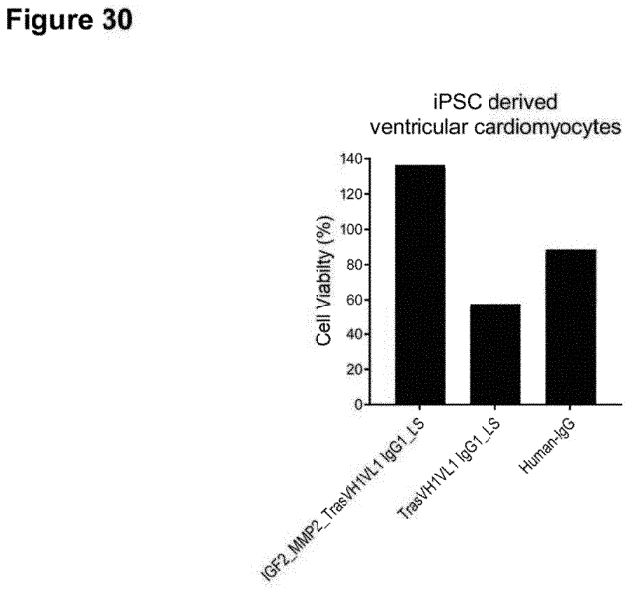

High dose administration of anti-HER2 antibody to patients may increase the risk of cardiac dysfunction, including heart failure, due to the presence of HER2 in cardiomyocytes. In order to show anti-HER2 shielded antibody with IGF2-based masking domain can have reduced risk of cardiotoxicity, an ex vivo cardiotoxicity model was established. Anti-HER2 antibody TrasVH1VL1_IgG1_LS and shielded anti-HER2 antibody with IGF2-based masking domain IGF2_MMP2_TrasVH1VL1_IgG1_LS were incubated with human induced pluripotent stem cell (iPSC) derived ventricular cardiomyocytes (AXOL Bioscience) for 6 days and their effects on the viability of cardiomyocytes were assessed. It was observed that the shielded antibody IGF2_MMP2_TrasVH1VL1_IgG1_LS had much less effect on ventricular cardiomyocytes viability due to the masking of anti-HER2 antibody activity by the IGF2-based masking domain, while the anti-HER2 antibody without the masking domain TrasVH1VL1 IgG1_LS showed significant toxicity on cardiomyocyte viability at 50 μg/mL (FIG. 30). Shielded and unshielded HER2 x VEGF bispecific antibodies may have similar effects on cardiomyocyte viability.

### Example 17: Characterization of HER2 x VEGF Bispecific Antibodies with ScFv of PD-L1 Antibody as the Homing Domain

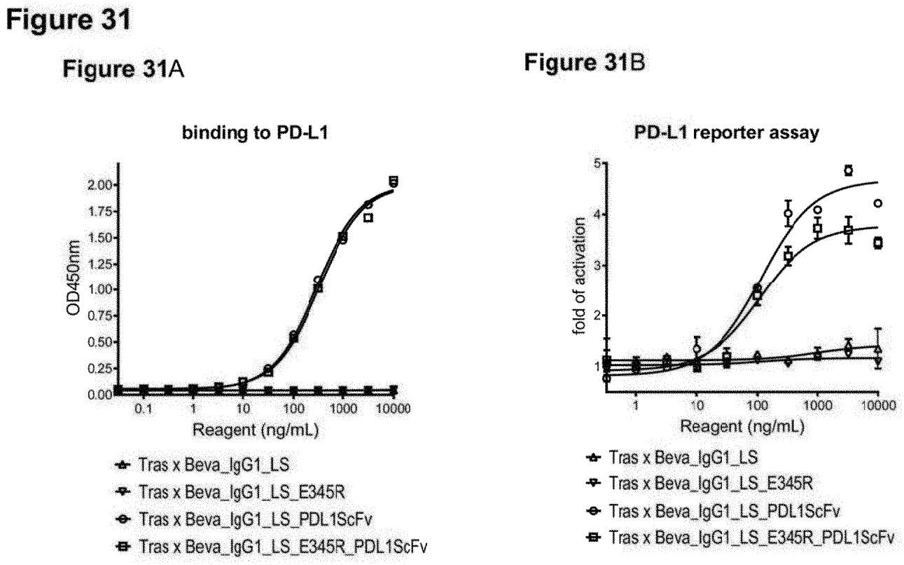

HER2 x VEGF bispecific antibodies with ScFv of an anti-PD-L1 antibody as a homing domain, Tras x Beva_IgG1_LS PDL1ScFv and Tras x Beva IgG1_LS_E345R_PDL1ScFv, were generated. ELISA-based binding assays were employed to evaluate the binding to PD-L1 by these bispecific antibodies (FIG. 31A). In these assays, 1 μg/mL recombinant human PD-L1 (R&D systems) were coated on ELISA plate. Increasing concentrations of exemplary HER2 x VEGF bispecific antibodies with ScFv of an anti-PD-L1 antibody as a homing domain were applied on the plate and their bindings to the recombinant human PD-L1 were detected by HRP-conjugated anti-human IgG secondary antibody. It was observed that Tras x Beva_IgG1_LS PDL1ScFv and Tras x Beva_IgG1_LS_E345R_PDL1ScFv dose-dependently bound to PD-L1 while Tras x Beva IgG1_LS and Tras x Beva IgG1_LS_E345R did not (FIG. 31A).

The activities of HER2 x VEGF bispecific antibodies with ScFv of an anti-PD-L1 antibody as a homing domain in the functional blockade of PD-L1/PD-1 interaction were also evaluated in a PD-L1 reporter assay. Increasing concentrations of exemplary HER2 x VEGF bispecific antibodies with ScFv of an anti-PD-L1 antibody as a homing domain were applied to PD-L1 aAPC cells and PD-1 effector cells (Promega). The stimulated expression of luciferase reporter gene due to the blockade of PD-1/PD-L1 interaction were quantitated. It was observed that Tras x Beva_IgG1_LS PDL1ScFv and Tras x Beva_IgG1_LS_E345R_PDL1ScFv dose-dependently stimulated reporter gene expression while Tras x Beva_IgG1_LS and Tras x Beva IgG1_LS_E345R did not (FIG. 31B).

Further exemplary embodiments are illustrated below.
1. A bispecific antibody that is capable of targeting a HER2 associated pathway and a VEGF associated pathway and is capable of inhibiting tumor cell proliferation, wherein the bispecific antibody comprises one or more shielding domains and/or one or more homing domains.
2. The bispecific antibody of embodiment 1, comprising a binding arm targeting the HER2 associated pathway, including a human IgG heavy chain fusion comprising amino acid sequences from the N- to the C-terminus, signal sequence A-shield A-linker A-protease sequence A-linker B-IgG heavy chain; and a human IgG light chain fusion comprising amino acid sequences from the N- to the C-terminus, signal sequence B-shield B-linker B-protease sequence B-linker C-IgG light chain.
3. The bispecific antibody of embodiment claim 1, comprising a binding arm targeting the HER2 associated pathway, including a human IgG heavy chain fusion comprising amino acid sequences from the N- to the C-terminus, signal sequence A-shield A-linker A-protease sequence A-linker B-IgG heavy chain-linker C-homing domain E; and a human IgG light chain fusion comprising amino acid sequences from the N- to the C-terminus, signal sequence B-shield B-linker B-protease sequence B-linker C-IgG light chain.
4. The bispecific antibody of embodiment 1, comprising a binding arm targeting the VEGF associated pathway, including a human IgG heavy chain fusion comprising amino acid sequences from the N- to the C-terminus, signal sequence A-shield A-linker A-protease sequence A-linker B-IgG heavy chain; and a human IgG light chain fusion comprising amino acid sequences from the N- to the C-terminus, signal sequence B-shield B-linker B-protease sequence B-linker C-IgG light chain.
5. The bispecific antibody of embodiment 1, comprising a binding arm targeting the VEGF associated pathway, including a human IgG heavy chain fusion comprising amino acid sequences from the N- to the C-terminus, signal sequence A-shield A-linker A-protease sequence A-linker B-IgG heavy chain-linker C-homing domain D; and human IgG light chain fusion comprising amino acid sequences from the N- to the C-terminus, signal sequence B-shield B-linker B-protease sequence B-linker C-IgG light chain.
6. The bispecific antibody of any one of embodiments 1-5, wherein the amino acid sequences of the IgG light chain and heavy chain recognize an HER2 antigenic epitope or antigen and comprise sequences chosen from SEQ ID NO: 23-34.
7. The bispecific antibody of any one of embodiments 1-6, wherein the amino acid sequences of the IgG light chain and heavy chain recognize a VEGF antigenic epitope or antigen and comprise sequences chosen from SEQ ID NO: 35-44.
8. The bispecific antibody of any one of embodiments 1-7, wherein the amino acid sequence of the shielding domains each independently comprises a sequence chosen from SEQ ID NO: 1-14.
9. The bispecific antibody of any one of embodiments 1-8, wherein the protease sequence A and protease sequence B each independently comprise a sequence chosen from SEQ ID NO: 15-22.
10. The bispecific antibody of any one of embodiments 1-9, wherein the amino acid sequence of the homing domains each independently comprises a sequence chosen from SEQ ID NO: 45-52 and 82.
11. The bispecific antibody of any one of embodiments 1-10, wherein the IgG heavy and light chain of the HER2 binding arm comprise sequences chose from SEQ ID NO: 53 to 65, and SEQ ID NO: 101 to 145.
12. The bispecific antibody of any one of embodiments 1-11, wherein the IgG heavy and light chain of the VEGF binding arm comprise sequences chosen from SEQ ID NO: 66 to 81, and SEQ ID NO: 201 to 248.
13. The bispecific antibody of any one of embodiments 1-12, wherein the IgG heavy chain and light chain of the HER2 binding arm comprise combinations of the heavy chain and light chain with IgG Fc listed in Table 2 and Table 5.
14. The bispecific antibody of any one of embodiments 1-13, wherein the IgG heavy chain and light chain of the VEGF binding arm comprise combinations of the heavy chain and light chain with IgG Fc listed in Table 3 and Table 6.
15. The bispecific antibody of any one of embodiments 1-14, wherein the IgG heavy chains and light chains of the HER2 binding arm and VEGF binding arm comprise combinations of heavy chains and light chains with IgG Fc listed in Table 4, Table 7 and Table 8.
16. The bispecific antibody of any one of embodiments 1-5, wherein the bispecific antibody comprises the binding arm targeting HER2 in embodiment 2 and the binding arm targeting VEGF in embodiment 4.
17. The bispecific antibody of any one of embodiments 1-5, wherein the bispecific antibody comprises the binding arm targeting HER2 in embodiment 3 and the binding arm targeting VEGF in embodiment 5.
18. The bispecific antibody of any one of embodiments 1-5, wherein the bispecific antibody comprises the binding arm targeting HER2 in embodiment 2 and the binding arm targeting VEGF in embodiment 5.
19. The bispecific antibody of any one of embodiments 1-5, wherein the bispecific antibody comprises the binding arm targeting HER2 in embodiment 3 and the binding arm targeting VEGF in embodiment 4.
20. A nucleic acid sequence encoding the bispecific antibody of any one of embodiments 1-19.
21. A recombinant expression vector comprising the nucleic acid sequence of embodiment 20.
22. A recombinant expression transformant comprising the recombinant expression vector of embodiment 21.
23. A method for preparing a bispecific antibody according to any one of embodiments 1-19, comprising: culturing the recombinant expression transformant of embodiment 22, and obtaining the bispecific antibody from the culture, optionally using controlled Fab arm exchange of culture supernatants.
24. A method for preparing a bispecific antibody according to any one of embodiments 2-5, comprising: culturing the recombinant expression transformant comprising a recombinant expression vector including a nucleic acid sequence encoding the bispecific antibody of any one of embodiments 2-5, and obtaining the bispecific antibody from the culture.
25. A pharmaceutical composition comprising the bispecific antibody according to any one of embodiments 1-19 and a pharmaceutical carrier.
26. A method for treating or preventing a cancer in a subject, comprising administering to the subject a pharmaceutically effective amount of the bispecific antibody of any one of embodiments 1-19 or pharmaceutical composition of embodiment 25.
27. The method for treating or preventing a cancer according to embodiment 26, wherein the cancer is lung cancer, breast cancer, or gastric cancer.
28. The method for treating or preventing a cancer according to any one of embodiments 26-27, where the subject suffers from relapsed HER2 positive cancer.
29. The method for treating or preventing a cancer according to any one of embodiments 26-27, wherein the treatment prevents or reduces metastasis in the subject.

Provided herein is a representative list of certain sequences included in embodiments provided herein.

TABLE 9
Sequences

| SEQ | | |
|---|---|---|
| ID | | |
| NO | Description | Sequence |
| 1 | FI1 | EIDQCIVDDITYNVQDTFHKKHEEGHMLNCTCFGQGRGRWK |
| | | CSPVDQCQDSETGTFYQIGDSWEKYVHGVRYQCYCYGRGIG |
| | | EWHCQPLQTYPSS |
| 2 | FI2 | EIDQCIVDDITYNVQDTFHKKHEEGHMLNCTCFGQGRGRWK |
| | | CEPVDQCQDSETGTFYQIGDSWEKYVHGVRYQCYCYGRGIG |
| | | EWHCQPLQTYPSS |
| 3 | FI3 | EIDQCIVDDITYNVQDTFHKKHEEGHMLQCTCFGQGRGRWK |
| | | CDPVDQCQDSETGTFYQIGDSWEKYVHGVRYQCYCYGRGIG |
| | | EWHCQPLQTYPSS |
| 4 | CO | EVGQRGVVGLPGQRGERGFPGLPGY |
| 5 | IgG1 hinge | EPKSCDKTHTCPPCP |
| 6 | Human IGF2 A chain | GIVEECCFRSCDLALLETYCA |
| 7 | Human IGF2 B chain | AYRPSETLCGGELVDTLQFVCGDRGFYF |
| 8 | Human IGF2 A chain | GILEECCFRSCDLALLETYCA |
| | with V43L mutation | |
| | (bold) | |
| 9 | Human IGF2 B chain | AYRPSETLCGGELVDTLQFVCGDRGFAF |
| | with Y27A mutation | |
| | (bold) | |
| 10 | VEGF epitope A | CAQTMRIKPHQGQHIGESC |
| 11 | HER2 epitope A | CNRDPPFDKC |
| 12 | Collagen peptide 1 | GQRGVVGLPGQRGERGFPGLPGY |
| 13 | Collagen peptide 2 | GQRGVVGLPGQRGER |
| 14 | Fibronectin peptide | DQCIVDDITYNVQDTFHKKHEEGHMLNCTCFGQGRGRWKC |
| | | DPVDQCQDSETGTFYQIGDSWEKYVHGVRYQCYCYGRGIGE |
| | | WHCQPLQTYPSS |
| 15 | Substrate sequence A | GPLGVR |
| | for MMP2 and | |
| | MMP9 | |
| 16 | Substrate sequence B | PLGLAR |
| | for MMP2 and | |
| | MMP9 | |
| 17 | Substrate sequence C | PLGLAG |
| | for MMP2 and | |
| | MMP9 | |
| 18 | Substrate sequence D | IPVSLRSG |
| | for MMP2 and | |
| | MMP9 | |
| 19 | Substrate sequence E | GPLGMLSQ |
| | for MMP2 and | |
| | MMP9 | |
| 20 | Substrate sequence F | RPKPVEVWRK |
| | for MMP3 | |
| 21 | Substrate sequence G | LSGRSDNH |
| | for uPA | |
| 22 | Substrate sequence H | TGRGPSWV |
| | for uPA | |
| 23 | Anti-HER2 | EVQLVESGGGLVQPGGSLRLSCAASGFTFTDYTMDWVRYAP |
| | pertuzumab heavy | GKGLEWVADVNPNSGGSIYNQRFKGRFTLSVDRSKNTLYLQ |
| | chain variable region | MNSLRAEDTAVYYCARNLGPSFYFDYWGQGTLVTVSS |
| 24 | Anti-HER2 | DIQMTQSPSSLSASVGDRVTITCKASQDVSIGVAWYQRKPGK |
| | pertuzumab light | APKLLIYSASYRYTGVPSRFSGSGSGTDFTLTISSLQPEDFATY |
| | chain variable region | YCQQYYIYPYTFGQGTKVEIK |
| 25 | Anti-HER2 | EVQLVESGGGLVQPGGSLRLSCAASGFNIKDTYIHWVRQAPG |
| | trastuzumab heavy | KGLEWVARIYPTNGYTRYADSVKGRFTISADTSKNTAYLQM |
| | chain variable region | NSLRAEDTAVYYCSRWGGDGFYAMDYWGQGTLVTVSS |
| 26 | Anti-HER2 | DIQMTQSPSSLSASVGDRVTITCRASQDVNTAVAWYQQKPG |
| | trastuzumab light | KAPKLLIYSASFLYSGVPSRFSGSRSGTDFTLTISSLQPEDFAT |
| | chain variable region | YYCQQHYTTPPTFGQGTKVEIK |
| 27 | Anti-HER2 | QVQLVQSGAEVKKPGSSVKVSCKASGFNIKDTYIHWVRQAP |
| | trastuzumab heavy | GQGLEWMGRIYPTNGYTRYAQKFQGRATITADESTSTAYME |
| | chain VH1 variant | VSSLRSEDTAVYYCSRWGGDGFYAMDYWGQGTLVTVSS |
| | (TrasVH1) variable | |
| | region | |
| 28 | Anti-HER2 | EVQLVESGGGLVQPGPSLRLSCTASGFNIKDTYIHWVRQAPG |
| | trastuzumab heavy | KGLEWVGRIYPTNGYTRYAASFKGRATISADDSKSIAYLQVN |
| | chain VH2 variant | SLKTEDTAVYYCTRWGGDGFYAMDYWGQGTLVTVSS |
| | (TrasVH2) variable | |
| | region | |
| 29 | Anti-HER2 | EVQLVQSGAEVKKPGESLKISCKGSGFNIKDTYIHWVRQMPG |
| | trastuzumab heavy | KGLEWMGRIYPTNGYTRYSPSFQGQATISADKSISTAYLQVS |
| | chain VH3 variant | SLKASDTAMYYCSRWGGDGFYAMDYWGQGTLVTVSS |
| | (TrasVH3) variable | |
| | region | |
| 30 | Anti-HER2 | QVQLVQSGSELKKPGASVKVSCKASGFNIKDTYIHWVRQAP |
| | trastuzumab heavy | GQGLEWMGRIYPTNGYTRYAQGFTGRFVFSLDTSVSTAYLQI |
| | chain VH4 variant | CSLKAEDTAVYYCSRWGGDGFYAMDYWGQGTLVTVSS |
| | (TrasVH4) variable | |
| | region | |
| 31 | Anti-HER2 | QVQLVQSGSELKKPGASVKVSCKASGFNIKDTYIHWVRQAP |
| | trastuzumab heavy | GQGLEWIGRIYPTNGYTRYAQGFTGRAVISADTSVSTAYLQV |
| | chain VH5 variant | CSLKAEDTAVYYCSRWGGDGFYAMDYWGQGTLVTVSS |
| | (TrasVH5) variable | |
| | region | |
| 32 | Anti-HER2 | DIVMTQTPLSLPVTPGEPASISCRASQDVNTAVAWYLQKPGQ |
| | trastuzumab light | SPQLLIYSASFRYTGVPDRFSGSGSGTDFTLKISRVEAEDVGV |
| | chain VL1 variant | YYCQQHYTTPPTFGQGTKVEIK |
| | (TrasVL1) variable | |
| | region | |
| 33 | Anti-HER2 | DIVMTQSPDSLAVSLGERVTINCKASQDVNTAVAWYQQKPG |
| | trastuzumab light | QSPKLLIYSASFRYSGVPDRFSGSGSGTDFTFTISSVQAEDLAV |
| | chain VL2 variant | YYCQQHYTTPPTFGQGTKVEIK |
| | (TrasVL2) variable | |
| | region | |
| 34 | Anti-HER2 | DIQMTQSPSSLSASVGDRVTITCRASQRVNTAVAWYQQKPG |
| | trastuzumab light | KAPKLLIYSASFLYSGVPSRFSGSGSGTDFTLTISSLQPEDFAT |
| | chain VL3 variant | YYCQQHYTTPPTFGQGTKVEIK |
| | (TrasVL3) variable | |
| | region | |
| 35 | Anti-VEGF | MEIVMTQSPS TLSASVGDRV IITCQASEII HSWLAWYQQK |
| | Brolucizumab single | PGKAPKLLIYLASTLASGVPSRFSGSGSGAEFTLTISSLQPDDF |
| | chain Fv | ATYYCQNVYLASTNGANFGQGTKLTVLGGGGGSGGGGSGG |
| | | GGSGGGGSEVQLVESGGGLVQPGGSLRLSCTASGFSLTDYY |
| | | YMTWVRQAPGKGLEWVGFIDPDDDPYYATWAKGRFTISRD |
| | | NSKNTLYLQMNSLRAEDTAVYYCAGGDHNSGWGLDIWGQ |
| | | GTLVTVSS |
| 36 | anti-VEGF | EVQLVESGGGLVQPGGSLRLSCAASGYTFTNYGMNWVRQA |
| | bevacizumab heavy | PGKGLEWVGWINTYTGEPTYAADFKRRFTFSLDTSKSTAYL |
| | chain variable region | QMNSLRAEDTAVYYCAKYPHYYGSSHWYFDVWGQGTLVT |
| | | VSS |
| 37 | anti-VEGF | DIQMTQSPSSLSASVGDRVTITCSASQDISNYLNWYQQKPGK |
| | bevacizumab light | APKVLIYFTSSLHSGVPSRFSGSGSGTDFTLTISSLQPEDFATY |
| | chain variable region | YCQQYSTVPWTFGQGTKVEIK |
| 38 | Anti-VEGF | QVQLVQSGSELKKPGASVKVSCKASGYTFTNYGMNWVRQA |
| | bevacizumab heavy | PGQGLEWMGWINTYTGEPTYAQGFTGRFVFSLDTSVSTAYL |
| | chain VH1 variant | QICSLKAEDTAVYFCAKYPHYYGSSHWYFDVWGQGTLVTV |
| | (BevaVH1) variable | SS |
| | region | |
| 39 | Anti-VEGF | QVQLVQSGAEVKKPGSSVKVSCKASGYTFTNYGMNWVRQA |
| | bevacizumab heavy | PGQGLEWMGWINTYTGEPTYAQKFQGRFTFTADESTSTAYM |
| | chain VH2 variant | ELSSLRSEDTAVYYCAKYPHYYGSSHWYFDVWGQGTLVTV |
| | (BevaVH2) variable | SS |
| | region | |
| 40 | Anti-VEGF | QVQLVQSGAEVKKPGSSVKVSCKASGYTFTNYGMNWVRQA |
| | bevacizumab heavy | PGQGLEWMGWINTYTGEPTYAQKFQGRVTITADESTSTAYM |
| | chain VH3 variant | ELSSLRSEDTAVYYCAKYPHYYGSSHWYFDVWGQGTLVTV |
| | (BevaVH3) variable | SS |
| | region | |
| 41 | Anti-VEGF | EVQLVQSGAEVKKPGESVKISCKGSGYTFTNYGMNWVRQM |
| | bevacizumab heavy | PGKGLEWMGWINTYTGEPTYSPSFQGQFTFSADKSISTAYLQI |
| | chain VH4 variant | SSLKASDTAMYYCAKYPHYYGSSHWYFDVWGQGTLVTVSS |
| | (BevaVH4) variable | |
| | region | |
| 42 | Anti-VEGF | QVQLVQSGAEVKKPGSSVKVSCKASGYDFTHYGMNWVRQA |
| | bevacizumab heavy | PGQGLEWMGWINTYTGEPTYAQKFQGRFTFTADESTSTAYM |
| | chain VH5 variant | ELSSLRSEDTAVYYCAKYPYYYGTSHWYFDVWGQGTLVTV |
| | (BevaVH5) variable | SS |
| | region | |
| 43 | Anti-VEGF | DIQMTQSPSSLSASVGDRVTITCRASQDISNYLNWYQQKPGK |
| | bevacizumab light | APKVLIYFTSSLHSGVPSRFSGSGSGTDFTLTISSLQPEDFATY |
| | chain VL1 variant | YCQQYSTVPWTFGQGTKLEIK |
| | (BevaVL1) variable | |
| | region | |
| 44 | Anti-VEGF | DIVMTQTPLSLPVTPGEPASISCRASQDISNYLNWYLQKPGQS |
| | bevacizumab light | PQVLIYFTSSLHSGVPDRFSGSGSGTDFTLKISRLEAEDVGVY |
| | chain VL2 variant | YCQQYSTVPWTFGQGTKLEIK |
| | (BevaVL2) variable | |
| | region | |
| 45 | F19-1 | EVQLLESGGGLVQPGGSLRLSCAASGFTFSSFSMSWVRQAPG |
| | | KGLEWVSSISGSSGTTYYADSVKGRFTISRDNSKNTLYLQMN |
| | | SLRAEDTAVYYCAKPFPYFDYWGQGTLVTVSSGDGSSGGSG |
| | | GASEIVLTQSPGTLSLSPGERATLSCRASQSVSSSFLAWYQQK |
| | | PGQAPRLLIYYASSRATGIPDRFSGSGSGTDFTLTISRLEPEDF |
| | | AVYYCQQTGRIPPTFGQGTKVEIK |
| 46 | F19-2 | EVQLLESGGGLVQPGGSLRLSCAASGFTFSSFSMSWVRQAPG |
| | | KGLEWVSSISGSSGTTYYADSVKGRFTISRDNSKNTLYLQMN |
| | | SLRAEDTAVYYCAKPFPYFDYWGQGTLVTVSSGGSGGGGSG |
| | | GGGSGGEIVLTQSPGTLSLSPGERATLSCRASQSVSSSFLAWY |
| | | QQKPGQAPRLLIYYASSRATGIPDRFSGSGSGTDFTLTISRLEP |
| | | EDFAVYYCQQTGRIPPTFGQGTKVEIK |
| 47 | FAP-1 | EVQLVESGGGLVQPGPSLRLSCTASRYTFTEYTIHWVRQAPG |
| | | KGLEWVGGINPNNGIPNYAASVKGRFTISVDDSKSIAYLQMN |
| | | SLKTEDTAVYFCARRRIAYGYDEGHAMDYWGQGTTVTVSS |
| | | GGSGGGGSGGGGSGGDIVMTQSPLSLPVTPGEPASMSCKSSQ |
| | | SLLYSRNQKNYLAWFLQKPGQSPQLLIFWASTRESGVPDRFS |
| | | GSGSGTDFTLKISRVEAEDVGVYDCQQYFSYPLTFGQGTKLE |
| | | IK |
| 48 | FAP-2 | EVQLVESGGGLVQPGPSLRLSCTASRYTFTEYTIHWVRQAPG |
| | | KGLEWVGGINPNNGIPNYAASVKGRFTISVDDSKSIAYLQMN |
| | | SLKTEDTAVYFCARRRIAYGYDEGHAMDYWGQGTTVTVSS |
| | | GGSGGGGSGGGGSGGAIRMTQSPSSFSVSTGDRVTMTCRSSQ |
| | | SLLYSRNQKNYLAWFQQKPGKAPKLLIFWASTRESGVPSRFS |
| | | GSGSGTDFTLTISCVQSEDFATYDCQQYFSYPLTFGQGTKLEI |
| | | K |
| 49 | FAP-3 | QMQLVQSGAEVKKTGSSVKVSCKTSRYTFTEYTIHWVRQAP |
| | | GQALEWIGGINPNNGIPNYAQKFQDRATLTVDRSMSTAYME |
| | | VSSLRSEDTAMYFCARRRIAYGYDEGHAMDYWGQGTTVTV |
| | | SSGGSGGGGSGGGGSGGDIVMTQSPLSLPVTPGEPASMSCKS |
| | | SQSLLYSRNQKNYLAWFLQKPGQSPQLLIFWASTRESGVPDR |
| | | FSGSGSGTDFTLKISRVEAEDVGVYDCQQYFSYPLTFGQGTK |
| | | LEIK |
| 50 | FAP-4 | QMQLVQSGAEVKKTGSSVKVSCKTSRYTFTEYTIHWVRQAP |
| | | GQALEWIGGINPNNGIPNYAQKFQDRATLTVDRSMSTAYME |
| | | VSSLRSEDTAMYFCARRRIAYGYDEGHAMDYWGQGTTVTV |
| | | SSGGSGGGGSGGGGSGGAIRMTQSPSSFSVSTGDRVTMTCRS |
| | | SQSLLYSRNQKNYLAWFQQKPGKAPKLLIFWASTRESGVPSR |
| | | FSGSGSGTDFTLTISCVQSEDFATYDCQQYFSYPLTFGQGTKL |
| | | EIK |
| 51 | FAP-5 | QVQLQESGPGLVKPSDTLSLTCAVSRYTFTEYTIHWVRQPPG |
| | | KGLEWIGGINPNNGIPNYNPSLKSRATMSVDTSKNQASLKLS |
| | | SVTAVDTAVYFCARRRIAYGYDEGHAMDYWGQGTTVTVSS |
| | | GGSGGGGSGGGGSGGDIVMTQSPLSLPVTPGEPASMSCKSSQ |
| | | SLLYSRNQKNYLAWFLQKPGQSPQLLIFWASTRESGVPDRFS |
| | | GSGSGTDFTLKISRVEAEDVGVYDCQQYFSYPLTFGQGTKLE |
| | | IK |
| 52 | FAP-6 | QVQLQESGPGLVKPSDTLSLTCAVSRYTFTEYTIHWVRQPPG |
| | | KGLEWIGGINPNNGIPNYNPSLKSRATMSVDTSKNQASLKLS |
| | | SVTAVDTAVYFCARRRIAYGYDEGHAMDYWGQGTTVTVSS |
| | | GGSGGGGSGGGGSGGAIRMTQSPSSFSVSTGDRVTMTCRSSQ |
| | | SLLYSRNQKNYLAWFQQKPGKAPKLLIFWASTRESGVPSRFS |
| | | GSGSGTDFTLTISCVQSEDFATYDCQQYFSYPLTFGQGTKLEI |
| | | K |
| 53 | EAC202: anti-HER2 | EVQLVESGGGLVQPGGSLRLSCAASGFNIKDTYIHWVRQAPG |
| | trastuzumab heavy | KGLEWVARIYPTNGYTRYADSVKGRFTISADTSKNTAYLQM |
| | chain with IgG1 Fc | NSLRAEDTAVYYCSRWGGDGFYAMDYWGQGTLVTVSSAST |
| | with E233P, F405L, | KGPSVFPLAPSSKSTSGGTAALGCLVKDYFPEPVTVSWNSGA |
| | M428L, N434S | LTSGVHTFPAVLQSSGLYSLSSVVTVPSSSLGTQTYICNVNHK |
| | mutations and G236 | PSNTKVDKKVEPKSCDKTHTCPPCPAPPLLGPSVFLFPPKPKD |
| | deleted | TLMISRTPEVTCVVVDVSHEDPEVKFNWYVDGVEVHNAKTK |
| | | PREEQYNSTYRVVSVLTVLHQDWLNGKEYKCKVSNKALPAP |
| | | IEKTISKAKGQPREPQVYTLPPSRDELTKNQVSLTCLVKGFYP |
| | | SDIAVEWESNGQPENNYKTTPPVLDSDGSFLLYSkLTVDKSR |
| | | WQQGNVFSCSVLHEALHSHYTQKSLSLSPGK |
| 54 | EAC203: anti-HER2 | DIQMTQSPSSLSASVGDRVTITCRASQDVNTAVAWYQQKPG |
| | trastuzumab light | KAPKLLIYSASFLYSGVPSRFSGSRSGTDFTLTISSLQPEDFAT |
| | chain | YYCQQHYTTPPTFGQGTKVEIKRTVAAPSVFIFPPSDEQLKSG |
| | | TASVVCLLNNFYPREAKVQWKVDNALQSGNSQESVTEQDSK |
| | | DSTYSLSSTLTLSKADYEKHKVYACEVTHQGLSSPVTKSFNR |
| | | GEC |
| 55 | EAC289: anti-HER2 | EVQLVESGGGLVQPGGSLRLSCAASGFNIKDTYIHWVRQAPG |
| | trastuzumab heavy | KGLEWVARIYPTNGYTRYADSVKGRFTISADTSKNTAYLQM |
| | chain with IgG1 Fc | NSLRAEDTAVYYCSRWGGDGFYAMDYWGQGTLVTVSSAST |
| | with F405L, M428L, | KGPSVFPLAPSSKSTSGGTAALGCLVKDYFPEPVTVSWNSGA |
| | N434S mutations | LTSGVHTFPAVLQSSGLYSLSSVVTVPSSSLGTQTYICNVNHK |
| | | PSNTKVDKKVEPKSCDKTHTCPPCPAPELLGGPSVFLFPPKPK |
| | | DTLMISRTPEVTCVVVDVSHEDPEVKFNWYVDGVEVHNAKT |
| | | KPREEQYNSTYRVVSVLTVLHQDWLNGKEYKCKVSNKALP |
| | | APIEKTISKAKGQPREPQVYTLPPSRDELTKNQVSLTCLVKGF |
| | | YPSDIAVEWESNGQPENNYKTTPPVLDSDGSFLLYSkLTVDK |
| | | SRWQQGNVFSCSVLHEALHSHYTQKSLSLSPGK |
| 56 | EAC505: anti-HER2 | EVQLVESGGGLVQPGGSLRLSCAASGFTFTDYTMDWVRQAP |
| | pertuzumab heavy | GKGLEWVADVNPNSGGSIYNQRFKGRFTLSVDRSKNTLYLQ |
| | chain with IgG1 Fc | MNSLRAEDTAVYYCARNLGPSFYFDYWGQGTLVTVSSASTK |
| | with K409R, M428L, | GPSVFPLAPSSKSTSGGTAALGCLVKDYFPEPVTVSWNSGAL |
| | N434S mutations | TSGVHTFPAVLQSSGLYSLSSVVTVPSSSLGTQTYICNVNHKP |
| | | SNTKVDKKVEPKSCDKTHTCPPCPAPELLGGPSVFLFPPKPKD |
| | | TLMISRTPEVTCVVVDVSHEDPEVKFNWYVDGVEVHNAKTK |
| | | PREEQYNSTYRVVSVLTVLHQDWLNGKEYKCKVSNKALPAP |
| | | IEKTISKAKGQPREPQVYTLPPSRDELTKNQVSLTCLVKGFYP |
| | | SDIAVEWESNGQPENNYKTTPPVLDSDGSFFLYSRLTVDKSR |
| | | WQQGNVFSCSVLHEALHSHYTQKSLSLSPGK |
| 57 | EAC506: anti-HER2 | DIQMTQSPSSLSASVGDRVTITCKASQDVSIGVAWYQQKPGK |
| | pertuzumab light | APKLLIYSASYRYTGVPSRFSGSGSGTDFTLTISSLQPEDFATY |
| | chain | YCQQYYIYPYTFGQGTKVEIKRTVAAPSVFIFPPSDEQLKSGT |
| | | ASVVCLLNNFYPREAKVQWKVDNALQSGNSQESVTEQDSK |
| | | DSTYSLSSTLTLSKADYEKHKVYACEVTHQGLSSPVTKSFNR |
| | | GEC |
| 58 | EAC509: anti-HER2 | EVQLVESGGGLVQPGGSLRLSCAASGFNIKDTYIHWVRQAPG |
| | trastuzumab heavy | KGLEWVARIYPTNGYTRYADSVKGRFTISADTSKNTAYLQM |
| | chain with IgG1 Fc | NSLRAEDTAVYYCSRWGGDGFYAMDYWGQGTLVTVSSAST |
| | with E345R, F405L, | KGPSVFPLAPSSKSTSGGTAALGCLVKDYFPEPVTVSWNSGA |
| | M428L, N434S | LTSGVHTFPAVLQSSGLYSLSSVVTVPSSSLGTQTYICNVNHK |
| | mutations | PSNTKVDKKVEPKSCDKTHTCPPCPAPELLGGPSVFLFPPKPK |
| | | DTLMISRTPEVTCVVVDVSHEDPEVKFNWYVDGVEVHNAKT |
| | | KPREEQYNSTYRVVSVLTVLHQDWLNGKEYKCKVSNKALP |
| | | APIEKTISKAKGQPRRPQVYTLPPSRDELTKNQVSLTCLVKGF |
| | | YPSDIAVEWESNGQPENNYKTTPPVLDSDGSFLLYSkLTVDK |
| | | SRWQQGNVFSCSVLHEALHSHYTQKSLSLSPGK |
| 59 | EAC562: anti-HER2 | QVQLVQSGAEVKKPGSSVKVSCKASGFNIKDTYIHWVRQAP |
| | trastuzumab heavy | GQGLEWMGRIYPTNGYTRYAQKFQGRATITADESTSTAYME |
| | chain VH1 variant | VSSLRSEDTAVYYCSRWGGDGFYAMDYWGQGTLVTVSSAS |
| | (TrasVH1) with IgG1 | TKGPSVFPLAPSSKSTSGGTAALGCLVKDYFPEPVTVSWNSG |
| | Fc with F405L, | ALTSGVHTFPAVLQSSGLYSLSSVVTVPSSSLGTQTYICNVNH |
| | M428L, N434S | KPSNTKVDKKVEPKSCDKTHTCPPCPAPELLGGPSVFLFPPKP |
| | mutations | KDTLMISRTPEVTCVVVDVSHEDPEVKFNWYVDGVEVHNA |
| | | KTKPREEQYNSTYRVVSVLTVLHQDWLNGKEYKCKVSNKA |
| | | LPAPIEKTISKAKGQPREPQVYTLPPSRDELTKNQVSLTCLVK |
| | | GFYPSDIAVEWESNGQPENNYKTTPPVLDSDGSFLLYSkLTV |
| | | DKSRWQQGNVFSCSVLHEALHSHYTQKSLSLSPGK |
| 60 | EAC563: anti-HER2 | QVQLVQSGAEVKKPGSSVKVSCKASGFNIKDTYIHWVRQAP |
| | trastuzumab heavy | GQGLEWMGRIYPTNGYTRYAQKFQGRATITADESTSTAYME |
| | chain VH1 variant | VSSLRSEDTAVYYCSRWGGDGFYAMDYWGQGTLVTVSSAS |
| | (TrasVH1) with IgG1 | TKGPSVFPLAPSSKSTSGGTAALGCLVKDYFPEPVTVSWNSG |
| | Fc with E345R, | ALTSGVHTFPAVLQSSGLYSLSSVVTVPSSSLGTQTYICNVNH |
| | F405L, M428L, | KPSNTKVDKKVEPKSCDKTHTCPPCPAPELLGGPSVFLFPPKP |
| | N434S mutations | KDTLMISRTPEVTCVVVDVSHEDPEVKFNWYVDGVEVHNA |
| | | KTKPREEQYNSTYRVVSVLTVLHQDWLNGKEYKCKVSNKA |
| | | LPAPIEKTISKAKGQPRRPQVYTLPPSRDELTKNQVSLTCLVK |
| | | GFYPSDIAVEWESNGQPENNYKTTPPVLDSDGSFLLYSkLTV |
| | | DKSRWQQGNVFSCSVLHEALHSHYTQKSLSLSPGK |
| 61 | EAC564: anti-HER2 | DIVMTQTPLSLPVTPGEPASISCRASQDVNTAVAWYLQKPGQ |
| | trastuzumab light | SPQLLIYSASFRYTGVPDRFSGSGSGTDFTLKISRVEAEDVGV |
| | chain VL1 variant | YYCQQHYTTPPTFGQGTKVEIKRTVAAPSVFIFPPSDEQLKSG |
| | (TrasVL1) | TASVVCLLNNFYPREAKVQWKVDNALQSGNSQESVTEQDSK |
| | | DSTYSLSSTLTLSKADYEKHKVYACEVTHQGLSSPVTKSFNR |
| | | GEC |
| 62 | EAC862: anti-HER2 | EVQLVESGGGLVQPGGSLRLSCAASGFNIKDTYIHWVRQAPG |
| | trastuzumab heavy | KGLEWVARIYPTNGYTRYADSVKGRFTISADTSKNTAYLQM |
| | chain with IgG1 Fc | NSLRAEDTAVYYCSRWGGDGFYAMDYWGQGTLVTVSSAST |
| | | KGPSVFPLAPSSKSTSGGTAALGCLVKDYFPEPVTVSWNSGA |
| | | LTSGVHTFPAVLQSSGLYSLSSVVTVPSSSLGTQTYICNVNHK |
| | | PSNTKVDKKVEPKSCDKTHTCPPCPAPELLGGPSVFLFPPKPK |
| | | DTLMISRTPEVTCVVVDVSHEDPEVKFNWYVDGVEVHNAKT |
| | | KPREEQYNSTYRVVSVLTVLHQDWLNGKEYKCKVSNKALP |
| | | APIEKTISKAKGQPREPQVYTLPPSREEMTKNQVSLTCLVKGF |
| | | YPSDIAVEWESNGQPENNYKTTPPVLDSDGSFFLYSKLTVDK |
| | | SRWQQGNVFSCSVMHEALHNHYTQKSLSLSPGK |
| 63 | EAC1040: anti-HER2 | QVQLVQSGAEVKKPGSSVKVSCKASGFNIKDTYIHWVRQAP |
| | trastuzumab heavy | GQGLEWMGRIYPTNGYTRYAQKFQGRATITADESTSTAYME |
| | chain VH1 variant | VSSLRSEDTAVYYCSRWGGDGFYAMDYWGQGTLVTVSSAS |
| | (TrasVH1) with IgG1 | TKGPSVFPLAPSSKSTSGGTAALGCLVKDYFPEPVTVSWNSG |
| | Fc with T366W, | ALTSGVHTFPAVLQSSGLYSLSSVVTVPSSSLGTQTYICNVNH |
| | M428L, N434S | KPSNTKVDKKVEPKSCDKTHTCPPCPAPELLGGPSVFLFPPKP |
| | mutations | KDTLMISRTPEVTCVVVDVSHEDPEVKFNWYVDGVEVHNA |
| | | KTKPREEQYNSTYRVVSVLTVLHQDWLNGKEYKCKVSNKA |
| | | LPAPIEKTISKAKGQPREPQVYTLPPSRDELTKNQVSLWCLVK |
| | | GFYPSDIAVEWESNGQPENNYKTTPPVLDSDGSFFLYSkLTVD |
| | | KSRWQQGNVFSCSVLHEALHSHYTQKSLSLSPGK |
| 64 | EAC1047: anti-HER2 | QVQLVQSGAEVKKPGSSVKVSCKASGFNIKDTYIHWVRQAP |
| | trastuzumab heavy | GQGLEWMGRIYPTNGYTRYAQKFQGRATITADESTSTAYME |
| | chain VH1 variant | VSSLRSEDTAVYYCSRWGGDGFYAMDYWGQGTLVTVSSAS |
| | (TrasVH1) with IgG1 | TKGPSVFPLAPSSKSTSGGTAALGCLVKDYFPEPVTVSWNSG |
| | Fc with F243L, | ALTSGVHTFPAVLQSSGLYSLSSVVTVPSSSLGTQTYICNVNH |
| | R292P, Y300L, | KPSNTKVDKKVEPKSCDKTHTCPPCPAPELLGGPSVFLLPPKP |
| | V305I, P396L | KDTLMISRTPEVTCVVVDVSHEDPEVKFNWYVDGVEVHNA |
| | mutations | KTKPPEEQYNSTLRVVSILTVLHQDWLNGKEYKCKVSNKAL |
| | | PAPIEKTISKAKGQPREPQVYTLPPSRDELTKNQVSLTCLVKG |
| | | FYPSDIAVEWESNGQPENNYKTTPLVLDSDGSFFLYSkLTVD |
| | | KSRWQQGNVFSCSVMHEALHNHYTQKSLSLSPGK |
| 65 | EAC1048: anti-HER2 | QVQLVQSGAEVKKPGSSVKVSCKASGFNIKDTYIHWVRQAP |
| | trastuzumab heavy | GQGLEWMGRIYPTNGYTRYAQKFQGRATITADESTSTAYME |
| | chain VH1 variant | VSSLRSEDTAVYYCSRWGGDGFYAMDYWGQGTLVTVSSAS |
| | (TrasVH1) with IgG1 | TKGPSVFPLAPSSKSTSGGTAALGCLVKDYFPEPVTVSWNSG |
| | Fc with F243L, | ALTSGVHTFPAVLQSSGLYSLSSVVTVPSSSLGTQTYICNVNH |
| | R292P, Y300L, | KPSNTKVDKKVEPKSCDKTHTCPPCPAPELLGGPSVFLLPPKP |
| | V305I, T366W, | KDTLMISRTPEVTCVVVDVSHEDPEVKFNWYVDGVEVHNA |
| | P396L mutations | KTKPPEEQYNSTLRVVSILTVLHQDWLNGKEYKCKVSNKAL |
| | | PAPIEKTISKAKGQPREPQVYTLPPSRDELTKNQVSLWCLVK |
| | | GFYPSDIAVEWESNGQPENNYKTTPLVLDSDGSFFLYSkLTV |
| | | DKSRWQQGNVFSCSVMHEALHNHYTQKSLSLSPGK |
| 66 | EAC206: anti-VEGF | EVQLVESGGGLVQPGGSLRLSCAASGYTFTNYGMNWVRQA |
| | bevacizumab heavy | PGKGLEWVGWINTYTGEPTYAADFKRRFTFSLDTSKSTAYL |
| | chain with IgG1 Fc | QMNSLRAEDTAVYYCAKYPHYYGSSHWYFDVWGQGTLVT |
| | with E233P, K409R, | VSSASTKGPSVFPLAPSSKSTSGGTAALGCLVKDYFPEPVTVS |
| | M428L, N434S | WNSGALTSGVHTFPAVLQSSGLYSLSSVVTVPSSSLGTQTYIC |
| | mutations and G236 | NVNHKPSNTKVDKKVEPKSCDKTHTCPPCPAPPLLGPSVFLF |
| | deleted | PPKPKDTLMISRTPEVTCVVVDVSHEDPEVKFNWYVDGVEV |
| | | HNAKTKPREEQYNSTYRVVSVLTVLHQDWLNGKEYKCKVS |
| | | NKALPAPIEKTISKAKGQPREPQVYTLPPSRDELTKNQVSLTC |
| | | LVKGFYPSDIAVEWESNGQPENNYKTTPPVLDSDGSFFLYSR |
| | | LTVDKSRWQQGNVFSCSVLHEALHSHYTQKSLSLSPGK |
| 67 | EAC207: anti-VEGF | DIQMTQSPSSLSASVGDRVTITCSASQDISNYLNWYQQKPGK |
| | bevacizumab light | APKVLIYFTSSLHSGVPSRFSGSGSGTDFTLTISSLQPEDFATY |
| | chain | YCQQYSTVPWTFGQGTKVEIKRTVAAPSVFIFPPSDEQLKSGT |
| | | ASVVCLLNNFYPREAKVQWKVDNALQSGNSQESVTEQDSK |
| | | DSTYSLSSTLTLSKADYEKHKVYACEVTHQGLSSPVTKSFNR |
| | | GEC |
| 68 | EAC291: anti-VEGF | EVQLVESGGGLVQPGGSLRLSCAASGYTFTNYGMNWVRQA |
| | bevacizumab heavy | PGKGLEWVGWINTYTGEPTYAADFKRRFTFSLDTSKSTAYL |
| | chain with IgG1 Fc | QMNSLRAEDTAVYYCAKYPHYYGSSHWYFDVWGQGTLVT |
| | with K409R, M428L, | VSSASTKGPSVFPLAPSSKSTSGGTAALGCLVKDYFPEPVTVS |
| | N434S mutations | WNSGALTSGVHTFPAVLQSSGLYSLSSVVTVPSSSLGTQTYIC |
| | | NVNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKV |
| | | SNKALPAPIEKTISKAKGQPREPQVYTLPPSRDELTKNQVSLT |
| | | CLVKGFYPSDIAVEWESNGQPENNYKTTPPVLDSDGSFFLYS |
| | | RLTVDKSRWQQGNVFSCSVLHEALHSHYTQKSLSLSPGK |
| 69 | EAC513: anti-VEGF | EVQLVESGGGLVQPGGSLRLSCAASGYTFTNYGMNWVRQA |
| | bevacizumab heavy | PGKGLEWVGWINTYTGEPTYAADFKRRFTFSLDTSKSTAYL |
| | chain with IgG1 Fc | QMNSLRAEDTAVYYCAKYPHYYGSSHWYFDVWGQGTLVT |
| | with E345R, K409R, | VSSASTKGPSVFPLAPSSKSTSGGTAALGCLVKDYFPEPVTVS |
| | M428L, N434S | WNSGALTSGVHTFPAVLQSSGLYSLSSVVTVPSSSLGTQTYIC |
| | mutations | NVNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKV |
| | | SNKALPAPIEKTISKAKGQPRRPQVYTLPPSRDELTKNQVSLT |
| | | CLVKGFYPSDIAVEWESNGQPENNYKTTPPVLDSDGSFFLYS |
| | | RLTVDKSRWQQGNVFSCSVLHEALHSHYTQKSLSLSPGK |
| 70 | EAC827: anti-VEGF | QVQLVQSGSELKKPGASVKVSCKASGYTFTNYGMNWVRQA |
| | bevacizumab heavy | PGQGLEWMGWINTYTGEPTYAQGFTGRFVFSLDTSVSTAYL |
| | chain VH1 variant | QICSLKAEDTAVYFCAKYPHYYGSSHWYFDVWGQGTLVTV |
| | (BevaVH1) with IgG1 | SSASTKGPSVFPLAPSSKSTSGGTAALGCLVKDYFPEPVTVSW |
| | Fc with K409R, | NSGALTSGVHTFPAVLQSSGLYSLSSVVTVPSSSLGTQTYICN |
| | M428L, N434S | VNHKPSNTKVDKKVEPKSCDKTHTCPPCPAPELLGGPSVFLF |
| | mutations | PPKPKDTLMISRTPEVTCVVVDVSHEDPEVKFNWYVDGVEV |
| | | HNAKTKPREEQYNSTYRVVSVLTVLHQDWLNGKEYKCKVS |
| | | NKALPAPIEKTISKAKGQPREPQVYTLPPSRDELTKNQVSLTC |
| | | LVKGFYPSDIAVEWESNGQPENNYKTTPPVLDSDGSFFLYSR |
| | | LTVDKSRWQQGNVFSCSVLHEALHSHYTQKSLSLSPGK |
| 71 | EAC828: anti-VEGF | QVQLVQSGAEVKKPGSSVKVSCKASGYTFTNYGMNWVRQA |
| | bevacizumab heavy | PGQGLEWMGWINTYTGEPTYAQKFQGRFTFTADESTSTAYM |
| | chain VH2 variant | ELSSLRSEDTAVYYCAKYPHYYGSSHWYFDVWGQGTLVTV |
| | (BevaVH2) with IgG1 | SSASTKGPSVFPLAPSSKSTSGGTAALGCLVKDYFPEPVTVSW |
| | Fc with K409R, | NSGALTSGVHTFPAVLQSSGLYSLSSVVTVPSSSLGTQTYICN |
| | M428L, N434S | VNHKPSNTKVDKKVEPKSCDKTHTCPPCPAPELLGGPSVFLF |
| | mutations | PPKPKDTLMISRTPEVTCVVVDVSHEDPEVKFNWYVDGVEV |
| | | HNAKTKPREEQYNSTYRVVSVLTVLHQDWLNGKEYKCKVS |
| | | NKALPAPIEKTISKAKGQPREPQVYTLPPSRDELTKNQVSLTC |
| | | LVKGFYPSDIAVEWESNGQPENNYKTTPPVLDSDGSFFLYSR |
| | | LTVDKSRWQQGNVFSCSVLHEALHSHYTQKSLSLSPGK |
| 72 | EAC829: anti-VEGF | QVQLVQSGAEVKKPGSSVKVSCKASGYTFTNYGMNWVRQA |
| | bevacizumab heavy | PGQGLEWMGWINTYTGEPTYAQKFQGRVTITADESTSTAYM |
| | chain VH3 variant | ELSSLRSEDTAVYYCAKYPHYYGSSHWYFDVWGQGTLVTV |
| | (BevaVH3) with IgG1 | SSASTKGPSVFPLAPSSKSTSGGTAALGCLVKDYFPEPVTVSW |
| | Fc with K409R, | NSGALTSGVHTFPAVLQSSGLYSLSSVVTVPSSSLGTQTYICN |
| | M428L, N434S | VNHKPSNTKVDKKVEPKSCDKTHTCPPCPAPELLGGPSVFLF |
| | mutations | PPKPKDTLMISRTPEVTCVVVDVSHEDPEVKFNWYVDGVEV |
| | | HNAKTKPREEQYNSTYRVVSVLTVLHQDWLNGKEYKCKVS |
| | | NKALPAPIEKTISKAKGQPREPQVYTLPPSRDELTKNQVSLTC |
| | | LVKGFYPSDIAVEWESNGQPENNYKTTPPVLDSDGSFFLYSR |
| | | LTVDKSRWQQGNVFSCSVLHEALHSHYTQKSLSLSPGK |
| 73 | EAC830: anti-VEGF | EVQLVQSGAEVKKPGESVKISCKGSGYTFTNYGMNWVRQM |
| | bevacizumab heavy | PGKGLEWMGWINTYTGEPTYSPSFQGQFTFSADKSISTAYLQI |
| | chain VH4 variant | SSLKASDTAMYYCAKYPHYYGSSHWYFDVWGQGTLVTVSS |
| | (BevaVH4) with IgG1 | ASTKGPSVFPLAPSSKSTSGGTAALGCLVKDYFPEPVTVSWN |
| | Fc with K409R, | SGALTSGVHTFPAVLQSSGLYSLSSVVTVPSSSLGTQTYICNV |
| | M428L, N434S | NHKPSNTKVDKKVEPKSCDKTHTCPPCPAPELLGGPSVFLFPP |
| | mutations | KPKDTLMISRTPEVTCVVVDVSHEDPEVKFNWYVDGVEVHN |
| | | AKTKPREEQYNSTYRVVSVLTVLHQDWLNGKEYKCKVSNK |
| | | ALPAPIEKTISKAKGQPREPQVYTLPPSRDELTKNQVSLTCLV |
| | | KGFYPSDIAVEWESNGQPENNYKTTPPVLDSDGSFFLYSRLT |
| | | VDKSRWQQGNVFSCSVLHEALHSHYTQKSLSLSPGK |
| 74 | EAC831: anti-VEGF | QVQLVQSGAEVKKPGSSVKVSCKASGYDFTHYGMNWVRQA |
| | bevacizumab heavy | PGQGLEWMGWINTYTGEPTYAQKFQGRFTFTADESTSTAYM |
| | chain VH5 variant | ELSSLRSEDTAVYYCAKYPYYYGTSHWYFDVWGQGTLVTV |
| | (BevaVH5) with IgG1 | SSASTKGPSVFPLAPSSKSTSGGTAALGCLVKDYFPEPVTVSW |
| | Fc with K409R, | NSGALTSGVHTFPAVLQSSGLYSLSSVVTVPSSSLGTQTYICN |
| | M428L, N434S | VNHKPSNTKVDKKVEPKSCDKTHTCPPCPAPELLGGPSVFLF |
| | mutations | PPKPKDTLMISRTPEVTCVVVDVSHEDPEVKFNWYVDGVEV |
| | | HNAKTKPREEQYNSTYRVVSVLTVLHQDWLNGKEYKCKVS |
| | | NKALPAPIEKTISKAKGQPREPQVYTLPPSRDELTKNQVSLTC |
| | | LVKGFYPSDIAVEWESNGQPENNYKTTPPVLDSDGSFFLYSR |
| | | LTVDKSRWQQGNVFSCSVLHEALHSHYTQKSLSLSPGK |
| 75 | EAC833: anti-VEGF | DIQMTQSPSSLSASVGDRVTITCRASQDISNYLNWYQQKPGK |
| | bevacizumab light | APKVLIYFTSSLHSGVPSRFSGSGSGTDFTLTISSLQPEDFATY |
| | chain VL1 variant | YCQQYSTVPWTFGQGTKLEIKRTVAAPSVFIFPPSDEQLKSGT |
| | (BevaVL1) | ASVVCLLNNFYPREAKVQWKVDNALQSGNSQESVTEQDSK |
| | | DSTYSLSSTLTLSKADYEKHKVYACEVTHQGLSSPVTKSFNR |
| | | GEC |
| 76 | EAC834: anti-VEGF | DIVMTQTPLSLPVTPGEPASISCRASQDISNYLNWYLQKPGQS |
| | bevacizumab light | PQVLIYFTSSLHSGVPDRFSGSGSGTDFTLKISRLEAEDVGVY |
| | chain VL2 variant | YCQQYSTVPWTFGQGTKLEIKRTVAAPSVFIFPPSDEQLKSGT |
| | (BevaVL1) | ASVVCLLNNFYPREAKVQWKVDNALQSGNSQESVTEQDSK |
| | | DSTYSLSSTLTLSKADYEKHKVYACEVTHQGLSSPVTKSFNR |
| | | GEC |
| 77 | EAC846: anti-VEGF | QVQLVQSGAEVKKPGSSVKVSCKASGYTFTNYGMNWVRQA |
| | bevacizumab heavy | PGQGLEWMGWINTYTGEPTYAQKFQGRVTITADESTSTAYM |
| | chain VH3 variant | ELSSLRSEDTAVYYCAKYPHYYGSSHWYFDVWGQGTLVTV |
| | (BevaVH3) with IgG1 | SSASTKGPSVFPLAPSSKSTSGGTAALGCLVKDYFPEPVTVSW |
| | Fc with E345R, | NSGALTSGVHTFPAVLQSSGLYSLSSVVTVPSSSLGTQTYICN |
| | K409R, M428L, | VNHKPSNTKVDKKVEPKSCDKTHTCPPCPAPELLGGPSVFLF |
| | N434S mutations | PPKPKDTLMISRTPEVTCVVVDVSHEDPEVKFNWYVDGVEV |
| | | HNAKTKPREEQYNSTYRVVSVLTVLHQDWLNGKEYKCKVS |
| | | NKALPAPIEKTISKAKGQPRRPQVYTLPPSRDELTKNQVSLTC |
| | | LVKGFYPSDIAVEWESNGQPENNYKTTPPVLDSDGSFFLYSR |
| | | LTVDKSRWQQGNVFSCSVLHEALHSHYTQKSLSLSPGK |
| 78 | EAC855: anti-VEGF | QVQLVQSGAEVKKPGSSVKVSCKASGYTFTNYGMNWVRQA |
| | bevacizumab heavy | PGQGLEWMGWINTYTGEPTYAQKFQGRFTFTADESTSTAYM |
| | chain VH2 variant | ELSSLRSEDTAVYYCAKYPHYYGSSHWYFDVWGQGTLVTV |
| | (BevaVH2) with IgG1 | SSASTKGPSVFPLAPSSKSTSGGTAALGCLVKDYFPEPVTVSW |
| | Fc with E345R, | NSGALTSGVHTFPAVLQSSGLYSLSSVVTVPSSSLGTQTYICN |
| | K409R, M428L, | VNHKPSNTKVDKKVEPKSCDKTHTCPPCPAPELLGGPSVFLF |
| | N434S mutations | PPKPKDTLMISRTPEVTCVVVDVSHEDPEVKFNWYVDGVEV |
| | | HNAKTKPREEQYNSTYRVVSVLTVLHQDWLNGKEYKCKVS |
| | | NKALPAPIEKTISKAKGQPRRPQVYTLPPSRDELTKNQVSLTC |
| | | LVKGFYPSDIAVEWESNGQPENNYKTTPPVLDSDGSFFLYSR |
| | | LTVDKSRWQQGNVFSCSVLHEALHSHYTQKSLSLSPGK |
| 79 | EAC863: anti-VEGF | EVQLVESGGGLVQPGGSLRLSCAASGYTFTNYGMNWVRQA |
| | bevacizumab heavy | PGKGLEWVGWINTYTGEPTYAADFKRRFTFSLDTSKSTAYL |
| | chain with IgG1 Fc | QMNSLRAEDTAVYYCAKYPHYYGSSHWYFDVWGQGTLVT |
| | | VSSASTKGPSVFPLAPSSKSTSGGTAALGCLVKDYFPEPVTVS |
| | | WNSGALTSGVHTFPAVLQSSGLYSLSSVVTVPSSLGTQTYIC |
| | | NVNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKV |
| | | SNKALPAPIEKTISKAKGQPREPQVYTLPPSREEMTKNQVSLT |
| | | CLVKGFYPSDIAVEWESNGQPENNYKTTPPVLDSDGSFFLYS |
| | | KLTVDKSRWQQGNVFSCSVMHEALHNHYTQKSLSLSPGK |
| 80 | EAC1042: anti-VEGF | QVQLVQSGAEVKKPGSSVKVSCKASGYTFTNYGMNWVRQA |
| | bevacizumab heavy | PGQGLEWMGWINTYTGEPTYAQKFQGRVTITADESTSTAYM |
| | chain VH3 variant | ELSSLRSEDTAVYYCAKYPHYYGSSHWYFDVWGQGTLVTV |
| | (BevaVH3) with IgG1 | SSASTKGPSVFPLAPSSKSTSGGTAALGCLVKDYFPEPVTVSW |
| | Fc with T366S, | NSGALTSGVHTFPAVLQSSGLYSLSSVVTVPSSSLGTQTYICN |
| | L368A, Y407V, | VNHKPSNTKVDKKVEPKSCDKTHTCPPCPAPELLGGPSVFLF |
| | M428L, N434S | PPKPKDTLMISRTPEVTCVVVDVSHEDPEVKFNWYVDGVEV |
| | mutations | HNAKTKPREEQYNSTYRVVSVLTVLHQDWLNGKEYKCKVS |
| | | NKALPAPIEKTIS KAKGQPREPQVYTLPPSRDELTKNQVSLSC |
| | | AVKGFYPSDIAVEWESNGQPENNYKTTPPVLDSDGSFFLVSK |
| | | LTVDKSRWQQGNVFSCSVLHEALHSHYTQKSLSLSPGK |
| 81 | EAC1049: anti-VEGF | QVQLVQSGAEVKKPGSSVKVSCKASGYTFTNYGMNWVRQA |
| | bevacizumab heavy | PGQGLEWMGWINTYTGEPTYAQKFQGRVTITADESTSTAYM |
| | chain VH3 variant | ELSSLRSEDTAVYYCAKYPHYYGSSHWYFDVWGQGTLVTV |
| | (BevaVH3) with IgG1 | SSASTKGPSVFPLAPSSKSTSGGTAALGCLVKDYFPEPVTVSW |
| | Fc with F243L, | NSGALTSGVHTFPAVLQSSGLYSLSSVVTVPSSSLGTQTYICN |
| | R292P, Y300L, | VNHKPSNTKVDKKVEPKSCDKTHTCPPCPAPELLGGPSVFLL |
| | V305I, P396L, | PPKPKDTLMISRTPEVTCVVVDVSHEDPEVKFNWYVDGVEV |
| | T366S, L368A, | HNAKTKPPEEQYNSTLRVVSILTVLHQDWLNGKEYKCKVSN |
| | Y407V mutations | KALPAPIEKTISKAKGQPREPQVYTLPPSRDELTKNQVSLSCA |
| | | VKGFYPSDIAVEWESNGQPENNYKTTPLVLDSDGSFFLVSKL |
| | | TVDKSRWQQGNVFSCSVMHEALHNHYTQKSLSLSPGK |
| 82 | ScFv of PD-L1 | EVQLVESGGGLVQPGGSLRLSCAASGFTFSDSWIHWVRQAP |
| | antibody | GKGLEWVAWISPYGGSTYYADSVKGRFTISADTSKNTAYLQ |
| | | MNSLRAEDTAVYYCARRHWPGGFDYWGQGTLVTVSSGGG |
| | | GSGGGGSGGGGSDIQMTQSPSSLSASVGDRVTITCRASQDVS |
| | | TAVAWYQQKPGKAPKLLIYSASFLYSGVPSRFSGSGSGTDFT |
| | | LTISSLQPEDFATYYCQQYLYHPATFGQGTKVEIK |
| 83 | EAC511: anti-HER2 | DIQMTQSPSSLSASVGDRVTITCRASQDVNTAVAWYQQKPG |
| | trastuzumab light | KAPKLLIYSASFLYSGVPSRFSGSRSGTDFTLTISSLQPEDFAT |
| | chain with ScFv of | YYCQQHYTTPPTFGQGTKVEIKRTVAAPSVFIFPPSDEQLKSG |
| | PD-L1 antibody | TASVVCLLNNFYPREAKVQWKVDNALQSGNSQESVTEQDSK |
| | | DSTYSLSSTLTLSKADYEKHKVYACEVTHQGLSSPVTKSFNR |
| | | GECGGGGSGGGGSGGGGSGGGGSEVQLVESGGGLVQPGGS |
| | | LRLSCAASGFTFSDSWIHWVRQAPGKGLEWVAWISPYGGST |
| | | YYADSVKGRFTISADTSKNTAYLQMNSLRAEDTAVYYCARR |
| | | HWPGGFDYWGQGTLVTVSSGGGGSGGGGSGGGGSDIQMTQ |
| | | SPSSLSASVGDRVTITCRASQDVSTAVAWYQQKPGKAPKLLI |
| | | YSASFLYSGVPSRFSGSGSGTDFTLTISSLQPEDFATYYCQQY |
| | | LYHPATFGQGTKVEIK |
| 84 | EAC514: anti-VEGF | DIQMTQSPSSLSASVGDRVTITCSASQDISNYLNWYQQKPGK |
| | bevacizumab light | APKVLIYFTSSLHSGVPSRFSGSGSGTDFTLTISSLQPEDFATY |
| | chain with ScFv of | YCQQYSTVPWTFGQGTKVEIKRTVAAPSVFIFPPSDEQLKSGT |
| | PD-L1 antibody | ASVVCLLNNFYPREAKVQWKVDNALQSGNSQESVTEQDSK |
| | | DSTYSLSSTLTLSKADYEKHKVYACEVTHQGLSSPVTKSFNR |
| | | GECGGGGSGGGGSGGGGSGGGGSEVQLVESGGGLVQPGGS |
| | | LRLSCAASGFTFSDSWIHWVRQAPGKGLEWVAWISPYGGST |
| | | YYADSVKGRFTISADTSKNTAYLQMNSLRAEDTAVYYCARR |
| | | HWPGGFDYWGQGTLVTVSSGGGGSGGGGSGGGGSDIQMTQ |
| | | SPSSLSASVGDRVTITCRASQDVSTAVAWYQQKPGKAPKLLI |
| | | YSASFLYSGVPSRFSGSGSGTDFTLTISSLQPEDFATYYCQQY |
| | | LYHPATFGQGTKVEIK |
| 101 | EAC204: Shielded | EPKSCDKTHTCPPCPGGGGSGPLGVRGGGGSEVQLVESGGG |
| | anti-HER2 | LVQPGGSLRLSCAASGFNIKDTYIHWVRQAPGKGLEWVARI |
| | trastuzumab heavy | YPTNGYTRYADSVKGRFTISADTSKNTAYLQMNSLRAEDTA |
| | chain with hinge | VYYCSRWGGDGFYAMDYWGQGTLVTVSSASTKGPSVFPLA |
| | masking domain, | PSSKSTSGGTAALGCLVKDYFPEPVTVSWNSGALTSGVHTFP |
| | MMP2 substrate | AVLQSSGLYSLSSVVTVPSSSLGTQTYICNVNHKPSNTKVDK |
| | sequence and IgG1 Fc | KVEPKSCDKTHTCPPCPAPPLLGPSVFLFPPKPKDTLMISRTPE |
| | with E233P, F405L, | VTCVVVDVSHEDPEVKFNWYVDGVEVHNAKTKPREEQYNS |
| | M428L, N434S | TYRVVSVLTVLHQDWLNGKEYKCKVSNKALPAPIEKTISKA |
| | mutations and G236 | KGQPREPQVYTLPPSRDELTKNQVSLTCLVKGFYPSDIAVEW |
| | deleted | ESNGQPENNYKTTPPVLDSDGSFLLYSkLTVDKSRWQQGNVF |
| | | SCSVLHEALHSHYTQKSLSLSPGK |
| 102 | EAC205: Shielded | EPKSCDKTHTCPPCPGGGGSGPLGVRGGGGSDIQMTQSPSSL |
| | anti-HER2 | SASVGDRVTITCRASQDVNTAVAWYQQKPGKAPKLLIYSAS |
| | trastuzumab light | FLYSGVPSRFSGSRSGTDFTLTISSLQPEDFATYYCQQHYTTPP |
| | chain with hinge | TFGQGTKVEIKRTVAAPSVFIFPPSDEQLKSGTASVVCLLNNF |
| | masking domain and | YPREAKVQWKVDNALQSGNSQESVTEQDSKDSTYSLSSTLT |
| | MMP2 substrate | LSKADYEKHKVYACEVTHQGLSSPVTKSFNRGEC |
| | sequence | |
| 103 | EAC224: Shielded | GIVEECCFRSCDLALLETYCAGGGGSPLGLAGGGGGSEVQLV |
| | anti-HER2 | ESGGGLVQPGGSLRLSCAASGFNIKDTYIHWVRQAPGKGLE |
| | trastuzumab heavy | WVARIYPTNGYTRYADSVKGRFTISADTSKNTAYLQMNSLR |
| | chain with IGF2 A | AEDTAVYYCSRWGGDGFYAMDYWGQGTLVTVSSASTKGPS |
| | chain masking | VFPLAPSSKSTSGGTAALGCLVKDYFPEPVTVSWNSGALTSG |
| | domain, MMP2 | VHTFPAVLQSSGLYSLSSVVTVPSSSLGTQTYICNVNHKPSNT |
| | substrate sequence | KVDKKVEPKSCDKTHTCPPCPAPPLLGPSVFLFPPKPKDTLMI |
| | and IgG1 Fc with | SRTPEVTCVVVDVSHEDPEVKFNWYVDGVEVHNAKTKPREE |
| | E233P, F405L, | QYNSTYRVVSVLTVLHQDWLNGKEYKCKVSNKALPAPIEKT |
| | M428L, N434S | ISKAKGQPREPQVYTLPPSRDELTKNQVSLTCLVKGFYPSDIA |
| | mutations and G236 | VEWESNGQPENNYKTTPPVLDSDGSFLLYSkLTVDKSRWQQ |
| | deleted | GNVFSCSVLHEALHSHYTQKSLSLSPGK |
| 104 | EAC225: Shielded | AYRPSETLCGGELVDTLQFVCGDRGFYFGGGGSPLGLAGGG |
| | anti-HER2 | GGSDIQMTQSPSSLSASVGDRVTITCRASQDVNTAVAWYQQ |
| | trastuzumab light | KPGKAPKLLIYSASFLYSGVPSRFSGSRSGTDFTLTISSLQPED |
| | chain with IGF2 B | FATYYCQQHYTTPPTFGQGTKVEIKRTVAAPSVFIFPPSDEQL |
| | chain masking | KSGTASVVCLLNNFYPREAKVQWKVDNALQSGNSQESVTEQ |
| | domain and MMP2 | DSKDSTYSLSSTLTLSKADYEKHKVYACEVTHQGLSSPVTKS |
| | substrate sequence | FNRGEC |
| 105 | EAC243: Shielded | GIVEECCFRSCDLALLETYCAGGGGSLSGRSDNHGGGGSEVQ |
| | anti-HER2 | LVESGGGLVQPGGSLRLSCAASGFNIKDTYIHWVRQAPGKGL |
| | trastuzumab heavy | EWVARIYPTNGYTRYADSVKGRFTISADTSKNTAYLQMNSL |
| | chain with IGF2 A | RAEDTAVYYCSRWGGDGFYAMDYWGQGTLVTVSSASTKGP |
| | chain masking | SVFPLAPSSKSTSGGTAALGCLVKDYFPEPVTVSWNSGALTS |
| | domain, uPA | GVHTFPAVLQSSGLYSLSSVVTVPSSSLGTQTYICNVNHKPSN |
| | substrate sequence | TKVDKKVEPKSCDKTHTCPPCPAPPLLGPSVFLFPPKPKDTL |
| | and IgG1 Fc with | MISRTPEVTCVVVDVSHEDPEVKFNWYVDGVEVHNAKTKPR |
| | E233P, F405L, | EEQYNSTYRVVSVLTVLHQDWLNGKEYKCKVSNKALPAPIE |
| | M428L, N434S | KTISKAKGQPREPQVYTLPPSRDELTKNQVSLTCLVKGFYPS |
| | mutations and G236 | DIAVEWESNGQPENNYKTTPPVLDSDGSFLLYSkLTVDKSRW |
| | deleted | QQGNVFSCSVLHEALHSHYTQKSLSLSPGK |
| 106 | EAC244: Shielded | AYRPSETLCGGELVDTLQFVCGDRGFYFGGGGSLSGRSDNH |
| | anti-HER2 | GGGGSDIQMTQSPSSLSASVGDRVTITCRASQDVNTAVAWY |
| | trastuzumab light | QQKPGKAPKLLIYSASFLYSGVPSRFSGSRSGTDFTLTISSLQP |
| | chain with uPA | EDFATYYCQQHYTTPPTFGQGTKVEIKRTVAAPSVFIFPPSDE |
| | masking domain and | QLKSGTASVVCLLNNFYPREAKVQWKVDNALQSGNSQESVT |
| | MMP2 substrate | EQDSKDSTYSLSSTLTLSKADYEKHKVYACEVTHQGLSSPVT |
| | sequence | KSFNRGEC |
| 107 | EAC260: Shielded | GQRGVVGLPGQRGERGFPGLPGYGGGGSPLGLAGGGGGSEV |
| | anti-HER2 | QLVESGGGLVQPGGSLRLSCAASGFNIKDTYIHWVRQAPGK |
| | trastuzumab heavy | GLEWVARIYPTNGYTRYADSVKGRFTISADTSKNTAYLQMN |
| | chain with collagen | SLRAEDTAVYYCSRWGGDGFYAMDYWGQGTLVTVSSASTK |
| | peptide 1 masking | GPSVFPLAPSSKSTSGGTAALGCLVKDYFPEPVTVSWNSGAL |
| | domain, MMP2 | TSGVHTFPAVLQSSGLYSLSSVVTVPSSSLGTQTYICNVNHKP |
| | substrate sequence | SNTKVDKKVEPKSCDKTHTCPPCPAPPLLGPSVFLFPPKPKDT |
| | and IgG1 Fc with | LMISRTPEVTCVVVDVSHEDPEVKFNWYVDGVEVHNAKTKP |
| | E233P, F405L, | REEQYNSTYRVVSVLTVLHQDWLNGKEYKCKVSNKALPAPI |
| | M428L, N434S | EKTISKAKGQPREPQVYTLPPSRDELTKNQVSLTCLVKGFYPS |
| | mutations and G236 | DIAVEWESNGQPENNYKTTPPVLDSDGSFLLYSkLTVDKSRW |
| | deleted | QQGNVFSCSVLHEALHSHYTQKSLSLSPGK |
| 108 | EAC261: Shielded | DQCIVDDITYNVQDTFHKKHEEGHMLNCTCFGQGRGRWKC |
| | anti-HER2 | DPVDQCQDSETGTFYQIGDSWEKYVHGVRYQCYCYGRGIGE |
| | trastuzumab light | WHCQPLQTYPSSGGGGSPLGLAGGGGGSDIQMTQSPSSLSAS |
| | chain with fibronectin | VGDRVTITCRASQDVNTAVAWYQQKPGKAPKLLIYSASFLY |
| | peptide masking | SGVPSRFSGSRSGTDFTLTISSLQPEDFATYYCQQHYTTPPTFG |
| | domain and MMP2 | QGTKVEIKRTVAAPSVFIFPPSDEQLKSGTASVVCLLNNFYPR |
| | substrate sequence | EAKVQWKVDNALQSGNSQESVTEQDSKDSTYSLSSTLTLSK |
| | | ADYEKHKVYACEVTHQGLSSPVTKSFNRGEC |
| 109 | EAC290: Shielded | GQRGVVGLPGQRGERGFPGLPGYGGGGSPLGLAGGGGGSEV |
| | anti-HER2 | QLVESGGGLVQPGGSLRLSCAASGFNIKDTYIHWVRQAPGK |
| | trastuzumab heavy | GLEWVARIYPTNGYTRYADSVKGRFTISADTSKNTAYLQMN |
| | chain with collagen | SLRAEDTAVYYCSRWGGDGFYAMDYWGQGTLVTVSSASTK |
| | peptide 1 masking | GPSVFPLAPSSKSTSGGTAALGCLVKDYFPEPVTVSWNSGAL |
| | domain, MMP2 | TSGVHTFPAVLQSSGLYSLSSVVTVPSSSLGTQTYICNVNHKP |
| | substrate sequence | SNTKVDKKVEPKSCDKTHTCPPCPAPELLGGPSVFLFPPKPKD |
| | and IgG1 Fc with | TLMISRTPEVTCVVVDVSHEDPEVKFNWYVDGVEVHNAKTK |
| | F405L, M428L, | PREEQYNSTYRVVSVLTVLHQDWLNGKEYKCKVSNKALPAP |
| | N434S mutations | IEKTISKAKGQPREPQVYTLPPSRDELTKNQVSLTCLVKGFYP |
| | | SDIAVEWESNGQPENNYKTTPPVLDSDGSFLLYSkLTVDKSR |
| | | WQQGNVFSCSVLHEALHSHYTQKSLSLSPGK |
| 110 | EAC293: Shielded | GILEECCFRSCDLALLETYCAGGGGSPLGLAGGGGGSEVQLV |
| | anti-HER2 | ESGGGLVQPGGSLRLSCAASGFNIKDTYIHWVRQAPGKGLE |
| | trastuzumab heavy | WVARIYPTNGYTRYADSVKGRFTISADTSKNTAYLQMNSLR |
| | chain with IGF2 A | AEDTAVYYCSRWGGDGFYAMDYWGQGTLVTVSSASTKGPS |
| | chain with V43L | VFPLAPSSKSTSGGTAALGCLVKDYFPEPVTVSWNSGALTSG |
| | mutation, MMP2 | VHTFPAVLQSSGLYSLSSVVTVPSSSLGTQTYICNVNHKPSNT |
| | substrate sequence | KVDKKVEPKSCDKTHTCPPCPAPELLGGPSVFLFPPKPKDTL |
| | and IgG1 Fc with | MISRTPEVTCVVVDVSHEDPEVKFNWYVDGVEVHNAKTKPR |
| | F405L, M428L, | EEQYNSTYRVVSVLTVLHQDWLNGKEYKCKVSNKALPAPIE |
| | N434S mutations | KTISKAKGQPREPQVYTLPPSRDELTKNQVSLTCLVKGFYPS |
| | | DIAVEWESNGQPENNYKTTPPVLDSDGSFLLYSkLTVDKSRW |
| | | QQGNVFSCSVLHEALHSHYTQKSLSLSPGK |
| 111 | EAC294: Shielded | AYRPSETLCGGELVDTLQFVCGDRGFAFGGGGSPLGLAGGG |
| | anti-HER2 | GGSDIQMTQSPSSLSASVGDRVTITCRASQDVNTAVAWYQQ |
| | trastuzumab light | KPGKAPKLLIYSASFLYSGVPSRFSGSRSGTDFTLTISSLQPED |
| | chain with IGF2 B | FATYYCQQHYTTPPTFGQGTKVEIKRTVAAPSVFIFPPSDEQL |
| | chain with Y27A | KSGTASVVCLLNNFYPREAKVQWKVDNALQSGNSQESVTEQ |
| | mutation and MMP2 | DSKDSTYSLSSTLTLSKADYEKHKVYACEVTHQGLSSPVTKS |
| | substrate sequence | FNRGEC |
| 112 | EAC507: Shielded | GILEECCFRSCDLALLETYCAGGGGSPLGLAGGGGGSEVQLV |
| | anti-HER2 | ESGGGLVQPGGSLRLSCAASGFTFTDYTMDWVRQAPGKGLE |
| | pertuzumab heavy | WVADVNPNSGGSIYNQRFKGRFTLSVDRSKNTLYLQMNSLR |
| | chain with IGF2 A | AEDTAVYYCARNLGPSFYFDYWGQGTLVTVSSASTKGPSVF |
| | chain with V43L | PLAPSSKSTSGGTAALGCLVKDYFPEPVTVSWNSGALTSGVH |
| | mutation, MMP2 | TFPAVLQSSGLYSLSSVVTVPSSSLGTQTYICNVNHKPSNTKV |
| | substrate sequence | DKKVEPKSCDKTHTCPPCPAPELLGGPSVFLFPPKPKDTLMIS |
| | and IgG1 Fc with | RTPEVTCVVVDVSHEDPEVKFNWYVDGVEVHNAKTKPREE |
| | F405L, M428L, | QYNSTYRVVSVLTVLHQDWLNGKEYKCKVSNKALPAPIEKT |
| | N434S mutations | ISKAKGQPREPQVYTLPPSRDELTKNQVSLTCLVKGFYPSDIA |
| | | VEWESNGQPENNYKTTPPVLDSDGSFFLYSRLTVDKSRWQQ |
| | | GNVFSCSVLHEALHSHYTQKSLSLSPGK |
| 113 | EAC508: Shielded | AYRPSETLCGGELVDTLQFVCGDRGFAFGGGGSPLGLAGGG |
| | anti-HER2 | GGSDIQMTQSPSSLSASVGDRVTITCKASQDVSIGVAWYQQK |
| | pertuzumab light | PGKAPKLLIYSASYRYTGVPSRFSGSGSGTDFTLTISSLQPEDF |
| | chain with IGF2 B | ATYYCQQYYIYPYTFGQGTKVEIKRTVAAPSVFIFPPSDEQLK |
| | chain with Y27A | SGTASVVCLLNNFYPREAKVQWKVDNALQSGNSQESVTEQD |
| | mutation and MMP2 | SKDSTYSLSSTLTLSKADYEKHKVYACEVTHQGLSSPVTKSF |
| | substrate sequence | NRGEC |
| 114 | EAC510: Shielded | GILEECCFRSCDLALLETYCAGGGGSPLGLAGGGGGSEVQLV |
| | anti-HER2 | ESGGGLVQPGGSLRLSCAASGFNIKDTYIHWVRQAPGKGLE |
| | trastuzumab heavy | WVARIYPTNGYTRYADSVKGRFTISADTSKNTAYLQMNSLR |
| | chain with IGF2 A | AEDTAVYYCSRWGGDGFYAMDYWGQGTLVTVSSASTKGPS |
| | chain with V43L | VFPLAPSSKSTSGGTAALGCLVKDYFPEPVTVSWNSGALTSG |
| | mutation, MMP2 | VHTFPAVLQSSGLYSLSSVVTVPSSSLGTQTYICNVNHKPSNT |
| | substrate sequence | KVDKKVEPKSCDKTHTCPPCPAPELLGGPSVFLFPPKPKDTL |
| | and IgG1 Fc with | MISRTPEVTCVVVDVSHEDPEVKFNWYVDGVEVHNAKTKPR |
| | E345R, F405L, | EEQYNSTYRVVSVLTVLHQDWLNGKEYKCKVSNKALPAPIE |
| | M428L, N434S | KTISKAKGQPRRPQVYTLPPSRDELTKNQVSLTCLVKGFYPS |
| | mutations | DIAVEWESNGQPENNYKTTPPVLDSDGSFLLYSkLTVDKSRW |
| | | QQGNVFSCSVLHEALHSHYTQKSLSLSPGK |
| 115 | EAC515: Shielded | GQRGVVGLPGQRGERGGGGSPLGLAGGGGGSEVQLVESGG |
| | anti-HER2 | GLVQPGGSLRLSCAASGFNIKDTYIHWVRQAPGKGLEWVAR |
| | trastuzumab heavy | IYPTNGYTRYADSVKGRFTISADTSKNTAYLQMNSLRAEDTA |
| | chain with collagen | VYYCSRWGGDGFYAMDYWGQGTLVTVSSASTKGPSVFPLA |
| | peptide 2 masking | PSSKSTSGGTAALGCLVKDYFPEPVTVSWNSGALTSGVHTFP |
| | domain, MMP2 | AVLQSSGLYSLSSVVTVPSSSLGTQTYICNVNHKPSNTKVDK |
| | substrate sequence | KVEPKSCDKTHTCPPCPAPELLGGPSVFLFPPKPKDTLMISRT |
| | and IgG1 Fc with | PEVTCVVVDVSHEDPEVKFNWYVDGVEVHNAKTKPREEQY |
| | F405L, M428L, | NSTYRVVSVLTVLHQDWLNGKEYKCKVSNKALPAPIEKTISK |
| | N434S mutations | AKGQPREPQVYTLPPSRDELTKNQVSLTCLVKGFYPSDIAVE |
| | | WESNGQPENNYKTTPPVLDSDGSFLLYSkLTVDKSRWQQGN |
| | | VFSCSVLHEALHSHYTQKSLSLSPGK |
| 116 | EAC527: Shielded | GILEECCFRSCDLALLETYCAGGGGSPLGLAGGGGGSQVQLV |
| | anti-HER2 | QSGAEVKKPGSSVKVSCKASGFNIKDTYIHWVRQAPGQGLE |
| | trastuzumab heavy | WMGRIYPTNGYTRYAQKFQGRATITADESTSTAYMEVSSLR |
| | chain VH1 variant | SEDTAVYYCSRWGGDGFYAMDYWGQGTLVTVSSASTKGPS |
| | (TrasVH1) with IGF2 | VFPLAPSSKSTSGGTAALGCLVKDYFPEPVTVSWNSGALTSG |
| | A chain with V43L | VHTFPAVLQSSGLYSLSSVVTVPSSSLGTQTYICNVNHKPSNT |
| | mutation, MMP2 | KVDKKVEPKSCDKTHTCPPCPAPELLGGPSVFLFPPKPKDTL |
| | substrate sequence | MISRTPEVTCVVVDVSHEDPEVKFNWYVDGVEVHNAKTKPR |
| | and IgG1 Fc with | EEQYNSTYRVVSVLTVLHQDWLNGKEYKCKVSNKALPAPIE |
| | F405L, M428L, | KTISKAKGQPREPQVYTLPPSRDELTKNQVSLTCLVKGFYPS |
| | N434S mutations | DIAVEWESNGQPENNYKTTPPVLDSDGSFLLYSkLTVDKSRW |
| | | QQGNVFSCSVLHEALHSHYTQKSLSLSPGK |
| 117 | EAC528: Shielded | GILEECCFRSCDLALLETYCAGGGGSPLGLAGGGGGSEVQLV |
| | anti-HER2 | ESGGGLVQPGPSLRLSCTASGFNIKDTYIHWVRQAPGKGLEW |
| | trastuzumab heavy | VGRIYPTNGYTRYAASFKGRATISADDSKSIAYLQVNSLKTE |
| | chain VH2 variant | DTAVYYCTRWGGDGFYAMDYWGQGTLVTVSSASTKGPSVF |
| | (TrasVH2) with IGF2 | PLAPSSKSTSGGTAALGCLVKDYFPEPVTVSWNSGALTSGVH |
| | A chain with V43L | TFPAVLQSSGLYSLSSVVTVPSSSLGTQTYICNVNHKPSNTKV |
| | mutation, MMP2 | DKKVEPKSCDKTHTCPPCPAPELLGGPSVFLFPPKPKDTLMIS |
| | substrate sequence | RTPEVTCVVVDVSHEDPEVKFNWYVDGVEVHNAKTKPREE |
| | and IgG1 Fc with | QYNSTYRVVSVLTVLHQDWLNGKEYKCKVSNKALPAPIEKT |
| | F405L, M428L, | ISKAKGQPREPQVYTLPPSRDELTKNQVSLTCLVKGFYPSDIA |
| | N434S mutations | VEWESNGQPENNYKTTPPVLDSDGSFLLYSkLTVDKSRWQQ |
| | | GNVFSCSVLHEALHSHYTQKSLSLSPGK |
| 118 | EAC529: Shielded | GILEECCFRSCDLALLETYCAGGGGSPLGLAGGGGGSEVQLV |
| | anti-HER2 | QSGAEVKKPGESLKISCKGSGFNIKDTYIHWVRQMPGKGLE |
| | trastuzumab heavy | WMGRIYPTNGYTRYSPSFQGQATISADKSISTAYLQVSSLKAS |
| | chain VH3 variant | DTAMYYCSRWGGDGFYAMDYWGQGTLVTVSSASTKGPSVF |
| | (TrasVH3) with IGF2 | PLAPSSKSTSGGTAALGCLVKDYFPEPVTVSWNSGALTSGVH |
| | A chain with V43L | TFPAVLQSSGLYSLSSVVTVPSSSLGTQTYICNVNHKPSNTKV |
| | mutation, MMP2 | DKKVEPKSCDKTHTCPPCPAPELLGGPSVFLFPPKPKDTLMIS |
| | substrate sequence | RTPEVTCVVVDVSHEDPEVKFNWYVDGVEVHNAKTKPREE |
| | and IgG1 Fc with | QYNSTYRVVSVLTVLHQDWLNGKEYKCKVSNKALPAPIEKT |
| | F405L, M428L, | ISKAKGQPREPQVYTLPPSRDELTKNQVSLTCLVKGFYPSDIA |
| | N434S mutations | VEWESNGQPENNYKTTPPVLDSDGSFLLYSkLTVDKSRWQQ |
| | | GNVFSCSVLHEALHSHYTQKSLSLSPGK |
| 119 | EAC532: Shielded | AYRPSETLCGGELVDTLQFVCGDRGFAFGGGGSPLGLAGGG |
| | anti-HER2 | GGSDIVMTQTPLSLPVTPGEPASISCRASQDVNTAVAWYLQK |
| | trastuzumab light | PGQSPQLLIYSASFRYTGVPDRFSGSGSGTDFTLKISRVEAED |
| | chain VL1 variant | VGVYYCQQHYTTPPTFGQGTKVEIKRTVAAPSVFIFPPSDEQL |
| | (TrasVL1) with IGF2 | KSGTASVVCLLNNFYPREAKVQWKVDNALQSGNSQESVTEQ |
| | B chain with Y27A | DSKDSTYSLSSTLTLSKADYEKHKVYACEVTHQGLSSPVTKS |
| | mutation and MMP2 | FNRGEC |
| | substrate sequence | |
| 120 | EAC533: Shielded | AYRPSETLCGGELVDTLQFVCGDRGFAFGGGGSPLGLAGGG |
| | anti-HER2 | GGSDIVMTQSPDSLAVSLGERVTINCKASQDVNTAVAWYQQ |
| | trastuzumab light | KPGQSPKLLIYSASFRYSGVPDRFSGSGSGTDFTFTISSVQAED |
| | chain VL2 variant | LAVYYCQQHYTTPPTFGQGTKVEIKRTVAAPSVFIFPPSDEQ |
| | (TrasVL2) with IGF2 | KSGTASVVCLLNNFYPREAKVQWKVDNALQSGNSQESVTEQ |
| | B chain with Y27A | DSKDSTYSLSSTLTLSKADYEKHKVYACEVTHQGLSSPVTKS |
| | mutation and MMP2 | FNRGEC |
| | substrate sequence | |
| 121 | EAC535: Shielded | GQRGVVGLPGQRGERGGGGSPLGLAGGGGGSQVQLVQSGA |
| | anti-HER2 | EVKKPGSSVKVSCKASGFNIKDTYIHWVRQAPGQGLEWMGR |
| | trastuzumab heavy | IYPTNGYTRYAQKFQGRATITADESTSTAYMEVSSLRSEDTA |
| | chain VH1 variant | VYYCSRWGGDGFYAMDYWGQGTLVTVSSASTKGPSVFPLA |
| | (TrasVH1) with | PSSKSTSGGTAALGCLVKDYFPEPVTVSWNSGALTSGVHTFP |
| | collagen peptide 2 | AVLQSSGLYSLSSVVTVPSSSLGTQTYICNVNHKPSNTKVDK |
| | masking domain, | KVEPKSCDKTHTCPPCPAPELLGGPSVFLFPPKPKDTLMISRT |
| | MMP2 substrate | PEVTCVVVDVSHEDPEVKFNWYVDGVEVHNAKTKPREEQY |
| | sequence and IgG1 Fc | NSTYRVVSVLTVLHQDWLNGKEYKCKVSNKALPAPIEKTISK |
| | with F405L, M428L, | AKGQPREPQVYTLPPSRDELTKNQVSLTCLVKGFYPSDIAVE |
| | N434S mutations | WESNGQPENNYKTTPPVLDSDGSFLLYSkLTVDKSRWQQGN |
| | | VFSCSVLHEALHSHYTQKSLSLSPGK |
| 122 | EAC536: Shielded | GQRGVVGLPGQRGERGGGGSPLGLAGGGGGSEVQLVESGG |
| | anti-HER2 | GLVQPGPSLRLSCTASGFNIKDTYIHWVRQAPGKGLEWVGRI |
| | trastuzumab heavy | YPTNGYTRYAASFKGRATISADDSKSIAYLQVNSLKTEDTAV |
| | chain VH2 variant | YYCTRWGGDGFYAMDYWGQGTLVTVSSASTKGPSVFPLAP |
| | (TrasVH2) with | SSKSTSGGTAALGCLVKDYFPEPVTVSWNSGALTSGVHTFPA |
| | collagen peptide 2 | VLQSSGLYSLSSVVTVPSSSLGTQTYICNVNHKPSNTKVDKK |
| | masking domain, | VEPKSCDKTHTCPPCPAPELLGGPSVFLFPPKPKDTLMISRTPE |
| | MMP2 substrate | VTCVVVDVSHEDPEVKFNWYVDGVEVHNAKTKPREEQYNS |
| | sequence and IgG1 Fc | TYRVVSVLTVLHQDWLNGKEYKCKVSNKALPAPIEKTISKA |
| | with F405L, M428L, | KGQPREPQVYTLPPSRDELTKNQVSLTCLVKGFYPSDIAVEW |
| | N434S mutations | ESNGQPENNYKTTPPVLDSDGSFLLYSkLTVDKSRWQQGNVF |
| | | SCSVLHEALHSHYTQKSLSLSPGK |
| 123 | EAC537: Shielded | GQRGVVGLPGQRGERGGGGSPLGLAGGGGGSEVQLVQSGA |
| | anti-HER2 | EVKKPGESLKISCKGSGFNIKDTYIHWVRQMPGKGLEWMGR |
| | trastuzumab heavy | IYPTNGYTRYSPSFQGQATISADKSISTAYLQVSSLKASDTAM |
| | chain VH3 variant | YYCSRWGGDGFYAMDYWGQGTLVTVSSASTKGPSVFPLAP |
| | (TrasVH3) with | SSKSTSGGTAALGCLVKDYFPEPVTVSWNSGALTSGVHTFPA |
| | collagen peptide 2 | VLQSSGLYSLSSVVTVPSSSLGTQTYICNVNHKPSNTKVDKK |
| | masking domain, | VEPKSCDKTHTCPPCPAPELLGGPSVFLFPPKPKDTLMISRTPE |
| | MMP2 substrate | VTCVVVDVSHEDPEVKFNWYVDGVEVHNAKTKPREEQYNS |
| | sequence and IgG1 Fc | TYRVVSVLTVLHQDWLNGKEYKCKVSNKALPAPIEKTISKA |
| | with F405L, M428L, | KGQPREPQVYTLPPSRDELTKNQVSLTCLVKGFYPSDIAVEW |
| | N434S mutations | ESNGQPENNYKTTPPVLDSDGSFLLYSkLTVDKSRWQQGNVF |
| | | SCSVLHEALHSHYTQKSLSLSPGK |
| 124 | EAC540: Shielded | DQCIVDDITYNVQDTFHKKHEEGHMLNCTCFGQGRGRWKC |
| | anti-HER2 | DPVDQCQDSETGTFYQIGDSWEKYVHGVRYQCYCYGRGIGE |
| | trastuzumab light | WHCQPLQTYPSSGGGGSPLGLAGGGGGSDIVMTQTPLSLPVT |
| | chain VL1 variant | PGEPASISCRASQDVNTAVAWYLQKPGQSPQLLIYSASFRYT |
| | (TrasVL1) with | GVPDRFSGSGSGTDFTLKISRVEAEDVGVYYCQQHYTTPPTF |
| | fibronectin peptide | GQGTKVEIKRTVAAPSVFIFPPSDEQLKSGTASVVCLLNNFYP |
| | masking domain and | REAKVQWKVDNALQSGNSQESVTEQDSKDSTYSLSSTLTLS |
| | MMP2 substrate | KADYEKHKVYACEVTHQGLSSPVTKSFNRGEC |
| | sequence | |
| 125 | EAC541: Shielded | DQCIVDDITYNVQDTFHKKHEEGHMLNCTCFGQGRGRWKC |
| | anti-HER2 | DPVDQCQDSETGTFYQIGDSWEKYVHGVRYQCYCYGRGIGE |
| | trastuzumab light | WHCQPLQTYPSSGGGGSPLGLAGGGGGSDIVMTQSPDSLAV |
| | chain VL2 variant | SLGERVTINCKASQDVNTAVAWYQQKPGQSPKLLIYSASFRY |
| | (TrasVL2) with | SGVPDRFSGSGSGTDFTFTISSVQAEDLAVYYCQQHYTTPPTF |
| | fibronectin peptide | GQGTKVEIKRTVAAPSVFIFPPSDEQLKSGTASVVCLLNNFYP |
| | masking domain and | REAKVQWKVDNALQSGNSQESVTEQDSKDSTYSLSSTLTLS |
| | MMP2 substrate | KADYEKHKVYACEVTHQGLSSPVTKSFNRGEC |
| | sequence | |
| 126 | EAC561: Shielded | GILEECCFRSCDLALLETYCAGGGGSPLGLAGGGGGSQVQLV |
| | anti-HER2 | QSGAEVKKPGSSVKVSCKASGFNIKDTYIHWVRQAPGQGLE |
| | trastuzumab heavy | WMGRIYPTNGYTRYAQKFQGRATITADESTSTAYMEVSSLR |
| | chain VH1 variant | SEDTAVYYCSRWGGDGFYAMDYWGQGTLVTVSSASTKGPS |
| | (TrasVH1) with IGF2 | VFPLAPSSKSTSGGTAALGCLVKDYFPEPVTVSWNSGALTSG |
| | A chain with V43L | VHTFPAVLQSSGLYSLSSVVTVPSSSLGTQTYICNVNHKPSNT |
| | mutation, MMP2 | KVDKKVEPKSCDKTHTCPPCPAPELLGGPSVFLFPPKPKDTL |
| | substrate sequence | MISRTPEVTCVVVDVSHEDPEVKFNWYVDGVEVHNAKTKPR |
| | and IgG1 Fc with | EEQYNSTYRVVSVLTVLHQDWLNGKEYKCKVSNKALPAPIE |
| | E345R, F405L, | KTISKAKGQPRRPQVYTLPPSRDELTKNQVSLTCLVKGFYPS |
| | M428L, N434S | DIAVEWESNGQPENNYKTTPPVLDSDGSFLLYSkLTVDKSRW |
| | mutations | QQGNVFSCSVLHEALHSHYTQKSLS LSPGK |
| 127 | EAC809: Shielded | GILEECCFRSCDLALLETYCAGGGGSRPKPVEVWRKGGGGS |
| | anti-HER2 | QVQLVQSGAEVKKPGSSVKVSCKASGFNIKDTYIHWVRQAP |
| | trastuzumab heavy | GQGLEWMGRIYPTNGYTRYAQKFQGRATITADESTSTAYME |
| | chain VH1 variant | VSSLRSEDTAVYYCSRWGGDGFYAMDYWGQGTLVTVSSAS |
| | (TrasVH1) with IGF2 | TKGPSVFPLAPSSKSTSGGTAALGCLVKDYFPEPVTVSWNSG |
| | A chain with V43L | ALTSGVHTFPAVLQSSGLYSLSSVVTVPSSSLGTQTYICNVNH |
| | mutation, MMP3 | KPSNTKVDKKVEPKSCDKTHTCPPCPAPELLGGPSVFLFPPKP |
| | substrate sequence | KDTLMISRTPEVTCVVVDVSHEDPEVKFNWYVDGVEVHNA |
| | and IgG1 Fc with | KTKPREEQYNSTYRVVSVLTVLHQDWLNGKEYKCKVSNKA |
| | F405L, M428L, | LPAPIEKTISKAKGQPREPQVYTLPPSRDELTKNQVSLTCLVK |
| | N434S mutations | GFYPSDIAVEWESNGQPENNYKTTPPVLDSDGSFLLYSkLTV |
| | | DKSRWQQGNVFSCSVLHEALHSHYTQKSLSLSPGK |
| 128 | EAC810: Shielded | AYRPSETLCGGELVDTLQFVCGDRGFAFGGGGSRPKPVEVW |
| | anti-HER2 | RKGGGGSDIVMTQTPLSLPVTPGEPASISCRASQDVNTAVAW |
| | trastuzumab light | YLQKPGQSPQLLIYSASFRYTGVPDRFSGSGSGTDFTLKISRV |
| | chain VL1 variant | EAEDVGVYYCQQHYTTPPTFGQGTKVEIKRTVAAPSVFIFPPS |
| | (TrasVL1) with IGF2 | DEQLKSGTASVVCLLNNFYPREAKVQWKVDNALQSGNSQES |
| | B chain with Y27A | VTEQDSKDSTYSLSSTLTLSKADYEKHKVYACEVTHQGLSSP |
| | mutation and MMP3 | VTKSFNRGEC |
| | substrate sequence | |
| 129 | EAC811: Shielded | GILEECCFRSCDLALLETYCAGGGGSLSGRSDNHGGGGSQVQ |
| | anti-HER2 | LVQSGAEVKKPGSSVKVSCKASGFNIKDTYIHWVRQAPGQG |
| | trastuzumab heavy | LEWMGRIYPTNGYTRYAQKFQGRATITADESTSTAYMEVSS |
| | chain VH1 variant | LRSEDTAVYYCSRWGGDGFYAMDYWGQGTLVTVSSASTKG |
| | (TrasVH1) with IGF2 | PSVFPLAPSSKSTSGGTAALGCLVKDYFPEPVTVSWNSGALT |
| | A chain with V43L | SGVHTFPAVLQSSGLYSLSSVVTVPSSSLGTQTYICNVNHKPS |
| | mutation, uPA | NTKVDKKVEPKSCDKTHTCPPCPAPELLGGPSVFLFPPKPKD |
| | substrate sequence G | TLMISRTPEVTCVVVDVSHEDPEVKFNWYVDGVEVHNAKTK |
| | and IgG1 Fc with | PREEQYNSTYRVVSVLTVLHQDWLNGKEYKCKVSNKALPAP |
| | F405L, M428L, | IEKTISKAKGQPREPQVYTLPPSRDELTKNQVSLTCLVKGFYP |
| | N434S mutations | SDIAVEWESNGQPENNYKTTPPVLDSDGSFLLYSkLTVDKSR |
| | | WQQGNVFSCSVLHEALHSHYTQKSLSLSPGK |
| 130 | EAC812: Shielded | GILEECCFRSCDLALLETYCAGGGGSLSGRSDNHGGGGSQVQ |
| | anti-HER2 | LVQSGAEVKKPGSSVKVSCKASGFNIKDTYIHWVRQAPGQG |
| | trastuzumab heavy | LEWMGRIYPTNGYTRYAQKFQGRATITADESTSTAYMEVSS |
| | chain VH1 variant | LRSEDTAVYYCSRWGGDGFYAMDYWGQGTLVTVSSASTKG |
| | (TrasVH1) with IGF2 | PSVFPLAPSSKSTSGGTAALGCLVKDYFPEPVTVSWNSGALT |
| | A chain with V43L | SGVHTFPAVLQSSGLYSLSSVVTVPSSSLGTQTYICNVNHKPS |
| | mutation, uPA | NTKVDKKVEPKSCDKTHTCPPCPAPELLGGPSVFLFPPKPKD |
| | substrate sequence G | TLMISRTPEVTCVVVDVSHEDPEVKFNWYVDGVEVHNAKTK |
| | and IgG1 Fc with | PREEQYNSTYRVVSVLTVLHQDWLNGKEYKCKVSNKALPAP |
| | E345R, F405L, | IEKTISKAKGQPRRPQVYTLPPSRDELTKNQVSLTCLVKGFYP |
| | M428L, N434S | SDIAVEWESNGQPENNYKTTPPVLDSDGSFLLYSkLTVDKSR |
| | mutations | WQQGNVFSCSVLHEALHSHYTQKSLSLSPGK |
| 131 | EAC813: Shielded | AYRPSETLCGGELVDTLQFVCGDRGFAFGGGGSLSGRSDNH |
| | anti-HER2 | GGGGSDIVMTQTPLSLPVTPGEPASISCRASQDVNTAVAWYL |
| | trastuzumab light | QKPGQSPQLLIYSASFRYTGVPDRFSGSGSGTDFTLKISRVEA |
| | chain VL1 variant | EDVGVYYCQQHYTTPPTFGQGTKVEIKRTVAAPSVFIFPPSDE |
| | (TrasVL1) with IGF2 | QLKSGTASVVCLLNNFYPREAKVQWKVDNALQSGNSQESVT |
| | B chain with Y27A | EQDSKDSTYSLSSTLTLSKADYEKHKVYACEVTHQGLSSPVT |
| | mutation and uPA | KSFNRGEC |
| | substrate sequence G | |
| 132 | EAC853: Shielded | AYRPSETLCGGELVDTLQFVCGDRGFAFGGGGSTGRGPSWV |
| | anti-HER2 | GGGGSDIVMTQTPLSLPVTPGEPASISCRASQDVNTAVAWYL |
| | trastuzumab light | QKPGQSPQLLIYSASFRYTGVPDRFSGSGSGTDFTLKISRVEA |
| | chain VL1 variant | EDVGVYYCQQHYTTPPTFGQGTKVEIKRTVAAPSVFIFPPSDE |
| | (TrasVL1) with IGF2 | QLKSGTASVVCLLNNFYPREAKVQWKVDNALQSGNSQESVT |
| | B chain with Y27A | EQDSKDSTYSLSSTLTLSKADYEKHKVYACEVTHQGLSSPVT |
| | mutation and uPA | KSFNRGEC |
| | substrate sequence H | |
| 133 | EAC1041: Shielded | GILEECCFRSCDLALLETYCAGGGGSPLGLAGGGGGSQVQLV |
| | anti-HER2 | QSGAEVKKPGSSVKVSCKASGFNIKDTYIHWVRQAPGQGLE |
| | trastuzumab heavy | WMGRIYPTNGYTRYAQKFQGRATITADESTSTAYMEVSSLR |
| | chain VH1 variant | SEDTAVYYCSRWGGDGFYAMDYWGQGTLVTVSSASTKGPS |
| | (TrasVH1) with IGF2 | VFPLAPSSKSTSGGTAALGCLVKDYFPEPVTVSWNSGALTSG |
| | A chain with V43L | VHTFPAVLQSSGLYSLSSVVTVPSSSLGTQTYICNVNHKPSNT |
| | mutation, MMP2 | KVDKKVEPKSCDKTHTCPPCPAPELLGGPSVFLFPPKPKDTL |
| | substrate sequence | MISRTPEVTCVVVDVSHEDPEVKFNWYVDGVEVHNAKTKPR |
| | and IgG1 Fc with | EEQYNSTYRVVSVLTVLHQDWLNGKEYKCKVSNKALPAPIE |
| | T366W, M428L, | KTISKAKGQPREPQVYTLPPSRDELTKNQVSLWCLVKGFYPS |
| | N434S mutations | DIAVEWESNGQPENNYKTTPPVLDSDGSFFLYSkLTVDKSRW |
| | | QQGNVFSCSVLHEALHSHYTQKSLSLSPGK |
| 134 | EAC1050: Shielded | GILEECCFRSCDLALLETYCAGGGGSPLGLAGGGGGSQVQLV |
| | anti-HER2 | QSGAEVKKPGSSVKVSCKASGFNIKDTYIHWVRQAPGQGLE |
| | trastuzumab heavy | WMGRIYPTNGYTRYAQKFQGRATITADESTSTAYMEVSSLR |
| | chain VH1 variant | SEDTAVYYCSRWGGDGFYAMDYWGQGTLVTVSSASTKGPS |
| | (TrasVH1) with IGF2 | VFPLAPSSKSTSGGTAALGCLVKDYFPEPVTVSWNSGALTSG |
| | A chain with V43L | VHTFPAVLQSSGLYSLSSVVTVPSSSLGTQTYICNVNHKPSNT |
| | mutation, MMP2 | KVDKKVEPKSCDKTHTCPPCPAPELLGGPSVFLLPPKPKDTL |
| | substrate sequence | MISRTPEVTCVVVDVSHEDPEVKFNWYVDGVEVHNAKTKPP |
| | and IgG1 Fc with | EEQYNSTLRVVSILTVLHQDWLNGKEYKCKVSNKALPAPIEK |
| | F243L, R292P, | TISKAKGQPREPQVYTLPPSRDELTKNQVSLWCLVKGFYPSDI |
| | Y300L, V305I, | AVEWESNGQPENNYKTTPLVLDSDGSFFLYSkLTVDKSRWQ |
| | P396L, T366W | QGNVFSCSVMHEALHNHYTQKSLSLSPGK |
| | mutations | |
| 135 | Shielded anti-HER2 | GQRGVVGLPGQRGERGFPGLPGYGGGGSPLGLAGGGGGSEV |
| | pertuzumab heavy | QLVESGGGLVQPGGSLRLSCAASGFTFTDYTMDWVRYAPGK |
| | chain with collagen | GLEWVADVNPNSGGSIYNQRFKGRFTLSVDRSKNTLYLQMN |
| | peptide 1 masking | SLRAEDTAVYYCARNLGPSFYFDYWGQGTLVTVSSASTKGP |
| | domain, MMP2 | SVFPLAPSSKSTSGGTAALGCLVKDYFPEPVTVSWNSGALTS |
| | substrate sequence | GVHTFPAVLQSSGLYSLSSVVTVPSSSLGTQTYICNVNHKPSN |
| | and IgG1 Fc with | TKVDKKVEPKSCDKTHTCPPCPAPELLGGPSVFLFPPKPKDTL |
| | F405L, M428L, | MISRTPEVTCVVVDVSHEDPEVKFNWYVDGVEVHNAKTKPR |
| | N434S mutations | EEQYNSTYRVVSVLTVLHQDWLNGKEYKCKVSNKALPAPIE |
| | | KTISKAKGQPREPQVYTLPPSRDELTKNQVSLTCLVKGFYPS |
| | | DIAVEWESNGQPENNYKTTPPVLDSDGSFLLYSkLTVDKSRW |
| | | QQGNVFSCSVLHEALHSHYTQKSLSLSPGK |
| 136 | Shielded anti-HER2 | DQCIVDDITYNVQDTFHKKHEEGHMLNCTCFGQGRGRWKC |
| | pertuzumab light | DPVDQCQDSETGTFYQIGDSWEKYVHGVRYQCYCYGRGIGE |
| | chain with fibronectin | WHCQPLQTYPSSGGGGSPLGLAGGGGGSDIQMTQSPSSLSAS |
| | peptide masking | VGDRVTITCKASQDVSIGVAWYQRKPGKAPKLLIYSASYRYT |
| | domain and MMP2 | GVPSRFSGSGSGTDFTLTISSLQPEDFATYYCQQYYIYPYTFG |
| | substrate sequence | QGTKVEIKRTVAAPSVFIFPPSDEQLKSGTASVVCLLNNFYPR |
| | | EAKVQWKVDNALQSGNSQESVTEQDSKDSTYSLSSTLTLSK |
| | | ADYEKHKVYACEVTHQGLSSPVTKSFNRGEC |
| 137 | Shielded anti-HER2 | EPKSCDKTHTCPPCPGGGGSGPLGVRGGGGSPLGLAGGGGG |
| | pertuzumab heavy | SEVQLVESGGGLVQPGGSLRLSCAASGFTFTDYTMDWVRYA |
| | chain with hinge | PGKGLEWVADVNPNSGGSIYNQRFKGRFTLSVDRSKNTLYL |
| | masking domain, | QMNSLRAEDTAVYYCARNLGPSFYFDYWGQGTLVTVSSAST |
| | MMP2 substrate | KGPSVFPLAPSSKSTSGGTAALGCLVKDYFPEPVTVSWNSGA |
| | sequence and IgG1 Fc | LTSGVHTFPAVLQSSGLYSLSSVVTVPSSSLGTQTYICNVNHK |
| | with F405L, M428L, | PSNTKVDKKVEPKSCDKTHTCPPCPAPELLGGPSVFLFPPKPK |
| | N434S mutations | DTLMISRTPEVTCVVVDVSHEDPEVKFNWYVDGVEVHNAKT |
| | | KPREEQYNSTYRVVSVLTVLHQDWLNGKEYKCKVSNKALP |
| | | APIEKTISKAKGQPREPQVYTLPPSRDELTKNQVSLTCLVKGF |
| | | YPSDIAVEWESNGQPENNYKTTPPVLDSDGSFLLYSkLTVDK |
| | | SRWQQGNVFSCSVLHEALHSHYTQKSLSLSPGK |
| 138 | Shielded anti-HER2 | EPKSCDKTHTCPPCPGGGGSGPLGVRGGGGSPLGLAGGGGG |
| | pertuzumab light | SDIQMTQSPSSLSASVGDRVTITCKASQDVSIGVAWYQRKPG |
| | chain with hinge | KAPKLLIYSASYRYTGVPSRFSGSGSGTDFTLTISSLQPEDFAT |
| | masking domain and | YYCQQYYIYPYTFGQGTKVEIKRTVAAPSVFIFPPSDEQLKSG |
| | MMP2 substrate | TASVVCLLNNFYPREAKVQWKVDNALQSGNSQESVTEQDSK |
| | sequence | DSTYSLSSTLTLSKADYEKHKVYACEVTHQGLSSPVTKSFNR |
| | | GEC |
| 139 | Shielded anti-HER2 | GPEADQQKACNHYKDPPFCTCGGGGSGGGGSGPLGVRGGG |
| | trastuzumab light | GSDIQMTQSPSSLSASVGDRVTITCRASQDVNTAVAWYQQK |
| | chain with epitope as | PGKAPKLLIYSASFLYSGVPSRFSGSRSGTDFTLTISSLQPEDF |
| | masking domain and | ATYYCQQHYTTPPTFGQGTKVEIKRTVAAPSVFIFPPSDEQLK |
| | MMP2 substrate | SGTASVVCLLNNFYPREAKVQWKVDNALQSGNSQESVTEQD |
| | sequence | SKDSTYSLSSTLTLSKADYEKHKVYACEVTHQGLSSPVTKSF |
| | | NRGEC |
| 140 | Shielded anti-HER2 | GPEADQQKACNHYKDPPFCTCGGGGSGGGGSGPLGVRGGG |
| | trastuzumab heavy | GSEVQLVESGGGLVQPGGSLRLSCAASGFNIKDTYIHWVRQA |
| | chain with epitope as | PGKGLEWVARIYPTNGYTRYADSVKGRFTISADTSKNTAYL |
| | masking domain, | QMNSLRAEDTAVYYCSRWGGDGFYAMDYWGQGTLVTVSS |
| | MMP2 substrate | ASTKGPSVFPLAPSSKSTSGGTAALGCLVKDYFPEPVTVSWN |
| | sequence and IgG1 Fc | SGALTSGVHTFPAVLQSSGLYSLSSVVTVPSSSLGTQTYICNV |
| | with F405L, M428L, | NHKPSNTKVDKKVEPKSCDKTHTCPPCPAPELLGGPSVFLFPP |
| | N434S mutations | KPKDTLMISRTPEVTCVVVDVSHEDPEVKFNWYVDGVEVHN |
| | | AKTKPREEQYNSTYRVVSVLTVLHQDWLNGKEYKCKVSNK |
| | | ALPAPIEKTISKAKGQPREPQVYTLPPSRDELTKNQVSLTCLV |
| | | KGFYPSDIAVEWESNGQPENNYKTTPPVLDSDGSFLLYSkLT |
| | | VDKSRWQQGNVFSCSVLHEALHSHYTQKSLSLSPGK |
| 141 | Shielded anti-HER2 | GPEADQQKACNHYKDPPFCTCGGGGSGGGGSGPLGVRGGG |
| | trastuzumab light | GSDIQMTQSPSSLSASVGDRVTITCRASQDVNTAVAWYQQK |
| | chain with epitope as | PGKAPKLLIYSASFLYSGVPSRFSGSRSGTDFTLTISSLQPEDF |
| | masking domain and | ATYYCQQHYTTPPTFGQGTKVEIKRTVAAPSVFIFPPSDEQLK |
| | MMP2 substrate | SGTASVVCLLNNFYPREAKVQWKVDNALQSGNSQESVTEQD |
| | sequence and F19-2 | SKDSTYSLSSTLTLSKADYEKHKVYACEVTHQGLSSPVTKSF |
| | homing domain | NRGECGGGGSGGGGSEVQLLESGGGLVQPGGSLRLSCAASG |
| | | FTFSSFSMSWVRQAPGKGLEWVSSISGSSGTTYYADSVKGRF |
| | | TISRDNSKNTLYLQMNSLRAEDTAVYYCAKPFPYFDYWGQG |
| | | TLVTVSSGGSGGGGSGGGGSGGEIVLTQSPGTLSLSPGERATL |
| | | SCRASQSVSSSFLAWYQQKPGQAPRLLIYYASSRATGIPDRFS |
| | | GSGSGTDFTLTISRLEPEDFAVYYCQQTGRIPPTFGQGTKVEI |
| | | K |
| 142 | Shielded anti-HER2 | GPEADQQKACNHYKDPPFCTCGGGGSGGGGSGPLGVRGGG |
| | trastuzumab heavy | GSEVQLVESGGGLVQPGGSLRLSCAASGFNIKDTYIHWVRQA |
| | chain with epitope as | PGKGLEWVARIYPTNGYTRYADSVKGRFTISADTSKNTAYL |
| | masking domain, | QMNSLRAEDTAVYYCSRWGGDGFYAMDYWGQGTLVTVSS |
| | MMP2 substrate | ASTKGPSVFPLAPSSKSTSGGTAALGCLVKDYFPEPVTVSWN |
| | sequence and IgG1 Fc | SGALTSGVHTFPAVLQSSGLYSLSSVVTVPSSSLGTQTYICNV |
| | with F405L, M428L, | NHKPSNTKVDKKVEPKSCDKTHTCPPCPAPELLGGPSVFLFPP |
| | N434S mutations and | KPKDTLMISRTPEVTCVVVDVSHEDPEVKFNWYVDGVEVHN |
| | F19-2 homing domain | AKTKPREEQYNSTYRVVSVLTVLHQDWLNGKEYKCKVSNK |
| | | ALPAPIEKTISKAKGQPREPQVYTLPPSRDELTKNQVSLTCLV |
| | | KGFYPSDIAVEWESNGQPENNYKTTPPVLDSDGSFLLYSkLT |
| | | VDKSRWQQGNVFSCSVLHEALHSHYTQKSLSLSPGKGGGGS |
| | | GGGGSEVQLLESGGGLVQPGGSLRLSCAASGFTFSSFSMSWV |
| | | RQAPGKGLEWVSSISGSSGTTYYADSVKGRFTISRDNSKNTL |
| | | YLQMNSLRAEDTAVYYCAKPFPYFDYWGQGTLVTVSSGGS |
| | | GGGGSGGGGSGGEIVLTQSPGTLSLSPGERATLSCRASQSVSS |
| | | SFLAWYQQKPGQAPRLLIYYASSRATGIPDRFSGSGSGTDFTL |
| | | TISRLEPEDFAVYYCQQTGRIPPTFGQGTKVEIK |
| 143 | Shielded anti-HER2 | GPEADQQKACNHYKDPPFCTCGGGGSGGGGSGPLGVRGGG |
| | trastuzumab light | GSDIQMTQSPSSLSASVGDRVTITCRASQDVNTAVAWYQQK |
| | chain with epitope as | PGKAPKLLIYSASFLYSGVPSRFSGSRSGTDFTLTISSLQPEDF |
| | masking domain and | ATYYCQQHYTTPPTFGQGTKVEIKRTVAAPSVFIFPPSDEQLK |
| | MMP2 substrate | SGTASVVCLLNNFYPREAKVQWKVDNALQSGNSQESVTEQD |
| | sequence and FAP-2 | SKDSTYSLSSTLTLSKADYEKHKVYACEVTHQGLSSPVTKSF |
| | homing domain | NRGECGGGGSGGGGSEVQLVESGGGLVQPGPSLRLSCTASR |
| | | YTFTEYTIHWVRQAPGKGLEWVGGINPNNGIPNYAASVKGR |
| | | FTISVDDSKSIAYLQMNSLKTEDTAVYFCARRRIAYGYDEGH |
| | | AMDYWGQGTTVTVSSGGSGGGGSGGGGSGGAIRMTQSPSSF |
| | | SVSTGDRVTMTCRSSQSLLYSRNQKNYLAWFQQKPGKAPKL |
| | | LIFWASTRESGVPSRFSGSGSGTDFTLTISCVQSEDFATYDCQ |
| | | QYFSYPLTFGQGTKLEIK |
| 144 | Shielded anti-HER2 | GPEADQQKACNHYKDPPFCTCGGGGSGGGGSGPLGVRGGG |
| | trastuzumab heavy | GSEVQLVESGGGLVQPGGSLRLSCAASGFNIKDTYIHWVRQA |
| | chain with epitope as | PGKGLEWVARIYPTNGYTRYADSVKGRFTISADTSKNTAYL |
| | masking domain, | QMNSLRAEDTAVYYCSRWGGDGFYAMDYWGQGTLVTVSS |
| | MMP2 substrate | ASTKGPSVFPLAPSSKSTSGGTAALGCLVKDYFPEPVTVSWN |
| | sequence and IgG1 Fc | SGALTSGVHTFPAVLQSSGLYSLSSVVTVPSSSLGTQTYICNV |
| | with F405L, M428L, | NHKPSNTKVDKKVEPKSCDKTHTCPPCPAPELLGGPSVFLFPP |
| | N434S mutations and | KPKDTLMISRTPEVTCVVVDVSHEDPEVKFNWYVDGVEVHN |
| | FAP-2 homing | AKTKPREEQYNSTYRVVSVLTVLHQDWLNGKEYKCKVSNK |
| | domain | ALPAPIEKTISKAKGQPREPQVYTLPPSRDELTKNQVSLTCLV |
| | | KGFYPSDIAVEWESNGQPENNYKTTPPVLDSDGSFLLYSkLT |
| | | VDKSRWQQGNVFSCSVLHEALHSHYTQKSLSLSPGKGGGGS |
| | | GGGGSEVQLVESGGGLVQPGPSLRLSCTASRYTFTEYTIHWV |
| | | RQAPGKGLEWVGGINPNNGIPNYAASVKGRFTISVDDSKSIA |
| | | YLQMNSLKTEDTAVYFCARRRIAYGYDEGHAMDYWGQGTT |
| | | VTVSSGGSGGGGSGGGGSGGAIRMTQSPSSFSVSTGDRVTMT |
| | | CRSSQSLLYSRNQKNYLAWFQQKPGKAPKLLIFWASTRESG |
| | | VPSRFSGSGSGTDFTLTISCVQSEDFATYDCQQYFSYPLTFGQ |
| | | GTKLEIK |
| 145 | EAC560: Shielded | AYRPSETLCGGELVDTLQFVCGDRGFAFGGGGSPLGLAGGG |
| | anti-HER2 | GGSDIVMTQTPLSLPVTPGEPASISCRASQDVNTAVAWYLQK |
| | trastuzumab light | PGQSPQLLIYSASFRYTGVPDRFSGSGSGTDFTLKISRVEAED |
| | chain VL1 variant | VGVYYCQQHYTTPPTFGQGTKVEIKRTVAAPSVFIFPPSDEQL |
| | (TrasVL1) with IGF2 | KSGTASVVCLLNNFYPREAKVQWKVDNALQSGNSQESVTEQ |
| | B chain with Y27A | DSKDSTYSLSSTLTLSKADYEKHKVYACEVTHQGLSSPVTKS |
| | mutation, MMP2 | FNRGECGGGGSGGGGSGGGGSGGGGSEVQLVESGGGLVQP |
| | substrate sequence | GGSLRLSCAASGFTFSDSWIHWVRQAPGKGLEWVAWISPYG |
| | and ScFv of PD-L1 | GSTYYADSVKGRFTISADTSKNTAYLQMNSLRAEDTAVYYC |
| | antibody | ARRHWPGGFDYWGQGTLVTVSSGGGGSGGGGSGGGGSDIQ |
| | | MTQSPSSLSASVGDRVTITCRASQDVSTAVAWYQQKPGKAP |
| | | KLLIYSASFLYSGVPSRFSGSGSGTDFTLTISSLQPEDFATYYC |
| | | QQYLYHPATFGQGTKVEIK |
| 201 | EAC208: Shielded | EPKSCDKTHTCPPCPGGGGSGPLGVRGGGGSEVQLVESGGG |
| | anti-VEGF | LVQPGGSLRLSCAASGYTFTNYGMNWVRQAPGKGLEWVG |
| | bevacizumab heavy | WINTYTGEPTYAADFKRRFTFSLDTSKSTAYLQMNSLRAEDT |
| | chain with hinge | AVYYCAKYPHYYGSSHWYFDVWGQGTLVTVSSASTKGPSV |
| | masking domain, | FPLAPSSKSTSGGTAALGCLVKDYFPEPVTVSWNSGALTSGV |
| | MMP2 substrate | HTFPAVLQSSGLYSLSSVVTVPSSSLGTQTYICNVNHKPSNTK |
| | sequence and IgG1 Fc | VDKKVEPKSCDKTHTCPPCPAPPLLGPSVFLFPPKPKDTLMIS |
| | with E233P, K409R, | RTPEVTCVVVDVSHEDPEVKFNWYVDGVEVHNAKTKPREE |
| | M428L, N434S | QYNSTYRVVSVLTVLHQDWLNGKEYKCKVSNKALPAPIEKT |
| | mutations and G236 | ISKAKGQPREPQVYTLPPSRDELTKNQVSLTCLVKGFYPSDIA |
| | deleted | VEWESNGQPENNYKTTPPVLDSDGSFFLYSRLTVDKSRWQQ |
| | | GNVFSCSVLHEALHSHYTQKSLSLSPGK |
| 202 | EAC209: Shielded | EPKSCDKTHTCPPCPGGGGSGPLGVRGGGGSDIQMTQSPSSL |
| | anti-VEGF | SASVGDRVTITCSASQDISNYLNWYQQKPGKAPKVLIYFTSSL |
| | bevacizumab light | HSGVPSRFSGSGSGTDFTLTISSLQPEDFATYYCQQYSTVPWT |
| | chain with hinge | FGQGTKVEIKRTVAAPSVFIFPPSDEQLKSGTASVVCLLNNFY |
| | masking domain and | PREAKVQWKVDNALQSGNSQESVTEQDSKDSTYSLSSTLTLS |
| | MMP2 substrate | KADYEKHKVYACEVTHQGLSSPVTKSFNRGEC |
| | sequence | |
| 203 | EAC259: Shielded | GIVEECCFRSCDLALLETYCAGGGGSPLGLAGGGGGSEVQLV |
| | anti-VEGF | ESGGGLVQPGGSLRLSCAASGYTFTNYGMNWVRQAPGKGL |
| | bevacizumab heavy | EWVGWINTYTGEPTYAADFKRRFTFSLDTSKSTAYLQMNSL |
| | chain with IGF2 A | RAEDTAVYYCAKYPHYYGSSHWYFDVWGQGTLVTVSSAST |
| | chain masking | KGPSVFPLAPSSKSTSGGTAALGCLVKDYFPEPVTVSWNSGA |
| | domain, MMP2 | LTSGVHTFPAVLQSSGLYSLSSVVTVPSSSLGTQTYICNVNHK |
| | substrate sequence | PSNTKVDKKVEPKSCDKTHTCPPCPAPPLLGPSVFLFPPKPKD |
| | and IgG1 Fc with | TLMISRTPEVTCVVVDVSHEDPEVKFNWYVDGVEVHNAKTK |
| | E233P, K409R, | PREEQYNSTYRVVSVLTVLHQDWLNGKEYKCKVSNKALPAP |
| | M428L, N434S | IEKTISKAKGQPREPQVYTLPPSRDELTKNQVSLTCLVKGFYP |
| | mutations and G236 | SDIAVEWESNGQPENNYKTTPPVLDSDGSFFLYSRLTVDKSR |
| | deleted | WQQGNVFSCSVLHEALHSHYTQKSLSLSPGK |
| 204 | EAC229: Shielded | AYRPSETLCGGELVDTLQFVCGDRGFYFGGGGSPLGLAGGG |
| | anti-VEGF | GGSDIQMTQSPSSLSASVGDRVTITCSASQDISNYLNWYQQK |
| | bevacizumab light | PGKAPKVLIYFTSSLHSGVPSRFSGSGSGTDFTLTISSLQPEDF |
| | chain with IGF2 B | ATYYCQQYSTVPWTFGQGTKVEIKRTVAAPSVFIFPPSDEQL |
| | chain masking | KSGTASVVCLLNNFYPREAKVQWKVDNALQSGNSQESVTEQ |
| | domain and MMP2 | DSKDSTYSLSSTLTLSKADYEKHKVYACEVTHQGLSSPVTKS |
| | substrate sequence | FNRGEC |
| 205 | EAC245: Shielded | GIVEECCFRSCDLALLETYCAGGGGSLSGRSDNHGGGGSEVQ |
| | anti-VEGF | LVESGGGLVQPGGSLRLSCAASGYTFTNYGMNWVRQAPGK |
| | bevacizumab heavy | GLEWVGWINTYTGEPTYAADFKRRFTFSLDTSKSTAYLQMN |
| | chain with IGF2 A | SLRAEDTAVYYCAKYPHYYGSSHWYFDVWGQGTLVTVSSA |
| | chain masking | STKGPSVFPLAPSSKSTSGGTAALGCLVKDYFPEPVTVSWNS |
| | domain, uPA | GALTSGVHTFPAVLQSSGLYSLSSVVTVPSSSLGTQTYICNVN |
| | substrate sequence | HKPSNTKVDKKVEPKSCDKTHTCPPCPAPPLLGPSVFLFPPKP |
| | and IgG1 Fc with | KDTLMISRTPEVTCVVVDVSHEDPEVKFNWYVDGVEVHNA |
| | E233P, F405L, | KTKPREEQYNSTYRVVSVLTVLHQDWLNGKEYKCKVSNKA |
| | M428L, N434S | LPAPIEKTISKAKGQPREPQVYTLPPSRDELTKNQVSLTCLVK |
| | mutations and G236 | GFYPSDIAVEWESNGQPENNYKTTPPVLDSDGSFLLYSkLTV |
| | deleted | DKSRWQQGNVFSCSVLHEALHSHYTQKSLSLSPGK |
| 206 | EAC246: Shielded | AYRPSETLCGGELVDTLQFVCGDRGFYFGGGGSLSGRSDNH |
| | anti-VEGF | GGGGSDIQMTQSPSSLSASVGDRVTITCSASQDISNYLNWYQ |
| | bevacizumab light | QKPGKAPKVLIYFTSSLHSGVPSRFSGSGSGTDFTLTISSLQPE |
| | chain with IGF2 B | DFATYYCQQYSTVPWTFGQGTKVEIKRTVAAPSVFIFPPSDE |
| | chain masking | QLKSGTASVVCLLNNFYPREAKVQWKVDNALQSGNSQESVT |
| | domain and uPA | EQDSKDSTYSLSSTLTLSKADYEKHKVYACEVTHQGLSSPVT |
| | substrate sequence | KSFNRGEC |
| 207 | EAC262: Shielded | GQRGVVGLPGQRGERGFPGLPGYGGGGSPLGLAGGGGGSEV |
| | anti-VEGF | QLVESGGGLVQPGGSLRLSCAASGYTFTNYGMNWVRQAPG |
| | bevacizumab heavy | KGLEWVGWINTYTGEPTYAADFKRRFTFSLDTSKSTAYLQM |
| | chain with collagen | NSLRAEDTAVYYCAKYPHYYGSSHWYFDVWGQGTLVTVSS |
| | peptide 1 masking | ASTKGPSVFPLAPSSKSTSGGTAALGCLVKDYFPEPVTVSWN |
| | domain, MMP2 | SGALTSGVHTFPAVLQSSGLYSLSSVVTVPSSSLGTQTYICNV |
| | substrate sequence | NHKPSNTKVDKKVEPKSCDKTHTCPPCPAPPLLGPSVFLFPPK |
| | and IgG1 Fc with | PKDTLMISRTPEVTCVVVDVSHEDPEVKFNWYVDGVEVHNA |
| | E233P, K409R, | KTKPREEQYNSTYRVVSVLTVLHQDWLNGKEYKCKVSNKA |
| | M428L, N434S | LPAPIEKTISKAKGQPREPQVYTLPPSRDELTKNQVSLTCLVK |
| | mutations and G236 | GFYPSDIAVEWESNGQPENNYKTTPPVLDSDGSFFLYSRLTV |
| | deleted | DKSRWQQGNVFSCSVLHEALHSHYTQKSLSLSPGK |
| 208 | EAC263: Shielded | DQCIVDDITYNVQDTFHKKHEEGHMLNCTCFGQGRGRWKC |
| | anti-VEGF | DPVDQCQDSETGTFYQIGDSWEKYVHGVRYQCYCYGRGIGE |
| | bevacizumab light | WHCQPLQTYPSSGGGGSPLGLAGGGGGSDIQMTQSPSSLSAS |
| | chain with fibronectin | VGDRVTITCSASQDISNYLNWYQQKPGKAPKVLIYFTSSLHS |
| | peptide masking | GVPSRFSGSGSGTDFTLTISSLQPEDFATYYCQQYSTVPWTFG |
| | domain and MMP2 | QGTKVEIKRTVAAPSVFIFPPSDEQLKSGTASVVCLLNNFYPR |
| | substrate sequence | EAKVQWKVDNALQSGNSQESVTEQDSKDSTYSLSSTLTLSK |
| | | ADYEKHKVYACEVTHQGLSSPVTKSFNRGEC |
| 209 | EAC292: Shielded | GQRGVVGLPGQRGERGFPGLPGYGGGGSPLGLAGGGGGSEV |
| | anti-VEGF | QLVESGGGLVQPGGSLRLSCAASGYTFTNYGMNWVRQAPG |
| | bevacizumab heavy | KGLEWVGWINTYTGEPTYAADFKRRFTFSLDTSKSTAYLQM |
| | chain with collagen | NSLRAEDTAVYYCAKYPHYYGSSHWYFDVWGQGTLVTVSS |
| | peptide 1 masking | ASTKGPSVFPLAPSSKSTSGGTAALGCLVKDYFPEPVTVSWN |
| | domain, MMP2 | SGALTSGVHTFPAVLQSSGLYSLSSVVTVPSSSLGTQTYICNV |
| | substrate sequence | NHKPSNTKVDKKVEPKSCDKTHTCPPCPAPELLGGPSVFLFPP |
| | and IgG1 Fc with | KPKDTLMISRTPEVTCVVVDVSHEDPEVKFNWYVDGVEVHN |
| | K409R, M428L, | AKTKPREEQYNSTYRVVSVLTVLHQDWLNGKEYKCKVSNK |
| | N434S mutations | ALPAPIEKTISKAKGQPREPQVYTLPPSRDELTKNQVSLTCLV |
| | | KGFYPSDIAVEWESNGQPENNYKTTPPVLDSDGSGSFFLYSR |
| | | LTVDKSRWQQGNVFSCSVLHEALHSHYTQKSLSLSPGK |
| 210 | EAC297: Shielded | GILEECCFRSCDLALLETYCAGGGGSPLGLAGGGGGSEVQLV |
| | anti-VEGF | ESGGGLVQPGGSLRLSCTASGFSLTDYYYMTWVRQAPGKGL |
| | brolucizumab heavy | EWVGFIDPDDDPYYATWAKGRFTISRDNSKNTLYLQMNSLR |
| | chain with IGF2 A | AEDTAVYYCAGGDHNSGWGLDIWGQGTLVTVSS |
| | chain with V43L | ASTKGPSVFPLAPSSKSTSGGTAALGCLVKDYFPEPVTVSWN |
| | mutation, MMP2 | SGALTSGVHTFPAVLQSSGLYSLSSVVTVPSSSLGTQTYICNV |
| | substrate sequence | NHKPSNTKVDKKVEPKSCDKTHTCPPCPAPELLGGPSVFLFPP |
| | and IgG1 Fc with | KPKDTLMISRTPEVTCVVVDVSHEDPEVKFNWYVDGVEVHN |
| | K409R, M428L, | AKTKPREEQYNSTYRVVSVLTVLHQDWLNGKEYKCKVSNK |
| | N434S mutations | ALPAPIEKTISKAKGQPREPQVYTLPPSRDELTKNQVSLTCLV |
| | | KGFYPSDIAVEWESNGQPENNYKTTPPVLDSDGSFFLYSRLT |
| | | VDKSRWQQGNVFSCSVLHEALHSHYTQKSLSLSPGK |
| 211 | EAC298: Shielded | AYRPSETLCGGELVDTLQFVCGDRGFAFGGGGSPLGLAGGG |
| | anti-VEGF | GGSEIVMTQSPSTLSASVGDRVIITCQASEIIHSWLAWYQQKP |
| | brolucizumab light | GKAPKLLIYLASTLASGVPSRFSGSGSGAEFTLTISSLQPDDFA |
| | chain with IGF2 B | TYYCQNVYLASTNGANFGQGTKLTVLGQPKAAPSVTLFPPSS |
| | chain with Y27A | EELQANKATLVCLISDFYPGAVTVAWKADSSPVKAGVETTT |
| | mutation and MMP2 | PSKQSNNKYAASSYLSLTPEQWKSHRSYSCQVTHE |
| | substrate sequence | GSTVEKTVAPTECS |
| 212 | EAC516: Shielded | GQRGVVGLPGQRGERGGGGSPLGLAGGGGGSEVQLVESGG |
| | anti-VEGF | GLVQPGGSLRLSCAASGYTFTNYGMNWVRQAPGKGLEWVG |
| | bevacizumab heavy | WINTYTGEPTYAADFKRRFTFSLDTSKSTAYLQMNSLRAEDT |
| | chain with collagen | AVYYCAKYPHYYGSSHWYFDVWGQGTLVTVSSASTKGPSV |
| | peptide 2 masking | FPLAPSSKSTSGGTAALGCLVKDYFPEPVTVSWNSGALTSGV |
| | domain, MMP2 | HTFPAVLQSSGLYSLSSVVTVPSSSLGTQTYICNVNHKPSNTK |
| | substrate sequence | VDKKVEPKSCDKTHTCPPCPAPELLGGPSVFLFPPKPKDTLMI |
| | and IgG1 Fc with | SRTPEVTCVVVDVSHEDPEVKFNWYVDGVEVHNAKTKPREE |
| | K409R, M428L, | QYNSTYRVVSVLTVLHQDWLNGKEYKCKVSNKALPAPIEKT |
| | N434S mutations | ISKAKGQPREPQVYTLPPSRDELTKNQVSLTCLVKGFYPSDIA |
| | | VEWESNGQPENNYKTTPPVLDSDGSGSFFLYSRLTVDKSRW |
| | | QQGNVFSCSVLHEALHSHYTQKSLSLSPGK |
| 213 | EAC530: Shielded | GILEECCFRSCDLALLETYCAGGGGSPLGLAGGGGGSQVQLV |
| | anti-VEGF | QSGSELKKPGASVKVSCKASGYTFTNYGMNWVRQAPGQGL |
| | bevacizumab heavy | EWMGWINTYTGEPTYAQGFTGRFVFSLDTSVSTAYLQICSLK |
| | chain VH1 variant | AEDTAVYFCAKYPHYYGSSHWYFDVWGQGTLVTVSSASTK |
| | (BevaVH1) with | GPSVFPLAPSSKSTSGGTAALGCLVKDYFPEPVTVSWNSGAL |
| | IGF2 A chain with | TSGVHTFPAVLQSSGLYSLSSVVTVPSSSLGTQTYICNVNHKP |
| | V43L mutation, | SNTKVDKKVEPKSCDKTHTCPPCPAPELLGGPSVFLFPPKPKD |
| | MMP2 substrate | TLMISRTPEVTCVVVDVSHEDPEVKFNWYVDGVEVHNAKTK |
| | sequence and IgG1 Fc | PREEQYNSTYRVVSVLTVLHQDWLNGKEYKCKVSNKALPAP |
| | with K409R, M428L, | IEKTISKAKGQPREPQVYTLPPSRDELTKNQVSLTCLVKGFYP |
| | N434S mutations | SDIAVEWESNGQPENNYKTTPPVLDSDGSFFLYSRLTVDKSR |
| | | WQQGNVFSCSVLHEALHSHYTQKSLSLSPGK |
| 214 | EAC531: Shielded | GILEECCFRSCDLALLETYCAGGGGSPLGLAGGGGGSQVQLV |
| | anti-VEGF | QSGAEVKKPGSSVKVSCKASGYTFTNYGMNWVRQAPGQGL |
| | bevacizumab heavy | EWMGWINTYTGEPTYAQKFQGRFTFTADESTSTAYMELSSL |
| | chain VH2 variant | RSEDTAVYYCAKYPHYYGSSHWYFDVWGQGTLVTVSSAST |
| | (BevaVH2) with | KGPSVFPLAPSSKSTSGGTAALGCLVKDYFPEPVTVSWNSGA |
| | IGF2 A chain with | LTSGVHTFPAVLQSSGLYSLSSVVTVPSSSLGTQTYICNVNHK |
| | V43L mutation, | PSNTKVDKKVEPKSCDKTHTCPPCPAPELLGGPSVFLFPPKPK |
| | MMP2 substrate | DTLMISRTPEVTCVVVDVSHEDPEVKFNWYVDGVEVHNAKT |
| | sequence and IgG1 Fc | KPREEQYNSTYRVVSVLTVLHQDWLNGKEYKCKVSNKALP |
| | with K409R, M428L, | APIEKTISKAKGQPREPQVYTLPPSRDELTKNQVSLTCLVKGF |
| | N434S mutations | YPSDIAVEWESNGQPENNYKTTPPVLDSDGSFFLYSRLTVDK |
| | | SRWQQGNVFSCSVLHEALHSHYTQKSLSLSPGK |
| 215 | EAC534: Shielded | AYRPSETLCGGELVDTLQFVCGDRGFAFGGGGSPLGLAGGG |
| | anti-VEGF | GGSDIQMTQSPSSLSASVGDRVTITCRASQDISNYLNWYQQK |
| | bevacizumab light | PGKAPKVLIYFTSSLHSGVPSRFSGSGSGTDFTLTISSLQPEDF |
| | chain VL1 variant | ATYYCQQYSTVPWTFGQGTKLEIKRTVAAPSVFIFPPSDEQL |
| | (BevaVL1) with IGF2 | KSGTASVVCLLNNFYPREAKVQWKVDNALQSGNSQESVTEQ |
| | B chain with Y27A | DSKDSTYSLSSTLTLSKADYEKHKVYACEVTHQGLSSPVTKS |
| | mutation and MMP2 | FNRGEC |
| | substrate sequence | |
| 216 | EAC538: Shielded | GQRGVVGLPGQRGERGGGGSPLGLAGGGGGSQVQLVQSGS |
| | anti-VEGF | ELKKPGASVKVSCKASGYTFTNYGMNWVRQAPGQGLEWM |
| | bevacizumab heavy | GWINTYTGEPTYAQGFTGRFVFSLDTSVSTAYLQICSLKAED |
| | chain VH1 variant | TAVYFCAKYPHYYGSSHWYFDVWGQGTLVTVSSASTKGPS |
| | (BevaVH1) with | VFPLAPSSKSTSGGTAALGCLVKDYFPEPVTVSWNSGALTSG |
| | collagen peptide 2 | VHTFPAVLQSSGLYSLSSVVTVPSSSLGTQTYICNVNHKPSNT |
| | masking domain, | KVDKKVEPKSCDKTHTCPPCPAPELLGGPSVFLFPPKPKDTL |
| | MMP2 substrate | MISRTPEVTCVVVDVSHEDPEVKFNWYVDGVEVHNAKTKPR |
| | sequence and IgG1 Fc | EEQYNSTYRVVSVLTVLHQDWLNGKEYKCKVSNKALPAPIE |
| | with K409R, M428L, | KTISKAKGQPREPQVYTLPPSRDELTKNQVSLTCLVKGFYPS |
| | N434S mutations | DIAVEWESNGQPENNYKTTPPVLDSDGSGSFFLYSRLTVDKS |
| | | RWQQGNVFSCSVLHEALHSHYTQKSLSLSPGK |
| 217 | EAC539: Shielded | GQRGVVGLPGQRGERGGGGSPLGLAGGGGGSQVQLVQSGA |
| | anti-VEGF | EVKKPGSSVKVSCKASGYTFTNYGMNWVRQAPGQGLEWM |
| | bevacizumab heavy | GWINTYTGEPTYAQKFQGRFTFTADESTSTAYMELSSLRSED |
| | chain VH2 variant | TAVYYCAKYPHYYGSSHWYFDVWGQGTLVTVSSASTKGPS |
| | (BevaVH2) with | VFPLAPSSKSTSGGTAALGCLVKDYFPEPVTVSWNSGALTSG |
| | collagen peptide 2 | VHTFPAVLQSSGLYSLSSVVTVPSSSLGTQTYICNVNHKPSNT |
| | masking domain, | KVDKKVEPKSCDKTHTCPPCPAPELLGGPSVFLFPPKPKDTL |
| | MMP2 substrate | MISRTPEVTCVVVDVSHEDPEVKFNWYVDGVEVHNAKTKPR |
| | sequence and IgG1 Fc | EEQYNSTYRVVSVLTVLHQDWLNGKEYKCKVSNKALPAPIE |
| | with K409R, M428L, | KTISKAKGQPREPQVYTLPPSRDELTKNQVSLTCLVKGFYPS |
| | N434S mutations | DIAVEWESNGQPENNYKTTPPVLDSDGSGSFFLYSRLTVDKS |
| | | RWQQGNVFSCSVLHEALHSHYTQKSLSLSPGK |
| 218 | EAC542: Shielded | DQCIVDDITYNVQDTFHKKHEEGHMLNCTCFGQGRGRWKC |
| | anti-VEGF | DPVDQCQDSETGTFYQIGDSWEKYVHGVRYQCYCYGRGIGE |
| | bevacizumab light | WHCQPLQTYPSSGGGGSPLGLAGGGGGSDIQMTQSPSSLSAS |
| | chain VL1 variant | VGDRVTITCRASQDISNYLNWYQQKPGKAPKVLIYFTSSLHS |
| | (BevaVL1) with | GVPSRFSGSGSGTDFTLTISSLQPEDFATYYCQQYSTVPWTFG |
| | fibronectin peptide | QGTKLEIKRTVAAPSVFIFPPSDEQLKSGTASVVCLLNNFYPR |
| | masking domain and | EAKVQWKVDNALQSGNSQESVTEQDSKDSTYSLSSTLTLSK |
| | MMP2 substrate | ADYEKHKVYACEVTHQGLSSPVTKSFNRGEC |
| | sequence | |
| 219 | EAC596: Shielded | GILEECCFRSCDLALLETYCAGGGGSPLGLAGGGGGSQVQLV |
| | anti-VEGF | QSGAEVKKPGSSVKVSCKASGYTFTNYGMNWVRQAPGQGL |
| | bevacizumab heavy | EWMGWINTYTGEPTYAQKFQGRVTITADESTSTAYMELSSL |
| | chain VH3 variant | RSEDTAVYYCAKYPHYYGSSHWYFDVWGQGTLVTVSSAST |
| | (BevaVH3) with | KGPSVFPLAPSSKSTSGGTAALGCLVKDYFPEPVTVSWNSGA |
| | IGF2 A chain with | LTSGVHTFPAVLQSSGLYSLSSVVTVPSSSLGTQTYICNVNHK |
| | V43L mutation, | PSNTKVDKKVEPKSCDKTHTCPPCPAPELLGGPSVFLFPPKPK |
| | MMP2 substrate | DTLMISRTPEVTCVVVDVSHEDPEVKFNWYVDGVEVHNAKT |
| | sequence and IgG1 Fc | KPREEQYNSTYRVVSVLTVLHQDWLNGKEYKCKVSNKALP |
| | with K409R, M428L, | APIEKTISKAKGQPREPQVYTLPPSRDELTKNQVSLTCLVKGF |
| | N434S mutations | YPSDIAVEWESNGQPENNYKTTPPVLDSDGSFLLYSkLTVDK |
| | | SRWQQGNVFSCSVLHEALHSHYTQKSLSLSPGK |
| 220 | EAC597: Shielded | GILEECCFRSCDLALLETYCAGGGGSPLGLAGGGGGSEVQLV |
| | anti-VEGF | QSGAEVKKPGESVKISCKGSGYTFTNYGMNWVRQMPGKGL |
| | bevacizumab heavy | EWMGWINTYTGEPTYSPSFQGQFTFSADKSISTAYLQISSLKA |
| | chain VH4 variant | SDTAMYYCAKYPHYYGSSHWYFDVWGQGTLVTVSSASTKG |
| | (BevaVH4) with | PSVFPLAPSSKSTSGGTAALGCLVKDYFPEPVTVSWNSGALT |
| | IGF2 A chain with | SGVHTFPAVLQSSGLYSLSSVVTVPSSSLGTQTYICNVNHKPS |
| | V43L mutation, | NTKVDKKVEPKSCDKTHTCPPCPAPELLGGPSVFLFPPKPKD |
| | MMP2 substrate | TLMISRTPEVTCVVVDVSHEDPEVKFNWYVDGVEVHNAKTK |
| | sequence and IgG1 Fc | PREEQYNSTYRVVSVLTVLHQDWLNGKEYKCKVSNKALPAP |
| | with K409R, M428L, | IEKTISKAKGQPREPQVYTLPPSRDELTKNQVSLTCLVKGFYP |
| | N434S mutations | SDIAVEWESNGQPENNYKTTPPVLDSDGSFLLYSkLTVDKSR |
| | | WQQGNVFSCSVLHEALHSHYTQKSLSLSPGK |
| 221 | EAC598: Shielded | GILEECCFRSCDLALLETYCAGGGGSPLGLAGGGGGSQVQLV |
| | anti-VEGF | QSGAEVKKPGSSVKVSCKASGYDFTHYGMNWVRQAPGQGL |
| | bevacizumab heavy | EWMGWINTYTGEPTYAQKFQGRFTFTADESTSTAYMELSSL |
| | chain VH5 variant | RSEDTAVYYCAKYPYYYGTSHWYFDVWGQGTLVTVSSAST |
| | (BevaVH5) with | KGPSVFPLAPSSKSTSGGTAALGCLVKDYFPEPVTVSWNSGA |
| | IGF2 A chain with | LTSGVHTFPAVLQSSGLYSLSSVVTVPSSSLGTQTYICNVNHK |
| | V43L mutation, | PSNTKVDKKVEPKSCDKTHTCPPCPAPELLGGPSVFLFPPKPK |
| | MMP2 substrate | DTLMISRTPEVTCVVVDVSHEDPEVKFNWYVDGVEVHNAKT |
| | sequence and IgG1 Fc | KPREEQYNSTYRVVSVLTVLHQDWLNGKEYKCKVSNKALP |
| | with K409R, M428L, | APIEKTISKAKGQPREPQVYTLPPSRDELTKNQVSLTCLVKGF |
| | N434S mutations | YPSDIAVEWESNGQPENNYKTTPPVLDSDGSFLLYSkLTVDK |
| | | SRWQQGNVFSCSVLHEALHSHYTQKSLSLSPGK |
| 222 | EAC599: Shielded | AYRPSETLCGGELVDTLQFVCGDRGFAFGGGGSPLGLAGGG |
| | anti-VEGF | GGSDIVMTQTPLSLPVTPGEPASISCRASQDISNYLNWYLQKP |
| | bevacizumab light | GQSPQVLIYFTSSLHSGVPDRFSGSGSGTDFTLKISRLEAEDV |
| | chain VL2 variant | GVYYCQQYSTVPWTFGQGTKLEIKRTVAAPSVFIFPPSDEQL |
| | (BevaVL2) with IGF2 | KSGTASVVCLLNNFYPREAKVQWKVDNALQSGNSQESVTEQ |
| | B chain with Y27A | DSKDSTYSLSSTLTLSKADYEKHKVYACEVTHQGLSSPVTKS |
| | mutation and MMP2 | FNRGEC |
| | substrate sequence | |
| 223 | EAC819: Shielded | GQRGVVGLPGQRGERGFPGLPGYGGGGSPLGLAGGGGGSQ |
| | anti-VEGF | VQLVQSGSELKKPGASVKVSCKASGYTFTNYGMNWVRQAP |
| | bevacizumab heavy | GQGLEWMGWINTYTGEPTYAQGFTGRFVFSLDTSVSTAYLQ |
| | chain VH1 variant | ICSLKAEDTAVYFCAKYPHYYGSSHWYFDVWGQGTLVTVSS |
| | (BevaVH1) with | ASTKGPSVFPLAPSSKSTSGGTAALGCLVKDYFPEPVTVSWN |
| | collagen peptide 1 | SGALTSGVHTFPAVLQSSGLYSLSSVVTVPSSSLGTQTYICNV |
| | masking domain, | NHKPSNTKVDKKVEPKSCDKTHTCPPCPAPELLGGPSVFLFPP |
| | MMP2 substrate | KPKDTLMISRTPEVTCVVVDVSHEDPEVKFNWYVDGVEVHN |
| | sequence and IgG1 Fc | AKTKPREEQYNSTYRVVSVLTVLHQDWLNGKEYKCKVSNK |
| | with K409R, M428L, | ALPAPIEKTISKAKGQPREPQVYTLPPSRDELTKNQVSLTCLV |
| | N434S mutations | KGFYPSDIAVEWESNGQPENNYKTTPPVLDSDGSGSFFLYSR |
| | | LTVDKSRWQQGNVFSCSVLHEALHSHYTQKSLSLSPGK |
| 224 | EAC820: Shielded | GQRGVVGLPGQRGERGFPGLPGYGGGGSPLGLAGGGGGSQ |
| | anti-VEGF | VQLVQSGAEVKKPGSSVKVSCKASGYTFTNYGMNWVRQAP |
| | bevacizumab heavy | GQGLEWMGWINTYTGEPTYAQKFQGRFTFTADESTSTAYME |
| | chain VH2 variant | LSSLRSEDTAVYYCAKYPHYYGSSHWYFDVWGQGTLVTVSS |
| | (BevaVH2) with | ASTKGPSVFPLAPSSKSTSGGTAALGCLVKDYFPEPVTVSWN |
| | collagen peptide 1 | SGALTSGVHTFPAVLQSSGLYSLSSVVTVPSSSLGTQTYICNV |
| | masking domain, | NHKPSNTKVDKKVEPKSCDKTHTCPPCPAPELLGGPSVFLFPP |
| | MMP2 substrate | KPKDTLMISRTPEVTCVVVDVSHEDPEVKFNWYVDGVEVHN |
| | sequence and IgG1 Fc | AKTKPREEQYNSTYRVVSVLTVLHQDWLNGKEYKCKVSNK |
| | with K409R, M428L, | ALPAPIEKTISKAKGQPREPQVYTLPPSRDELTKNQVSLTCLV |
| | N434S mutations | KGFYPSDIAVEWESNGQPENNYKTTPPVLDSDGSGSFFLYSR |
| | | LTVDKSRWQQGNVFSCSVLHEALHSHYTQKSLSLSPGK |
| 225 | EAC821: Shielded | GQRGVVGLPGQRGERGFPGLPGYGGGGSPLGLAGGGGGSQ |
| | anti-VEGF | VQLVQSGAEVKKPGSSVKVSCKASGYTFTNYGMNWVRQAP |
| | bevacizumab heavy | GQGLEWMGWINTYTGEPTYAQKFQGRVTITADESTSTAYME |
| | chain VH3 variant | LSSLRSEDTAVYYCAKYPHYYGSSHWYFDVWGQGTLVTVSS |
| | (BevaVH3) with | ASTKGPSVFPLAPSSKSTSGGTAALGCLVKDYFPEPVTVSWN |
| | collagen peptide 1 | SGALTSGVHTFPAVLQSSGLYSLSSVVTVPSSSLGTQTYICNV |
| | masking domain, | NHKPSNTKVDKKVEPKSCDKTHTCPPCPAPELLGGPSVFLFPP |
| | MMP2 substrate | KPKDTLMISRTPEVTCVVVDVSHEDPEVKFNWYVDGVEVHN |
| | sequence and IgG1 Fc | AKTKPREEQYNSTYRVVSVLTVLHQDWLNGKEYKCKVSNK |
| | with K409R, M428L, | ALPAPIEKTISKAKGQPREPQVYTLPPSRDELTKNQVSLTCLV |
| | N434S mutations | KGFYPSDIAVEWESNGQPENNYKTTPPVLDSDGSGSFFLYSR |
| | | LTVDKSRWQQGNVFSCSVLHEALHSHYTQKSLSLSPGK |
| 226 | EAC822: Shielded | GQRGVVGLPGQRGERGFPGLPGYGGGGSPLGLAGGGGGSEV |
| | anti-VEGF | QLVQSGAEVKKPGESVKISCKGSGYTFTNYGMNWVRQMPG |
| | bevacizumab heavy | KGLEWMGWINTYTGEPTYSPSFQGQFTFSADKSISTAYLQISS |
| | chain VH4 variant | LKASDTAMYYCAKYPHYYGSSHWYFDVWGQGTLVTVSSAS |
| | (BevaVH4) with | TKGPSVFPLAPSSKSTSGGTAALGCLVKDYFPEPVTVSWNSG |
| | collagen peptide 1 | ALTSGVHTFPAVLQSSGLYSLSSVVTVPSSSLGTQTYICNVNH |
| | masking domain, | KPSNTKVDKKVEPKSCDKTHTCPPCPAPELLGGPSVFLFPPKP |
| | MMP2 substrate | KDTLMISRTPEVTCVVVDVSHEDPEVKFNWYVDGVEVHNA |
| | sequence and IgG1 Fc | KTKPREEQYNSTYRVVSVLTVLHQDWLNGKEYKCKVSNKA |
| | with K409R, M428L, | LPAPIEKTISKAKGQPREPQVYTLPPSRDELTKNQVSLTCLVK |
| | N434S mutations | GFYPSDIAVEWESNGQPENNYKTTPPVLDSDGSGSFFLYSRLT |
| | | VDKSRWQQGNVFSCSVLHEALHSHYTQKSLSLSPGK |
| 227 | EAC823: Shielded | GQRGVVGLPGQRGERGFPGLPGYGGGGSPLGLAGGGGGSQ |
| | anti-VEGF | VQLVQSGAEVKKPGSSVKVSCKASGYDFTHYGMNWVRQAP |
| | bevacizumab heavy | GQGLEWMGWINTYTGEPTYAQKFQGRFTFTADESTSTAYME |
| | chain VH5 variant | LSSLRSEDTAVYYCAKYPYYYGTSHWYFDVWGQGTLVTVS |
| | (BevaVH5) with | SASTKGPSVFPLAPSSKSTSGGTAALGCLVKDYFPEPVTVSW |
| | collagen peptide 1 | NSGALTSGVHTFPAVLQSSGLYSLSSVVTVPSSSLGTQTYICN |
| | masking domain, | VNHKPSNTKVDKKVEPKSCDKTHTCPPCPAPELLGGPSVFLF |
| | MMP2 substrate | PPKPKDTLMISRTPEVTCVVVDVSHEDPEVKFNWYVDGVEV |
| | sequence and IgG1 Fc | HNAKTKPREEQYNSTYRVVSVLTVLHQDWLNGKEYKCKVS |
| | with K409R, M428L, | NKALPAPIEKTISKAKGQPREPQVYTLPPSRDELTKNQVSLTC |
| | N434S mutations | LVKGFYPSDIAVEWESNGQPENNYKTTPPVLDSDGSGSFFLY |
| | | SRLTVDKSRWQQGNVFSCSVLHEALHSHYTQKSLSLSPGK |
| 228 | EAC824: Shielded | GQRGVVGLPGQRGERGGGGSPLGLAGGGGGSQVQLVQSGA |
| | anti-VEGF | EVKKPGSSVKVSCKASGYTFTNYGMNWVRQAPGQGLEWM |
| | bevacizumab heavy | GWINTYTGEPTYAQKFQGRVTITADESTSTAYMELSSLRSED |
| | chain VH3 variant | TAVYYCAKYPHYYGSSHWYFDVWGQGTLVTVSSASTKGPS |
| | (BevaVH3) with | VFPLAPSSKSTSGGTAALGCLVKDYFPEPVTVSWNSGALTSG |
| | collagen peptide 2 | VHTFPAVLQSSGLYSLSSVVTVPSSSLGTQTYICNVNHKPSNT |
| | masking domain, | KVDKKVEPKSCDKTHTCPPCPAPELLGGPSVFLFPPKPKDTL |
| | MMP2 substrate | MISRTPEVTCVVVDVSHEDPEVKFNWYVDGVEVHNAKTKPR |
| | sequence and IgG1 Fc | EEQYNSTYRVVSVLTVLHQDWLNGKEYKCKVSNKALPAPIE |
| | with K409R, M428L, | KTISKAKGQPREPQVYTLPPSRDELTKNQVSLTCLVKGFYPS |
| | N434S mutations | DIAVEWESNGQPENNYKTTPPVLDSDGSGSFFLYSRLTVDKS |
| | | RWQQGNVFSCSVLHEALHSHYTQKSLSLSPGK |
| 229 | EAC825: Shielded | GQRGVVGLPGQRGERGGGGSPLGLAGGGGGSEVQLVQSGA |
| | anti-VEGF | EVKKPGESVKISCKGSGYTFTNYGMNWVRQMPGKGLEWMG |
| | bevacizumab heavy | WINTYTGEPTYSPSFQGQFTFSADKSISTAYLQISSLKASDTA |
| | chain VH4 variant | MYYCAKYPHYYGSSHWYFDVWGQGTLVTVSSASTKGPSVF |
| | (BevaVH4) with | PLAPSSKSTSGGTAALGCLVKDYFPEPVTVSWNSGALTSGVH |
| | collagen peptide 2 | TFPAVLQSSGLYSLSSVVTVPSSSLGTQTYICNVNHKPSNTKV |
| | masking domain, | DKKVEPKSCDKTHTCPPCPAPELLGGPSVFLFPPKPKDTLMIS |
| | MMP2 substrate | RTPEVTCVVVDVSHEDPEVKFNWYVDGVEVHNAKTKPREE |
| | sequence and IgG1 Fc | QYNSTYRVVSVLTVLHQDWLNGKEYKCKVSNKALPAPIEKT |
| | with K409R, M428L, | ISKAKGQPREPQVYTLPPSRDELTKNQVSLTCLVKGFYPSDIA |
| | N434S mutations | VEWESNGQPENNYKTTPPVLDSDGSGSFFLYSRLTVDKSRW |
| | | QQGNVFSCSVLHEALHSHYTQKSLSLSPGK |
| 230 | EAC826: Shielded | GQRGVVGLPGQRGERGGGGSPLGLAGGGGGSQVQLVQSGA |
| | anti-VEGF | EVKKPGSSVKVSCKASGYDFTHYGMNWVRQAPGQGLEWM |
| | bevacizumab heavy | GWINTYTGEPTYAQKFQGRFTFTADESTSTAYMELSSLRSED |
| | chain VH5 variant | TAVYYCAKYPYYYGTSHWYFDVWGQGTLVTVSSASTKGPS |
| | (BevaVH5) with | VFPLAPSSKSTSGGTAALGCLVKDYFPEPVTVSWNSGALTSG |
| | collagen peptide 2 | VHTFPAVLQSSGLYSLSSVVTVPSSSLGTQTYICNVNHKPSNT |
| | masking domain, | KVDKKVEPKSCDKTHTCPPCPAPELLGGPSVFLFPPKPKDTL |
| | MMP2 substrate | MISRTPEVTCVVVDVSHEDPEVKFNWYVDGVEVHNAKTKPR |
| | sequence and IgG1 Fc | EEQYNSTYRVVSVLTVLHQDWLNGKEYKCKVSNKALPAPIE |
| | with K409R, M428L, | KTISKAKGQPREPQVYTLPPSRDELTKNQVSLTCLVKGFYPS |
| | N434S mutations | DIAVEWESNGQPENNYKTTPPVLDSDGSGSFFLYSRLTVDKS |
| | | RWQQGNVFSCSVLHEALHSHYTQKSLSLSPGK |
| 231 | EAC832: Shielded | DQCIVDDITYNVQDTFHKKHEEGHMLNCTCFGQGRGRWKC |
| | anfi-VEGF | DPVDQCQDSETGTFYQIGDSWEKYVHGVRYQCYCYGRGIGE |
| | bevacizumab light | WHCQPLQTYPSSGGGGSPLGLAGGGGGSDIVMTQTPLSLPVT |
| | chain VL2 variant | PGEPASISCRASQDISNYLNWYLQKPGQSPQVLIYFTSSLHSG |
| | (BevaVL2) with | VPDRFSGSGSGTDFTLKISRLEAEDVGVYYCQQYSTVPWTFG |
| | fibronectin peptide | QGTKLEIKRTVAAPSVFIFPPSDEQLKSGTASVVCLLNNFYPR |
| | masking domain and | EAKVQWKVDNALQSGNSQESVTEQDSKDSTYSLSSTLTLSK |
| | MMP2 substrate | ADYEKHKVYACEVTHQGLSSPVTKSFNRGEC |
| | sequence | |
| 232 | EAC835: Shielded | GQRGVVGLPGQRGERGGGGSPLGLAGGGGGSQVQLVQSGA |
| | anti-VEGF | EVKKPGSSVKVSCKASGYTFTNYGMNWVRQAPGQGLEWM |
| | bevacizumab heavy | GWINTYTGEPTYAQKFQGRFTFTADESTSTAYMELSSLRSED |
| | chain VH2 variant | TAVYYCAKYPHYYGSSHWYFDVWGQGTLVTVSSASTKGPS |
| | (BevaVH2) with | VFPLAPSSKSTSGGTAALGCLVKDYFPEPVTVSWNSGALTSG |
| | collagen peptide 2 | VHTFPAVLQSSGLYSLSSVVTVPSSSLGTQTYICNVNHKPSNT |
| | masking domain, | KVDKKVEPKSCDKTHTCPPCPAPELLGGPSVFLFPPKPKDTL |
| | MMP2 substrate | MISRTPEVTCVVVDVSHEDPEVKFNWYVDGVEVHNAKTKPR |
| | sequence and IgG1 Fc | EEQYNSTYRVVSVLTVLHQDWLNGKEYKCKVSNKALPAPIE |
| | with E345R, K409R, | KTISKAKGQPRRPQVYTLPPSRDELTKNQVSLTCLVKGFYPS |
| | M428L, N434S | DIAVEWESNGQPENNYKTTPPVLDSDGSFFLYSRLTVDKSRW |
| | mutations | QQGNVFSCSVLHEALHSHYTQKSLSLSPGK |
| 233 | EAC845: Shielded | GILEECCFRSCDLALLETYCAGGGGSPLGLAGGGGGSQVQLV |
| | anti-VEGF | QSGAEVKKPGSSVKVSCKASGYTFTNYGMNWVRQAPGQGL |
| | bevacizumab heavy | EWMGWINTYTGEPTYAQKFQGRVTITADESTSTAYMELSSL |
| | chain VH3 variant | RSEDTAVYYCAKYPHYYGSSHWYFDVWGQGTLVTVSSAST |
| | (BevaVH3) with | KGPSVFPLAPSSKSTSGGTAALGCLVKDYFPEPVTVSWNSGA |
| | IGF2 A chain with | LTSGVHTFPAVLQSSGLYSLSSVVTVPSSSLGTQTYICNVNHK |
| | V43L mutation, | PSNTKVDKKVEPKSCDKTHTCPPCPAPELLGGPSVFLFPPKPK |
| | MMP2 substrate | DTLMISRTPEVTCVVVDVSHEDPEVKFNWYVDGVEVHNAKT |
| | sequence and IgG1 Fc | KPREEQYNSTYRVVSVLTVLHQDWLNGKEYKCKVSNKALP |
| | with E345R, K409R, | APIEKTISKAKGQPRRPQVYTLPPSRDELTKNQVSLTCLVKGF |
| | M428L, N434S | YPSDIAVEWESNGQPENNYKTTPPVLDSDGSFFLYSRLTVDK |
| | mutations | SRWQQGNVFSCSVLHEALHSHYTQKSLSLSPGK |
| 234 | EAC847: Shielded | GILEECCFRSCDLALLETYCAGGGGSPLGLAGGGGGSEVQLV |
| | anti-VEGF | ESGGGLVQPGGSLRLSCAASGYTFTNYGMNWVRQAPGKGL |
| | bevacizumab heavy | EWVGWINTYTGEPTYAADFKRRFTFSLDTSKSTAYLQMNSL |
| | chain with IGF2 A | RAEDTAVYYCAKYPHYYGSSHWYFDVWGQGTLVTVSSAST |
| | chain with V43L | KGPSVFPLAPSSKSTSGGTAALGCLVKDYFPEPVTVSWNSGA |
| | mutation, MMP2 | LTSGVHTFPAVLQSSGLYSLSSVVTVPSSSLGTQTYICNVNHK |
| | substrate sequence | PSNTKVDKKVEPKSCDKTHTCPPCPAPELLGGPSVFLFPPKPK |
| | and IgG1 Fc with | DTLMISRTPEVTCVVVDVSHEDPEVKFNWYVDGVEVHNAKT |
| | K409R, M428L, | KPREEQYNSTYRVVSVLTVLHQDWLNGKEYKCKVSNKALP |
| | N434S mutations | APIEKTISKAKGQPREPQVYTLPPSRDELTKNQVSLTCLVKGF |
| | | YPSDIAVEWESNGQPENNYKTTPPVLDSDGSFFLYSRLTVDK |
| | | SRWQQGNVFSCSVLHEALHSHYTQKSLSLSPGK |
| 235 | EAC848: Shielded | AYRPSETLCGGELVDTLQFVCGDRGFAFGGGGSPLGLAGGG |
| | anti-VEGF | GGSDIQMTQSPSSLSASVGDRVTITCSASQDISNYLNWYQQK |
| | bevacizumab light | PGKAPKVLIYFTSSLHSGVPSRFSGSGSGTDFTLTISSLQPEDF |
| | chain with IGF2 B | ATYYCQQYSTVPWTFGQGTKVEIKRTVAAPSVFIFPPSDEQL |
| | chain with Y27A | KSGTASVVCLLNNFYPREAKVQWKVDNALQSGNSQESVTEQ |
| | mutation and MMP2 | DSKDSTYSLSSTLTLSKADYEKHKVYACEVTHQGLSSPVTKS |
| | substrate sequence | FNRGEC |
| 236 | EAC849: Shielded | AYRPSETLCGGELVDTLQFVCGDRGFAFGGGGSLSGRSDNH |
| | anti-VEGF | GGGGSDIQMTQSPSSLSASVGDRVTITCRASQDISNYLNWYQ |
| | bevacizumab light | QKPGKAPKVLIYFTSSLHSGVPSRFSGSGSGTDFTLTISSLQPE |
| | chain VL1 variant | DFATYYCQQYSTVPWTFGQGTKLEIKRTVAAPSVFIFPPSDEQ |
| | (BevaVL1) with IGF2 | LKSGTASVVCLLNNFYPREAKVQWKVDNALQSGNSQESVTE |
| | B chain with Y27A | QDSKDSTYSLSSTLTLSKADYEKHKVYACEVTHQGLSSPVTK |
| | mutation and uPA | SFNRGEC |
| | substrate sequence | |
| 237 | EAC851: Shielded | DQCIVDDITYNVQDTFHKKHEEGHMLNCTCFGQGRGRWKC |
| | anti-VEGF | DPVDQCQDSETGTFYQIGDSWEKYVHGVRYQCYCYGRGIGE |
| | bevacizumab light | WHCQPLQTYPSSGGGGSLSGRSDNHGGGGSDIQMTQSPSSLS |
| | chain VL1 variant | ASVGDRVTITCRASQDISNYLNWYQQKPGKAPKVLIYFTSSL |
| | (BevaVL1) with | HSGVPSRFSGSGSGTDFTLTISSLQPEDFATYYCQQYSTVPWT |
| | fibronectin peptide | FGQGTKLEIKRTVAAPSVFIFPPSDEQLKSGTASVVCLLNNFY |
| | masking domain and | PREAKVQWKVDNALQSGNSQESVTEQDSKDSTYSLSSTLTLS |
| | uPA substrate | KADYEKHKVYACEVTHQGLSSPVTKSFNRGEC |
| | sequence | |
| 238 | EAC1043: Shielded | GILEECCFRSCDLALLETYCAGGGGSPLGLAGGGGGSQVQLV |
| | anti-VEGF | QSGAEVKKPGSSVKVSCKASGYTFTNYGMNWVRQAPGQGL |
| | bevacizumab heavy | EWMGWINTYTGEPTYAQKFQGRVTITADESTSTAYMELSSL |
| | chain VH3 variant | RSEDTAVYYCAKYPHYYGSSHWYFDVWGQGTLVTVSSAST |
| | (BevaVH3) with | KGPSVFPLAPSSKSTSGGTAALGCLVKDYFPEPVTVSWNSGA |
| | IGF2 A chain with | LTSGVHTFPAVLQSSGLYSLSSVVTVPSSSLGTQTYICNVNHK |
| | V43L mutation, | PSNTKVDKKVEPKSCDKTHTCPPCPAPELLGGPSVFLFPPKPK |
| | MMP2 substrate | DTLMISRTPEVTCVVVDVSHEDPEVKFNWYVDGVEVHNAKT |
| | sequence and IgG1 Fc | KPREEQYNSTYRVVSVLTVLHQDWLNGKEYKCKVSNKALP |
| | with T3665, L368A, | APIEKTISKAKGQPREPQVYTLPPSRDELTKNQVSLSCAVKGF |
| | Y407V, M428L, | YPSDIAVEWESNGQPENNYKTTPPVLDSDGSFFLVSKLTVDK |
| | N434S mutations | SRWQQGNVFSCSVLHEALHSHYTQKSLSLSPGK |
| 239 | EAC1051: Shielded | GILEECCFRSCDLALLETYCAGGGGSPLGLAGGGGGSQVQLV |
| | anti-VEGF | QSGAEVKKPGSSVKVSCKASGYTFTNYGMNWVRQAPGQGL |
| | bevacizumab heavy | EWMGWINTYTGEPTYAQKFQGRVTITADESTSTAYMELSSL |
| | chain VH3 variant | RSEDTAVYYCAKYPHYYGSSHWYFDVWGQGTLVTVSSAST |
| | (BevaVH3) with | KGPSVFPLAPSSKSTSGGTAALGCLVKDYFPEPVTVSWNSGA |
| | IGF2 A chain with | LTSGVHTFPAVLQSSGLYSLSSVVTVPSSSLGTQTYICNVNHK |
| | V43L mutation, | PSNTKVDKKVEPKSCDKTHTCPPCPAPELLGGPSVFLLPPKPK |
| | MMP2 substrate | DTLMISRTPEVTCVVVDVSHEDPEVKFNWYVDGVEVHNAKT |
| | sequence and IgG1 Fc | KPPEEQYNSTLRVVSILTVLHQDWLNGKEYKCKVSNKALPA |
| | with F243L, R292P, | PIEKTISKAKGQPREPQVYTLPPSRDELTKNQVSLSCAVKGFY |
| | Y300L, V3051, | PSDIAVEWESNGQPENNYKTTPLVLDSDGSFFLVSKLTVDKS |
| | P396L, T366S, | RWQQGNVFSCSVMHEALHNHYTQKSLSLSPGK |
| | L368A, Y407V | |
| | mutations | |
| 240 | EAC1052: Shielded | EVGQRGVVGLPGQRGERGGGGSPLGLAGGGGGSQVQLVQS |
| | anti-VEGF | GAEVKKPGSSVKVSCKASGYTFTNYGMNWVRQAPGQGLEW |
| | bevacizumab heavy | MGWINTYTGEPTYAQKFQGRVTITADESTSTAYMELSSLRSE |
| | chain VH3 variant | DTAVYYCAKYPHYYGSSHWYFDVWGQGTLVTVSSASTKGP |
| | (BevaVH3) with | SVFPLAPSSKSTSGGTAALGCLVKDYFPEPVTVSWNSGALTS |
| | collagen peptide 2 | GVHTFPAVLQSSGLYSLSSVVTVPSSSLGTQTYICNVNHKPSN |
| | masking domain, | TKVDKKVEPKSCDKTHTCPPCPAPELLGGPSVFLLPPKPKDTL |
| | MMP2 substrate | MISRTPEVTCVVVDVSHEDPEVKFNWYVDGVEVHNAKTKPP |
| | sequence and IgG1 Fc | EEQYNSTLRVVSILTVLHQDWLNGKEYKCKVSNKALPAPIEK |
| | with F243L, R292P, | TISKAKGQPREPQVYTLPPSRDELTKNQVSLSCAVKGFYPSDI |
| | Y300L, V3051, | AVEWESNGQPENNYKTTPLVLDSDGSFFLVSKLTVDKSRWQ |
| | P396L, T3665, | QGNVFSCSVMHEALHNHYTQKSLSLSPGK |
| | L368A, Y407V | |
| | mutations | |
| 241 | Shielded anti-VEGF | CAQTMRIKPHQGQHIGESCGGGGSGGGGSGPLGVRGGGGSD |
| | bevacizumab light | IQMTQSPSSLSASVGDRVTITCSASQDISNYLNWYQQKPGKA |
| | chain with epitope as | PKVLIYFTSSLHSGVPSRFSGSGSGTDFTLTISSLQPEDFATYY |
| | masking domain and | CQQYSTVPWTFGQGTKVEIKRTVAAPSVFIFPPSDEQLKSGTA |
| | MMP2 substrate | SVVCLLNNFYPREAKVQWKVDNALQSGNSQESVTEQDSKDS |
| | sequence | TYSLSSTLTLSKADYEKHKVYACEVTHQGLSSPVTKSFNRGE |
| | | C |
| 242 | Shielded anti-VEGF | GGGGSCAQTMRIKPHQGQHIGESCGGGGSGGGGSGPLGVRG |
| | bevacizumab heavy | GGGSEVQLVESGGGLVQPGGSLRLSCAASGYTFTNYGMNW |
| | chain with epitope as | VRQAPGKGLEWVGWINTYTGEPTYAADFKRRFTFSLDTSKS |
| | masking domain, | TAYLQMNSLRAEDTAVYYCAKYPHYYGSSHWYFDVWGQG |
| | MMP2 substrate | TLVTVSSASTKGPSVFPLAPSSKSTSGGTAALGCLVKDYFPEP |
| | sequence and IgG1 Fc | VTVSWNSGALTSGVHTFPAVLQSSGLYSLSSVVTVPSSSLGT |
| | with K409R, M428L, | QTYICNVNHKPSNTKVDKKVEPKSCDKTHTCPPCPAPELLGG |
| | N434S mutations | PSVFLFPPKPKDTLMISRTPEVTCVVVDVSHEDPEVKFNWYV |
| | | DGVEVHNAKTKPREEQYNSTYRVVSVLTVLHQDWLNGKEY |
| | | KCKVSNKALPAPIEKTISKAKGQPREPQVYTLPPSRDELTKNQ |
| | | VSLTCLVKGFYPSDIAVEWESNGQPENNYKTTPPVLDSDGSF |
| | | FLYSRLTVDKSRWQQGNVFSCSVLHEALHSHYTQKSLSLSPG |
| | | K |
| 243 | Shielded anti-VEGF | CAQTMRIKPHQGQHIGESCGGGGSGGGGSGPLGVRGGGGSD |
| | bevacizumab light | IQMTQSPSSLSASVGDRVTITCSASQDISNYLNWYQQKPGKA |
| | chain with epitope as | PKVLIYFTSSLHSGVPSRFSGSGSGTDFTLTISSLQPEDFATYY |
| | masking domain, | CQQYSTVPWTFGQGTKVEIKRTVAAPSVFIFPPSDEQLKSGTA |
| | MMP2 substrate | SVVCLLNNFYPREAKVQWKVDNALQSGNSQESVTEQDSKDS |
| | sequence and F19-2 | TYSLSSTLTLSKADYEKHKVYACEVTHQGLSSPVTKSFNRGE |
| | homing domain | CGGGGSGGGGSEVQLLESGGGLVQPGGSLRLSCAASGFTFSS |
| | | FSMSWVRQAPGKGLEWVSSISGSSGTTYYADSVKGRFTISRD |
| | | NSKNTLYLQMNSLRAEDTAVYYCAKPFPYFDYWGQGTLVT |
| | | VSSGGSGGGGSGGGGSGGEIVLTQSPGTLSLSPGERATLSCRA |
| | | SQSVSSSFLAWYQQKPGQAPRLLIYYASSRATGIPDRFSGSGS |
| | | GTDFTLTISRLEPEDFAVYYCQQTGRIPPTFGQGTKVEIK |
| 244 | Shielded anti-VEGF | EVQLVESGGGLVQPGGSLRLSCAASGYTFTNYGMNWVRQA |
| | bevacizumab heavy | PGKGLEWVGWINTYTGEPTYAADFKRRFTFSLDTSKSTAYL |
| | chain with epitope as | QMNSLRAEDTAVYYCAKYPHYYGSSHWYFDVWGQGTLVT |
| | masking domain, | VSSASTKGPSVFPLAPSSKSTSGGTAALGCLVKDYFPEPVTVS |
| | MMP2 substrate | WNSGALTSGVHTFPAVLQSSGLYSLSSVVTVPSSSLGTQTYIC |
| | sequence, IgG1 Fc | NVNHKPSNTKVDKKVEPKSCDKTHTCPPCPAPELLGGPSVFL |
| | with K409R, M428L, | FPPKPKDTLMISRTPEVTCVVVDVSHEDPEVKFNWYVDGVE |
| | N434S mutations and | VHNAKTKPREEQYNSTYRVVSVLTVLHQDWLNGKEYKCKV |
| | F19-2 homing domain | SNKALPAPIEKTISKAKGQPREPQVYTLPPSRDELTKNQVSLT |
| | | CLVKGFYPSDIAVEWESNGQPENNYKTTPPVLDSDGSFFLYS |
| | | RLTVDKSRWQQGNVFSCSVLHEALHSHYTQKSLSLSPGKGG |
| | | GGSGGGGSEVQLLESGGGLVQPGGSLRLSCAASGFTFSSFSM |
| | | SWVRQAPGKGLEWVSSISGSSGTTYYADSVKGRFTISRDNSK |
| | | NTLYLQMNSLRAEDTAVYYCAKPFPYFDYWGQGTLVTVSS |
| | | GGSGGGGSGGGGSGGEIVLTQSPGTLSLSPGERATLSCRASQS |
| | | VSSSFLAWYQQKPGQAPRLLIYYASSRATGIPDRFSGSGSGTD |
| | | FTLTISRLEPEDFAVYYCQQTGRIPPTFGQGTKVEIK |
| 245 | Shielded anti-VEGF | CAQTMRIKPHQGQHIGESCGGGGSGGGGSGPLGVRGGGGSD |
| | bevacizumab light | IQMTQSPSSLSASVGDRVTITCSASQDISNYLNWYQQKPGKA |
| | chain with epitope as | PKVLIYFTSSLHSGVPSRFSGSGSGTDFTLTISSLQPEDFATYY |
| | masking domain, | CQQYSTVPWTFGQGTKVEIKRTVAAPSVFIFPPSDEQLKSGTA |
| | MMP2 substrate | SVVCLLNNFYPREAKVQWKVDNALQSGNSQESVTEQDSKDS |
| | sequence and FAP-2 | TYSLSSTLTLSKADYEKHKVYACEVTHQGLSSPVTKSFNRGE |
| | homing domain | CGGGGSGGGGSEVQLVESGGGLVQPGPSLRLSCTASRYTFTE |
| | | YTIHWVRQAPGKGLEWVGGINPNNGIPNYAASVKGRFTISVD |
| | | DSKSIAYLQMNSLKTEDTAVYFCARRRIAYGYDEGHAMDY |
| | | WGQGTTVTVSSGGSGGGGSGGGGSGGAIRMTQSPSSFSVST |
| | | GDRVTMTCRSSQSLLYSRNQKNYLAWFQQKPGKAPKLLIFW |
| | | ASTRESGVPSRFSGSGSGTDFTLTISCVQSEDFATYDCQQYFS |
| | | YPLTFGQGTKLEIK |
| 246 | Shielded anti-VEGF | CAQTMRIKPHQGQHIGESCGGGGSGGGGSGPLGVRGGGGSE |
| | bevacizumab heavy | VQLVESGGGLVQPGGSLRLSCAASGYTFTNYGMNWVRQAP |
| | chain with epitope as | GKGLEWVGWINTYTGEPTYAADFKRRFTFSLDTSKSTAYLQ |
| | masking domain, | MNSLRAEDTAVYYCAKYPHYYGSSHWYFDVWGQGTLVTV |
| | MMP2 substrate | SSASTKGPSVFPLAPSSKSTSGGTAALGCLVKDYFPEPVTVSW |
| | sequence, IgG1 Fc | NSGALTSGVHTFPAVLQSSGLYSLSSVVTVPSSSLGTQTYICN |
| | with K409R, M428L, | VNHKPSNTKVDKKVEPKSCDKTHTCPPCPAPELLGGPSVFLF |
| | N434S mutations and | PPKPKDTLMISRTPEVTCVVVDVSHEDPEVKFNWYVDGVEV |
| | FAP-2 homing | HNAKTKPREEQYNSTYRVVSVLTVLHQDWLNGKEYKCKVS |
| | domain | NKALPAPIEKTISKAKGQPREPQVYTLPPSRDELTKNQVSLTC |
| | | LVKGFYPSDIAVEWESNGQPENNYKTTPPVLDSDGSFFLYSR |
| | | LTVDKSRWQQGNVFSCSVLHEALHSHYTQKSLSLSPGKGGG |
| | | GSGGGGSEVQLVESGGGLVQPGPSLRLSCTASRYTFTEYTIH |
| | | WVRQAPGKGLEWVGGINPNNGIPNYAASVKGRFTISVDDSK |
| | | SIAYLQMNSLKTEDTAVYFCARRRIAYGYDEGHAMDYWGQ |
| | | GTTVTVSSGGSGGGGSGGGGSGGAIRMTQSPSSFSVSTGDRV |
| | | TMTCRSSQSLLYSRNQKNYLAWFQQKPGKAPKLLIFWASTR |
| | | ESGVPSRFSGSGSGTDFTLTISCVQSEDFATYDCQQYFSYPLT |
| | | FGQGTKLEIK |
| 247 | EAC844: Shielded | AYRPSETLCGGELVDTLQFVCGDRGFAFGGGGSPLGLAGGG |
| | anti-VEGF | GGSDIQMTQSPSSLSASVGDRVTITCRASQDISNYLNWYQQK |
| | bevacizumab light | PGKAPKVLIYFTSSLHSGVPSRFSGSGSGTDFTLTISSLQPEDF |
| | chain VL1 variant | ATYYCQQYSTVPWTFGQGTKLEIKRTVAAPSVFIFPPSDEQL |
| | (BevaVL1) with IGF2 | KSGTASVVCLLNNFYPREAKVQWKVDNALQSGNSQESVTEQ |
| | B chain with Y27A | DSKDSTYSLSSTLTLSKADYEKHKVYACEVTHQGLSSPVTKS |
| | mutation, MMP2 | FNRGECGGGGSGGGGSGGGGSGGGGSEVQLVESGGGLVQP |
| | substrate sequence | GGSLRLSCAASGFTFSDSWIHWVRQAPGKGLEWVAWISPYG |
| | and ScFv of PD-L1 | GSTYYADSVKGRFTISADTSKNTAYLQMNSLRAEDTAVYYC |
| | antibody | ARRHWPGGFDYWGQGTLVTVSSGGGGSGGGGSGGGGSDIQ |
| | | MTQSPSSLSASVGDRVTITCRASQDVSTAVAWYQQKPGKAP |
| | | KLLIYSASFLYSGVPSRFSGSGSGTDFTLTISSLQPEDFATYYC |
| | | QQYLYHPATFGQGTKVEIK |
| 248 | EAC836: Shielded | DQCIVDDITYNVQDTFHKKHEEGHMLNCTCFGQGRGRWKC |
| | anti-VEGF | DPVDQCQDSETGTFYQIGDSWEKYVHGVRYQCYCYGRGIGE |
| | bevacizumab light | WHCQPLQTYPSSGGGGSPLGLAGGGGGSDIQMTQSPSSLSAS |
| | chain VL1 variant | VGDRVTITCRASQDISNYLNWYQQKPGKAPKVLIYFTSSLHS |
| | (BevaVL1) with | GVPSRFSGSGSGTDFTLTISSLQPEDFATYYCQQYSTVPWTFG |
| | fibronectin peptide | QGTKLEIKRTVAAPSVFIFPPSDEQLKSGTASVVCLLNNFYPR |
| | masking domain, | EAKVQWKVDNALQSGNSQESVTEQDSKDSTYSLSSTLTLSK |
| | MMP2 substrate | ADYEKHKVYACEVTHQGLSSPVTKSFNRGECGGGGSGGGGS |
| | sequence and ScFv of | GGGGSGGGGSEVQLVESGGGLVQPGGSLRLSCAASGFTFSDS |
| | PD-L1 antibody | WIHWVRQAPGKGLEWVAWISPYGGSTYYADSVKGRFTISAD |
| | | TSKNTAYLQMNSLRAEDTAVYYCARRHWPGGFDYWGQGT |
| | | LVTVSSGGGGSGGGGSGGGGSDIQMTQSPSSLSASVGDRVTI |
| | | TCRASQDVSTAVAWYQQKPGKAPKLLIYSASFLYSGVPSRFS |
| | | GSGSGTDFTLTISSLQPEDFATYYCQQYLYHPATFGQGTKVEI |
| | | K |

### REFERENCES

Abramson, V. and C. L. Arteaga (2011). “New strategies in HER2-overexpressing breast cancer: many combinations of targeted drugs available.” Clin Cancer Res 17(5): 952-958.
Alessi, P., C. Ebbinghaus and D. Neri (2004). “Molecular targeting of angiogenesis.” Biochim Biophys Acta 1654(1): 39-49.
Arteaga, C. L., M. X. Sliwkowski, C. K. Osborne, E. A. Perez, F. Puglisi and L. Gianni (2011). “Treatment of HER2-positive breast cancer: current status and future perspectives.” Nat Rev Clin Oncol 9(1): 16-32.
Autio, K. A., V. Boni, R. W. Humphrey and A. Naing (2020). “Probody Therapeutics: An Emerging Class of Therapies Designed to Enhance On-Target Effects with Reduced Off-Tumor Toxicity for Use in Immuno-Oncology.” Clin Cancer Res 26(5): 984-989.
Bair, S. M., T. K. Choueiri and J. Moslehi (2013). “Cardiovascular complications associated with novel angiogenesis inhibitors: emerging evidence and evolving perspectives.” Trends Cardiovasc Med 23(4): 104-113.
Baliga, R. R., D. R. Pimental, Y. Y. Zhao, W. W. Simmons, M. A. Marchionni, D. B. Sawyer and R. A. Kelly (1999). “NRG-1-induced cardiomyocyte hypertrophy. Role of PI-3-kinase, p70(S6K), and MEK-MAPK-RSK.” Am J Physiol 277(5): H2026-2037.
Banys-Paluchowski, M., I. Witzel, B. Aktas, P. A. Fasching, A. Hartkopf, W. Janni, S. Kasimir-Bauer, K. Pantel, G. Schon, B. Rack, S. Riethdorf, E. F. Solomayer, T. Fehm and V. Muller (2019). “The prognostic relevance of urokinase-type plasminogen activator (uPA) in the blood of patients with metastatic breast cancer.” Sci Rep 9(1): 2318.
Baselga, J., J. Cortes, S. B. Kim, S. A. Im, R. Hegg, Y. H. Im, L. Roman, J. L. Pedrini, T. Pienkowski, A. Knott, E. Clark, M. C. Benyunes, G. Ross, S. M. Swain and C. S. Group (2012). “Pertuzumab plus trastuzumab plus docetaxel for metastatic breast cancer.” N Engl J Med 366(2): 109-119.
Bersell, K., S. Arab, B. Haring and B. Kuhn (2009). “Neuregulinl/ErbB4 signaling induces cardiomyocyte proliferation and repair of heart injury.” Cell 138(2): 257-270.
Bolt, S., E. Routledge, I. Lloyd, L. Chatenoud, H. Pope, S. D. Gorman, M. Clark and H. Waldmann (1993). “The generation of a humanized, non-mitogenic CD3 monoclonal antibody which retains in vitro immunosuppressive properties.” Eur J Immunol 23(2): 403-411.
Bostrom, J., S. F. Yu, D. Kan, B. A. Appleton, C. V. Lee, K. Billeci, W. Man, F. Peale, S. Ross, C. Wiesmann and G. Fuh (2009). “Variants of the antibody herceptin that interact with HER2 and VEGF at the antigen binding site.” Science 323(5921): 1610-1614.
Bowles, E. J., R. Wellman, H. S. Feigelson, A. A. Onitilo, A. N. Freedman, T. Delate, L. A. Allen, L. Nekhlyudov, K. A. Goddard, R. L. Davis, L. A. Habel, M. U. Yood, C. McCarty, D. J. Magid, E. H. Wagner and T. Pharmacovigilance Study (2012). “Risk of heart failure in breast cancer patients after anthracycline and trastuzumab treatment: a retrospective cohort study.” J Natl Cancer Inst 104(17): 1293-1305.
Camprecios, G., J. Lorita, E. Pardina, J. Peinado-Onsurbe, M. Soley and I. Ramirez (2011). “Expression, localization, and regulation of the neuregulin receptor ErbB3 in mouse heart.” J Cell Physiol 226(2): 450-455.
Carnemolla, B., E. Balza, A. Siri, L. Zardi, M. R. Nicotra, A. Bigotti and P. G. Natali (1989). “A tumor-associated fibronectin isoform generated by alternative splicing of messenger RNA precursors.” J Cell Biol 108(3): 1139-1148.
Castellani, P., G. Viale, A. Dorcaratto, G. Nicolo, J. Kaczmarek, G. Querze and L. Zardi (1994). “The fibronectin isoform containing the ED-B oncofetal domain: a marker of angiogenesis.” Int J Cancer 59(5): 612-618.
Chen, I. J., C. H. Chuang, Y. C. Hsieh, Y. C. Lu, W. W. Lin, C. C. Huang, T. C. Cheng, Y. A. Cheng, K. W. Cheng, Y. T. Wang, F. M. Chen, T. L. Cheng and S. C. Tzou (2017). “Selective antibody activation through protease-activated pro-antibodies that mask binding sites with inhibitory domains.” Sci Rep 7(1): 11587.
Chen, X., J. L. Zaro and W. C. Shen (2013). “Fusion protein linkers: property, design and functionality.” Adv Drug Deliv Rev 65(10): 1357-1369.
Chiosea, S. I., L. Williams, C. C. Griffith, L. D. Thompson, I. Weinreb, J. E. Bauman, A. Luvison, S. Roy, R. R. Seethala and M. N. Nikiforova (2015). “Molecular characterization of apocrine salivary duct carcinoma.” Am J Surg Pathol 39(6): 744-752.
Chothia, C. and A. M. Lesk (1987). “Canonical structures for the hypervariable regions of immunoglobulins.” J Mol Biol 196(4): 901-917.
Choueiri, T. K., E. L. Mayer, Y. Je, J. E. Rosenberg, P. L. Nguyen, G. R. Azzi, J. Bellmunt, H. J. Burstein and F. A. Schutz (2011). “Congestive heart failure risk in patients with breast cancer treated with bevacizumab.” J Clin Oncol 29(6): 632-638.
Chu, S. Y., I. Vostiar, S. Karki, G. L. Moore, G. A. Lazar, E. Pong, P. F. Joyce, D. E. Szymkowski and J. R. Desjarlais (2008). “Inhibition of B cell receptor-mediated activation of primary human B cells by coengagement of CD19 and FcgammaRIIb with Fc-engineered antibodies.” Mol Immunol 45(15): 3926-3933.
Chu, T. F., M. A. Rupnick, R. Kerkela, S. M. Dallabrida, D. Zurakowski, L. Nguyen, K. Woulfe, E. Pravda, F. Cassiola, J. Desai, S. George, J. A. Morgan, D. M. Harris, N. S. Ismail, J. H. Chen, F. J. Schoen, A. D. Van den Abbeele, G. D. Demetri, T. Force and M. H. Chen (2007). “Cardiotoxicity associated with tyrosine kinase inhibitor sunitinib.” Lancet 370(9604): 2011-2019.
Cortes, J., P. Fumoleau, G. V. Bianchi, T. M. Petrella, K. Gelmon, X. Pivot, S. Verma, J. Albanell, P. Conte, A. Lluch, S. Salvagni, V. Servent, L. Gianni, M. Scaltriti, G. A. Ross, J. Dixon, T. Szado and J. Baselga (2012). “Pertuzumab monotherapy after trastuzumab-based treatment and subsequent reintroduction of trastuzumab: activity and tolerability in patients with advanced human epidermal growth factor receptor 2-positive breast cancer.” J Clin Oncol 30(14): 1594-1600.
Cretella, D., F. Saccani, F. Quaini, C. Frati, C. Lagrasta, M. Bonelli, C. Caffarra, A. Cavazzoni, C. Fumarola, M. Galetti, S. La Monica, L. Ampollini, M. Tiseo, A. Ardizzoni, P. G. Petronini and R. R. Alfieri (2014). “Trastuzumab emtansine is active on HER-2 overexpressing NSCLC cell lines and overcomes gefitinib resistance.” Mol Cancer 13: 143.
Crone, S. A., Y. Y. Zhao, L. Fan, Y. Gu, S. Minamisawa, Y. Liu, K. L. Peterson, J. Chen, R. Kahn, G. Condorelli, J. Ross, Jr., K. R. Chien and K. F. Lee (2002). “ErbB2 is essential in the prevention of dilated cardiomyopathy.” Nat Med 8(5): 459-465.
Dall'Acqua, W. F., P. A. Kiener and H. Wu (2006). “Properties of human IgGls engineered for enhanced binding to the neonatal Fc receptor (FcRn).” J Biol Chem 281(33): 23514-23524.
de Jesus-Gonzalez, N., E. Robinson, J. Moslehi and B. D. Humphreys (2012). “Management of antiangiogenic therapy-induced hypertension.” Hypertension 60(3): 607-615.
de Melo Gagliato, D., D. L. Jardim, M. S. Marchesi and G. N. Hortobagyi (2016). “Mechanisms of resistance and sensitivity to anti-HER2 therapies in HER2+ breast cancer.” Oncotarget 7(39): 64431-64446.
DeSantis, C., J. Ma, L. Bryan and A. Jemal (2014). “Breast cancer statistics, 2013.” CA Cancer J Clin 64(1): 52-62.
Diebolder, C. A., F. J. Beurskens, R. N. de Jong, R. I. Koning, K. Strumane, M. A. Lindorfer, M. Voorhorst, D. Ugurlar, S. Rosati, A. J. Heck, J. G. van de Winkel, I. A. Wilson, A. J. Koster, R. P. Taylor, E. O. Saphire, D. R. Burton, J. Schuurman, P. Gros and P. W. Parren (2014). “Complement is activated by IgG hexamers assembled at the cell surface.” Science 343(6176): 1260-1263.
Donaldson, J. M., C. Kari, R. C. Fragoso, U. Rodeck and J. C. Williams (2009). “Design and development of masked therapeutic antibodies to limit off-target effects: application to anti-EGFR antibodies.” Cancer Biol Ther 8(22): 2147-2152.
Dvorak, H. F., L. F. Brown, M. Detmar and A. M. Dvorak (1995). “Vascular permeability factor/vascular endothelial growth factor, microvascular hyperpermeability, and angiogenesis.” Am J Pathol 146(5): 1029-1039.
Economopoulou, P., A. Kotsakis, I. Kapiris and N. Kentepozidis (2015). “Cancer therapy and cardiovascular risk: focus on bevacizumab.” Cancer Manag Res 7: 133-143.
Ewer, M. S. and S. M. Lippman (2005). “Type II chemotherapy-related cardiac dysfunction: time to recognize a new entity.” J Clin Oncol 23(13): 2900-2902.
Falls, D. L. (2003). “Neuregulins: functions, forms, and signaling strategies.” Exp Cell Res 284(1): 14-30.
Ferrara, N. and T. Davis-Smyth (1997). “The biology of vascular endothelial growth factor.” Endocr Rev 18(1): 4-25.
Folkman, J. (1995). “Angiogenesis in cancer, vascular, rheumatoid and other disease.” Nat Med 1(1): 27-31.
Folkman, J. and Y. Shing (1992). “Angiogenesis.” J Biol Chem 267(16): 10931-10934.
Fukumura, D., R. Xavier, T. Sugiura, Y. Chen, E. C. Park, N. Lu, M. Selig, G. Nielsen, T. Taksir, R. K. Jain and B. Seed (1998). “Tumor induction of VEGF promoter activity in stromal cells.” Cell 94(6): 715-725.
Garin-Chesa, P., L. J. Old and W. J. Rettig (1990). “Cell surface glycoprotein of reactive stromal fibroblasts as a potential antibody target in human epithelial cancers.” Proc Natl Acad Sci USA 87(18): 7235-7239.
Gianni, L., G. H. Romieu, M. Lichinitser, S. V. Serrano, M. Mansutti, X. Pivot, P. Mariani, F. Andre, A. Chan, O. Lipatov, S. Chan, A. Wardley, R. Greil, N. Moore, S. Prot, C. Pallaud and V. Semiglazov (2013). “AVEREL: a randomized phase III Trial evaluating bevacizumab in combination with docetaxel and trastuzumab as first-line therapy for HER2-positive locally recurrent/metastatic breast cancer.” J Clin Oncol 31(14): 1719-1725.
Gunasekaran, K., M. Pentony, M. Shen, L. Garrett, C. Forte, A. Woodward, S. B. Ng, T. Born, M. Retter, K. Manchulenko, H. Sweet, I. N. Foltz, M. Wittekind and W. Yan (2010). “Enhancing antibody Fc heterodimer formation through electrostatic steering effects: applications to bispecific molecules and monovalent IgG.” J Biol Chem 285(25): 19637-19646.
Hedhli, N., Q. Huang, A. Kalinowski, M. Palmeri, X. Hu, R. R. Russell and K. S. Russell (2011). “Endothelium-derived neuregulin protects the heart against ischemic injury.” Circulation 123(20): 2254-2262.
Higgins, M. J. and J. Baselga (2011). “Targeted therapies for breast cancer.” J Clin Invest 121(10): 3797-3803.
Hinton, P. R., J. M. Xiong, M. G. Johlfs, M. T. Tang, S. Keller and N. Tsurushita (2006). “An engineered human IgG1 antibody with longer serum half-life.” J Immunol 176(1): 346-356.
Holmgren, L., M. S. O'Reilly and J. Folkman (1995). “Dormancy of micrometastases: balanced proliferation and apoptosis in the presence of angiogenesis suppression.” Nat Med 1(2): 149-153.
Horak, E. R., R. Leek, N. Klenk, S. LeJeune, K. Smith, N. Stuart, M. Greenall, K. Stepniewska and A. L. Harris (1992). “Angiogenesis, assessed by platelet/endothelial cell adhesion molecule antibodies, as indicator of node metastases and survival in breast cancer.” Lancet 340(8828): 1120-1124.
Hu, S., W. Fu, W. Xu, Y. Yang, M. Cruz, S. D. Berezov, D. Jorissen, H. Takeda and W. Zhu (2015). “Four-in-one antibodies have superior cancer inhibitory activity against EGFR, HER2, HER3, and VEGF through disruption of HER/MET crosstalk.” Cancer Res 75(1): 159-170.
Hudis, C. A. (2007). “Trastuzumab—mechanism of action and use in clinical practice.” N Engl J Med 357(1): 39-51.
Iwamoto, R., S. Yamazaki, M. Asakura, S. Takashima, H. Hasuwa, K. Miyado, S. Adachi, M. Kitakaze, K. Hashimoto, G. Raab, D. Nanba, S. Higashiyama, M. Hori, M. Klagsbrun and E. Mekada (2003). “Heparin-binding EGF-like growth factor and ErbB signaling is essential for heart function.” Proc Natl Acad Sci USA 100(6): 3221-3226.
Juillerat-Jeanneret, L., P. Tafelmeyer and D. Golshayan (2017). “Fibroblast activation protein-alpha in fibrogenic disorders and cancer: more than a prolyl-specific peptidase?” Expert Opin Ther Targets 21(10): 977-991.
Kaumaya, P. T. P. and K. C. Foy (2012). “Peptide vaccines and peptidomimetics targeting HER and VEGF proteins may offer a potentially new paradigm in cancer immunotherapy.” Future Oncol. 8(8): 961-987.
Kavanaugh, W. M. (2020). “Antibody prodrugs for cancer.” Expert Opin Biol Ther 20(2): 163-171.
Kim, K. J., B. Li, J. Winer, M. Armanini, N. Gillett, H. S. Phillips and N. Ferrara (1993). “Inhibition of vascular endothelial growth factor-induced angiogenesis suppresses tumour growth in vivo.” Nature 362(6423): 841-844.
Kinder, M., A. R. Greenplate, K. D. Grugan, K. L. Soring, K. A. Heeringa, S. G. McCarthy, G. Bannish, M. Perpetua, F. Lynch, R. E. Jordan, W. R. Strohl and R. J. Brerski (2013). “Engineered protease-resistant antibodies with selectable cell-killing functions.” J Biol Chem 288(43): 30843-30854.
Klagsbrun, M. and P. A. D'Amore (1991). “Regulators of angiogenesis.” Annu Rev Physiol 53: 217-239.
Labrijn, A. F., J. I. Meesters, B. E. de Goeij, E. T. van den Bremer, J. Neijssen, M. D. van Kampen, K. Strumane, S. Verploegen, A. Kundu, M. J. Gramer, P. H. van Berkel, J. G. van de Winkel, J. Schuurman and P. W. Parren (2014). “Efficient generation of stable bispecific IgG1 by controlled Fab-arm exchange.” Proc Natl Acad Sci USA 110(13): 5145-5150.
Le, X. F., W. Mao, C. Lu, A. Thornton, J. V. Heymach, A. K. Sood and R. C. Bast, Jr. (2008). “Specific blockade of VEGF and HER2 pathways results in greater growth inhibition of breast cancer xenografts that overexpress HER2.” Cell Cycle 7(23): 3747-3758.
Lefranc, M. P., C. Pommie, M. Ruiz, V. Giudicelli, E. Foulquier, L. Truong, V. Thouvenin-Contet and G. Lefranc (2003). “IMGT unique numbering for immunoglobulin and T cell receptor variable domains and Ig superfamily V-like domains.” Dev Comp Immunol 27(1): 55-77.
Lemmens, K., K. Doggen and G. W. De Keulenaer (2007). “Role of neuregulin-1/ErbB signaling in cardiovascular physiology and disease: implications for therapy of heart failure.” Circulation 116(8): 954-960.
Li, M. and D. L. Kroetz (2018). “Bevacizumab-induced hypertension: Clinical presentation and molecular understanding.” Pharmacol Ther 182: 152-160.
Lin, N. U., D. S. Seah, R. Gelman, S. Desantis, E. L. Mayer, S. Isakoff, P. Dipiro, I. E. Krop, S. E. Come, D. Weckstein, E. P. Winer and H. J. Burstein (2013). “A phase II study of bevacizumab in combination with vinorelbine and trastuzumab in HER2-positive metastatic breast cancer.” Breast Cancer Res Treat 139(2): 403-410.
Lin, W. W., Y. C. Lu, C. H. Chuang and T. L. Cheng (2020). “Ab locks for improving the selectivity and safety of antibody drugs.” J Biomed Sci 27(1): 76.
Lu, Y. C., C. H. Chuang, K. H. Chuang, I. J. Chen, B. C. Huang, W. H. Lee, H. E. Wang, J. J. Li, Y. A. Cheng, K. W. Cheng, J. Y. Wang, Y. C. Hsieh, W. W. Lin and T. L. Cheng (2019). “Specific activation of pro-Infliximab enhances selectivity and safety of rheumatoid arthritis therapy.” PLoS Biol 17(6): e3000286.
Luque-Cabal, M., P. Garcia-Teijido, Y. Fernandez-Perez, L. Sanchez-Lorenzo and I. Palacio-Vazquez (2016). “Mechanisms Behind the Resistance to Trastuzumab in HER2-Amplified Breast Cancer and Strategies to Overcome It.” Clin Med Insights Oncol 10(Suppl 1): 21-30.
Macchiarini, P., G. Fontanini, M. J. Hardin, F. Squartini and C. A. Angeletti (1992). “Relation of neovascularisation to metastasis of non-small-cell lung cancer.” Lancet 340(8812): 145-146.
Martin, M., A. Makhson, J. Gligorov, M. Lichinitser, A. Lluch, V. Semiglazov, N. Scotto, L. Mitchell and S. Tjulandin (2012). “Phase II study of bevacizumab in combination with trastuzumab and capecitabine as first-line treatment for HER-2-positive locally recurrent or metastatic breast cancer.” Oncologist 17(4): 469-475.
May, D., D. Gilon, V. Djonov, A. Itin, A. Lazarus, O. Gordon, C. Rosenberger and E. Keshet (2008). “Transgenic system for conditional induction and rescue of chronic myocardial hibernation provides insights into genomic programs of hibernation.” Proc Natl Acad Sci USA 105(1): 282-287.
Metz, S., C. Panke, A. K. Haas, J. Schanzer, W. Lau, R. Croasdale, E. Hoffmann, B. Schneider, J. Auer, C. Gassner, B. Bossenmaier, P. Umana, C. Sustmann and U. Brinkmann (2012). “Bispecific antibody derivatives with restricted binding functionalities that are activated by proteolytic processing.” Protein Eng Des Sel 25(10): 571-580.
Meza-Junco, J., H. J. Au and M. B. Sawyer (2011). “Critical appraisal of trastuzumab in treatment of advanced stomach cancer.” Cancer Manag Res 3: 57-64.
Nami, B. and Z. Wang (2017). “HER2 in Breast Cancer Stemness: A Negative Feedback Loop towards Trastuzumab Resistance.” Cancers (Basel) 9(5).
Nazer, B., B. D. Humphreys and J. Moslehi (2011). “Effects of novel angiogenesis inhibitors for the treatment of cancer on the cardiovascular system: focus on hypertension.” Circulation 124(15): 1687-1691.
Olayioye, M. A. (2001). “Update on HER-2 as a target for cancer therapy: intracellular signaling pathways of ErbB2/HER-2 and family members.” Breast Cancer Res 3(6): 385-389.
Oliveira, M. (2015). “Managing common toxicities with new tyrosine kinase inhibitors.” CancerWorld: 31-37.
Pentassuglia, L. and D. B. Sawyer (2009). “The role of Neuregulin-1beta/ErbB signaling in the heart.” Exp Cell Res 315(4): 627-637.

Petkova, S. B., S. Akilesh, T. J. Sproule, G. J. Christianson, H. Al Khabbaz, A. C. Brown, L. G. Presta, Y. G. Meng and D. C. Roopenian (2006). “Enhanced half-life of genetically engineered human IgG1 antibodies in a humanized FcRn mouse model: potential application in humorally mediated autoimmune disease.” Int Immunol 18(12): 1759-1769.
Pierga, J. Y., T. Petit, T. Delozier, J. M. Ferrero, M. Campone, J. Gligorov, F. Lerebours, H. Roche, T. Bachelot, E. Charafe-Jauffret, M. Pavlyuk, S. Kraemer, F. C. Bidard and P. Viens (2012). “Neoadjuvant bevacizumab, trastuzumab, and chemotherapy for primary inflammatory HER2-positive breast cancer (BEVERLY-2): an open-label, single-arm phase 2 study.” Lancet Oncol 13(4): 375-384.
Pohlmann, P. R., I. A. Mayer and R. Mernaugh (2009). “Resistance to Trastuzumab in Breast Cancer.” Clin Cancer Res 15(24): 7479-7491.
Procter, M., T. M. Suter, E. de Azambuja, U. Dafni, V. van Dooren, S. Muehlbauer, M. A. Climent, E. Rechberger, W. T. Liu, M. Toi, R. C. Coombes, D. Dodwell, O. Pagani, J. Madrid, M. Hall, S. C. Chen, C. Focan, M. Muschol, D. J. van Veldhuisen and M. J. Piccart-Gebhart (2010). “Longer-term assessment of trastuzumab-related cardiac adverse events in the Herceptin Adjuvant (HERA) trial.” J Clin Oncol 28(21): 3422-3428.
Rettig, W. J., P. Garin-Chesa, J. H. Healey, S. L. Su, H. L. Ozer, M. Schwab, A. P. Albino and L. J. Old (1993). “Regulation and heteromeric structure of the fibroblast activation protein in normal and transformed cells of mesenchymal and neuroectodermal origin.” Cancer Res 53(14): 3327-3335.
Rexer, B. N. and C. L. Arteaga (2012). “Intrinsic and acquired resistance to HER2-targeted therapies in HER2 gene-amplified breast cancer: mechanisms and clinical implications.” Crit Rev Oncog 17(1): 1-16.
Ridgway, J. B., L. G. Presta and P. Carter (1996). “‘Knobs-into-holes’ engineering of antibody CH3 domains for heavy chain heterodimerization.” Protein Eng 9(7): 617-621.
Rubin, I. and Y. Yarden (2001). “The basic biology of HER2.” Ann Oncol 12 Suppl 1: S3-8. Ruschoff, J., W. Hanna, M. Bilous, M. Hofmann, R. Y. Osamura, F. Penault-Llorca, M. van de Vijver and G. Viale (2012). “HER2 testing in gastric cancer: a practical approach.” Mod Pathol 25(5): 637-650.
Santimaria, M., G. Moscatelli, G. L. Viale, L. Giovannoni, G. Neri, F. Viti, A. Leprini, L. Borsi, P. Castellani, L. Zardi, D. Neri and P. Riva (2003). “Immunoscintigraphic detection of the ED-B domain of fibronectin, a marker of angiogenesis, in patients with cancer.” Clin Cancer Res 9(2): 571-579.
Scheraga, H. A. (2008). “From helix-coil transitions to protein folding.” Biopolymers 89(5): 479-485.

Schmidinger, M., C. C. Zielinski, U. M. Vogl, A. Bojic, M. Bojic, C. Schukro, M. Ruhsam, M. Hejna and H. Schmidinger (2008). “Cardiac toxicity of sunitinib and sorafenib in patients with metastatic renal cell carcinoma.” J Clin Oncol 26(32): 5204-5212.
Shields, R. L., A. K. Namenuk, K. Hong, Y. G. Meng, J. Rae, J. Briggs, D. Xie, J. Lai, A. Stadlen, B. Li, J. A. Fox and L. G. Presta (2001). “High resolution mapping of the binding site on human IgG1 for Fc gamma RI, Fc gamma RII, Fc gamma RIII, and FcRn and design of IgG1 variants with improved binding to the Fc gamma R.” J Biol Chem 276(9): 6591-6604.
Siegel, R., C. DeSantis, K. Virgo, K. Stein, A. Mariotto, T. Smith, D. Cooper, T. Gansler, C. Lerro, S. Fedewa, C. Lin, C. Leach, R. S. Cannady, H. Cho, S. Scoppa, M. Hachey, R. Kirch, A. Jemal and E. Ward (2012). “Cancer treatment and survivorship statistics, 2012.” CA Cancer J Clin 62(4): 220-241.
Singh, R., W. J. Kim, P. H. Kim and H. J. Hong (2013). “Combined blockade of HER2 and VEGF exerts greater growth inhibition of HER2-overexpressing gastric cancer xenografts than individual blockade.” Exp Mol Med 45: e52.
Slamon, D. J., G. M. Clark, S. G. Wong, W. J. Levin, A. Ullrich and W. L. McGuire (1987). “Human breast cancer: correlation of relapse and survival with amplification of the HER-2/neu oncogene.” Science 235(4785): 177-182.
Stavenhagen, J. B., S. Gorlatov, N. Tuaillon, C. T. Rankin, H. Li, S. Burke, L. Huang, S. Johnson, S. Koenig and E. Bonvini (2008). “Enhancing the potency of therapeutic monoclonal antibodies via Fc optimization.” Adv Enzyme Regul 48: 152-164.
Suter, T. M., M. Procter, D. J. van Veldhuisen, M. Muscholl, J. Bergh, C. Carlomagno, T. Perren, R. Passalacqua, C. Bighin, J. G. Klijn, F. T. Ageev, E. Hitre, J. Groetz, H. Iwata, M. Knap, M. Gnant, S. Muehlbauer, A. Spence, R. D. Gelber and M. J. Piccart-Gebhart (2007). “Trastuzumab-associated cardiac adverse effects in the herceptin adjuvant trial.” J Clin Oncol 25(25): 3859-3865.
Tam, S. H., S. G. McCarthy, A. A. Armstrong, S. Somani, S. J. Wu, X. Liu, A. Gervais, R. Ernst, D. Saro, R. Decker, J. Luo, G. L. Gilliland, M. L. Chiu and B. J. Scallon (2017). “Functional, Biophysical, and Structural Characterization of Human IgG1 and IgG4 Fc Variants with Ablated Immune Functionality.” Antibodies (Basel) 6(3).
Tarli, L., E. Balza, F. Viti, L. Borsi, P. Castellani, D. Berndorff, L. Dinkelborg, D. Neri and L. Zardi (1999). “A high-affinity human antibody that targets tumoral blood vessels.” Blood 94(1): 192-198.
Trang, V. H., X. Zhang, R. C. Yumul, W. Zeng, I. J. Stone, S. W. Wo, M. M. Dominguez, J. H. Cochran, J. K. Simmons, M. C. Ryan, R. P. Lyon, P. D. Senter and M. R. Levengood (2019). “A coiled-coil masking domain for selective activation of therapeutic antibodies.” Nat Biotechnol 37(7): 761-765.
Troiani, T., E. Martinelli, M. Orditura, F. De Vita, F. Ciardiello and F. Morgillo (2012). “Beyond bevacizumab: new anti-VEGF strategies in colorectal cancer.” Expert Opin Investig Drugs 21(7): 949-959.
Vafa, O., G. L. Gilliland, R. J. Brerski, B. Strake, T. Wilkinson, E. R. Lacy, B. Scallon, A. Teplyakov, T. J. Malia and W. R. Strohl (2014). “An engineered Fc variant of an IgG eliminates all immune effector functions via structural perturbations.” Methods 65(1): 114-126.
Vaklavas, C., D. Lenihan, R. Kurzrock and A. M. Tsimberidou (2010). “Anti-vascular endothelial growth factor therapies and cardiovascular toxicity: what are the important clinical markers to target?” Oncologist 15(2): 130-141.
Verma, S., D. Miles, L. Gianni, I. E. Krop, M. Welslau, J. Baselga, M. Pegram, D. Y. Oh, V. Dieras, E. Guardino, L. Fang, M. W. Lu, S. Olsen, K. Blackwell and E. S. Group (2012). “Trastuzumab emtansine for HER2-positive advanced breast cancer.” N Engl J Med 367(19): 1783-1791.
Viti, F., L. Tarli, L. Giovannoni, L. Zardi and D. Neri (1999). “Increased binding affinity and valence of recombinant antibody fragments lead to improved targeting of tumoral angiogenesis.” Cancer Res 59(2): 347-352.
Warren, R. S., H. Yuan, M. R. Matli, N. A. Gillett and N. Ferrara (1995). “Regulation by vascular endothelial growth factor of human colon cancer tumorigenesis in a mouse model of experimental liver metastasis.” J Chin Invest 95(4): 1789-1797.
Weidner, N., J. P. Semple, W. R. Welch and J. Folkman (1991). “Tumor angiogenesis and metastasis—correlation in invasive breast carcinoma.” N Engl J Med 324(1): 1-8.
Worn, A. and A. Pluckthun (2001). “Stability engineering of antibody single-chain Fv fragments.” J Mol Biol 305(5): 989-1010.
Wu, T. T. and E. A. Kabat (1970). “An analysis of the sequences of the variable regions of Bence Jones proteins and myeloma light chains and their implications for antibody complementarity.” J Exp Med 132(2): 211-250.
Xu, D., M. L. Alegre, S. S. Varga, A. L. Rothermel, A. M. Collins, V. L. Pulito, L. S. Hanna, K. P. Dolan, P. W. Parren, J. A. Bluestone, L. K. Jolliffe and R. A. Zivin (2000). “In vitro characterization of five humanized OKT3 effector function variant antibodies.” Cell Immunol 200(1): 16-26.
Yang, L., Y. Li, A. Bhattacharya and Y. Zhang (2019). “A recombinant human protein targeting HER2 overcomes drug resistance in HER2-positive breast cancer.” Sci Transl Med 11(476).
Yin, W., J. Zhu, D. Gonzalez-Rivas, M. Okumura, G. Rocco, H. Pass, G. Jiang and Y. Yang (2018). “Construction of a Novel Bispecific Antibody to Enhance Antitumor Activity against Lung Cancer.” Adv Mater 30(51): e1805437.
Zalevsky, J., A. K. Chamberlain, H. M. Horton, S. Karki, I. W. Leung, T. J. Sproule, G. A. Lazar, D. C. Roopenian and J. R. Desjarlais (2010). “Enhanced antibody half-life improves in vivo activity.” Nat Biotechnol 28(2): 157-159.
Zardi, L., B. Carnemolla, A. Ski, T. E. Petersen, G. Paolella, G. Sebastio and F. E. Baralle (1987). “Transformed human cells produce a new fibronectin isoform by preferential alternative splicing of a previously unobserved exon.” EMBO J 6(8): 2337-2342.
Zhang, D., A. A. Armstrong, S. H. Tam, S. G. McCarthy, J. Luo, G. L. Gilliland and M. L. Chiu (2017). “Functional optimization of agonistic antibodies to OX40 receptor with novel Fc mutations to promote antibody multimerization.” MAbs 9: 1129-1142.
Zhao, Y. Y., D. R. Sawyer, R. R. Baliga, D. J. Opel, X. Han, M. A. Marchionni and R. A. Kelly (1998). “Neuregulins promote survival and growth of cardiac myocytes. Persistence of ErbB2 and ErbB4 expression in neonatal and adult ventricular myocytes.” J Biol Chem 273(17): 10261-10269.

We claim:

1. A bispecific antibody that is capable of targeting a HER2 associated pathway and a VEGF associated pathway and is capable of inhibiting tumor cell proliferation, wherein the bispecific antibody comprises one or more shielding domains and/or one or more homing domains.

2. The bispecific antibody of claim 1, comprising a binding arm targeting the HER2 associated pathway, including a human IgG heavy chain fusion comprising amino acid sequences from the N- to the C-terminus, signal sequence A-shield A-linker A-protease sequence A-linker B-IgG heavy chain; and a human IgG light chain fusion comprising amino acid sequences from the N- to the C-terminus, signal sequence B-shield B-linker B-protease sequence B-linker C-IgG light chain.

3. The bispecific antibody of claim 1, comprising a binding arm targeting the HER2 associated pathway, including a human IgG heavy chain fusion comprising amino acid sequences from the N- to the C-terminus, signal sequence A-shield A-linker A-protease sequence A-linker B-IgG heavy chain-linker C-homing domain E; and a human IgG light chain fusion comprising amino acid sequences from the N- to the C-terminus, signal sequence B-shield B-linker B-protease sequence B-linker C-IgG light chain.

4. The bispecific antibody of claim 1, comprising a binding arm targeting the VEGF associated pathway, including a human IgG heavy chain fusion comprising amino acid sequences from the N- to the C-terminus, signal sequence A-shield A-linker A-protease sequence A-linker B-IgG heavy chain; and a human IgG light chain fusion comprising amino acid sequences from the N- to the C-terminus, signal sequence B-shield B-linker B-protease sequence B-linker C-IgG light chain.

5. The bispecific antibody of claim 1, comprising a binding arm targeting the VEGF associated pathway, including a human IgG heavy chain fusion comprising amino acid sequences from the N- to the C-terminus, signal sequence A-shield A-linker A-protease sequence A-linker B-IgG heavy chain-linker C-homing domain D; and human IgG light chain fusion comprising amino acid sequences from the N- to the C-terminus, signal sequence B-shield B-linker B-protease sequence B-linker C-IgG light chain.

6. The bispecific antibody of claim 1, wherein the amino acid sequences of the IgG light chain and heavy chain recognize an HER2 antigenic epitope or antigen and comprise sequences chosen from SEQ ID NO: 23-34.

7. The bispecific antibody of claim 1, wherein the amino acid sequences of the IgG light chain and heavy chain recognize a VEGF antigenic epitope or antigen and comprise sequences chosen from SEQ ID NO: 35-44.

8. The bispecific antibody of claim 1, wherein the amino acid sequence of the shielding domains each independently comprises a sequence chosen from SEQ ID NO: 1-14.

9. The bispecific antibody of claim 1, wherein the protease sequence A and protease sequence B each independently comprise a sequence chosen from SEQ ID NO: 15-22.

10. The bispecific antibody of claim 1, wherein the amino acid sequence of the homing domains each independently comprises a sequence chosen from SEQ ID NO: 45-52 and 82.

11. The bispecific antibody of claim 1, wherein the IgG heavy and light chain of the HER2 binding arm comprise sequences chose from SEQ ID NO: 53 to 65, and SEQ ID NO: 101 to 145.

12. The bispecific antibody of claim 1, wherein the IgG heavy and light chain of the VEGF binding arm comprise sequences chosen from SEQ ID NO: 66 to 81, and SEQ ID NO: 201 to 248.

13. The bispecific antibody of claim 1, wherein the IgG heavy chain and light chain of the HER2 binding arm comprise combinations of the heavy chain and light chain with IgG Fc listed in Table 2 and Table 5.

14. The bispecific antibody of claim 1, wherein the IgG heavy chain and light chain of the VEGF binding arm comprise combinations of the heavy chain and light chain with IgG Fc listed in Table 3 and Table 6.

15. The bispecific antibody of claim 1, wherein the IgG heavy chains and light chains of the HER2 binding arm and VEGF binding arm comprise combinations of heavy chains and light chains with IgG Fc listed in Table 4, Table 7 and Table 8.

16. A nucleic acid sequence encoding the bispecific antibody of claim 1.

17. A recombinant expression vector comprising the nucleic acid sequence of claim 16.

18. A recombinant expression transformant comprising the recombinant expression vector of claim 17.

19. A method for preparing a bispecific antibody of claim 1, comprising: culturing a recombinant expression transformant comprising a nucleic acid sequence encoding the bispecific antibody, and obtaining the bispecific antibody from the culture, optionally using controlled Fab arm exchange of culture supernatants.

20. A pharmaceutical composition comprising the bispecific antibody of claim 1 and a pharmaceutical carrier.

21. A method for treating or preventing a cancer in a subject, comprising administering to the subject a pharmaceutically effective amount of the bispecific antibody of claim 1.

22. The method for treating or preventing a cancer according to claim 21, wherein the cancer is lung cancer, breast cancer, or gastric cancer.

23. The method for treating or preventing a cancer according to claim 21, where the subject suffers from relapsed HER2 positive cancer.

24. The method for treating or preventing a cancer according to claim 21, wherein the treatment prevents or reduces metastasis in the subject.


SEQUENCE LISTING (262 sequences)

SEQ ID NO: 1 - protein, 95 aa, Artificial Sequence
  Description of Artificial Sequence: Synthetic polypeptide
EIDQCIVDDITYNVQDTFHKKHEEGHMLNCTCFGQGRGRWKCSPVDQCQDSETGTFYQIGDSWEKYVHGVRYQCYCYGRGIGEWHCQPLQTYPSS

SEQ ID NO: 2 - protein, 95 aa, Artificial Sequence
  Description of Artificial Sequence: Synthetic polypeptide
EIDQCIVDDITYNVQDTFHKKHEEGHMLNCTCFGQGRGRWKCEPVDQCQDSETGTFYQIGDSWEKYVHGVRYQCYCYGRGIGEWHCQPLQTYPSS

SEQ ID NO: 3 - protein, 95 aa, Artificial Sequence
  Description of Artificial Sequence: Synthetic polypeptide
EIDQCIVDDITYNVQDTFHKKHEEGHMLQCTCFGQGRGRWKCDPVDQCQDSETGTFYQIGDSWEKYVHGVRYQCYCYGRGIGEWHCQPLQTYPSS

SEQ ID NO: 4 - protein, 25 aa, Artificial Sequence
  Description of Artificial Sequence: Synthetic peptide
EVGQRGVVGLPGQRGERGFPGLPGY

SEQ ID NO: 5 - protein, 15 aa, Homo sapiens
EPKSCDKTHTCPPCP

SEQ ID NO: 6 - protein, 21 aa, Homo sapiens
GIVEECCFRSCDLALLETYCA

SEQ ID NO: 7 - protein, 28 aa, Homo sapiens
AYRPSETLCGGELVDTLQFVCGDRGFYF

SEQ ID NO: 8 - protein, 21 aa, Artificial Sequence
  Description of Artificial Sequence: Synthetic peptide
GILEECCFRSCDLALLETYCA

SEQ ID NO: 9 - protein, 28 aa, Artificial Sequence
  Description of Artificial Sequence: Synthetic peptide
AYRPSETLCGGELVDTLQFVCGDRGFAF

SEQ ID NO: 10 - protein, 19 aa, Unknown
  Description of Unknown: VEGF epitope A sequence
CAQTMRIKPHQGQHIGESC

SEQ ID NO: 11 - protein, 10 aa, Homo sapiens
CNRDPPFDKC

SEQ ID NO: 12 - protein, 23 aa, Artificial Sequence
  Description of Artificial Sequence: Synthetic peptide
GQRGVVGLPGQRGERGFPGLPGY

SEQ ID NO: 13 - protein, 15 aa, Artificial Sequence
  Description of Artificial Sequence: Synthetic peptide
GQRGVVGLPGQRGER

SEQ ID NO: 14 - protein, 93 aa, Artificial Sequence
  Description of Artificial Sequence: Synthetic polypeptide
DQCIVDDITYNVQDTFHKKHEEGHMLNCTCFGQGRGRWKCDPVDQCQDSETGTFYQIGDSWEKYVHGVRYQCYCYGRGIGEWHCQPLQTYPSS

SEQ ID NO: 15 - protein, 6 aa, Artificial Sequence
  Description of Artificial Sequence: Synthetic peptide
GPLGVR

SEQ ID NO: 16 - protein, 6 aa, Artificial Sequence
  Description of Artificial Sequence: Synthetic peptide
PLGLAR

SEQ ID NO: 17 - protein, 6 aa, Artificial Sequence
  Description of Artificial Sequence: Synthetic peptide
PLGLAG

SEQ ID NO: 18 - protein, 8 aa, Artificial Sequence
  Description of Artificial Sequence: Synthetic peptide
IPVSLRSG

SEQ ID NO: 19 - protein, 8 aa, Artificial Sequence
  Description of Artificial Sequence: Synthetic peptide
GPLGMLSQ

SEQ ID NO: 20 - protein, 10 aa, Artificial Sequence
  Description of Artificial Sequence: Synthetic peptide
RPKPVEVWRK

SEQ ID NO: 21 - protein, 8 aa, Artificial Sequence
  Description of Artificial Sequence: Synthetic peptide
LSGRSDNH

SEQ ID NO: 22 - protein, 8 aa, Artificial Sequence
  Description of Artificial Sequence: Synthetic peptide
TGRGPSWV

SEQ ID NO: 23 - protein, 119 aa, Artificial Sequence
  Description of Artificial Sequence: Synthetic polypeptide
EVQLVESGGGLVQPGGSLRLSCAASGFTFTDYTMDWVRYAPGKGLEWVADVNPNSGGSIYNQRFKGRFTLSVDRSKNTLYLQMNSLRAEDTAVYYCARNLGPSFYFDYWGQGTLVTVSS

SEQ ID NO: 24 - protein, 107 aa, Artificial Sequence
  Description of Artificial Sequence: Synthetic polypeptide
DIQMTQSPSSLSASVGDRVTITCKASQDVSIGVAWYQRKPGKAPKLLIYSASYRYTGVPSRFSGSGSGTDFTLTISSLQPEDFATYYCQQYYIYPYTFGQGTKVEIK

SEQ ID NO: 25 - protein, 120 aa, Artificial Sequence [sequence omitted]

SEQ ID NO: 26 - protein, 107 aa, Artificial Sequence [sequence omitted]

SEQ ID NO: 27 - protein, 120 aa, Artificial Sequence
  Description of Artificial Sequence: Synthetic polypeptide
QVQLVQSGAEVKKPGSSVKVSCKASGFNIKDTYIHWVRQAPGQGLEWMGRIYPTNGYTRYAQKFQGRATITADESTSTAYMEVSSLRSEDTAVYYCSRWGGDGFYAMDYWGQGTLVTVSS

SEQ ID NO: 28 - protein, 120 aa, Artificial Sequence
  Description of Artificial Sequence: Synthetic polypeptide
EVQLVESGGGLVQPGPSLRLSCTASGFNIKDTYIHWVRQAPGKGLEWVGRIYPTNGYTRYAASFKGRATISADDSKSIAYLQVNSLKTEDTAVYYCTRWGGDGFYAMDYWGQGTLVTVSS

SEQ ID NO: 29 - protein, 120 aa, Artificial Sequence
  Description of Artificial Sequence: Synthetic polypeptide
EVQLVQSGAEVKKPGESLKISCKGSGFNIKDTYIHWVRQMPGKGLEWMGRIYPTNGYTRYSPSFQGQATISADKSISTAYLQVSSLKASDTAMYYCSRWGGDGFYAMDYWGQGTLVTVSS

SEQ ID NO: 30 - protein, 120 aa, Artificial Sequence
  Description of Artificial Sequence: Synthetic polypeptide
QVQLVQSGSELKKPGASVKVSCKASGFNIKDTYIHWVRQAPGQGLEWMGRIYPTNGYTRYAQGFTGRFVFSLDTSVSTAYLQICSLKAEDTAVYYCSRWGGDGFYAMDYWGQGTLVTVSS

SEQ ID NO: 31 - protein, 120 aa, Artificial Sequence
  Description of Artificial Sequence: Synthetic polypeptide
QVQLVQSGSELKKPGASVKVSCKASGFNIKDTYIHWVRQAPGQGLEWIGRIYPTNGYTRYAQGFTGRAVISADTSVSTAYLQVCSLKAEDTAVYYCSRWGGDGFYAMDYWGQGTLVTVSS

SEQ ID NO: 32 - protein, 107 aa, Artificial Sequence
  Description of Artificial Sequence: Synthetic polypeptide
DIVMTQTPLSLPVTPGEPASISCRASQDVNTAVAWYLQKPGQSPQLLIYSASFRYTGVPDRFSGSGSGTDFTLKISRVEAEDVGVYYCQQHYTTPPTFGQGTKVEIK

SEQ ID NO: 33 - protein, 107 aa, Artificial Sequence
  Description of Artificial Sequence: Synthetic polypeptide
DIVMTQSPDSLAVSLGERVTINCKASQDVNTAVAWYQQKPGQSPKLLIYSASFRYSGVPDRFSGSGSGTDFTFTISSVQAEDLAVYYCQQHYTTPPTFGQGTKVEIK

SEQ ID NO: 34 - protein, 107 aa, Artificial Sequence
  Description of Artificial Sequence: Synthetic polypeptide
DIQMTQSPSSLSASVGDRVTITCRASQRVNTAVAWYQQKPGKAPKLLIYSASFLYSGVPSRFSGSGSGTDFTLTISSLQPEDFATYYCQQHYTTPPTFGQGTKVEIK

SEQ ID NO: 35 - protein, 252 aa, Artificial Sequence
  Description of Artificial Sequence: Synthetic polypeptide
MEIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLGGGGGSGGGGSGGGGSGGGGSEVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSS

SEQ ID NO: 36 - protein, 123 aa, Artificial Sequence [sequence omitted]

SEQ ID NO: 37 - protein, 107 aa, Artificial Sequence [sequence omitted]

SEQ ID NO: 38 - protein, 123 aa, Artificial Sequence
  Description of Artificial Sequence: Synthetic polypeptide
QVQLVQSGSELKKPGASVKVSCKASGYTFTNYGMNWVRQAPGQGLEWMGWINTYTGEPTYAQGFTGRFVFSLDTSVSTAYLQICSLKAEDTAVYFCAKYPHYYGSSHWYFDVWGQGTLVTVSS

SEQ ID NO: 39 - protein, 123 aa, Artificial Sequence
  Description of Artificial Sequence: Synthetic polypeptide
QVQLVQSGAEVKKPGSSVKVSCKASGYTFTNYGMNWVRQAPGQGLEWMGWINTYTGEPTYAQKFQGRFTFTADESTSTAYMELSSLRSEDTAVYYCAKYPHYYGSSHWYFDVWGQGTLVTVSS

SEQ ID NO: 40 - protein, 123 aa, Artificial Sequence
  Description of Artificial Sequence: Synthetic polypeptide
QVQLVQSGAEVKKPGSSVKVSCKASGYTFTNYGMNWVRQAPGQGLEWMGWINTYTGEPTYAQKFQGRVTITADESTSTAYMELSSLRSEDTAVYYCAKYPHYYGSSHWYFDVWGQGTLVTVSS

SEQ ID NO: 41 - protein, 123 aa, Artificial Sequence
  Description of Artificial Sequence: Synthetic polypeptide
EVQLVQSGAEVKKPGESVKISCKGSGYTFTNYGMNWVRQMPGKGLEWMGWINTYTGEPTYSPSFQGQFTFSADKSISTAYLQISSLKASDTAMYYCAKYPHYYGSSHWYFDVWGQGTLVTVSS

SEQ ID NO: 42 - protein, 123 aa, Artificial Sequence
  Description of Artificial Sequence: Synthetic polypeptide
QVQLVQSGAEVKKPGSSVKVSCKASGYDFTHYGMNWVRQAPGQGLEWMGWINTYTGEPTYAQKFQGRFTFTADESTSTAYMELSSLRSEDTAVYYCAKYPYYYGTSHWYFDVWGQGTLVTVSS

SEQ ID NO: 43 - protein, 107 aa, Artificial Sequence
  Description of Artificial Sequence: Synthetic polypeptide
DIQMTQSPSSLSASVGDRVTITCRASQDISNYLNWYQQKPGKAPKVLIYFTSSLHSGVPSRFSGSGSGTDFTLTISSLQPEDFATYYCQQYSTVPWTFGQGTKLEIK

SEQ ID NO: 44 - protein, 107 aa, Artificial Sequence
  Description of Artificial Sequence: Synthetic polypeptide
DIVMTQTPLSLPVTPGEPASISCRASQDISNYLNWYLQKPGQSPQVLIYFTSSLHSGVPDRFSGSGSGTDFTLKISRLEAEDVGVYYCQQYSTVPWTFGQGTKLEIK

SEQ ID NO: 45 - protein, 236 aa, Artificial Sequence
  Description of Artificial Sequence: Synthetic polypeptide
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 46 - protein, 239 aa, Artificial Sequence
  Description of Artificial Sequence: Synthetic polypeptide
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGGSGGGGSGGGGSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 47 - protein, 252 aa, Artificial Sequence
  Description of Artificial Sequence: Synthetic polypeptide
EVQLVESGGGLVQPGPSLRLSCTASRYTFTEYTIHWVRQAPGKGLEWVGGINPNNGIPNYAASVKGRFTISVDDSKSIAYLQMNSLKTEDTAVYFCARRRIAYGYDEGHAMDYWGQGTTVTVSSGGSGGGGSGGGGSGGDIVMTQSPLSLPVTPGEPASMSCKSSQSLLYSRNQKNYLAWFLQKPGQSPQLLIFWASTRESGVPDRFSGSGSGTDFTLKISRVEAEDVGVYDCQQYFSYPLTFGQGTKLEIK

SEQ ID NO: 48 - protein, 252 aa, Artificial Sequence
  Description of Artificial Sequence: Synthetic polypeptide
EVQLVESGGGLVQPGPSLRLSCTASRYTFTEYTIHWVRQAPGKGLEWVGGINPNNGIPNYAASVKGRFTISVDDSKSIAYLQMNSLKTEDTAVYFCARRRIAYGYDEGHAMDYWGQGTTVTVSSGGSGGGGSGGGGSGGAIRMTQSPSSFSVSTGDRVTMTCRSSQSLLYSRNQKNYLAWFQQKPGKAPKLLIFWASTRESGVPSRFSGSGSGTDFTLTISCVQSEDFATYDCQQYFSYPLTFGQGTKLEIK

SEQ ID NO: 49 - protein, 252 aa, Artificial Sequence
  Description of Artificial Sequence: Synthetic polypeptide
QMQLVQSGAEVKKTGSSVKVSCKTSRYTFTEYTIHWVRQAPGQALEWIGGINPNNGIPNYAQKFQDRATLTVDRSMSTAYMEVSSLRSEDTAMYFCARRRIAYGYDEGHAMDYWGQGTTVTVSSGGSGGGGSGGGGSGGDIVMTQSPLSLPVTPGEPASMSCKSSQSLLYSRNQKNYLAWFLQKPGQSPQLLIFWASTRESGVPDRFSGSGSGTDFTLKISRVEAEDVGVYDCQQYFSYPLTFGQGTKLEIK

SEQ ID NO: 50 - protein, 252 aa, Artificial Sequence
  Description of Artificial Sequence: Synthetic polypeptide
QMQLVQSGAEVKKTGSSVKVSCKTSRYTFTEYTIHWVRQAPGQALEWIGGINPNNGIPNYAQKFQDRATLTVDRSMSTAYMEVSSLRSEDTAMYFCARRRIAYGYDEGHAMDYWGQGTTVTVSSGGSGGGGSGGGGSGGAIRMTQSPSSFSVSTGDRVTMTCRSSQSLLYSRNQKNYLAWFQQKPGKAPKLLIFWASTRESGVPSRFSGSGSGTDFTLTISCVQSEDFATYDCQQYFSYPLTFGQGTKLEIK

SEQ ID NO: 51 - protein, 252 aa, Artificial Sequence
  Description of Artificial Sequence: Synthetic polypeptide
QVQLQESGPGLVKPSDTLSLTCAVSRYTFTEYTIHWVRQPPGKGLEWIGGINPNNGIPNYNPSLKSRATMSVDTSKNQASLKLSSVTAVDTAVYFCARRRIAYGYDEGHAMDYWGQGTTVTVSSGGSGGGGSGGGGSGGDIVMTQSPLSLPVTPGEPASMSCKSSQSLLYSRNQKNYLAWFLQKPGQSPQLLIFWASTRESGVPDRFSGSGSGTDFTLKISRVEAEDVGVYDCQQYFSYPLTFGQGTKLEIK

SEQ ID NO: 52 - protein, 252 aa, Artificial Sequence
  Description of Artificial Sequence: Synthetic polypeptide
QVQLQESGPGLVKPSDTLSLTCAVSRYTFTEYTIHWVRQPPGKGLEWIGGINPNNGIPNYNPSLKSRATMSVDTSKNQASLKLSSVTAVDTAVYFCARRRIAYGYDEGHAMDYWGQGTTVTVSSGGSGGGGSGGGGSGGAIRMTQSPSSFSVSTGDRVTMTCRSSQSLLYSRNQKNYLAWFQQKPGKAPKLLIFWASTRESGVPSRFSGSGSGTDFTLTISCVQSEDFATYDCQQYFSYPLTFGQGTKLEIK

SEQ ID NO: 53 - protein, 449 aa, Artificial Sequence
  Description of Artificial Sequence: Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPLLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 54 - protein, 214 aa, Artificial Sequence
  Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 450 aa, Artificial Sequence
  Description of Artificial Sequence: Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 56 - protein, 449 aa, Artificial Sequence
  Description of Artificial Sequence: Synthetic polypeptide
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 57 - protein, 214 aa, Artificial Sequence
  Description of Artificial Sequence: Synthetic polypeptide
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 450 aa, Artificial Sequence
  Description of Artificial Sequence: Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 59 - protein, 450 aa, Artificial Sequence
  Description of Artificial Sequence: Synthetic polypeptide
QVQLVQSGAEVKKPGSSVKVSCKASGFNIKDTYIHWVRQAPGQGLEWMGRIYPTNGYTRYAQKFQGRATITADESTSTAYMEVSSLRSEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 60 - protein, 450 aa, Artificial Sequence
  Description of Artificial Sequence: Synthetic polypeptide
QVQLVQSGAEVKKPGSSVKVSCKASGFNIKDTYIHWVRQAPGQGLEWMGRIYPTNGYTRYAQKFQGRATITADESTSTAYMEVSSLRSEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 61 - protein, 214 aa, Artificial Sequence
  Description of Artificial Sequence: Synthetic polypeptide
DIVMTQTPLSLPVTPGEPASISCRASQDVNTAVAWYLQKPGQSPQLLIYSASFRYTGVPDRFSGSGSGTDFTLKISRVEAEDVGVYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 450 aa, Artificial Sequence
  Description of Artificial Sequence: Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 450 aa, Artificial Sequence
  Description of Artificial Sequence: Synthetic polypeptide
QVQLVQSGAEVKKPGSSVKVSCKASGFNIKDTYIHWVRQAPGQGLEWMGRIYPTNGYTRYAQKFQGRATITADESTSTAYMEVSSLRSEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 64 - protein, 450 aa, Artificial Sequence
  Description of Artificial Sequence: Synthetic polypeptide
QVQLVQSGAEVKKPGSSVKVSCKASGFNIKDTYIHWVRQAPGQGLEWMGRIYPTNGYTRYAQKFQGRATITADESTSTAYMEVSSLRSEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 65 - protein, 450 aa, Artificial Sequence
  Description of Artificial Sequence: Synthetic polypeptide
QVQLVQSGAEVKKPGSSVKVSCKASGFNIKDTYIHWVRQAPGQGLEWMGRIYPTNGYTRYAQKFQGRATITADESTSTAYMEVSSLRSEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSRDELTKNQVSLWCLVKGFYPSDIAVEWESNGQPENNYKTTPLVLDSDGSFFLYSKLTVDKSRWQQGNVFSCSVMHEALHNHYTQKSLSLSPGK

SEQ ID NO: 66 - protein, 452 aa, Artificial Sequence
  Description of Artificial Sequence: Synthetic polypeptide
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPLLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 67 - protein, 214 aa, Artificial Sequence
  Description of Artificial Sequence: Synthetic polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 453 aa, Artificial Sequence
  Description of Artificial Sequence: Synthetic polypeptide
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 69 - protein, 453 aa, Artificial Sequence
  Description of Artificial Sequence: Synthetic polypeptide
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 70 - protein, 453 aa, Artificial Sequence
  Description of Artificial Sequence: Synthetic polypeptide
QVQLVQSGSELKKPGASVKVSCKASGYTFTNYGMNWVRQAPGQGLEWMGWINTYTGEPTYAQGFTGRFVFSLDTSVSTAYLQICSLKAEDTAVYFCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 71 - protein, 453 aa, Artificial Sequence
  Description of Artificial Sequence: Synthetic polypeptide
QVQLVQSGAEVKKPGSSVKVSCKASGYTFTNYGMNWVRQAPGQGLEWMGWINTYTGEPTYAQKFQGRFTFTADESTSTAYMELSSLRSEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 72 - protein, 453 aa, Artificial Sequence
  Description of Artificial Sequence: Synthetic polypeptide
QVQLVQSGAEVKKPGSSVKVSCKASGYTFTNYGMNWVRQAPGQGLEWMGWINTYTGEPTYAQKFQGRVTITADESTSTAYMELSSLRSEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 73 - protein, 453 aa, Artificial Sequence
  Description of Artificial Sequence: Synthetic polypeptide
EVQLVQSGAEVKKPGESVKISCKGSGYTFTNYGMNWVRQMPGKGLEWMGWINTYTGEPTYSPSFQGQFTFSADKSISTAYLQISSLKASDTAMYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 74 - protein, 453 aa, Artificial Sequence
  Description of Artificial Sequence: Synthetic polypeptide
QVQLVQSGAEVKKPGSSVKVSCKASGYDFTHYGMNWVRQAPGQGLEWMGWINTYTGEPTYAQKFQGRFTFTADESTSTAYMELSSLRSEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 75 - protein, 214 aa, Artificial Sequence
  Description of Artificial Sequence: Synthetic polypeptide
DIQMTQSPSSLSASVGDRVTITCRASQDISNYLNWYQQKPGKAPKVLIYFTSSLHSGVPSRFSGSGSGTDFTLTISSLQPEDFATYYCQQYSTVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 214 aa, Artificial Sequence
  Description of Artificial Sequence: Synthetic polypeptide
DIVMTQTPLSLPVTPGEPASISCRASQDISNYLNWYLQKPGQSPQVLIYFTSSLHSGVPDRFSGSGSGTDFTLKISRLEAEDVGVYYCQQYSTVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 453 aa, Artificial Sequence
  Description of Artificial Sequence: Synthetic polypeptide
QVQLVQSGAEVKKPGSSVKVSCKASGYTFTNYGMNWVRQAPGQGLEWMGWINTYTGEPTYAQKFQGRVTITADESTSTAYMELSSLRSEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 78 - protein, 453 aa, Artificial Sequence
  Description of Artificial Sequence: Synthetic polypeptide
QVQLVQSGAEVKKPGSSVKVSCKASGYTFTNYGMNWVRQAPGQGLEWMGWINTYTGEPTYAQKFQGRFTFTADESTSTAYMELSSLRSEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 79 - protein, 453 aa, Artificial Sequence
  Description of Artificial Sequence: Synthetic polypeptide
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 453 aa, Artificial Sequence
  Description of Artificial Sequence: Synthetic polypeptide
QVQLVQSGAEVKKPGSSVKVSCKASGYTFTNYGMNWVRQAPGQGLEWMGWINTYTGEPTYAQKFQGRVTITADESTSTAYMELSSLRSEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 81 - protein, 453 aa, Artificial Sequence
  Description of Artificial Sequence: Synthetic polypeptide
QVQLVQSGAEVKKPGSSVKVSCKASGYTFTNYGMNWVRQAPGQGLEWMGWINTYTGEPTYAQKFQGRVTITADESTSTAYMELSSLRSEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSRDELTKNQVSLSCAVKGFYPSDIAVEWESNGQPENNYKTTPLVLDSDGSFFLVSKLTVDKSRWQQGNVFSCSVMHEALHNHYTQKSLSLSPGK

SEQ ID NO: 82 - protein, 240 aa, Artificial Sequence
  Description of Artificial Sequence: Synthetic polypeptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 83 - protein, 474 aa, Artificial Sequence
  Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 84 - protein, 474 aa, Artificial Sequence
  Description of Artificial Sequence: Synthetic polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 85-100 - skipped (16 entries)

SEQ ID NO: 101 - protein, 480 aa, Artificial Sequence
  Description of Artificial Sequence: Synthetic polypeptide
EPKSCDKTHTCPPCPGGGGSGPLGVRGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPLLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 102 - protein, 245 aa, Artificial Sequence
  Description of Artificial Sequence: Synthetic polypeptide
EPKSCDKTHTCPPCPGGGGSGPLGVRGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 486 aa, Artificial Sequence
  Description of Artificial Sequence: Synthetic polypeptide
GIVEECCFRSCDLALLETYCAGGGGSPLGLAGGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPLLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 104 - protein, 258 aa, Artificial Sequence
  Description of Artificial Sequence: Synthetic polypeptide
AYRPSETLCGGELVDTLQFVCGDRGFYFGGGGSPLGLAGGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 488 aa, Artificial Sequence
  Description of Artificial Sequence: Synthetic polypeptide
GIVEECCFRSCDLALLETYCAGGGGSLSGRSDNHGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPLLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 106 - protein, 260 aa, Artificial Sequence
  Description of Artificial Sequence: Synthetic polypeptide
AYRPSETLCGGELVDTLQFVCGDRGFYFGGGGSLSGRSDNHGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 488 aa, Artificial Sequence
  Description of Artificial Sequence: Synthetic polypeptide
GQRGVVGLPGQRGERGFPGLPGYGGGGSPLGLAGGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPLLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 108 - protein, 323 aa, Artificial Sequence
  Description of Artificial Sequence: Synthetic polypeptide
DQCIVDDITYNVQDTFHKKHEEGHMLNCTCFGQGRGRWKCDPVDQCQDSETGTFYQIGDSWEKYVHGVRYQCYCYGRGIGEWHCQPLQTYPSSGGGGSPLGLAGGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 489 aa, Artificial Sequence
  Description of Artificial Sequence: Synthetic polypeptide
GQRGVVGLPGQRGERGFPGLPGYGGGGSPLGLAGGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 110 - protein, 487 aa, Artificial Sequence
  Description of Artificial Sequence: Synthetic polypeptide
GILEECCFRSCDLALLETYCAGGGGSPLGLAGGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 111 - protein, 258 aa, Artificial Sequence
  Description of Artificial Sequence: Synthetic polypeptide
AYRPSETLCGGELVDTLQFVCGDRGFAFGGGGSPLGLAGGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 486 aa, Artificial Sequence
  Description of Artificial Sequence: Synthetic polypeptide
GILEECCFRSCDLALLETYCAGGGGSPLGLAGGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 113 - protein, 258 aa, Artificial Sequence
  Description of Artificial Sequence: Synthetic polypeptide
AYRPSETLCGGELVDTLQFVCGDRGFAFGGGGSPLGLAGGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 487 aa, Artificial Sequence
  Description of Artificial Sequence: Synthetic polypeptide
GILEECCFRSCDLALLETYCAGGGGSPLGLAGGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 115 - protein, 481 aa, Artificial Sequence
  Description of Artificial Sequence: Synthetic polypeptide
GQRGVVGLPGQRGERGGGGSPLGLAGGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 116 - protein, 487 aa, Artificial Sequence
  Description of Artificial Sequence: Synthetic polypeptide
GILEECCFRSCDLALLETYCAGGGGSPLGLAGGGGGSQVQLVQSGAEVKKPGSSVKVSCKASGFNIKDTYIHWVRQAPGQGLEWMGRIYPTNGYTRYAQKFQGRATITADESTSTAYMEVSSLRSEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 117 - protein, 487 aa, Artificial Sequence
  Description of Artificial Sequence: Synthetic polypeptide
GILEECCFRSCDLALLETYCAGGGGSPLGLAGGGGGSEVQLVESGGGLVQPGPSLRLSCTASGFNIKDTYIHWVRQAPGKGLEWVGRIYPTNGYTRYAASFKGRATISADDSKSIAYLQVNSLKTEDTAVYYCTRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 118 - protein, 487 aa, Artificial Sequence
  Description of Artificial Sequence: Synthetic polypeptide
GILEECCFRSCDLALLETYCAGGGGSPLGLAGGGGGSEVQLVQSGAEVKKPGESLKISCKGSGFNIKDTYIHWVRQMPGKGLEWMGRIYPTNGYTRYSPSFQGQATISADKSISTAYLQVSSLKASDTAMYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 119 - protein, 258 aa, Artificial Sequence
  Description of Artificial Sequence: Synthetic polypeptide
AYRPSETLCGGELVDTLQFVCGDRGFAFGGGGSPLGLAGGGGGSDIVMTQTPLSLPVTPGEPASISCRASQDVNTAVAWYLQKPGQSPQLLIYSASFRYTGVPDRFSGSGSGTDFTLKISRVEAEDVGVYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 258 aa, Artificial Sequence
  Description of Artificial Sequence: Synthetic polypeptide
AYRPSETLCGGELVDTLQFVCGDRGFAFGGGGSPLGLAGGGGGSDIVMTQSPDSLAVSLGERVTINCKASQDVNTAVAWYQQKPGQSPKLLIYSASFRYSGVPDRFSGSGSGTDFTFTISSVQAEDLAVYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 481 aa, Artificial Sequence
  Description of Artificial Sequence: Synthetic polypeptide
GQRGVVGLPGQRGERGGGGSPLGLAGGGGGSQVQLVQSGAEVKKPGSSVKVSCKASGFNIKDTYIHWVRQAPGQGLEWMGRIYPTNGYTRYAQKFQGRATITADESTSTAYMEVSSLRSEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 122 - protein, 481 aa, Artificial Sequence
  Description of Artificial Sequence: Synthetic polypeptide
GQRGVVGLPGQRGERGGGGSPLGLAGGGGGSEVQLVESGGGLVQPGPSLRLSCTASGFNIKDTYIHWVRQAPGKGLEWVGRIYPTNGYTRYAASFKGRATISADDSKSIAYLQVNSLKTEDTAVYYCTRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 123 - protein, 481 aa, Artificial Sequence
  Description of Artificial Sequence: Synthetic polypeptide
GQRGVVGLPGQRGERGGGGSPLGLAGGGGGSEVQLVQSGAEVKKPGESLKISCKGSGFNIKDTYIHWVRQMPGKGLEWMGRIYPTNGYTRYSPSFQGQATISADKSISTAYLQVSSLKASDTAMYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 124 - protein, 323 aa, Artificial Sequence
  Description of Artificial Sequence: Synthetic polypeptide
DQCIVDDITYNVQDTFHKKHEEGHMLNCTCFGQGRGRWKCDPVDQCQDSETGTFYQIGDSWEKYVHGVRYQCYCYGRGIGEWHCQPLQTYPSSGGGGSPLGLAGGGGGSDIVMTQTPLSLPVTPGEPASISCRASQDVNTAVAWYLQKPGQSPQLLIYSASFRYTGVPDRFSGSGSGTDFTLKISRVEAEDVGVYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 323 aa, Artificial Sequence
  Description of Artificial Sequence: Synthetic polypeptide
DQCIVDDITYNVQDTFHKKHEEGHMLNCTCFGQGRGRWKCDPVDQCQDSETGTFYQIGDSWEKYVHGVRYQCYCYGRGIGEWHCQPLQTYPSSGGGGSPLGLAGGGGGSDIVMTQSPDSLAVSLGERVTINCKASQDVNTAVAWYQQKPGQSPKLLIYSASFRYSGVPDRFSGSGSGTDFTFTISSVQAEDLAVYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - protein, 487 aa, Artificial Sequence
  Description of Artificial Sequence: Synthetic polypeptide
GILEECCFRSCDLALLETYCAGGGGSPLGLAGGGGGSQVQLVQSGAEVKKPGSSVKVSCKASGFNIKDTYIHWVRQAPGQGLEWMGRIYPTNGYTRYAQKFQGRATITADESTSTAYMEVSSLRSEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 127 - protein, 491 aa, Artificial Sequence
  Description of Artificial Sequence: Synthetic polypeptide
GILEECCFRSCDLALLETYCAGGGGSRPKPVEVWRKGGGGSQVQLVQSGAEVKKPGSSVKVSCKASGFNIKDTYIHWVRQAPGQGLEWMGRIYPTNGYTRYAQKFQGRATITADESTSTAYMEVSSLRSEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 128 - protein, 262 aa, Artificial Sequence
  Description of Artificial Sequence: Synthetic polypeptide
AYRPSETLCGGELVDTLQFVCGDRGFAFGGGGSRPKPVEVWRKGGGGSDIVMTQTPLSLPVTPGEPASISCRASQDVNTAVAWYLQKPGQSPQLLIYSASFRYTGVPDRFSGSGSGTDFTLKISRVEAEDVGVYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 489 aa, Artificial Sequence
  Description of Artificial Sequence: Synthetic polypeptide
GILEECCFRSCDLALLETYCAGGGGSLSGRSDNHGGGGSQVQLVQSGAEVKKPGSSVKVSCKASGFNIKDTYIHWVRQAPGQGLEWMGRIYPTNGYTRYAQKFQGRATITADESTSTAYMEVSSLRSEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 130 - protein, 489 aa, Artificial Sequence
  Description of Artificial Sequence: Synthetic polypeptide
GILEECCFRSCDLALLETYCAGGGGSLSGRSDNHGGGGSQVQLVQSGAEVKKPGSSVKVSCKASGFNIKDTYIHWVRQAPGQGLEWMGRIYPTNGYTRYAQKFQGRATITADESTSTAYMEVSSLRSEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 131 - protein, 260 aa, Artificial Sequence
  Description of Artificial Sequence: Synthetic polypeptide
AYRPSETLCGGELVDTLQFVCGDRGFAFGGGGSLSGRSDNHGGGGSDIVMTQTPLSLPVTPGEPASISCRASQDVNTAVAWYLQKPGQSPQLLIYSASFRYTGVPDRFSGSGSGTDFTLKISRVEAEDVGVYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 260 aa, Artificial Sequence
  Description of Artificial Sequence: Synthetic polypeptide
AYRPSETLCGGELVDTLQFVCGDRGFAFGGGGSTGRGPSWVGGGGSDIVMTQTPLSLPVTPGEPASISCRASQDVNTAVAWYLQKPGQSPQLLIYSASFRYTGVPDRFSGSGSGTDFTLKISRVEAEDVGVYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 487 aa, Artificial Sequence
  Description of Artificial Sequence: Synthetic polypeptide
GILEECCFRSCDLALLETYCAGGGGSPLGLAGGGGGSQVQLVQSGAEVKKPGSSVKVSCKASGFNIKDTYIHWVRQAPGQGLEWMGRIYPTNGYTRYAQKFQGRATITADESTSTAYMEVSSLRSEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 134 - protein, 487 aa, Artificial Sequence
  Description of Artificial Sequence: Synthetic polypeptide
GILEECCFRSCDLALLETYCAGGGGSPLGLAGGGGGSQVQLVQSGAEVKKPGSSVKVSCKASGFNIKDTYIHWVRQAPGQGLEWMGRIYPTNGYTRYAQKFQGRATITADESTSTAYMEVSSLRSEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSRDELTKNQVSLWCLVKGFYPSDIAVEWESNGQPENNYKTTPLVLDSDGSFFLYSKLTVDKSRWQQGNVFSCSVMHEALHNHYTQKSLSLSPGK

SEQ ID NO: 135 - protein, 488 aa, Artificial Sequence
  Description of Artificial Sequence: Synthetic polypeptide
GQRGVVGLPGQRGERGFPGLPGYGGGGSPLGLAGGGGGSEVQLVESGGGLVQPGGSLRLSCAASGFTFTDYTMDWVRYAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 136 - protein, 323 aa, Artificial Sequence
  Description of Artificial Sequence: Synthetic polypeptide
DQCIVDDITYNVQDTFHKKHEEGHMLNCTCFGQGRGRWKCDPVDQCQDSETGTFYQIGDSWEKYVHGVRYQCYCYGRGIGEWHCQPLQTYPSSGGGGSPLGLAGGGGGSDIQMTQSPSSLSASVGDRVTITCKASQDVSIGVAWYQRKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 491 aa, Artificial Sequence
  Description of Artificial Sequence: Synthetic polypeptide
EPKSCDKTHTCPPCPGGGGSGPLGVRGGGGSPLGLAGGGGGSEVQLVESGGGLVQPGGSLRLSCAASGFTFTDYTMDWVRYAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 138 - protein, 256 aa, Artificial Sequence
  Description of Artificial Sequence: Synthetic polypeptide
EPKSCDKTHTCPPCPGGGGSGPLGVRGGGGSPLGLAGGGGGSDIQMTQSPSSLSASVGDRVTITCKASQDVSIGVAWYQRKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - protein, 256 aa, Artificial Sequence
  Description of Artificial Sequence: Synthetic polypeptide
GPEADQQKACNHYKDPPFCTCGGGGSGGGGSGPLGVRGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 492 aa, Artificial Sequence
  Description of Artificial Sequence: Synthetic polypeptide
GPEADQQKACNHYKDPPFCTCGGGGSGGGGSGPLGVRGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 141 - protein, 505 aa, Artificial Sequence
  Description of Artificial Sequence: Synthetic polypeptide
GPEADQQKACNHYKDPPFCTCGGGGSGGGGSGPLGVRGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGGSGGGGSGGGGSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 142 - protein, 741 aa, Artificial Sequence
  Description of Artificial Sequence: Synthetic polypeptide
GPEADQQKACNHYKDPPFCTCGGGGSGGGGSGPLGVRGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGKGGGGSGGGGSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGGSGGGGSGGGGSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 143 - protein, 518 aa, Artificial Sequence
  Description of Artificial Sequence: Synthetic polypeptide
GPEADQQKACNHYKDPPFCTCGGGGSGGGGSGPLGVRGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLVESGGGLVQPGPSLRLSCTASRYTFTEYTIHWVRQAPGKGLEWVGGINPNNGIPNYAASVKGRFTISVDDSKSIAYLQMNSLKTEDTAVYFCARRRIAYGYDEGHAMDYWGQGTTVTVSSGGSGGGGSGGGGSGGAIRMTQSPSSFSVSTGDRVTMTCRSSQSLLYSRNQKNYLAWFQQKPGKAPKLLIFWASTRESGVPSRFSGSGSGTDFTLTISCVQSEDFATYDCQQYFSYPLTFGQGTKLEIK

SEQ ID NO: 144 - protein, 754 aa, Artificial Sequence
  Description of Artificial Sequence: Synthetic polypeptide
GPEADQQKACNHYKDPPFCTCGGGGSGGGGSGPLGVRGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGKGGGGSGGGGSEVQLVESGGGLVQPGPSLRLSCTASRYTFTEYTIHWVRQAPGKGLEWVGGINPNNGIPNYAASVKGRFTISVDDSKSIAYLQMNSLKTEDTAVYFCARRRIAYGYDEGHAMDYWGQGTTVTVSSGGSGGGGSGGGGSGGAIRMTQSPSSFSVSTGDRVTMTCRSSQSLLYSRNQKNYLAWFQQKPGKAPKLLIFWASTRESGVPSRFSGSGSGTDFTLTISCVQSEDFATYDCQQYFSYPLTFGQGTKLEIK

SEQ ID NO: 145 - protein, 518 aa, Artificial Sequence
  Description of Artificial Sequence: Synthetic polypeptide
AYRPSETLCGGELVDTLQFVCGDRGFAFGGGGSPLGLAGGGGGSDIVMTQTPLSLPVTPGEPASISCRASQDVNTAVAWYLQKPGQSPQLLIYSASFRYTGVPDRFSGSGSGTDFTLKISRVEAEDVGVYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 146-200 - skipped (55 entries)

SEQ ID NO: 201 - protein, 483 aa, Artificial Sequence
  Description of Artificial Sequence: Synthetic polypeptide
EPKSCDKTHTCPPCPGGGGSGPLGVRGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPLLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 202 - protein, 245 aa, Artificial Sequence
  Description of Artificial Sequence: Synthetic polypeptide
EPKSCDKTHTCPPCPGGGGSGPLGVRGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 203 - protein, 489 aa, Artificial Sequence
  Description of Artificial Sequence: Synthetic polypeptide
GIVEECCFRSCDLALLETYCAGGGGSPLGLAGGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPLLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 204 - protein, 258 aa, Artificial Sequence
  Description of Artificial Sequence: Synthetic polypeptide
AYRPSETLCGGELVDTLQFVCGDRGFYFGGGGSPLGLAGGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 205 - protein, 491 aa, Artificial Sequence
  Description of Artificial Sequence: Synthetic polypeptide
GIVEECCFRSCDLALLETYCAGGGGSLSGRSDNHGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPLLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 206 - protein, 260 aa, Artificial Sequence
  Description of Artificial Sequence: Synthetic polypeptide
AYRPSETLCGGELVDTLQFVCGDRGFYFGGGGSLSGRSDNHGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 207 - protein, 491 aa, Artificial Sequence
  Description of Artificial Sequence: Synthetic polypeptide
GQRGVVGLPGQRGERGFPGLPGYGGGGSPLGLAGGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPLLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 208 - protein, 323 aa, Artificial Sequence
  Description of Artificial Sequence: Synthetic polypeptide
DQCIVDDITYNVQDTFHKKHEEGHMLNCTCFGQGRGRWKCDPVDQCQDSETGTFYQIGDSWEKYVHGVRYQCYCYGRGIGEWHCQPLQTYPSSGGGGSPLGLAGGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 209 - protein, 494 aa, Artificial Sequence
  Description of Artificial Sequence: Synthetic polypeptide
GQRGVVGLPGQRGERGFPGLPGYGGGGSPLGLAGGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSGSFFLYSRLTVDKSRWQQGNVFSCSVLHEALHSHYTQKSLSLSPGK

SEQ ID NO: 210 - protein, 487 aa, Artificial Sequence
  Description of Artificial Sequence: Synthetic polypeptide
GILEECCFRSCDLALLETYCAGGGGSPLGLAGGGGGSEVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 211 - protein, 260 aa, Artificial Sequence
  Description of Artificial Sequence: Synthetic polypeptide
AYRPSETLCGGELVDTLQFVCGDRGFAFGGGGSPLGLAGGGGGSEIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 212 - protein, 486 aa, Artificial Sequence
  Description of Artificial Sequence: Synthetic polypeptide
GQRGVVGLPGQRGERGGGGSPLGLAGGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSGSFFLYSRLTVDKSRWQQGNVFSCSVLHEALHSHYTQKSLSLSPGK

SEQ ID NO: 213 - protein, 490 aa, Artificial Sequence
  Description of Artificial Sequence: Synthetic polypeptide
GILEECCFRSCDLALLETYCAGGGGSPLGLAGGGGGSQVQLVQSGSELKKPGASVKVSCKASGYTFTNYGMNWVRQAPGQGLEWMGWINTYTGEPTYAQGFTGRFVFSLDTSVSTAYLQICSLKAEDTAVYFCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 214 - protein, 490 aa, Artificial Sequence
  Description of Artificial Sequence: Synthetic polypeptide
GILEECCFRSCDLALLETYCAGGGGSPLGLAGGGGGSQVQLVQSGAEVKKPGSSVKVSCKASGYTFTNYGMNWVRQAPGQGLEWMGWINTYTGEPTYAQKFQGRFTFTADESTSTAYMELSSLRSEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 215 - protein, 258 aa, Artificial Sequence
  Description of Artificial Sequence: Synthetic polypeptide
AYRPSETLCGGELVDTLQFVCGDRGFAFGGGGSPLGLAGGGGGSDIQMTQSPSSLSASVGDRVTITCRASQDISNYLNWYQQKPGKAPKVLIYFTSSLHSGVPSRFSGSGSGTDFTLTISSLQPEDFATYYCQQYSTVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 216 - protein, 486 aa, Artificial Sequence
  Description of Artificial Sequence: Synthetic polypeptide
GQRGVVGLPGQRGERGGGGSPLGLAGGGGGSQVQLVQSGSELKKPGASVKVSCKASGYTFTNYGMNWVRQAPGQGLEWMGWINTYTGEPTYAQGFTGRFVFSLDTSVSTAYLQICSLKAEDTAVYFCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSGSFFLYSRLTVDKSRWQQGNVFSCSVLHEALHSHYTQKSLSLSPGK

SEQ ID NO: 217 - protein, 486 aa, Artificial Sequence
  Description of Artificial Sequence: Synthetic polypeptide
GQRGVVGLPGQRGERGGGGSPLGLAGGGGGSQVQLVQSGAEVKKPGSSVKVSCKASGYTFTNYGMNWVRQAPGQGLEWMGWINTYTGEPTYAQKFQGRFTFTADESTSTAYMELSSLRSEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSGSFFLYSRLTVDKSRWQQGNVFSCSVLHEALHSHYTQKSLSLSPGK

SEQ ID NO: 218 - protein, 323 aa, Artificial Sequence
  Description of Artificial Sequence: Synthetic polypeptide
DQCIVDDITYNVQDTFHKKHEEGHMLNCTCFGQGRGRWKCDPVDQCQDSETGTFYQIGDSWEKYVHGVRYQCYCYGRGIGEWHCQPLQTYPSSGGGGSPLGLAGGGGGSDIQMTQSPSSLSASVGDRVTITCRASQDISNYLNWYQQKPGKAPKVLIYFTSSLHSGVPSRFSGSGSGTDFTLTISSLQPEDFATYYCQQYSTVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 219 - protein, 490 aa, Artificial Sequence
  Description of Artificial Sequence: Synthetic polypeptide
GILEECCFRSCDLALLETYCAGGGGSPLGLAGGGGGSQVQLVQSGAEVKKPGSSVKVSCKASGYTFTNYGMNWVRQAPGQGLEWMGWINTYTGEPTYAQKFQGRVTITADESTSTAYMELSSLRSEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 220 - protein, 490 aa, Artificial Sequence
  Description of Artificial Sequence: Synthetic polypeptide
GILEECCFRSCDLALLETYCAGGGGSPLGLAGGGGGSEVQLVQSGAEVKKPGESVKISCKGSGYTFTNYGMNWVRQMPGKGLEWMGWINTYTGEPTYSPSFQGQFTFSADKSISTAYLQISSLKASDTAMYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 221 - protein, 490 aa, Artificial Sequence
  Description of Artificial Sequence: Synthetic polypeptide
GILEECCFRSCDLALLETYCAGGGGSPLGLAGGGGGSQVQLVQSGAEVKKPGSSVKVSCKASGYDFTHYGMNWVRQAPGQGLEWMGWINTYTGEPTYAQKFQGRFTFTADESTSTAYMELSSLRSEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 222 - protein, 258 aa, Artificial Sequence
  Description of Artificial Sequence: Synthetic polypeptide
AYRPSETLCGGELVDTLQFVCGDRGFAFGGGGSPLGLAGGGGGSDIVMTQTPLSLPVTPGEPASISCRASQDISNYLNWYLQKPGQSPQVLIYFTSSLHSGVPDRFSGSGSGTDFTLKISRLEAEDVGVYYCQQYSTVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 223 - protein, 494 aa, Artificial Sequence
  Description of Artificial Sequence: Synthetic polypeptide
GQRGVVGLPGQRGERGFPGLPGYGGGGSPLGLAGGGGGSQVQLVQSGSELKKPGASVKVSCKASGYTFTNYGMNWVRQAPGQGLEWMGWINTYTGEPTYAQGFTGRFVFSLDTSVSTAYLQICSLKAEDTAVYFCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSGSFFLYSRLTVDKSRWQQGNVFSCSVLHEALHSHYTQKSLSLSPGK

SEQ ID NO: 224 - protein, 494 aa, Artificial Sequence
  Description of Artificial Sequence: Synthetic polypeptide
GQRGVVGLPGQRGERGFPGLPGYGGGGSPLGLAGGGGGSQVQLVQSGAEVKKPGSSVKVSCKASGYTFTNYGMNWVRQAPGQGLEWMGWINTYTGEPTYAQKFQGRFTFTADESTSTAYMELSSLRSEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSGSFFLYSRLTVDKSRWQQGNVFSCSVLHEALHSHYTQKSLSLSPGK

SEQ ID NO: 225 - protein, 494 aa, Artificial Sequence
  Description of Artificial Sequence: Synthetic polypeptide
GQRGVVGLPGQRGERGFPGLPGYGGGGSPLGLAGGGGGSQVQLVQSGAEVKKPGSSVKVSCKASGYTFTNYGMNWVRQAPGQGLEWMGWINTYTGEPTYAQKFQGRVTITADESTSTAYMELSSLRSEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSGSFFLYSRLTVDKSRWQQGNVFSCSVLHEALHSHYTQKSLSLSPGK

SEQ ID NO: 226 - protein, 494 aa, Artificial Sequence
  Description of Artificial Sequence: Synthetic polypeptide
GQRGVVGLPGQRGERGFPGLPGYGGGGSPLGLAGGGGGSEVQLVQSGAEVKKPGESVKISCKGSGYTFTNYGMNWVRQMPGKGLEWMGWINTYTGEPTYSPSFQGQFTFSADKSISTAYLQISSLKASDTAMYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSGSFFLYSRLTVDKSRWQQGNVFSCSVLHEALHSHYTQKSLSLSPGK

SEQ ID NO: 227 - protein, 494 aa, Artificial Sequence
  Description of Artificial Sequence: Synthetic polypeptide
GQRGVVGLPGQRGERGFPGLPGYGGGGSPLGLAGGGGGSQVQLVQSGAEVKKPGSSVKVSCKASGYDFTHYGMNWVRQAPGQGLEWMGWINTYTGEPTYAQKFQGRFTFTADESTSTAYMELSSLRSEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSGSFFLYSRLTVDKSRWQQGNVFSCSVLHEALHSHYTQKSLSLSPGK

SEQ ID NO: 228 - protein, 486 aa, Artificial Sequence
  Description of Artificial Sequence: Synthetic polypeptide
GQRGVVGLPGQRGERGGGGSPLGLAGGGGGSQVQLVQSGAEVKKPGSSVKVSCKASGYTFTNYGMNWVRQAPGQGLEWMGWINTYTGEPTYAQKFQGRVTITADESTSTAYMELSSLRSEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSGSFFLYSRLTVDKSRWQQGNVFSCSVLHEALHSHYTQKSLSLSPGK

SEQ ID NO: 229 - protein, 486 aa, Artificial Sequence
  Description of Artificial Sequence: Synthetic polypeptide
GQRGVVGLPGQRGERGGGGSPLGLAGGGGGSEVQLVQSGAEVKKPGESVKISCKGSGYTFTNYGMNWVRQMPGKGLEWMGWINTYTGEPTYSPSFQGQFTFSADKSISTAYLQISSLKASDTAMYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSGSFFLYSRLTVDKSRWQQGNVFSCSVLHEALHSHYTQKSLSLSPGK

SEQ ID NO: 230 - protein, 486 aa, Artificial Sequence
  Description of Artificial Sequence: Synthetic polypeptide
GQRGVVGLPGQRGERGGGGSPLGLAGGGGGSQVQLVQSGAEVKKPGSSVKVSCKASGYDFTHYGMNWVRQAPGQGLEWMGWINTYTGEPTYAQKFQGRFTFTADESTSTAYMELSSLRSEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSGSFFLYSRLTVDKSRWQQGNVFSCSVLHEALHSHYTQKSLSLSPGK

SEQ ID NO: 231 - protein, 323 aa, Artificial Sequence
  Description of Artificial Sequence: Synthetic polypeptide
DQCIVDDITYNVQDTFHKKHEEGHMLNCTCFGQGRGRWKCDPVDQCQDSETGTFYQIGDSWEKYVHGVRYQCYCYGRGIGEWHCQPLQTYPSSGGGGSPLGLAGGGGGSDIVMTQTPLSLPVTPGEPASISCRASQDISNYLNWYLQKPGQSPQVLIYFTSSLHSGVPDRFSGSGSGTDFTLKISRLEAEDVGVYYCQQYSTVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 232 - protein, 484 aa, Artificial Sequence
  Description of Artificial Sequence: Synthetic polypeptide
GQRGVVGLPGQRGERGGGGSPLGLAGGGGGSQVQLVQSGAEVKKPGSSVKVSCKASGYTFTNYGMNWVRQAPGQGLEWMGWINTYTGEPTYAQKFQGRFTFTADESTSTAYMELSSLRSEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 233 - protein, 490 aa, Artificial Sequence
  Description of Artificial Sequence: Synthetic polypeptide
GILEECCFRSCDLALLETYCAGGGGSPLGLAGGGGGSQVQLVQSGAEVKKPGSSVKVSCKASGYTFTNYGMNWVRQAPGQGLEWMGWINTYTGEPTYAQKFQGRVTITADESTSTAYMELSSLRSEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 234 - protein, 490 aa, Artificial Sequence
  Description of Artificial Sequence: Synthetic polypeptide
GILEECCFRSCDLALLETYCAGGGGSPLGLAGGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 235 - protein, 258 aa, Artificial Sequence
  Description of Artificial Sequence: Synthetic polypeptide
AYRPSETLCGGELVDTLQFVCGDRGFAFGGGGSPLGLAGGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 236 - protein, 260 aa, Artificial Sequence
  Description of Artificial Sequence: Synthetic polypeptide
AYRPSETLCGGELVDTLQFVCGDRGFAFGGGGSLSGRSDNHGGGGSDIQMTQSPSSLSASVGDRVTITCRASQDISNYLNWYQQKPGKAPKVLIYFTSSLHSGVPSRFSGSGSGTDFTLTISSLQPEDFATYYCQQYSTVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 237 - protein, 325 aa, Artificial Sequence
  Description of Artificial Sequence: Synthetic polypeptide
DQCIVDDITYNVQDTFHKKHEEGHMLNCTCFGQGRGRWKCDPVDQCQDSETGTFYQIGDSWEKYVHGVRYQCYCYGRGIGEWHCQPLQTYPSSGGGGSLSGRSDNHGGGGSDIQMTQSPSSLSASVGDRVTITCRASQDISNYLNWYQQKPGKAPKVLIYFTSSLHSGVPSRFSGSGSGTDFTLTISSLQPEDFATYYCQQYSTVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 238 - protein, 490 aa, Artificial Sequence
  Description of Artificial Sequence: Synthetic polypeptide
GILEECCFRSCDLALLETYCAGGGGSPLGLAGGGGGSQVQLVQSGAEVKKPGSSVKVSCKASGYTFTNYGMNWVRQAPGQGLEWMGWINTYTGEPTYAQKFQGRVTITADESTSTAYMELSSLRSEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 239 - protein, 490 aa, Artificial Sequence
  Description of Artificial Sequence: Synthetic polypeptide
GILEECCFRSCDLALLETYCAGGGGSPLGLAGGGGGSQVQLVQSGAEVKKPGSSVKVSCKASGYTFTNYGMNWVRQAPGQGLEWMGWINTYTGEPTYAQKFQGRVTITADESTSTAYMELSSLRSEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSRDELTKNQVSLSCAVKGFYPSDIAVEWESNGQPENNYKTTPLVLDSDGSFFLVSKLTVDKSRWQQGNVFSCSVMHEALHNHYTQKSLSLSPGK

SEQ ID NO: 240 - protein, 486 aa, Artificial Sequence
  Description of Artificial Sequence: Synthetic polypeptide
EVGQRGVVGLPGQRGERGGGGSPLGLAGGGGGSQVQLVQSGAEVKKPGSSVKVSCKASGYTFTNYGMNWVRQAPGQGLEWMGWINTYTGEPTYAQKFQGRVTITADESTSTAYMELSSLRSEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSRDELTKNQVSLSCAVKGFYPSDIAVEWESNGQPENNYKTTPLVLDSDGSFFLVSKLTVDKSRWQQGNVFSCSVMHEALHNHYTQKSLSLSPGK

SEQ ID NO: 241 - protein, 254 aa, Artificial Sequence
  Description of Artificial Sequence: Synthetic polypeptide
CAQTMRIKPHQGQHIGESCGGGGSGGGGSGPLGVRGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 242 - protein, 498 aa, Artificial Sequence
  Description of Artificial Sequence: Synthetic polypeptide
GGGGSCAQTMRIKPHQGQHIGESCGGGGSGGGGSGPLGVRGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 243 - protein, 503 aa, Artificial Sequence
  Description of Artificial Sequence: Synthetic polypeptide
CAQTMRIKPHQGQHIGESCGGGGSGGGGSGPLGVRGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGGSGGGGSGGGGSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 244 - protein, 702 aa, Artificial Sequence
  Description of Artificial Sequence: Synthetic polypeptide
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGKGGGGSGGGGSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGGSGGGGSGGGGSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 245 - protein, 516 aa, Artificial Sequence
  Description of Artificial Sequence: Synthetic polypeptide
CAQTMRIKPHQGQHIGESCGGGGSGGGGSGPLGVRGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLVESGGGLVQPGPSLRLSCTASRYTFTEYTIHWVRQAPGKGLEWVGGINPNNGIPNYAASVKGRFTISVDDSKSIAYLQMNSLKTEDTAVYFCARRRIAYGYDEGHAMDYWGQGTTVTVSSGGSGGGGSGGGGSGGAIRMTQSPSSFSVSTGDRVTMTCRSSQSLLYSRNQKNYLAWFQQKPGKAPKLLIFWASTRESGVPSRFSGSGSGTDFTLTISCVQSEDFATYDCQQYFSYPLTFGQGTKLEIK

SEQ ID NO: 246 - protein, 755 aa, Artificial Sequence
  Description of Artificial Sequence: Synthetic polypeptide
CAQTMRIKPHQGQHIGESCGGGGSGGGGSGPLGVRGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGKGGGGSGGGGSEVQLVESGGGLVQPGPSLRLSCTASRYTFTEYTIHWVRQAPGKGLEWVGGINPNNGIPNYAASVKGRFTISVDDSKSIAYLQMNSLKTEDTAVYFCARRRIAYGYDEGHAMDYWGQGTTVTVSSGGSGGGGSGGGGSGGAIRMTQSPSSFSVSTGDRVTMTCRSSQSLLYSRNQKNYLAWFQQKPGKAPKLLIFWASTRESGVPSRFSGSGSGTDFTLTISCVQSEDFATYDCQQYFSYPLTFGQGTKLEIK

SEQ ID NO: 247 - protein, 518 aa, Artificial Sequence
  Description of Artificial Sequence: Synthetic polypeptide
AYRPSETLCGGELVDTLQFVCGDRGFAFGGGGSPLGLAGGGGGSDIQMTQSPSSLSASVGDRVTITCRASQDISNYLNWYQQKPGKAPKVLIYFTSSLHSGVPSRFSGSGSGTDFTLTISSLQPEDFATYYCQQYSTVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 248 - protein, 583 aa, Artificial Sequence
  Description of Artificial Sequence: Synthetic polypeptide
DQCIVDDITYNVQDTFHKKHEEGHMLNCTCFGQGRGRWKCDPVDQCQDSETGTFYQIGDSWEKYVHGVRYQCYCYGRGIGEWHCQPLQTYPSSGGGGSPLGLAGGGGGSDIQMTQSPSSLSASVGDRVTITCRASQDISNYLNWYQQKPGKAPKVLIYFTSSLHSGVPSRFSGSGSGTDFTLTISSLQPEDFATYYCQQYSTVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 249 - protein, 20 aa, Artificial Sequence
  Description of Artificial Sequence: Synthetic peptide
  SITE (1)..(20): This sequence may encompass 1-20 “Gly” residues
GGGGGGGGGGGGGGGGGGGG

SEQ ID NO: 250 - protein, 40 aa, Artificial Sequence
  Description of Artificial Sequence: Synthetic polypeptide
  SITE (1)..(40): This sequence may encompass 1-20 “Gly Ser” repeating units
GSGSGSGSGSGSGSGSGSGSGSGSGSGSGSGSGSGSGSGS

SEQ ID NO: 251 - protein, 100 aa, Artificial Sequence
  Description of Artificial Sequence: Synthetic polypeptide
  SITE (1)..(100): This sequence may encompass 1-20 “Gly Ser Gly Gly Ser” repeating units
GSGGSGSGGSGSGGSGSGGSGSGGSGSGGSGSGGSGSGGSGSGGSGSGGSGSGGSGSGGSGSGGSGSGGSGSGGSGSGGSGSGGSGSGGSGSGGSGSGGS

SEQ ID NO: 252 - protein, 80 aa, Artificial Sequence
  Description of Artificial Sequence: Synthetic polypeptide
  SITE (1)..(80): This sequence may encompass 1-20 “Gly Gly Gly Ser” repeating units
GGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 253 - protein, 4 aa, Artificial Sequence
  Description of Artificial Sequence: Synthetic peptide
GGSG

SEQ ID NO: 254 - protein, 5 aa, Artificial Sequence
  Description of Artificial Sequence: Synthetic peptide
GGSGG

SEQ ID NO: 255 - protein, 5 aa, Artificial Sequence
  Description of Artificial Sequence: Synthetic peptide
GGGGS

SEQ ID NO: 256 - protein, 5 aa, Artificial Sequence
  Description of Artificial Sequence: Synthetic peptide
GSGSG

SEQ ID NO: 257 - protein, 5 aa, Artificial Sequence
  Description of Artificial Sequence: Synthetic peptide
GSGGG

SEQ ID NO: 258 - protein, 5 aa, Artificial Sequence
  Description of Artificial Sequence: Synthetic peptide
GGGSG

SEQ ID NO: 259 - protein, 5 aa, Artificial Sequence
  Description of Artificial Sequence: Synthetic peptide
GSSSG

SEQ ID NO: 260 - protein, 40 aa, Artificial Sequence
  Description of Artificial Sequence: Synthetic polypeptide
  SITE (1)..(40): This sequence may encompass 1-20 “Ala Pro” repeating units
APAPAPAPAPAPAPAPAPAPAPAPAPAPAPAPAPAPAPAP

SEQ ID NO: 261 - protein, 100 aa, Artificial Sequence
  Description of Artificial Sequence: Synthetic polypeptide
  SITE (1)..(100): This sequence may encompass 1-20 “Glu Ala Ala Ala Lys” repeating units
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 262 - protein, 6 aa, Artificial Sequence
  Description of Artificial Sequence: Synthetic 6xHis tag
HHHHHH